(19) United States
(12) Patent Application Publication
(10) Pub. No.: US 2026/0168010 A1
(43) Pub. Date: Jun. 18, 2026

(54) Ultrasensitive CRISPR Biosensors Assisted by Nucleic Acid Mediators

(71) Applicant: NewSouth Innovations Pty Limited, University of New South Wales, NSW (AU)

(72) Inventors: Yi Li, University of New South Wales, NSW (AU); Fei Deng, University of New South Wales, NSW (AU); Ewa Goldys, University of New South Wales, NSW (AU); Biyao Yang, University of New South Wales, NSW (AU); Rui Sang, University of New South Wales, NSW (AU)

(21) Appl. No.: 19/125,458

(22) PCT Filed: Nov. 10, 2023
(86) PCT No.: PCT/AU2023/051144

§ 371 Date: Apr. 29, 2025

(30) Foreign Application Priority Data
Nov. 11, 2022 (AU) 2022903394
Apr. 3, 2023 (AU) 2023900941
Aug. 11, 2023 (AU) 2023902558

(51) Int. Cl. C12Q 1/6823; C12N 9/22; C12N 15/11; C12Q 1/34; C12Q 1/6804; C12Q 1/6806; C12Q 1/6825

(52) U.S. Cl. CPC C12Q 1/6823; C12N 9/226; C12N 15/11; C12Q 1/34; C12Q 1/6804; C12Q 1/6806; C12Q 1/6825; G01N 2333/922

(57) ABSTRACT

The present invention relates to CRISPR/Cas-based biosensing materials, assays and methods. In particular, the technology relates to ultrasensitive CRISPR/Cas-based detection methods for nucleic acid assays using special molecular constructs including constructs termed nucleic acid mediators comprising both single-, and double-stranded nucleic acid sequences in circular conformation, as well as palindromic oligonucleotides. The materials and methods according to the invention may also be used to enhance the sensitivity of existing bioassays.

### CROSS-REFERENCE TO RELATED APPLICATIONS

The present application claims priority to Australian Provisional Application No. 2022903394 filed on 11 Nov. 2022, Australian Provisional Application No. 2023900941 3 Apr. 2023, and Australian Provisional Application No. 2023902558 filed 11 Aug. 2023. The entire content of each of these applications is herein incorporated by reference.

### FIELD

The present invention relates to CRISPR/Cas-based biosensing materials, assays and methods. In particular, the technology relates to ultrasensitive CRISPR/Cas-based methods using special molecular constructs including constructs termed circular mediators (Cir-mediators) comprising both single-, and double-stranded nucleic acid sequences, as well as palindromic oligonucleotides. The materials and methods according to the invention may also be used to enhance the sensitivity of existing bioassays.

### BACKGROUND

The newly emerged programmable nucleases, Clustered Regularly Interspaced Short Palindromic Repeats (CRISPR) and CRISPR-associated protein (CRISPR/Cas), provided an evolutional approach for nucleic acid manipulation. Unlike the zinc-finger nucleases (ZFNs) or transcription activator-like effector nucleases (TALENs) systems require customized design for each target, CRISPR/Cas systems exhibited remarkable universality for target nucleic acid sequence. With a simple change of the guiding RNA (gRNA) spacer region, CRISPR/Cas ribonucleoproteins (RNP) can be widely applied in various in vivo or in vitro environments to specifically degrade its designated target DNA sequences with single nucleotide specificity. Intriguingly, beyond the sequence-dependent nuclease activity (cis-cleavage), various CRISPR/Cas RNPs, such as LbaCas12a, possess a unique sequence-independent nuclease activity (trans-cleavage), which can continuously cut surrounding single strand (ssDNA) molecules with a catalytic efficiency of ˜17 turnover per second. The unique trans-cleavage activity in CRISPR/Cas effector proteins provide great potential for novel biosensor development. Each CRISPR/Cas ribonucleoprotein can be treated as a micro-biosensor with target recognition function due to its sequence-dependent nuclease activity and integrated signal amplification ability due to its trans-cleavage.

As described above this trans-cleavage capability has been exploited for use in methods of biosensing, and signal amplification. However, methods that have been developed in the art to date have had limitations including: reliance upon the integration of additional polymerase-based amplification strategies (such as PCR, LAMP, RPA, etc.), separate reaction procedures, varied reaction temperatures, compromised sensitivity into pM-nM ranges, requirement for customized devices for signal measurement, or specific material modifications such as specially designed hairpin RNA oligos or hybrid DNA-RNA molecule to release additional gRNAs so as to form from more functional CRISPR/Cas12a RNPs, all of which lead to overall system complexity, excessive reaction time, reduced system stability and/or reliability. As indicated in the literature, target amplification poses additional risks of carryover contamination, especially in clinical settings where assays are often run in close proximity in dedicated small spaces. Since high rates of false positivity are unacceptable in clinical applications, commercial platforms resort to integrated design features such as enclosed housings to limit cross-contamination, adding to assay cost and complexity. There remains a need to provide simplified biosensing, and signal amplification methods which rely on the trans-cleavage capability of CRISPR/Cas RNPs and which are ultra-sensitive for clinical or research applications, which can be performed rapidly, without additional amplification reactions (e.g. PCR based amplification), under isothermal conditions.

### Abbreviations

ssDNA—single strand DNA
dsDNA—double strand DNA
cDNA—complementary ssDNA
XNA—Xeno nucleic acid
Cir-ssDNA—circular ssDNA
Cir-ssRNA—circular ssRNA
Cir-mediator—circular DNA or RNA molecular construct comprising either (a) both a ssDNA region and a dsDNA sequence, or (b) both an ssRNA region and a ssDNA sequence or a dsDNA sequence. Additionally any of the nucleotides in such construct may be replaced by XNA.
L-ssDNA—linear-ssDNA
L-dsDNA—linear-dsDNA
T-strand—target ssDNA which has complementary sequence to the spacer region of gRNA
C-strand—complementary ssDNA for the T-strand
Lg-linker—ssDNA linker for T4 ligase
DANCER—DNA amplifier enhanced CRISPR/Cas autocatalytic sensor
RNP ribonucleoprotein
gRNA—guiding RNA or presenting the crRNA (CRISPR RNA), sgRNA (single guiding RNA) for Type V or Type VI Cas effector
target-C—target DNA for classic CRISPR/Cas12a sensor
gRNA-C—gRNA for classic CRISPR/Cas12a sensor
target-D—target DNA for DANCER
gRNA-D—gRNA for DANCER
cfDNA—cell-free DNA
gRNA-cf—gRNA for cfDNA detection sensor
Cir-amplifier—a circular DNA comprising a dsDNA sequence and a ssDNA region which was created by a fluorescent labelled cDNA bound to a circular ssDNA
CRC-mouse mice bearing with human colorectal cancer
LFA—Lateral flow assay
PCR—Polymerase Chain Reaction
RPA—Recombinase polymerase amplification
LAMP—Loop-mediated isothermal amplification
SERS—Surface-enhanced Raman spectroscopy
LOD—Limit of detection
PAM—protospacer adjacent motif

### SUMMARY OF INVENTION

According to a first aspect, the present invention provides a method for the detection of a target nucleic acid in a sample, the method comprising:
(a) contacting the sample with: (i) a first type V or type VI CRISPR/Cas effector protein; (ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence; iv) a second type V or type VI CRISPR/Cas effector protein; (v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA; (vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; and
(b) measuring a detectable signal produced following cleavage of the labelled nucleic acid reporter by the first and second type V or type VI CRISPR/Cas effector protein, thereby detecting the target in the sample.

According to a second aspect, the present invention provides a method for the detection of a target in a sample, the method comprising:
(a) contacting the sample with: (i) a first type V or type VI CRISPR/Cas effector protein; (ii) a trigger nucleic acid sequence; (iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein; (iv) a circular DNA molecular construct comprising both a ssDNA region and a dsDNA sequence, or a circular RNA molecular construct comprising both an ssRNA region and either a ssDNA sequence or a dsDNA sequence; (v) a second type V or type VI CRISPR/Cas effector protein; (vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA; (vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; (viii) a target binding construct; and
(b) measuring a detectable signal produced following cleavage of the labelled nucleic acid reporter construct by the first and second type V or type VI CRISPR/Cas effector protein, thereby detecting the target in the sample; wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.

According to a third aspect, the present invention provides a method of enhancing a type V or type VI CRISPR/Cas detection system comprising adding to a reaction mixture comprising the type V or Type VI CRISPR/Cas effector of the system: (i) a circular DNA molecular construct comprising both a ssDNA region and a dsDNA sequence, or a circular RNA molecular construct comprising both an ssRNA region and either a ssDNA sequence or a dsDNA sequence; (ii) a second type V or type VI CRISPR/Cas effector protein; (iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the guide RNA and the dsDNA or ssDNA sequence occurs following cleavage of the ssDNA or ssRNA region of the circular DNA or RNA molecular construct, respectively, by the type V or type VI CRISPR/Cas effector protein of the detection system and further activates the nuclease activity of the second type V or type VI CRISPR/Cas effector protein; and (iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein; and measuring a detectable signal produced following cleavage of the labelled nucleic acid reporter by the second type V or type VI CRISPR/Cas effector protein.

According to a fourth aspect, the present invention provides a kit for detecting a target in a sample, the kit comprising: (i) a first type V or type VI CRISPR/Cas effector protein; (ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence; (iv) a second type V or type VI CRISPR/Cas effector protein; (v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and (vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.

According to a fifth aspect, the present invention provides a reaction mixture comprising: (i) a first type V or type VI CRISPR/Cas effector protein; (ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence; (iv) a second type V or type VI CRISPR/Cas effector protein; (v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and (vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.

Numbered statements of the invention are as follows:
1. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence; iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA sequence or DNA/RNA hybrid sequence first occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA;
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; and
(b) measuring a detectable signal produced following cleavage of the labelled nucleic acid reporter by the first and second type V or type VI CRISPR/Cas effector protein, thereby detecting the target in the sample.
2. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence
(v) a second type V or type VI CRISPR/Cas effector protein;
(vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA sequence or DNA/RNA hybrid sequence first occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first and second type V or type VI CRISPR/Cas effector protein, thereby detecting the target in the sample;
wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.
3. The method of statement 2, wherein the trigger nucleic acid sequence is a ssDNA or dsDNA or ssRNA sequence, preferably not having full (100%) complementarity to an existing genomic sequence, more preferably with the length of at least 10 nucleotides.
4. The method of statement 2 or 3, wherein the target binding construct is an antibody or antigen binding fragment thereof.
5. The method of any one of statements 2 to 4, wherein the target binding construct is immobilized on a substrate or conjugated to a magnetic bead or a microparticle or a nanoparticle.
6. The method of statement 5, further comprising the step of performing magnetic separation of the captured target bound to the first target binding construct from the sample when the first target binding construct is conjugated to a magnetic bead.
7. The method of any one of statements 1 to 6, wherein the first and second type V or type VI CRISPR/Cas effector proteins are the same.
8. The method of any one of statements 1 to 6, wherein the first and second type V or type VI CRISPR/Cas effector proteins are different.
9. The method according to statement 8, wherein said first type V or type VI CRISPR/Cas effector protein is a Type V CRISPR/Cas effector protein and said second type V or type VI CRISPR/Cas effector protein is a Type VI CRISPR/Cas effector protein.
10. The method of any one of statements 1 to 9, wherein contacting the sample with the second effector type V or type VI CRISPR/Cas effector protein and the second guide RNA occurs simultaneously with the sample being contacted with the first effector type V or type VI CRISPR/Cas effector protein and the first guide RNA.
11. The method of any one of statements 1 to 9, wherein contacting the sample with the second effector type V or type VI CRISPR/Cas effector protein and the second guide RNA occurs after the sample has been contacted with the first effector type V or type VI CRISPR/Cas effector protein and the first guide RNA.
12. The method of statement 11, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.
13. The method of any one of statements 1 to 12, wherein the first and/or second guide RNA is bound to the first and/or second type V or type VI CRISPR/Cas effector protein, respectively.
14. A method of enhancing a type V or type VI CRISPR/Cas detection system comprising
   adding to a reaction mixture comprising the type V or Type VI CRISPR/Cas effector protein, a first guide RNA, and a labelled nucleic acid reporter of the system:
(i) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(ii) a second type V or type VI CRISPR/Cas effector protein;
(iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the guide RNA and the dsDNA or ssDNA or dsRNA or DNA/RNA hybrid sequence first occurs following cleavage of the ssDNA or ssRNA region of the circular DNA or RNA molecular construct, respectively, by the type V or type VI CRISPR/Cas effector protein of the detection system and further activates the nuclease activity of the second type V or type VI CRISPR/Cas effector protein; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein;
and measuring a detectable signal produced following cleavage of the labelled nucleic acid reporter, and/or the labelled reporter construct when added, by the second type V or type VI CRISPR/Cas effector protein.
15. The method of statement 14, wherein the guide RNA is bound to the second type V or type VI CRISPR/Cas effector protein.
16. The method of any one of statements 1 to 15, wherein the circular DNA molecular construct or circular RNA molecular construct has the total length (circumference) from 15 to 30 nucleotides.
17. The method of statement 16 wherein the circular DNA molecular construct or circular RNA molecular construct has a total length of 16, 17, 18, 19, 20, or 21 nucleotides.
18. The method of statement 16 or 17, wherein the circular DNA molecular construct or circular RNA molecular construct comprises two parts including a 2-5 nucleotides ssDNA or ssRNA, respectively, and the remaining part of the molecular construct is dsDNA or ssDNA or dsRNA or DNA/RNA with a complementary sequence to the second guide RNA or the guide RNA which binds to the second type V or type VI CRISPR/Cas effector protein.
19. The method of any one of statements 1 to 18, wherein the sequences of dsDNA or ssDNA are random nucleic acid sequences, preferably not forming complex secondary structures, preferably and not fully (100%) complementary to any naturally existing genomic sequences.
20. The method of any one of statements 1 to 19, wherein the circular DNA molecular construct or circular RNA molecular construct completely or significantly blocks CRISPR/Cas activation for at least 1.5 hours when non-linearized.
21. The method of any one of statements 1 to 20, wherein any one or more of the guide RNA, circular DNA molecular construct or circular RNA molecular construct, the reporter construct, and trigger nucleic acid, when present, comprises at least one nucleotide containing a non-natural modification/substitution.
22. The method of any one of statements 1 to 21, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.
23. The method of any one of statements 1 to 21, wherein the type V or type VI CRISPR/Cas effector protein is selected from: Cas12a (Cpf1), Cas12b (C2c1), Cas 13a and Cas 13b.
24. The method of any one of statements 1 to 23, wherein the reporter construct is a labelled RNA
25. The method of any one of statements 1 to 23, wherein the reporter construct is a labelled DNA.
26. The method of any one of statements 1 to 25, wherein the steps of the method are conducted at a temperature ranging from 18 to 42 degrees Celsius, preferably, from 25 to 37 degrees Celsius.
27. The method of any one of statements 1 to 26, wherein the sample is also contacted with at least one sulfhydryl reductant.
28. The method of statement 27, wherein the sulfhydryl reductant is selected from the group consisting of Dithiothreitol (DTT), Tris(2-carboxyethyl) phosphine (TCEP) and 2-Mercaptoethanol (2-ME).
29. The method of statement 28, wherein the sulfhydryl reductant is DTT.
30. The method of statement 29, wherein said contacting occurs at a temperature of about 37° C.
31. The method of any one of statements 1 to 30, wherein the sample is also contacted with at least one non-ionic surfactant.
32. The method of statement 31, wherein the non-ionic surfactant is selected from the group consisting of Brij L23 and poly(vinyl alcohol) (PVA).
33. The method of statement 32, wherein the non-ionic surfactant is PVA.
34. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
wherein the dsDNA sequence of the circular DNA molecular construct or the dsDNA or ssDNA or dsRNA or DNA/RNA hybrid sequence of the circular RNA molecular construct specifically hybridizes with the first guide RNA following linearization of circular DNA or RNA molecular construct, respectively, by the nuclease activity of said first type V or type VI CRISPR/Cas effector protein and also activates the nuclease activity of first type V or type VI CRISPR/Cas effector proteins bound to said first guide RNA;
(b) measuring a detectable signal produced following linearization of the circular DNA molecular construct, or the circular RNA molecular construct, by the first and/or second type V or type VI CRISPR/Cas effector protein, thereby detecting the target nucleic acid in the sample.
35. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct;
wherein hybridization between the guide sequence of the second guide RNA and the dsDNA or ssDNA sequence first occurs following linearization of circular DNA or RNA molecular construct, respectively, by the nuclease activity of said first type V or type VI CRISPR/Cas effector protein and further activates the nuclease activity of the second type V or type VI CRISPR/Cas effector protein bound to said second guide RNA;
(b) measuring a detectable signal produced following linearization of the circular DNA molecular construct, or the circular RNA molecular construct, by the first and/or second type V or type VI CRISPR/Cas effector protein, thereby detecting the target nucleic in the sample.
36. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
wherein the dsDNA of the circular DNA molecular construct or the dsDNA or ssDNA or dsRNA or DNA/RNA hybrid sequence of the circular RNA molecular construct specifically hybridizes with the first guide RNA following linearization of the circular DNA or RNA molecular construct, respectively, by the nuclease activity of said first type V or type VI CRISPR/Cas effector protein and also activates the nuclease activity of first type V or type VI CRISPR/Cas effector proteins bound to said first guide RNA;
(v) a target binding construct; and
(b) measuring a detectable signal produced following linearization of the circular DNA molecular construct, or the circular RNA molecular construct, by the first type V or type VI CRISPR/Cas effector protein, thereby detecting the target in the sample;
wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.
37. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
(v) a second type V or type VI CRISPR/Cas effector protein;
(vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA or DNA/RNA hybrid sequence first occurs following linearization of ssDNA or ssRNA sequence of the circular DNA or RNA molecular construct, respectively, by said first type V or type VI CRISPR/Cas effector protein and further activates the nuclease activity of the second type V or type VI CRISPR/Cas effector protein bound to said second guide RNA;
(viii) a target binding construct; and
(b) measuring a detectable signal produced following linearization of the circular DNA molecular construct, or the circular RNA molecular construct, by the first and/or second type V or type VI CRISPR/Cas effector protein, thereby detecting the target in the sample; wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.
38. The method of statement 36 or 37, wherein the trigger nucleic acid sequence is a ssDNA or dsDNA or ssDNA sequence, preferably not having full (100%) complementarity to an existing genomic sequence, more preferably with the length of at least 10 nucleotides.
39. The method of any one of statements 36-38, wherein the target binding construct is an antibody or antigen binding fragment thereof.
40. The method of any one of statements 36 to 39, wherein the target binding construct is immobilized on a substrate or conjugated to a magnetic bead or a microparticle or a nanoparticle.
41. The method of statement 40, further comprising the step of performing magnetic separation of the captured target bound to the first target binding construct from the sample when the first target binding construct is conjugated to a magnetic bead.
42. The method of any one of statements 35 or 37 to 41, wherein the first and second type V or type VI CRISPR/Cas effector proteins are the same.
43. The method of any one of statements 35 or 37 to 41, wherein the first and second type V or type VI CRISPR/Cas effector proteins are different.
44. The method according to statement 43, wherein said first type V or type VI CRISPR/Cas effector protein is a Type V CRISPR/Cas effector protein and said second type V or type VI CRISPR/Cas effector protein is a Type VI CRISPR/Cas effector protein.
45. The method of any one of statements 35 or 37 to 44, wherein contacting the sample with the second effector type V or type VI CRISPR/Cas effector protein and the second guide RNA occurs at the same time when the sample has been contacted with the first effector type V or type VI CRISPR/Cas effector protein and the first guide RNA.
46. The method of any one of statements 35 or 37 to 44, wherein contacting the sample with the second effector type V or type VI CRISPR/Cas effector protein and the second guide RNA occurs after the sample has been contacted with the first effector type V or type VI CRISPR/Cas effector protein and the first guide RNA.
47. The method of statement 46, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.
48. The method of any one of statements 34 to 47, wherein the first guide RNA is bound to the first type V or type VI CRISPR/Cas effector protein.
49. The method of any one of statements 35 or 37 to 48, wherein the second guide RNA is bound to the second type V or type VI CRISPR/Cas effector protein.
50. A method of enhancing a type V or type VI CRISPR/Cas detection system which comprises a type V or Type VI CRISPR/Cas effector protein, comprising:
   adding to a reaction mixture comprising a type V or Type VI CRISPR/Cas effector protein of the detection system, a circular DNA molecular construct comprising both a ssDNA region and a dsDNA sequence, wherein the 5′ end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled; wherein the dsDNA of the circular DNA molecular construct or the dsDNA or ssDNA sequence or dsRNA sequence or DNA/RNA hybrid sequence of the circular RNA molecular construct hybridizes with a guide sequence of a guide RNA of said type V or type VI CRISPR/Cas detection system, and hybridization occurs following linearization of the circular DNA or RNA molecular construct, respectively, by the nuclease activity of the type V or type VI CRISPR/Cas effector protein of the detection system and further activates the nuclease activity of the type V or type VI CRISPR/Cas effector protein; and
   measuring a detectable signal produced following linearization of the circular DNA molecular construct, or the circular RNA molecular construct by the type V or type VI CRISPR/Cas effector protein of said detection system.
51. A method of enhancing a type V or type VI CRISPR/Cas detection system which comprises a type V or Type VI CRISPR/Cas effector protein, comprising:
   adding to a reaction mixture comprising a type V or Type VI CRISPR/Cas effector protein of the detection system:
(i) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ss DNA or dsDNA or dsRNA or DNA/RNA hybrid sequence are detectably labelled;
(ii) a second type V or type VI CRISPR/Cas effector protein; and
(iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the guide RNA and the dsDNA or ssDNA or dsRNA or DNA/RNA sequence occurs following linearization of the circular DNA or RNA molecular construct, respectively, by the nuclease activity of the type V or type VI CRISPR/Cas effector protein of the detection system and further activates the nuclease activity of the second type V or type VI CRISPR/Cas effector protein or the type V or type VI CRISPR/Cas effector protein of said detection system; and
   measuring a detectable signal produced following linearization of the circular DNA molecular construct, or the circular RNA molecular construct by the second type V or type VI CRISPR/Cas effector protein or the type V or type VI CRISPR/Cas of said detection system.
52. The method of any one of statements 34 to 51, wherein the, wherein the circular DNA molecular construct or circular RNA molecular construct has the total length (circumference) from 15 to 30 nucleotides.
53. The method of statement 52 wherein the circular DNA molecular construct or circular RNA molecular construct has a total length of 16, 17, 18, 19, 20, 21, 22, or 23 nucleotides.
54. The method of statement 52 or 53, wherein the circular DNA molecular construct or circular RNA molecular construct comprises two parts including a 2-5 nucleotides ssDNA, or ssRNA, respectively, and the remaining part of the molecular construct is dsDNA or ssDNA or dsRNA or DNA/RNA hybrid with a complementary sequence to either a guide RNA of said type V or type VI CRISPR/Cas detection system or the guide RNA which binds to the second type V or type VI CRISPR/Cas effector protein (when present).
55. The method of any one of statements 34 to 55, wherein any one or more of the guide RNA, circular DNA molecular construct or circular RNA molecular construct, the reporter construct, and trigger nucleic acid, when present, comprises at least one nucleotide containing a non-natural modification/substitution.
56. The method of any one of statements 34 to 55, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.
57. The method of any one of statements 34 to 56, wherein the type V or type VI CRISPR/Cas effector protein is selected from: Cas12a (Cpf1), Cas12b (C2c1), Cas 13a and Cas 13b.
58. The method of any one of statements 34 to 57, wherein the steps of the method are conducted at a temperature ranging from 18 to 42 degrees Celsius, preferably, from 25 to 37 degrees Celsius.
59. The method of any one of statements 34 to 58, wherein the sample is also contacted with at least one sulfhydryl reductant.
60. The method of statement 59, wherein the sulfhydryl reductant is selected from the group consisting of Dithiothreitol (DTT), Tris(2-carboxyethyl) phosphine (TCEP) and 2-Mercaptoethanol (2-ME).
61. The method of statement 60, wherein the sulfhydryl reductant is DTT.
62. The method of statement 61, wherein said contacting occurs at a temperature of about 37° C.
63. The method of any one of statements 34 to 62, wherein the sample is also contacted with at least one non-ionic surfactant.
64. The method of statement 63, wherein the non-ionic surfactant is selected from the group consisting of Brij L23 and poly(vinyl alcohol) (PVA).
65. The method of statement 64, wherein the non-ionic surfactant is PVA.
66. A method for the detection of a target nucleic acid in a sample, the method comprising:
(a) contacting the sample with:
(i) a first type II or type V or type VI CRISPR/Cas effector protein;
(ii) a trigger nucleic acid sequence;
(iii) a first guide RNA, optionally wherein the first guide RNA is in association with said first type II or type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type II or type V or type VI CRISPR/Cas effector protein;
(iv) a second type II or type V or type VI CRISPR/Cas effector protein;
(v) a second guide RNA, wherein the second guide RNA is circular and is susceptible to trans-cleavage nuclease activity of a type II or type V or type VI CRISPR/Cas effector protein, and comprises a region that binds to said second type II or type V or type VI CRISPR/Cas effector protein, and a guide sequence that hybridizes with the trigger nucleic acid sequence, wherein hybridization between the guide sequence and the trigger nucleic acid sequence, first occurs following linearization of the second guide RNA by the nuclease activity of the first type TT or type V or type VI CRISPR/Cas effector protein and further activates the trans-cleavage nuclease activity of the second type II or type V or type VI CRISPR/Cas effector protein;
(vi) a labelled reporter construct, wherein said reporter construct comprises a nucleic acid that is cleavable by the nuclease activity of the activated type II or type V or type VI CRISPR/Cas effector protein; and
(b) measuring a detectable signal produced following cleavage of the labelled nucleic acid reporter by the first and/or second type II or type V or type VI CRISPR/Cas effector protein, thereby detecting the target in the sample.
67. A method for the detection of a target in a sample, the method comprising:
(a) contacting the sample with:
(i) a first type II or type V or type VI CRISPR/Cas effector protein;
(ii) a first trigger nucleic acid sequence;
(iii) a first guide RNA, optionally wherein the first guide RNA is bound to said first type II or type V or type VI CRISPR/Cas effector protein, comprising: a region that binds to said first type II or type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type II or type V or type VI CRISPR/Cas effector protein;
(iv) a second type II or type V or type VI CRISPR/Cas effector protein;
(v) a second trigger nucleic acid sequence;
(vi) a second guide RNA, wherein the second guide RNA is circular and is susceptible to trans-cleavage nuclease activity of a type II or type V or type VI CRISPR/Cas effector protein, and comprises a region that binds to said second type II or type V or type VI CRISPR/Cas effector protein, and a guide sequence that hybridizes with the second trigger nucleic acid sequence, wherein hybridization between the guide sequence and the second trigger nucleic acid sequence, first occurs following linearization of the second guide RNA by the nuclease activity of the first type II or type V or type VI CRISPR/Cas effector protein and further activates the trans-cleavage nuclease activity of the second type II or type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type II or type V or type VI CRISPR/Cas effector protein;
(viii) a target binding construct; and
(b) measuring a detectable signal produced following cleavage of the labelled nucleic acid reporter construct by the first and second type II or type V or type VI CRISPR/Cas effector protein, thereby detecting the target in the sample;
wherein the first type II or type V or type VI CRISPR/Cas effector protein, the first trigger nucleic acid sequence, the first guide RNA, the first type II or type V or type VI CRISPR/Cas effector protein in combination with the first guide RNA, or the first type II or type V or type VI CRISPR/Cas effector protein in combination with the first guide RNA and the first trigger nucleic acid is linked or conjugated to the target binding construct to thereby co-locate the first type II or type V or type VI CRISPR/Cas effector protein and the target when present in the sample.
68. A method of enhancing a type II or type V or type VI CRISPR/Cas detection system, which comprises a first type II or type V or Type VI CRISPR/Cas effector protein, a first guide RNA, and a labelled nucleic acid reporter, comprising:
   adding to a reaction mixture comprising at least a first type II or type V or Type VI CRISPR/Cas effector of the system:
(i) a second type II or type V or type VI CRISPR/Cas effector protein;
(ii) a trigger nucleic acid sequence; and
(iii) a circular guide RNA which is susceptible to cis-cleavage and trans-cleavage nuclease activity of a type II or type V or type VI CRISPR/Cas effector protein, wherein the circular guide RNA comprises a region that binds to said second type II or type V or type VI CRISPR/Cas effector protein, and a guide sequence that hybridizes with the trigger nucleic acid sequence, wherein hybridization between the guide sequence and the trigger nucleic acid sequence, first occurs following linearization of the circular guide RNA by the nuclease activity of at least a first type II or type V or Type VI CRISPR/Cas effector of the system and further activates the trans-cleavage nuclease activity of the second type II or type V or type VI CRISPR/Cas effector protein; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type II or type V or type VI CRISPR/Cas effector protein;
and measuring a detectable signal produced following cleavage of the labelled nucleic acid reporter of the detection system, and/or the labelled reporter construct when added, by the second type II or type V or type VI CRISPR/Cas effector protein.
69. The method of statement 67, wherein the target binding construct is an antibody or antigen binding fragment thereof.
70. The method of any one of statements 67 or 69, wherein the target binding construct is immobilized on a substrate or conjugated to a magnetic bead or a microparticle or a nanoparticle.
71. The method of statement 70, further comprising the step of performing magnetic separation of the captured target bound to the first target binding construct from the sample when the first target binding construct is conjugated to a magnetic bead.
72. The method of any one of statements 66 to 71, wherein the trigger nucleic acid sequence is a ssRNA, ssDNA or dsDNA sequence, preferably not having full (100%) complementarity to an existing genomic sequence, more preferably with the length of at least 10 nucleotides.
73. The method of any one of statements 66 to 72, wherein the first and second type II or type V or type VI CRISPR/Cas effector proteins are the same.
74. The method of any one of statements 66 to 72, wherein the first and second type II or type V or type VI CRISPR/Cas effector proteins are different.
75. The method according to statement 74, wherein said first type II or type V or type VI CRISPR/Cas effector protein is a Type V CRISPR/Cas effector protein and said second type II or type V or type VI CRISPR/Cas effector protein is a Type VI CRISPR/Cas effector protein.
76. The method of any one of statements 66 to 75, wherein contacting the sample with the second effector type II or type V or type VI CRISPR/Cas effector protein and the second guide RNA occurs at the same time when the sample has been contacted with the first effector type II or type V or type VI CRISPR/Cas effector protein and the first guide RNA.
77. The method of any one of statements 66 to 75, wherein contacting the sample with the second effector type II or type V or type VI CRISPR/Cas effector protein and the second guide RNA occurs after the sample has been contacted with the first effector type II or type V or type VI CRISPR/Cas effector protein and the first guide RNA.
78. The method of statement 77, wherein contacting the sample with the second effector type II or type V or type VI CRISPR/Cas effector protein and the second guide RNA occurs at between 1 minute and 1 hour after the sample has been contacted with the first effector type II or type V or type VI CRISPR/Cas effector protein and the first guide RNA.
79. The method of any one of statements 66 to 78, wherein the circular guide RNA has the total length (circumference) from 40-50 nucleotides.
80. The method of any one of statements 66 to 79, wherein any one or more of the guide RNA, the reporter construct, and trigger nucleic acid, comprises at least one nucleotide containing a non-natural modification/substitution.
81. The method of any one of statements 66 to 80, wherein the type II or type V or type VI CRISPR/Cas effector protein is selected from Cas 9 family: Cas9; Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.
82. The method of any one of statements 66 to 81, wherein the type II or type V or type VI CRISPR/Cas effector protein is selected from: Cas9, Cas12a (Cpf1), Cas12b (C2c1), Cas 13a and Cas 13b.
83. The method of any one of statements 66 to 82, wherein the reporter construct is a labelled RNA
84. The method of any one of statements 66 to 82, wherein the reporter construct is a labelled DNA.
85. The method of any one of statements 66 to 84, wherein the steps of the method are conduct at a temperature ranging from 18 to 42 degrees Celsius, preferably, from 25 to 37 degrees Celsius.
86. The method of any one of statements 66 to 85, wherein the sample is also contacted with at least one sulfhydryl reductant.
87. The method of statement 86, wherein the sulfhydryl reductant is selected from the group consisting of Dithiothreitol (DTT), Tris(2-carboxyethyl) phosphine (TCEP) to and 2-Mercaptoethanol (2-ME).
88. The method of statement 87, wherein the sulfhydryl reductant is DTT.
89. The method of statement 88, wherein said contacting occurs at a temperature of about 37° C.
90. The method of any one of statements 66 to 89, wherein the sample is also contacted with at least one non-ionic surfactant.
91. The method of statement 90, wherein the non-ionic surfactant is selected from the group consisting of Brij L23 and poly(vinyl alcohol) (PVA).
92. The method of statement 91, wherein the non-ionic surfactant is PVA.
93. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a labelled reporter construct, wherein said reporter construct comprises a nucleic acid that is cleavable by the nuclease activity of the activated type V or type VI CRISPR/Cas effector protein; and
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the first guide RNA and also activates the nuclease activity of first type V or type VI CRISPR/Cas effector proteins bound to said first guide RNA following cleavage of the sealed end by the nuclease activity of said first type V or type VI CRISPR/Cas effector protein, and;
(b) measuring a detectable signal produced following cleavage of the labelled nucleic acid reporter construct by the first and second type V or type VI CRISPR/Cas effector protein, thereby detecting the target in the sample.
94. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
iii) a second type V or type VI CRISPR/Cas effector protein;
(iv) a second guide RNA, optionally in association with said second type V or type VI CRISPR/Cas effector protein, comprising: a region that binds to said second type V or type VI CRISPR/Cas effector protein, and a guide sequence;
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vi) a labelled reporter construct, wherein said reporter construct comprises a nucleic acid that is cleavable by the nuclease activity of the activated type V or type VI CRISPR/Cas effector protein; and
(b) measuring a detectable signal produced following cleavage of the labelled nucleic acid reporter by the first and/or second type V or type VI CRISPR/Cas effector protein, thereby detecting the target in the sample.
95. A method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the first guide RNA and also activates the nuclease activity of said first type V or type VI CRISPR/Cas effector proteins bound to said first guide RNA following cleavage of the sealed end by the nuclease activity of said first type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.
96. A method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence,
(vi) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally, wherein the double stranded structure includes a PAM sequence which is distal to the sealed end; and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first and second type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.
97. The method of statement 94 or 96, wherein the trigger nucleic acid sequence is a ssDNA or dsDNA or ssDNA sequence, preferably not having full (100%) complementarity to an existing genomic sequence, more preferably with the length of at least 10 nucleotides.
98. The method of any one of statements 95-97, wherein the target binding construct is an antibody or antigen binding fragment thereof.
99. The method of any one of statements 94 to 98, wherein the target binding construct is immobilized on a substrate or conjugated to a magnetic bead or a microparticle or a nanoparticle.
100. The method of statement 99, further comprising the step of performing magnetic separation of the captured target bound to the first target binding construct from the sample when the first target binding construct is conjugated to a magnetic bead.
101. The method of any one of statements 94 or 96 to 100, wherein the first and second type V or type VI CRISPR/Cas effector proteins are the same.
102. The method of any one of statements 94 or 96 to 100, wherein the first and second type V or type VI CRISPR/Cas effector proteins are different.
103. The method according to statement 102, wherein said first type V or type VI CRISPR/Cas effector protein is a Type V CRISPR/Cas effector protein and said second type V or type VI CRISPR/Cas effector protein is a Type VI CRISPR/Cas effector protein.
104. The method of any one of statements 94 or 96 to 103, wherein contacting the sample with the second effector type V or type VI CRISPR/Cas effector protein and the second guide RNA occurs at the same time when the sample has been contacted with the first effector type V or type VI CRISPR/Cas effector protein and the first guide RNA.
105. The method of any one of statements 94 or 96 to 104, wherein contacting the sample with the second effector type V or type VI CRISPR/Cas effector protein and the second guide RNA occurs after the sample has been contacted with the first effector type V or type VI CRISPR/Cas effector protein and the first guide RNA.
106. The method of statement 105, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.
107. The method of any one of statements 93 to 106, wherein the first guide RNA is bound to the first type V or type VI CRISPR/Cas effector protein.
108. The method of any one of statements 94 or 96 to 107, wherein the second guide RNA is bound to the second type V or type VI CRISPR/Cas effector protein.
109. A method of enhancing a type V or type VI CRISPR/Cas detection system, which comprises a type V or Type VI CRISPR/Cas effector protein, a first guide RNA, and a labelled nucleic acid reporter, comprising:
   adding to a reaction mixture comprising a Type V or Type VI CRISPR/Cas effector protein of the detection system a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with a guide sequence of a guide RNA of said type V or type VI CRISPR/Cas detection system and following cleavage of the sealed end by the nuclease activity of said first type V or type VI CRISPR/Cas effector protein further activates the nuclease activity of said type V or type VI CRISPR/Cas effector proteins bound to said guide RNA; and
   measuring a detectable signal produced following linearization of the circular DNA molecular construct, or the circular RNA molecular construct by the type V or type VI CRISPR/Cas effector protein of said detection system.
110. A method of enhancing a type V or type VI CRISPR/Cas detection system, which comprises a first type V or Type VI CRISPR/Cas effector protein, a first guide RNA, and a labelled nucleic acid reporter, comprising:
   adding to a reaction mixture comprising at least a first type V or Type VI CRISPR/Cas effector of the system:
(i) a second type V or type VI CRISPR/Cas effector protein;
(ii) a second guide RNA, optionally bound to the second type V or type VI CRISPR/Cas effector protein, said guide RNA comprising: a region that binds to the second type V or type VI CRISPR/Cas effector protein, and a guide sequence,
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with a guide sequence of the second guide RNA and following cleavage of the sealed end by the nuclease activity of said first type V or type VI CRISPR/Cas effector protein further activates the nuclease activity of said second type V or type VI CRISPR/Cas effector proteins bound to said second guide RNA; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein;
and measuring a detectable signal produced following cleavage of the labelled nucleic acid reporter of the detection system, and/or the labelled reporter construct when added, by the first and/or second type V or type VI CRISPR/Cas effector protein.
111. The method of any one of statements 93 to 110, wherein said first sequence of nucleotides and/or said second sequence of nucleotides of the palindromic oligonucleotide is from 10 to 30 nucleotides in length.
112. The method of statement 111, wherein said first sequence of nucleotides and/or said second sequence of nucleotides of the palindromic oligonucleotide is 15 nucleotides in length.
113. The method of any one of statements 93 to 112, wherein said first sequence of nucleotides and said second sequence of nucleotides of the palindromic oligonucleotide are 100% complementary to one another.
114. The method of any one of statements 93 to 113, wherein any one or more of the guide RNA, the palindromic oligonucleotide, and labelled nucleic acid, and the nucleic acid reporter construct and/or trigger nucleic acid, when present, comprises at least one nucleotide containing a non-natural modification/substitution.
115. The method of any one of statements 93 to 114, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.
116. The method of any one of statements 93 to 115, wherein the type V or type VI CRISPR/Cas effector protein is selected from: Cas12a (Cpf1), Cas12b (C2c1), Cas 13a and Cas 13b.
117. The method of any one of statements 93 to 116, wherein the steps of the method are conducted at a temperature ranging from 10 to 48 degrees Celsius, preferably, from 25 to 42 degrees Celsius.
118. The method of any one of statements 93 to 117, wherein the sample is also contacted with at least one sulfhydryl reductant.
119. The method of statement 118, wherein the sulfhydryl reductant is selected from the group consisting of Dithiothreitol (DTT), Tris(2-carboxyethyl) phosphine (TCEP) to and 2-Mercaptoethanol (2-ME).
120. The method of statement 119, wherein the sulfhydryl reductant is DTT.
121. The method of statement 118, wherein said contacting occurs at a temperature of above 10° C.
122. The method of any one of statements 93 to 121, wherein the sample is also contacted with at least one non-ionic surfactant.
123. The method of statement 122, wherein the non-ionic surfactant is selected from the group consisting of Brij L23 and poly(vinyl alcohol) (PVA).
124. The method of statement 123, wherein the non-ionic surfactant is PVA.
125. The method of any one of the preceding statements wherein the labelled nucleic acid reporter, the labelled reporter construct, or labelled circular DNA molecular construct or labelled circular RNA molecular construct comprises a fluorophore and a quencher of the fluorophore.
126. The method of statement 125, wherein the labelled nucleic acid reporter, the labelled reporter construct, or labelled circular DNA molecular construct or labelled circular RNA molecular construct comprises a fluorophore at the 5′ end and a quencher of the fluorophore at the 3′ end.
127. The method of statement 125 or 126, wherein the fluorophore is FAM and the quencher is BHQ1, or wherein the fluorophore is Texas Red and the quencher is BHQ2.
128. The method of any one of statements 1 to 127, wherein said contacting occurs in a reaction mixture comprising a buffer.
129. The method of any one of statements 1 to 128, wherein the target is detected at attomolar sensitivity or lower.
130. The method of any one of statements 1 to 129, wherein the sample is a biological sample or an environmental sample.
131. The method of statement 130, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.
132. The method of statement 131, wherein the sample is blood, plasma, serum or a biopsy obtained from a human patient.
133. The method of statement 130, wherein the sample is a water sample.
134. The method of statement 130, wherein the sample is a crude sample.
135. The method of any of statements 1-133, wherein the sample is a concentrated or purified sample.
136. A circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence, wherein said circular DNA molecular or circular RNA molecular construct comprises a sequence complementary to a guide RNA sequence which binds to a type V or type VI CRISPR/Cas effector protein, and wherein said circular DNA molecular or circular RNA molecular construct only hybridizes with the guide sequence of the guide RNA following linearization by cleavage of the ssDNA region in said circular DNA molecular construct or the ssRNA region in said circular RNA molecular construct.
137. The circular DNA molecular construct or circular RNA molecular construct of statement 104, wherein the 5′end and/or the 3′ end of the dsDNA sequence of the circular DNA molecular construct are detectably labelled; or wherein the 5′end and/or the 3′ end of the ssDNA or dsDNA or dsRNA or DNA/RNA hybrid sequence of the circular RNA molecular construct are detectably labelled.
138. The circular DNA molecular construct or circular RNA molecular construct of statement 136 or 137, wherein the, wherein the construct has the total length (circumference) from 15 to 30 nucleotides.
139. The circular DNA molecular construct or circular RNA molecular construct of 138, wherein the circular DNA molecular construct or circular RNA molecular construct has a total length of 16, 17, 18, 19, 20, 21, 22, or 23 nucleotides.
140. The circular DNA molecular construct or circular RNA molecular construct of any one of statements 136 to 139, wherein the circular DNA molecular construct comprises two parts: i) a region comprising 1-5 nucleotides ssDNA, and ii) the remaining part of the molecular construct is dsDNA with a complementary sequence to a guide RNA sequence which binds to a type V or type VI CRISPR/Cas effector protein; and wherein the circular RNA molecular construct comprises two parts: i) a region comprising 0-5 nucleotides ssRNA, and ii) the remaining part of the molecular construct is ssDNA or dsDNA or dsRNA or DNA/RNA hybrid with a complementary sequence to a guide RNA sequence which binds to a type V or type VI CRISPR/Cas effector protein.
141. The circular DNA molecular construct or circular RNA molecular construct of any one of statements 104 to 108, comprising at least one nucleotide containing a non-natural modification/substitution.
142. The circular DNA molecular construct or circular RNA molecular construct of any one of statements 137 to 141, comprising a fluorophore and a quencher of the fluorophore.
143. The circular DNA molecular construct or circular RNA molecular construct of statement 110, wherein the labelled nucleic acid reporter, the labelled reporter construct, or 142 circular DNA molecular construct or labelled circular RNA molecular construct comprises a fluorophore at the 5′ end and a quencher of the fluorophore at the 3′ end.
144. The circular DNA molecular construct or circular RNA molecular construct of statement 142 or 143, wherein the fluorophore is FAM and the quencher is BHQ1, or wherein the fluorophore is Texas Red and the quencher is BHQ2.
145. A circular guide RNA, wherein the circular guide RNA is susceptible to trans-cleavage nuclease activity of a type II or type V or type VI CRISPR/Cas effector protein and comprises a region that binds to a type II or type V or type VI CRISPR/Cas effector protein, and a guide sequence that hybridizes with a target nucleic acid sequence or a trigger nucleic acid sequence, wherein hybridization between the guide sequence and the trigger nucleic acid sequence, only occurs following linearization of the circular guide RNA by the cis-cleavage or trans-cleavage nuclease activity of a type II or type V or type VI CRISPR/Cas effector protein.
146. The circular guide RNA of statement 145, comprising a total length (circumference) from 40-80 nucleotides.
147. The circular guide RNA of statement 145 or 146, wherein the comprising two parts including a 2-5 ssDNA nucleotides or a 14-24 dsDNA nucleotides, and the remaining part of the guide RNA comprises a complementary sequence to a target nucleic acid or trigger nucleic acid sequence a sequence which binds to a type II or type V or type VI CRISPR/Cas effector protein.
148. The circular guide RNA of any one of statements 145 to 146, wherein any one or more of the comprising at least one nucleotide containing a non-natural modification/substitution.
149. A kit for detecting a target in a sample, the kit comprising:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the second guide RNA and the dsDNA or ssDNA or dsRNA or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.
150. A kit for amplifying enhancing a type V or type VI CRISPR/Cas detection system, which comprises a first type V or Type VI CRISPR/Cas effector protein, a guide RNA which binds to the type V or type VI CRISPR/Cas effector protein, and a labelled nucleic acid reporter, the kit comprising: the circular DNA molecular construct or circular RNA molecular construct of any one of statements 136 to 144, or the circular guide RNA of any one of statements 145 to 148, or a palindromic oligonucleotide as described in any one of statements 93 to 114.
151. The kit of statement 150, further comprising one or more of: a type V or Type VI CRISPR/Cas effector protein, a guide RNA, and a labelled nucleic acid reporter.
152. The kit of statement 149 to 151, further comprising one or more of: a target binding construct, a reaction buffer, a washing buffer, and reagents for recovering or releasing immobilized or captured target.
153. The kit of statement 152, wherein said reaction buffer, washing buffer, and reagents for recovering or releasing immobilized or captured target comprise a sulfhydryl reductant, and/or a non-ionic surfactant.
154. The kit of statement 153, wherein the sulfhydryl reductant is selected from the group consisting of Dithiothreitol (DTT), Tris(2-carboxyethyl) phosphine (TCEP) to and 2-Mercaptoethanol (2-ME); and wherein the non-ionic surfactant is selected from the group consisting of Brij L23 and poly(vinyl alcohol) (PVA).
155. The kit of statement 154, wherein the sulfhydryl reductant is DTT and the non-ionic surfactant is PVA.
156. The kit of any one of statements 149 to 155, when used for a method for detecting a target in a sample.
157. A reaction mixture comprising:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the second guide RNA and the dsDNA or ssDNA or dsRNA or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.
158. A reaction mixture comprising the circular DNA molecular construct or circular RNA molecular construct of any one of statements 136 to 144, or the circular guide RNA of any one of statements 145 to 148, or the palindromic oligonucleotide as described in any one of statements 93 to 114.
159. The reaction mixture of statement 158, further comprising one or more of: a type V or Type VI CRISPR/Cas effector protein, a guide RNA, and a labelled nucleic acid reporter.
160. The reaction mixture of any one of statements 158 to 159, further comprising a sample.
161. The reaction mixture of any one of statements 158 to 160, further comprising a reaction buffer.
162. The reaction mixture of statement 161, further comprising a sulfhydryl reductant, and/or a non-ionic surfactant.
163. The reaction mixture of statement 162, wherein the sulfhydryl reductant is selected from the group consisting of Dithiothreitol (DTT), Tris(2-carboxyethyl) phosphine (TCEP) to and 2-Mercaptoethanol (2-ME)), and wherein the non-ionic surfactant is selected from the group consisting of Brij L23 and poly(vinyl alcohol) (PVA).
164. The reaction mixture of statement 163, wherein the sulfhydryl reductant is DTT, and wherein the non-ionic surfactant is PVA.
165. The method according of any one of statements 1 to 135, the kit of any one of statements 149 to 156, or the reaction mixture of any one of statements 157 to 164, wherein the first or second type V or type VI CRISPR/Cas effector protein is immobilized on a substrate or conjugated to a magnetic bead or a microparticle or a nanoparticle.
166. The method of statement 35, wherein the second guide RNA comprises a guide sequence that hybridizes with the dsDNA of the circular DNA molecular construct or the dsDNA or ssDNA sequence of the circular RNA molecular construct, but not the target sequence.

### BRIEF DESCRIPTION OF DRAWINGS

FIG. 1 Fabrication of Cir-mediators with ligase, e.g. T4 DNA ligase. (A) Schematics for synthesis of Cir-mediators using T4 ligase. (B) Verification of the CRISPR/Cas12a activation ability of 5′-phosphorylated linear ssDNA. (C) agarose gel electrophoresis assay shows the formation of circular ssDNA (Cir-ssDNA) construct; 1=10 bp DNA ladder, 2=Negative, 3=linear ssDNA, 4=linker oligo, 5=linear ssDNA+linker, 6=1× synthesized Cir-ssDNA, 7=2× synthesized Cir-ssDNA, 8=3× synthesized circular ssDNA, 9=4× synthesized Cir-ssDNA. (D) Exonuclease III treatment for linear DNA digestion. 1=10 bp DNA ladder, 2=linear ssDNA+exo III, 3=linear ssDNA, 4=linear ssDNA+linker+exo III, 5=linear ssDNA+linker, 6=synthesized Cir-ssDNA+exo III, 7=synthesized Cir-ssDNA. (E) Formation of the cir-mediator. 1=10 bp ladder, 2=10 bp ladder, 3=linear ssDNA, 4=linker, 5=cDNA, 6=linear ssDNA+linker, 7=circular ssDNA, 8=Cir-mediator.

FIG. 2 Fabrication of Cir-mediators by using click-chemistry. (A) Schematics for synthesis the Cir-ssDNA using click chemistry, where the 3′-CHCH linked to the dT nucleotide with Azide. (B) agarose gel electrophoresis assay verifying the formation of a circular ssDNA structure; 1 and 10=10 bp DNA ladder, 2-3=linear ssDNA, 4-5=linear ssDNA with exonuclease III treatment, 6-7=Cir-ssDNA, 8-9=Cir-ssDNA after exonuclease III treatment. (C) Formation of Cir-mediators with the addition of its complementary DNA (cDNA). (D) Verification that the Cir-mediators produced by the click chemistry do not activate trans-cleavage in Cas12a (Neg=negative control using the same volume of PBS for oligos to the standard CRISPR/Cas12a reaction mixture).

FIG. 3 Cir-mediators suppressed Cas12a trans-cleavage activity. (A) Demonstration that the Cir-mediators do not induce Cas12a trans-cleavage. Cir-Med=Cir-mediator, Neg−=negative control using the same volume of PBS for oligos to the standard CRISPR/Cas12a reaction mixture. (B) Exonuclease III reduces the unwanted background of trans-cleavage activation of Cas12a by the residual linear nucleic acids which were in excess. Exo III treated=Cir-mediator prepared with exonuclease III treated Cir-ssDNA+ its cDNA, and used for standard CRISPR/Cas12a reaction mixture; Pos+=triggering linear ssDNA used for standard CRISPR/Cas12a reaction mixture; Neg−=negative control using the same volume of PBS for oligos to the standard CRISPR/Cas12a reaction mixture. The Cir-mediator tested here was prepared using ligase approach as presented in FIG. 1.

FIG. 4 Restoration of Cas12a RNP trans-cleavage activation by cleaved (linearized) Cir-mediators. (A) Specificity test for the first round of CRISPR/Cas12a RNP activation targeting a specific ssDNA sequence (the “target”). Oligo=triggering ssDNA for first CRISPR/Cas12a RNP, Cir-Mediator=adding the same volume of Cir-mediator to the first round of CRISPR/Cas12a reaction mixture, genome=random extracted genome DNA, Neg−=negative control using the same volume of PBS to the first round of standard CRISPR/Cas12a reaction mixture. (B) Specificity test for the second round of CRISPR/Cas12a RNP activation targeting the dsDNA region of the Cir-mediator. Oligo=random triggering ssDNA not complementary to any of gRNAs in this test, Cir-Mediator=adding the same volume of triggering ssDNA to the enhanced CRISPR/Cas12a reaction mixture (gRNA for triggering ssDNA+Cir-mediator), genome=random extracted genome DNA, Neg−=negative control using the same volume of PBS for oligos to the enhanced CRISPR/Cas12a reaction mixture. (C) Restoration of Cas12a RNP trans-cleavage activation by cleaved (linearized) Cir-mediators. 10 uL act-Cas12a=transferring 10 μL of pre-activated standard CRISPR/Cas12a reaction mixture (Cir-mediator added) into the standard CRISPR/Cas12a reaction mixture with gRNA targeting Cir-mediator, 5 uL act-Cas12a=transferring 5 μL of pre-activated standard CRISPR/Cas12a reaction mixture (Cir-mediator added) into the standard CRISPR/Cas12a reaction mixture with gRNA targeting Cir-mediator, No act-Cas12a=transferring 5 μL of non-activated standard CRISPR/Cas12a reaction mixture (Cir-mediator added) into the standard CRISPR/Cas12a reaction mixture with gRNA targeting Cir-mediator, Neg−=adding 5 μL of PBS into the standard CRISPR/Cas12a reaction mixture with gRNA targeting Cir-mediator. The Cir-mediator tested here was prepared using ligase approach as presented in FIG. 1.

FIG. 5 shows schematics of embodiments of signal amplification using Cir-mediators. (A) The principle of Cir-mediator-induced cascade of CRISPR/Cas12a activation of trans-cleavage leading to assay signal amplification. (B) Schematic of Cir-mediator-induced cascade utilizing and RNA-DNA Cir mediator and two different CRISPR/Cas effector proteins.

FIG. 6 Cir-mediator-induced CRISPR/Cas12a trans-cleavage cascade reaction leads to increased sensitivity in a DNA assay. (A) Demonstration of the application of Cir-mediators for increasing the final signal output (Cir-mediators made by the ligase method). 0, 5, 10 indicated 0, 5, 10 μL of triggering ssDNA added into either the standard CRISPR/Cas12a reaction mixture (Normal) or the Cir-mediator enhanced CRISPR/Cas12a reaction mixture (cir-AMP), Neg−=0, 5, 10 μL of PBS added into the standard CRISPR/Cas12a reaction mixture. (B) Cir-mediators lead to a faster reaction speed allowing to reach signal saturation more quickly (Cir-mediators made by the ligase method). Normal=the standard CRISPR/Cas12a reaction mixture, cir-AMP=the Cir-mediator enhanced CRISPR/Cas12a reaction mixture, cir-AMP 2×=the Cir-mediator enhanced CRISPR/Cas12a reaction mixture with 2 times concentration of Cas12a RNPs. (C) Standard CRISPR/Cas12a reaction without the presence of Cir-mediator. The system response to the presence of target DNA at minimum concentration of 1 pM shows a significantly higher fluorescence intensity compared to negative control. (Cir-mediators made by the click chemistry) “0” indicates the added same volume of sample has no target DNA presented. (D) The Cir-mediator induced CRISPR/Cas12a amplification cascade reaction. This Cir-mediator enhanced system response to the presence of target DNA at minimum concentration of 1 aM with a significantly higher fluorescence intensity compared to negative control. 0 indicated the added same volume of sample has no target DNA presented. (Cir-mediators made by the click chemistry).

FIG. 7 shows activation of Cas12a with linear ssDNA of different lengths (15 nt, 18 nt and 21 nt). Neg is negative control—adding the same amount of PBS as of the linear ssDNA.

FIG. 8 shows formation of circular ssDNA with oligos with different lengths (the lettering in the figure refers to the lengths of the dsDNA region. The total length of the nucleotides is the length of the double strand region+2 additional single strand nucleotides.

FIG. 9 shows Exo III treatment is necessary to reduce free linear ssDNA activating Cas12a. Neg—is the negative control: the same amount of PBS as of the trigger DNA added to the standard CRISPR/Cas reaction mixture.

FIG. 10 shows unwanted Cas12a activation with free linear ssDNA at different reaction times. Demonstration that Exo III treatment is necessary to reduce free linear ssDNA activating Cas12a. Neg—is the negative control: the same amount of PBS as of the trigger DNA added to the standard CRISPR/Cas reaction mixture.

FIG. 11 shows activation of Cas12a trans-cleavage activity with linear dsDNA for different lengths (total length=labelled length+2 nt). Labels on the horizontal axis refer to the double strand DNA region. Negative control is the same amount of PBS as the amount of trigger DNA added to the standard CRISPR/Cas reaction. N/A is the background signal from the 96 well plate.

FIG. 12 shows activation of Cas12a trans-cleavage activity with Cir-mediator at different length (the total length=labelled length+2 nt). Labels on the horizontal axis refer to the double strand DNA region. Negative control is the same amount of PBS as the amount of trigger DNA added to the standard CRISPR/Cas reaction. N/A is the background signal from the 96 well plate.

FIG. 13 shows results of a specificity test. Pos+ is a positive first round of CRISPR/Cas12a reaction product (with Cir-mediator) added into the second round of CRISPR/Cas12a reaction mixture (gRNA for Cir-mediator); 1AMP-/Wrong gRNA is negative first round of CRISPR/Cas12a reaction due to the use of a mismatched first guide RNA (with Cir-mediator), and then added into the second round of CRISPR/Cas12a reaction mixture (gRNA for Cir-mediator); 1 AMP-/no Cas12a is negative first round of CRISPR/Cas12a reaction due to no Cas 12a RNP (with Cir-mediator), and then added into the second round of CRISPR/Cas12a reaction mixture (gRNA for Cir-mediator); 1 AMP-/no cir mediator is a positive first round of CRISPR/Cas12a reaction but without the use of Cir-mediator, and then added into the second round of CRISPR/Cas12a reaction mixture (gRNA for Cir-mediator); Neg− is the unactivated standard CRISPR/Cas12a reaction mixture. (5 uL of 100 fM triggering ssDNA was used to activate of 100 μL of first round of CRISPR/Cas12a reaction mixture, and 5 μL of first round of product was transferred into 100 μL of second round of CRISPR/Cas12a reaction mixture).

FIG. 14 shows a schematic of the DANCER system (a) and a classic CRISPR/Cas12a detection system (b).

FIG. 15 shows synthesis and characterization of Cir-ssDNA using click chemistry. (a) Schematic for the synthesis of Cir-ssDNA using a bead-based click chemistry method. Biotin-ssDNA with specific modifications (5′-Azide (N3); 3′-CHCH; internal-Biotin); (b) Demonstration of the formation of Cir-ssDNA using denaturing polyacrylamide gel (dPAGE) electrophoresis (from left to right: 1. 10 bp ladder; 2. 19 nt linear ssDNA; 3. 19 nt Cir-ssDNA; 4. 10 bp ladder.); (c) Optimization of Cir-ssDNA synthesis efficiency using a bead-based click chemistry method; (d) Reproducibility of Cir-ssDNA synthesis using a bead-based click chemistry method.

FIG. 16 shows performance of Cir-amplifiers as reporters in a classic CRISPR/Cas12a biosensing system. (a) Schematics for the investigation of reporter performance of Cir-amplifier; (b) Comparison of the fluorescence signals of a Cir-amplifier and linearized Cir-amplifier (L-Cir-amplifier) (18 nt dsDNA with 3 nt ssDNA); (c) Background signals of Cir-amplifiers with different linker lengths (18 nt dsDNA). Here L-x represents the linker length is x nt; (d) Investigation of the Cir-amplifier linker length in a classic CRISPR/Cas12a biosensing system (18 nt dsDNA, 100 pM target DNA). Here L-x represents the linker length is x nt; (e) Comparison of the detection limits of classic CRISPR/Cas12a biosensors with Cir-amplifiers (18 nt dsDNA with 3 nt ssDNA) and with linear ssDNA reporters (TTATT) with identical fluorophore-quencher pairs.

FIG. 17 shows RNP activation efficiency of Cir-amplifiers and linearized Cir-amplifiers in a CRISPR/Cas12a biosensing system. (a) Schematics for the application of Cir-reporters as activators for Cas12a RNP; (b) Evaluation of the dsDNA length in Cir-amplifier for the activation of a classic CRISPR/Cas12a biosensing system (with a 3 nt ssDNA linker); (c) Evaluation of the linker length in Cir-amplifier for the activation of a classic CRISPR/Cas12a biosensing system (with 18 nt dsDNA “fake target”). Here L-x represents the linker length is x nt; (d) Schematic for the application of linearized Cir-amplifier as activators for Cas12a RNP; (e) Evaluation of the RNP activation efficiency of linearized Cir-amplifiers; (f) Comparison of the RNP activation efficiency by linearized Cir-amplifiers and by corresponding linear dsDNA.

FIG. 18 shows characterization of the DANCER sensor. (a) The DANCER fluorescent signal as a function of Cir-amplifier concentration (20 nM of Cas12a RNP, and 1 pM of target DNA); (b) The DANCER fluorescent signal as a function of Cas12a RNP concentration (200 nM of Cir-amplifier, and 1 pM of target DNA); (c) The calibration curve of DANCER (60 nM of Cas12a RNPs, and 200 nM of Cir-amplifiers).

FIG. 19 shows the application of DANCER for the point-of-care detection of cfDNA from mouse plasma. (a) Biosensing performance of DANCER in PBS and mouse plasma; (b) The calibration curve of DANCER in mouse plasma; (c) The procedure of DANCER for cfDNA detection from mouse plasma; (d) The application of DANCER for cfDNA detection in mouse plasma (n=3); (e) The establishment of a colorimetric Cir-amplifier through extending of the cDNA with 5 nt CCCCC and a biotin on 3′ end; (f) The schematic of the colorimetric lateral flow assay for the detection of biotin-DNA-FAM reporter (control line: streptavidin; test line: secondary antibody); (g) The application of colorimetric Cir-amplifier-based DANCER for cfDNA detection in mouse plasma with a lateral flow assay.

FIG. 20 shows standard calibration curve for the calculation of Cir-ssDNA concentration using Nanodrop (ThermoFisher).

FIG. 21 shows evaluation of Cir-amplifier as fluorescent reporters in a classic CRISPR/Cas12a biosensing system. (a) Comparison of the biosensing performance of Cir-amplifier and ssDNA reporter in a classic CRISPR/Cas12a biosensing system; (b) Comparison of the biosensing performance of Cir-amplifier at RT and 37° C.

FIG. 22 shows investigation of the ssDNA linker length in the Cir-amplifier.

FIG. 23 shows the calibration curve of a classic CRISPR/Cas12a biosensing system.

FIG. 24 shows the development of Cir-amplifier assisted autocatalysis biosensing system using two different types of Cas12a RNPs (DANCER-2). (a) The schematic for the development of DANCER-2; (b) The investigation of Cas12a RNP1 and RNP2 ratio (Cir-amplifier 200 nM, and 1 pM target-C); (c) The amplified signals of Cas12a RNP1-RNP2 biosensing system (Cir-amplifier 200 nM, and 1 pM target-C); (d) The comparison of DANCER-2 (two Cas12a RNPs) and DANCER (One Cas12a RNP); (e) The limit of detection of DANCER-2.

FIG. 25 shows the schematic of Cir-gRNA mediated Cas12a autocatalysis biosensing system.

FIG. 26 shows the formation of Cir-gRNA. The figure shows that a linear gRNA based CRISPR/Cas12a biosensing system is able to function properly, with increased fluorescence, while Cir-gRNA-based CRISPR/Cas12a biosensing system is suppressed with limited fluorescence increase, demonstrating the formation of of Cir-gRNA.

FIG. 27 shows the establishment of Cir-gRNA mediated Cas12a autocatalysis biosensing system. 1 pM trigger ssDNA was utilized to activate the Cas12a autocatalysis system.

FIG. 28 shows the biosensing performance of Cir-gRNA mediated CRISPR/Cas12a biosensing system.

FIG. 29 shows schematics for T-locker DNA nanostructure and its function. (A) The exemplary figure of Cas12a RuvC enzymatic domain and the R-loop structure between gRNA and its target dsDNA sequence. (B) A typical structure of a dsDNA target for Cas12a RNP. (C) The schematic figure of the T-locker molecule structure, and its function of restricted Cas12a activation due to uncomplete R-loop formation.

FIG. 30 shows T-locker lead to restricted Cas12a activation. (A) The Cas12a activation efficiencies between T-locker (T-lock-0) and a normal dsDNA molecule (Full) with same length of targeted sequence. (B) The Cas12a activation patterns for T-locker (T-lock-0) and its normal dsDNA formation (Full) for a 90 mins reaction at room temperature.

FIG. 31 shows restored Cas12a activation through pre-activated trans-cleavage. (A) The Cas12a activation efficiency between trans-cleavage treated T-locker (Pre-act) and also the untreated T-locker (No act) molecules. (B) comparison of Cas12a activation efficiency of fully reopened T-locker to its normal dsDNA formation with same targeted sequence length.

FIG. 32 shows different T-locker status led to different Cas12a interactions. (A) The Cas12a activation levels for T-locker at different concentrations. (B) Comparison of the Cas12a activation efficiency changes of T-locker at different concentrations along with pre-treatment of trans-cleavage. (C) The Cas12a activation level changed due to T-locker synthesis temperatures.

FIG. 33 shows characterization of T-locker to Cas12a trans-cleavage. (A) The interaction between T-locker to Cas12a trans-cleavage activation in different buffering systems. (B) Exonuclease III treated T-locker shows moderate resistance. (C) Temperature has a significant effect to T-locker synthesis.

FIG. 34 shows T-locker induced autocatalysis reaction for DNA detection. (A) The schematic figures for T-locker induced autocatalysis reaction. (B) The sensitivity of ssDNA detection using T-locker induced autocatalysis reaction.

FIG. 35 shows that Cir-mediator-induced Cas12a autocatalysis amplifies trans-cleavage in Autocatalytic Cas12a Circular DNA Amplification Reaction (AutoCAR)-1 system. (A) The AutoCAR-1 scheme for amplified ssDNA cleavage using ssDNA-linked fluorescence-quenched reporters. (B) The fluorescence signal intensity differences between reporter trans-cleavage in a standard CRISPR/Cas12a and the AutoCAR-1 systems measured by the fluorescent signal produced by cleavage of fluorescent quenched ssDNA reporters (n=3). In comparison with a standard Cas12a catalytic system without Cir-mediators, the reporter trans-cleavage increased 7.3 times over 3600 sec (60 mins) and 14.4 times over 7200 sec (120 mins) reactions at room temperature. (Method 8.1a). (C) The reporter trans-cleavage in AutoCAR-1 increased with increasing Cir-mediator concentration (n=3). (Method 8.1b). (D) Time dependence of the fluorescence signal for AutoCAR-1 in comparison to a standard CRISPR/Cas12a reaction (n=3). Our data show differences in reporter trans-cleavage kinetics in a standard Cas12a catalytic system (linear trend, y=0.014x+9.1129, goodness of fit R²=0.9584) and Cir-mediator-assisted Cas12a autocatalysis reaction (super-linear trend, exponential fit, y=12.102e^{0.0023}x, goodness of fit R²=0.9844). These results indicate that instead of the well-established linear trend, AutoCAR-1 produces a nonlinearly increasing signal (Method 8.1a).

FIG. 36 shows AutoCAR-1 is capable of ultra-sensitive DNA and RNA diagnostics with no amplification and no reverse transcription. (A) The calibration curve for AutoCAR-1 DNA detection (n=3). The system has 3 orders of magnitude linear range, with DNA detection sensitivity down to 1 aM. two-tailed t-test. (Method 8.1a). (B) Detection of H. pylori bacterial genome DNA using the AutoCAR-1 system targeting the glm gene (n=3). (Method 9.1a, 9.1b). (C) The AutoCAR-1 calibration curve for RNA detection (n=3). The system investigated here shows 3 orders of magnitude linear range with RNA detection sensitivity of 1 aM. two-tailed t-test. (Methods 9.1c). (D) Detection of the N-gene fragment of SARS-CoV-2 viral genome RNA using the AutoCAR-1 system alone, without reverse transcription (n=3). (Method 9.1c). The 1 aM LOD is consistent with exponential growth in the number of Cas12a proteins activated by a sing target 3 orders of magnitude in ˜25 minutes, over approximately twice the duration (˜1 hour) yields the LOD increase of ˜3 orders of magnitude squared, so 6 orders of magnitude lower compared with well-established LOD values for pre-amplification free Cas12a detection systems (1-10 pM). (*P<0.05, **P<0.005, ***P<0.001).

FIG. 37 shows application of AutoCAR (AutoCAR-3) for ctDNA detection from blood plasma. Plasma samples from patients with advanced cancers harboring the PIK3CA H1047R mutation as determined in tumour biopsies (PIK3CA H1047R+n=6, and PIK3CA H1047R−n=4) were subject to detection of circulating PIK3CA mutations in blood plasma using AutoCAR-2 testing. Dashed lines represent the averages of the positive (light) and negative (dark) groups. (*P<0.05).

FIG. 38 shows background signal due to PIK3CA wild type gene fragments in patient plasma samples. The fluorescence signal for the PIK3CA+ (light) and PIK3CA− (dark) patient groups shown here are indicative of the background signal level due to the wild type PIK3CA gene fragments in the tested plasma samples. The lack of significant difference between two patient groups confirms that our AutoCAR-3 system can specifically detect the PIK3CA H1047R mutation in ctDNA from the patient samples, as indicated in FIG. 37. (n=3, ns=non-significant).

FIG. 39 shows feasibility of AutoCAR-3 detection in saliva. N=40 saliva samples were tested as collected (black) and after spiking with 1 fM of the PIK3CA H1047R mutation sequence (light). Saliva has undergone no preparation other than freezing at −20° C. degrees. Broken lines indicate averages of both groups (as collected and spiked). The data shows statistical difference between these two groups.

FIG. 40 shows establishment of AutoCAR-2 using AsCas12a protein. (A) Comparison of the biosensing performance of AsCas12a based AutoCAR-2 with a standard AsCas12a biosensing system, with trigger DNA concentration at 1 pM. (B) The limit of detection of AsCas12a based AutoCAR-2.

FIG. 41 shows the AutoCAR-1 system enables specific detection of DNA in the attomolar concentration range, down to 1 aM. While the standard CRISPR/Cas12a system without additional amplification strategy shows no detectable signal differences between the same target concentration ranges (*P<0.05, **P<0.005, ***P<0.001).

FIG. 42 shows performance of AutoCAR-1 for DNA detection at low concentration levels. AutoCAR-1 system under these conditions is able to differentiate even small target concentration changes at low concentration level, here between 1 aM to 5 aM target DNA. (*P<0.05, **P<0.005, ***P<0.001).

FIG. 43 shows kinetic fluorescence signal profiles for AutoCAR-1 detecting DNA and RNA, (A) AutoCAR-1 for DNA detection; (B) AutoCAR-1 for RNA detection. (n=3) Methods 8.1a and 9.1c.

FIG. 44 shows AutoCAR-1 trans-cleavage pattern. After the autocatalysis loop of AutoCAR-1 has been activated, the fluorescence signal intensity increased strongly with reaction time following a non-linear growth pattern, in response to addition of 1 pM ssDNA. “Neg−” represents an inactive AutoCAR-1 reaction mixture, without trigger ssDNA. (Method 8.1a)

FIG. 45 shows aM-level Rapid DNA detection. The AutoCAR-1 is capable of detecting the presence of target DNA at 10 aM sensitivity in a 10 min reaction, and 1 aM sensitivity in a 20 mins reaction at room temperature (Method 9.1a)

FIG. 46 shows aM-level Rapid RNA detection. The AutoCAR-1 is capable of detecting the presence of target RNA at 5 aM sensitivity in a 10 mins reaction, and 1 aM sensitivity in a 30 mins reaction at room temperature (Method 9.1c).

FIG. 47 shows investigation of the basic properties of CRISPR/Cas13a biosensing system. (A) Investigation of trigger ratio; (B) Investigation of gRNA to Cas13a ratio; (C) Investigation of reporter ratio; (D) Investigation of buffer; (E) Investigation of temperature; (F) Investigation of limit of detection.

FIG. 48 shows single strand trigger for Cas13a. (A) Different trigger types for Cas13a; (B) Investigation of different trigger length of ssRNA for Cas13a; (C) Investigation of extended trigger ssRNA for Cas13a.

FIG. 49 shows double strand trigger for Cas13a RNP. (A) Schematic for dsRNA as trigger for Cas13a; (B) Investigation of different types of double strand trigger for Cas13a; (C) Investigation of the length of double strand RNA trigger for Cas13a.

FIG. 50 shows investigation of the trigger mechanism of dsRNA for Cas13a RNP. (A) Schematic of FRET approach for the investigation of trigger mechanism; (B) FRET approach for the investigation of trigger mechanism; (C) Schematic of locker strategy for the investigation of trigger mechanism; (D) Locker strategy for the investigation of trigger mechanism; (E) Investigation of the locker size.

FIG. 51 shows trigger ability of circular RNA. (A) Demonstration the formation of Cir-ssRNA. 1). 10 bp ladder; 2). Linear ssRNA; 3) Circular ssRNA. (B) Investigation of the trigger ability of circular ssRNA; (C) Investigation of the trigger ability of circular dsRNA.

FIG. 52 shows investigation of the trans-cleavage targets of Cas13a RNP. (A) Schematic of the trans-cleavage activity of Cas13a RNP; (B) Different types of single strand targets; (C) Different ssRNA targets; (D) Investigation of the trans-cleavage activity on dsRNA targets. 1). 10 bp ladder; 2). dsRNA; 3) dsRNA with CRISPR/Cas13a; (E) Investigation of the trans-cleavage activity on ssRNA and dsRNA targets.

FIG. 53 shows the development of RNA Cir-reporter. (A) Schematic of RNA Cir-reporter; (B) The background of Cir-reporter and linear reporter; (C) The biosensing application of Cir-reporter.

FIG. 54 shows the development of RNA Cir-amplifier based autocatalysis sensor. (A) Schematic of RNA Cir-amplifier based autocatalysis sensor; (B) Investigation of the autocatalysis activity of RNA autosensor; (C) Sensitivity of RNA autosensor; (D) Specificity of RNA autosensor; (E) Stability of RNA autosensor.

FIG. 55 shows a schematic of H-locker mediated CRISPR/Cas tandem biosensing system.

FIG. 56 shows the establishment of H-locker mediated CRISPR/Cas tandem biosensing system. (A) Investigation of the trigger ability of H-locker on Cas13a RNP; (B) Investigation of the reporter ability of H-locker; (C) Investigation of the trigger ability of cleaved H-locker.

FIG. 57 shows investigation of the biosensing performance of H-locker mediated CRISPR/Cas tandem biosensing system.

FIG. 58 shows AutoCAR-2 based electrochemical biosensing system. (A) Electrochemical detection in a standard CRISPR/Cas12a biosensing system without any trigger (Control); (B) Electrochemical detection in a standard standard CRISPR/Cas12a biosensing system with 1 nM trigger ssDNA; (C) Electrochemical detection in an AutoCAR-2 biosensing system with 1 nM trigger ssDNA.

FIG. 59 demonstrates synthesis and application of circular RNA-NA based RNA target recognition.: (A) Schematic of circular RNA-DNA based RNA target recognition using Cas13a and Cas12a. (B) Electrophoresis gel highlights the difference in mobility pattern between linear and circular RNA-DNA facilitated by click chemistry. (C) LbCas12a activation by products of LwCas13a reaction linear and circular ssRNA-DNA (L3) and cleaved circular RNA-DNA using Cas13a (D) Detection of target RNA concentration using circular RNA-DNA.

### DESCRIPTION OF EMBODIMENTS

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

Reference throughout this specification to “one embodiment,” “an example embodiment,” means that a particular feature, structure, or characteristic described in connection with the embodiment is included in at least one embodiment of the present invention. Thus, the appearances of the phrases “in one embodiment,” “in an embodiment,” or “exemplary embodiment” in various places throughout this specification are not necessarily all referring to the same embodiment, but may. Furthermore, the particular features, structures, or characteristics may be combined in any suitable manner in one or more embodiments, as will be apparent to those of ordinary skill in the art from this disclosure. Furthermore, although some embodiments described herein include some but not other features included in other embodiments, combinations of features of different embodiments are intended to be within the scope of the invention. For example, in the appended claims, any of the claimed embodiments may be used in any combination.

In order that the present technology may be more clearly understood, preferred embodiments will be described with reference to the following examples. The present disclosure is not to be limited in scope by the specific examples described herein, which are intended for the purpose of exemplification only. Functionally-equivalent methods and systems are clearly within the scope of the disclosure, as described herein.

### Overview

The capability of type V CRISPR/Cas proteins, e.g., Cas12 proteins such as Cpf1 (Cas12a) and C2c1 (Cas12b) to promiscuously cleave non-targeted single stranded DNA/RNA (ssDNA/ssRNA) once activated by detection of a target DNA (double or single stranded) has been reported previously. Similar capabilities have previously been reported for Type VI Cas effectors e.g. Cas13a, Cas13b Cas 13c etc., but their trans-cleavage is only effective on ssRNA. Once a type V/VI CRISPR/Cas effector protein (e.g., a Cas12 protein such as Cas12a, Cas12b, Cas12c, Cas12d, Cas12e, Cas 12g, Cas12j, Cas 12 k or Cas 13 protein such as Cas 13a, Cas13b Cas 13c and more recently discovered variants) is activated by a guide RNA (also known as the crispr RNA or crRNA), resulting from hybridization of the guide RNA to a target sequence of a target DNA/RNA (e.g. a targeted DNA/RNA sequence in a sample), the protein becomes a nuclease that promiscuously cleaves nucleic acids (i.e. ssDNA, dsDNA or ssRNA for Type V effectors, or ssRNA for type VI effectors) present (e.g. to which the guide sequence of the guide RNA does not hybridize). Thus, when the target DNA is present in the sample (e.g., in some cases above a threshold amount), the result is cleavage of nucleic acids, e.g. ssDNAs in the sample, which can be detected using any convenient detection method (e.g., using a labelled single stranded detector DNA).

As described above this capability has been exploited for use in methods of biosensing, and signal amplification. However, methods that have been developed in the art to date have had limitations relating to compromised sensitivity, excessive reaction time, overall system complexity or reduced stability and reliability.

Provided herein are methods, kits and compositions which enable the markedly enhanced detection of a target in a sample by utilizing the non-specific nuclease activity (i.e. cleavage of ssDNA, dsDNA, or ssRNA) of an activated type V or type VI CRISPR/Cas effector protein in combination with short circular DNA or RNA molecular constructs as mediators to control the trans-cleavage activation of CRISPR/Cas effector proteins which results in a self-amplification loop, designated herein as a circular DNA or RNA mediator-induced CRISPR/Cas12a self-amplification loop (CISAL) system. CISAL can be triggered by a single target DNA or RNA, and lead to a dual-amplification scheme including an exponential cleavage-based chain reaction. Such methods can include (a) contacting the sample with (i) a first type V or type VI CRISPR/Cas effector protein; (ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence; (iv) a second type V or type VI CRISPR/Cas effector protein; (v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA- or RNA molecular construct, respectively, by said first type V or type VI CRISPR/Cas effector protein and further activates the nuclease activity of the second type V or type VI CRISPR/Cas effector protein bound to said second guide RNA; (vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; and (b) measuring a detectable signal produced following cleavage of the labelled reporter construct by the first and/or second type V or type VI CRISPR/Cas effector protein, thereby detecting the target in the sample.

The methods of the invention may also be employed for the enhanced detection of target in a sample, wherein the target is not a nucleic acid, through the use of agents that bind to the target (e.g. a target binding construct comprising an antibody and DNA) where binding of the target to the target binding construct permits the activation of a first CRISPR/Cas effector protein which then initiates a CISAL mechanism. In such methods, the activation of a first CRISPR/Cas effector protein can occur through the use of a synthetic trigger nucleic acid sequence not naturally occurring in the sample and which is specifically designed to hybridize with a guide RNA. Such methods can include (a) contacting the sample with (i) a first type V or type VI CRISPR/Cas effector protein; ii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence; (iv) a second type V or type VI CRISPR/Cas effector protein; (v) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA or ssDNA sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; (vi) a labelled reporter construct, wherein said reporter construct comprises a nucleic acid that may or may not hybridize with the guide sequence of the first guide RNA or the guide sequence of the second guide RNA and is cleavable by the nuclease trans-cleavage activity of the activated type V or type VI CRISPR/Cas effector protein; (vii) a target binding construct; and (b) measuring a detectable signal produced following cleavage of the labelled reporter construct by the first and/or second type V or type VI CRISPR/Cas effector protein, thereby detecting the target in the sample; wherein the first type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the target binding construct to thereby co-locate the type V or type VI CRISPR/Cas effector protein and the target when present in the sample.

Also described herein are enhancements to type V and VI CRISPR/Cas-mediated bio-sensing methods. Such methods can include enhancing a type V or type VI CRISPR/Cas detection system comprising adding to a reaction mixture comprising the type V or Type VI CRISPR/Cas effector, and a first guide RNA, of the system: (i) a circular DNA molecular construct comprising both a ssDNA region and a dsDNA sequence, or a circular RNA molecular construct comprising both an ssRNA region and either a ssDNA sequence or a dsDNA sequence; (ii) a second type V or type VI CRISPR/Cas effector protein; (iii) a guide RNA, optionally bound to the second type V or type VI CRISPR/Cas effector protein, said guide RNA comprising: a region that binds to the type V or type VI CRISPR/Cas effector protein, and a guide sequence that hybridizes with the dsDNA sequence of the circular DNA molecular construct or the dsDNA or ssDNA sequence of the circular RNA molecular construct, respectively, wherein hybridization between the guide sequence of the guide RNA and the dsDNA or ssDNA sequence occurs following cleavage of the ssDNA or ssRNA region of the circular DNA or RNA molecular construct, respectively, by the type V or type VI CRISPR/Cas effector protein of the detection system and further activates the nuclease activity of the second type V or type VI CRISPR/Cas effector protein; and (iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein; and measuring a detectable signal produced following cleavage of the labelled reporter construct by the second type V or type VI CRISPR/Cas effector protein. Such methods can include arrangements as described above and in more detail below, wherein a nucleic acid target is detected utilizing a guide RNA designed to hybridize with the target nucleic acid or wherein a non-nucleic acid target is being detected (such as through the use of a target binding construct).

### Type V and Type VI CRISPR/Cas Effector Proteins

Trans-cleavage based programmable nucleases are well known in the literature. They include type V CRISPR/Cas systems and their effector proteins which are a subtype of Class 2 CRISPR/Cas effector proteins (e.g., Cas12 family proteins such as Cas12a), see, e.g., Kira S. Makarova et al., Nat Rev Microbiol. 2020 February; 18, 67-83: “Evolutionary classification of CRISPR-Cas systems: a burst of class 2 and derived variants” and Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e), CasY (Cas12d), Cas12g, Cas12j and Cas12k. Also see, e.g., Kira S. Makarova et al., Nat Rev Microbiol. 2020 February; 18, 67-83: “Evolutionary classification of CRISPR-Cas systems: a burst of class 2 and derived variants” and Koonin et al., Curr Opin Microbiol. 2017 June; 37:67-78: “Diversity, classification and evolution of CRISPR-Cas systems.”. as well as Winston X. Yan et al; “Functionally diverse type V CRISPR-Cas systems”, Science, vol 363, number 6422, pages 88-91 (2019), doi=10.1126/science.aav7271, Type VI CRISPR/Cas systems and their effector proteins (e.g., Cas13 family proteins such as Cas13a), are also described see, e.g., Mol Cell. 2022 January; 82(2):333-347: “The CRISPR-Cas toolbox and gene editing technologies” and Nat Rev Microbiol. 2017 March; 15(3):169-182: “Diversity and evolution of class 2 CRISPR-Cas systems.” Examples include, but are not limited to: Cas13 family (e.g. Cas13a, Cas13b, Cas13c, Cas13d, Cas13X, Cas13Y, and Cas13bt). Such effector proteins are contemplated for use in the present invention.

As such in some embodiments, a subject type V CRISPR/Cas effector protein is a Cas12 protein (e.g., Cas12a, Cas12b, Cas12c). In some embodiments, a subject type V CRISPR/Cas effector protein is a Cas12 protein such as Cas12a, Cas12b, Cas12c, Cas12d, Cas12e, Cas12d, Cas12e, Cas12g, Cas12j or Cas12k. In some embodiments, a subject type V CRISPR/Cas effector protein is a Cas12a protein. In some embodiments, a subject type V CRISPR/Cas effector protein is a Cas12b protein. In some embodiments, a subject type V CRISPR/Cas effector protein is a Cas12c protein. In some embodiments, a subject type V CRISPR/Cas effector protein is a Cas12d protein. In some embodiments, a subject type V CRISPR/Cas effector protein is a Cas12e protein. In some embodiments, a subject type V CRISPR/Cas effector protein is a Cas12g protein. In some embodiments, a subject type V CRISPR/Cas effector protein is a Cas12j protein. In some embodiments, a subject type V CRISPR/Cas effector protein is a Cas12k protein. In some embodiments, a subject type V CRISPR/Cas effector protein is protein selected from: Cas12 (e.g., Cas12a, Cas12b, Cas12c, Cas12d, Cas12e, Cas 12g, Cas12j and Cas12k), C2c4, C2c8, C2c5, C2c10, and C2c9. In some embodiments, a subject type V CRISPR/Cas effector protein is protein selected from: C2c4, C2c8, C2c5, C2c10, and C2c9. In some embodiments, a subject type V CRISPR/Cas effector protein is protein selected from: C2c4, C2c8, and C2c5. In some embodiments, a subject type V CRISPR/Cas effector protein is protein selected from: C2c10 and C2c9. In some embodiments, a subject type VI CRISPR/Cas effector protein is a Cas13 protein (e.g., Cas13a, Cas13b, Cas13c). In some embodiments, a subject type VI CRISPR/Cas effector protein is a Cas13 protein such as Cas13a, Cas13b, Cas13c. In some embodiments, a subject type VI CRISPR/Cas effector protein is a Cas13a protein. In some embodiments, a subject type VI CRISPR/Cas effector protein is a Cas13b protein. In some embodiments, a subject type VI CRISPR/Cas effector protein is a Cas13c protein. In some embodiments, a subject type VI CRISPR/Cas effector protein is a Cas13 protein (e.g., Cas13d, Cas13X, Cas13Y, and Cas13bt).

A variety of Cas12a orthologs originating from different organisms have been identified. For example, Cas12a from Lachnospiraceae bacterium ND2006 (LbCas12a) is the most widely used orthologue for targeted mutagenesis. The Cas12a from Acidaminococcus spec. BV3L6 (AsCas12a) shows high temperature sensitivity. A temperature-insensitive enhanced AsCas12a (enAsCas12a), shows on average a twofold increase in activity at lower temperatures compared with wild-type AsCas12a in human cells. A novel Cas12a nuclease from Coprococcus eutactus (CeCas12a) was identified to be more restrictive in the selection of PAM sequences in vitro and in vivo than AsCas12a and LbCas12a (Chen, P., Zhou. J., Wan. Y. et al. Genome Biol 21, 78 (2020). https://doi.org/10.1186/s13059-020-01989-2). Recently, 16 different ortologs of Cas12a have been identified (Zetsche B, Abudayyeh O O, Gootenberg J S, Scott D A, Zhang F. A Survey of Genome Editing Activity for 16 Cas12a Orthologs. Keio J Med. 2020 Sep. 25; 69(3):59-65. doi: 10.2302/kjm.2019-0009-OA. Epub 2019 Nov. 14. PMID: 31723075; PMCID: PMC7220826.) Similarly, 21 ortologs of Cas13 have been reported (Cox DBT, Gootenberg J S, Abudayyeh O O, Franklin B, Kellner M J, Joung J, Zhang F. RNA editing with CRISPR-Cas13. Science. 2017 Nov. 24; 358(6366):1019-1027. doi: 10.1126/science.aaq0180. Epub 2017 Oct. 25. PMID: 29070703; PMCID: PMC5793859.). In some embodiments, the CRISPR/Cas effector protein is one of the aforementioned orthologs. In another embodiment, the CRISPR/Cas effector protein is a genetically engineered Cas protein with trans-cleavage activity.

In some embodiments, the subject type V or type VI CRISPR/Cas effector protein is a naturally-occurring protein (e.g., naturally occurs in prokaryotic cells). In other embodiments, the Type V or type VI CRISPR/Cas effector protein is not a naturally-occurring polypeptide (e.g., the effector protein is a variant protein, a chimeric protein, includes a fusion partner, and the like). Examples of naturally occurring Type V or type VI CRISPR/Cas effector proteins include, but are not limited to, those described in PCT/US2018/062052. Any Type V or type VI CRISPR/Cas effector protein can be suitable for the methods, compositions, kits, etc. and methods of the present disclosure provided the Type V or type VI CRISPR/Cas effector protein forms a complex with a guide RNA and exhibits nonspecific nuclease activity of a single stranded nucleic acid reporter construct or circular DNA/RNA molecular construct as described herein once it is activated (by hybridization of and associated guide RNA to a trigger nucleic acid sequence).

In some embodiments the Type V or type VI CRISPR/Cas effector protein is immobilized, or otherwise conjugated to a solid surface or substrate. A solid surface or substrate may refer to any material that is suitable for, or may be modified to, the attachment of a polypeptide or polynucleotide. Possible substrates include, but are not limited to, glass and modified functionalized glass, plastic (including acrylics, polystyrene and copolymers of styrene with other materials, polypropylene, polyethylene, polybutylene, polyurethane, Teflon etc.), polysaccharides, nylon or nitrocellulose, ceramics, resins, silica or silica-based materials (including silicon and modified silicon), carbon, metals, inorganic glass, plastics, fiber optic strands, and various other polymers. In some embodiments, the solid support comprises a patterned surface suitable for immobilizing molecules in an ordered pattern. In certain embodiments, a patterned surface refers to an arrangement of distinct regions in or on an exposed layer of a solid support. In some embodiments, the solid support comprises an array of wells (e.g. a microtiter plate) or recesses in the surface. The composition and geometry of the solid support may vary depending on its use. In some embodiments, the solid support is a planar structure, such as a slide, chip, microchip and/or array. Thus, the surface of the substrate may be in the form of a planar layer. In some embodiments, the solid support comprises one or more surfaces of a flow cell. In some embodiments, the solid support or surface thereof is non-planar, such as an inner or outer surface of a tube or container. In some embodiments, the solid support comprises a bead, or a microsphere, or a microparticle, or a nanoparticle. “microsphere,” “bead,” “microparticle”, and “nanoparticle” is intended to mean, in the context of a solid substrate, small discrete particles made from a variety of materials including, but not limited to, metals, plastics, ceramics, glass, and polystyrene or combinations thereof. In certain embodiments, the microspheres are magnetic microspheres or beads. Alternatively, or additionally, the beads may be porous. The beads range in size from nanometers (e.g., 30 nm) to millimeters (e.g., 1 mm). In a preferred embodiment, the bead, microparticle or nanoparticle is magnetic. In embodiments where more than one Type V or type VI CRISPR/Cas effector protein is utilized, either or both of the CRISPR/CAS effector proteins may be immobilized or conjugated to the bead, microparticle or nanoparticle.

### Guide RNA

As used herein, the term “guide sequence”, “guide RNA”, “gRNA”, “CRISPR RNA”, “crRNA” or “guide molecule” refers to a polynucleotide comprising any polynucleotide sequence (including but not limited to modified polynucleotide components, e.g., XNA molecular construct) having sufficient complementarity with either a target or trigger nucleic acid sequence or a dsDNA or ssDNA sequence within a circular DNA or RNA molecular construct (Cir mediator) as described herein, wherein hybridization between with guide RNA and the target or trigger nucleic acid sequence or the dsDNA or ssDNA nucleic acid sequence of the circular DNA or RNA molecular construct activates the nuclease trans-cleavage activity of a CRISPR effector protein complexed with the guide RNA (termed: CRISPR ribonucleoprotein, CRISPR RNP, Cas ribonucleoprotein, Cas RNP).

Where the analyte being detected is not a target nucleic acid sequence within a cell, the guide RNA and the trigger nucleic acid may each be specifically engineered, modified and/or optimized for binding to each other or to the CRISPR/Cas effector protein (in the case of the guide sequence, e.g. guide RNA) or for the activation of the CRISPR/Cas effector protein since there are no constraints imparted by the specific sequence of the target to be selected. Accordingly, in some example embodiments, the degree of complementarity, when optimally aligned using a suitable alignment algorithm, is 99% or more. A guide sequence, and hence a nucleic acid-targeting guide may be selected to target any trigger nucleic acid sequence.

In another embodiment, for methods involving the detection of nucleic acid, the guide RNA is specifically engineered, modified and/or optimized for binding to the desired target nucleic acid sequence.

In some embodiments, a target or trigger nucleic acid-targeting guide RNA is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is rnFold, as described by Zuker and Stiegler (Nucleic Acids Res. 9 (1981), 133-148). Another example folding algorithm is the online Webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, the guide RNA comprises a stem loop, preferably a single stem loop. In certain embodiments, the direct repeat sequence of CRISPR array forms a stem loop, preferably a single stem loop.

Effective guide RNA length, for Cas12a, requires a spacer sequence of at least 10 nucleotides to activate the nuclease function (Cell Research (2018) 28:491-493). The spacer length in gRNA can also affect reaction intensity. For example, the optimal length of a spacer for Cas13b is 26 nt to 34 nt (J. S. Gootenberg et al., Science 10.1126/science.aaq0179 (2018)). Changing the length of guide RNA can also lead to changes of Cas enzymatic activity. For example, extending the 5′-end of Cas12a guide RNA leads to an increase of enzymatic activity (Hyo Min Park et al., Nature Communications (2018) 9:3313; Uyanga Ganbaatar et al., Sensors and Actuators B: Chemical (2022) 369(15) 132296). A truncated Cas9 gRNA leads to improved specificity (Yanfang Fu et al., Nature Biotechnology (2014) 32(3) 279-284). By modifying the guide RNA secondary structures of Cas effectors, such as Cas9 or Cas13a may be also modified, which can increase the system specificity for target nucleic acid sequences (D. D. Kocak et al., Nature Biotechnology (2019) 37, 657-666).

In one embodiment the guide RNA is at least 10 nucleotides in spacer length (Shiyuan Li et al., Cell Research (2018) 28, 491-493). In a preferred embodiment, the guide RNA sequence is 42 nucleotides in length.

The actual sequence of guide RNA can be modified at the terminal, or interval nucleotide. For example, modification of the 5′ or 3′ of Cas9 crRNA leads to improved nuclease stability or activity (McMahon et al., Molecular Therapy (2018) 26(5) 1228-1240, Moon et al., Trends in Biotechnology (2019) 37(8) 870-881, Nguyen et al., Nature Communications (2020) 11, 4906).

In a preferred embodiment the guide RNA comprises a sequence which is sufficiently complementary, including 100% complementary, to the sequence of any of the cDNA or Cir Mediator sequences mentioned in Table 1 below. In another embodiment, the guide RNA comprises a sequence which is sufficiently complementary, including 100% complementary, to the sequence of any of the triggering sequences mentioned below.

In another embodiment, the guide RNA a nucleic acid at least one nucleotide having a different sugar backbone than the naturally occurring nucleic acids in DNA or RNA, that is, at least one nucleotide containing a non-natural sugar (e.g. an XNA).

In one embodiment the guide RNA is circular guide RNA, wherein the circular guide RNA is susceptible to trans-cleavage nuclease activity of a type V or type VI CRISPR/Cas effector protein and comprises a region that binds to a type V or type VI CRISPR/Cas effector protein, and a guide sequence that hybridizes with a target nucleic acid sequence or a trigger nucleic acid sequence, wherein hybridization between the guide sequence and the trigger nucleic acid sequence, only occurs following linearization of the circular guide RNA by the trans-cleavage nuclease activity of a type V or type VI CRISPR/Cas effector protein.

In one embodiment, the circular guide RNA, comprises a total length (circumference) from 40-50 nucleotides. In another embodiment, the guide RNA sequence is 42 nucleotides in length. In a preferred embodiment, the guide RNA sequence is 44 nucleotides in length.

In one embodiment, the circular guide RNA comprises two parts including a 2-5 DNA nucleotides, and the remaining part of the guide RNA comprises a complementary sequence to a target nucleic acid or trigger nucleic acid sequence a sequence which binds to a type V or type VI CRISPR/Cas effector protein. In one embodiment at least two deoxynucleotides are thymidine.

In another embodiment, the circular guide RNA comprises at least one nucleotide containing a non-natural modification/substitution.

### Trigger Nucleic Acid Sequences

As described above, in addition to the utilization of CRISPR/Cas biosensor systems for the detection of target DNA sequences in a sample, the present invention is directed towards the detection of non-nucleic acid targets. In such cases, the sequences of the nucleic acid molecules employed for both the guide RNA and the counterpart trigger nucleic acids which activates the nuclease activity of the CRISPR/Cas effector protein are not dictated by the non-nucleic acid molecules being detected.

The inventors have determined that Cas12a trans-cleavage activity is not significantly suppressed despite terminal modifications to the triggering nucleic acid sequence (e.g. DNA), such as 5′ or/and 3′ attachments, or conjugation of triggering nucleic acid sequences to other molecules such as antibodies (e.g. IgG protein).

Without any additional components or buffer modifications, triggering dsDNA requires the PAM sequence (TTTN, TTN, etc.) to efficiently activate the Cas12 protein, but triggering ssDNA does not require the existence of PAM sequence (Cell Research (2018) 28:491-493). In certain circumstances, triggering of Cas12 proteins by dsDNA leads to higher trans-cleavage activity (Chen et al., Science 360, 436-439 (2018), Cell Research (2018) 28:491-493).

In one embodiment, the triggering nucleic acid sequence has a length of from about 18 nucleotides to about 30 nucleotides in length. In another embodiment, the length of the triggering nucleic acid sequences is about 24 nucleotides. In a preferred embodiment, the length of the triggering nucleic acid sequences is 24 nucleotides. In another embodiment, the length of the triggering nucleic acid sequences is about 30 nucleotides. In a preferred embodiment, the length of the triggering nucleic acid sequences is 30 nucleotides. A length of triggering nucleic acid sequence of greater than 30 nucleotides may still be effective to trigger trans-cleavage of Cas protein and may not impact Cas protein activity unless detrimental secondary structures are formed by the sequence. A length of triggering nucleic acid sequence of shorter than 12 nucleotides may not be effective to trigger the enzymatic activity of a Cas protein.

In one embodiment, the trigger nucleic acid sequence is a double-stranded DNA sequence or RNA sequence.

In one embodiment the trigger nucleic acid sequence comprises a double-stranded DNA sequence. In another embodiment, the trigger nucleic acid sequence comprises a single-stranded RNA sequence. In another embodiment, the trigger nucleic acid sequence comprises a double-stranded RNA sequence, or a hybrid DNA-RNA double strand construct.

In one embodiment, the triggering nucleic acid sequence comprises a nucleic acid sequence where at least one of the nucleotides has a different sugar backbone than the naturally occurring nucleic acids DNA or RNA. That is, at least one nucleotide containing a non-natural sugar (e.g. an XNA).

In a preferred embodiment, the triggering nucleic acid sequence is single stranded DNA.

In one embodiment, the triggering nucleic acid sequence may be the same as a target nucleic acid. In some instances, as the skilled person may readily determine, the terms “target nucleic acid sequence” and “trigger nucleic acid sequence” or “triggering nucleic acid sequence” may be used interchangeably. In another preferred embodiment, the triggering nucleic acid comprises a sequence which is not fully (100%) complementary to any genomic sequences existing in Nature, including but not limited to 5′-CT ATG TGC TAT GTC TAAA A-3′ (SEQ ID NO: 1), 5′-GAA GAC ACC CTA CCA ACC CCC CCC-3′ (SEQ ID NO: 2), and 5′-GAA GAC ACC CTA CCA ACC CCC CCC TAA ACC-3′ (SEQ ID NO: 3).

### Circular DNA or RNA Molecular Constructs (Cir-Mediators and Cir-Amplifiers)

As described herein, the inventors have through extensive studies developed short circular DNA, or hybrid DNA/RNA molecular constructs comprising a ssDNA region and a dsDNA region, or a ssRNA region and either a dsDNA, ssDNA, dsRNA or DNA/RNA hybrid region which may be employed as mediators (Cir-mediators) to control the activation of an additional CRISPR/Cas RNP by generating triggering dsDNA or ssDNA when the ssDNA or ssRNA region of the circular DNA or RNA molecular construct is linearized through the trans-cleavage activity of an already activated CRISPR/Cas RNP (for example: through hybridization of a guide RNA to a target nucleic acid sequence or a synthetic trigger nucleic acid sequence in a sample). The circular DNA, or hybrid DNA/RNA molecular constructs are short and simple structures and do not rely on secondary “blocking” structures or moieties. Accordingly, in other embodiments, the circular DNA, or hybrid DNA/RNA molecular construct as described herein (e.g. Cir-mediator or Cir-amplifier) does not comprise a secondary “blocking” structure or moiety.

Without wishing to be bound by theory, the key property of CRISPR/Cas RNPs which facilitate autocatalysis is its inability to bind and be-activated by such Cir-mediators (and Cir-amplifiers also described herein) in their circular topology—which changes when the topology barrier is overcome by trans-cleavage, and they become linearized. For example, the process of Cas12a RNP activation requires the unwinding of the double helix structure of the target DNA, which is known to be torsionally regulated. RNA-guided DNA recognition occurs by strand separation of a protospacer target to allow Watson-Crick base pairing between the DNA targeted strand and the spacer sequence of a gRNA, and the unwinding of a non-targeted strand. After cis-cleavage, the Cas12a RNP remains bound to the PAM-proximal cleavage product and the RNP undergoes a conformational change enabling trans-cleavage. Thus, the trans-cleavage process is predicated on the formation and dissociation of the R-loop—which requires torque. The Cir-mediators and Cir-amplifiers in the present invention are short, corresponding to approximately one or two coils of the double-helix and about 7 nm long for the circular constructs which are 20 nt. High torsional stress is expected due to small radius of curvature along the length of circle. Furthermore, the closed loop in the dsDNA-containing Cir-mediator or Cir-amplifier makes it rotationally constrained, as the initiation of dsDNA unwinding in one location requires increasing of the winding in adjacent locations, and/or in the ssDNA region—unlike in a corresponding linear structure. Thus, a topological barrier in the Cir-mediator prevents it from releasing torsional stress in a perpendicular direction. At the same time the Cir-mediators are too short to writhe or to supercoil which requires DNA length of ˜20 nm or more. Because the Cir-mediator is expected to have a greater torsional stiffness to its corresponding linear form, it requires more energy to unwind and form the required R-loop structure between gRNA and target DNA for Cas12a RNP activation compared to topologically different standard linear-dsDNA targets.

According to one embodiment, the Cir-mediator is circular DNA molecular construct comprising both a ssDNA region and a dsDNA sequence. In another embodiment, the Cir-mediator is circular RNA molecular construct comprising an ssRNA region and a ssDNA sequence. In another embodiment, the Cir-mediator is a circular RNA molecular construct comprising an ssRNA region and a dsDNA sequence. In another embodiment, the Cir-mediator is a circular RNA molecular construct comprising an ssRNA region and a dsRNA sequence. In another embodiment, the Cir-mediator is a circular RNA molecular construct comprising an ssRNA region and a DNA/RNA hybrid sequence (a double stranded region comprising a DNA strands and a complementary RNA strand). The circular DNA molecular constructs may comprise a circular single strand DNA (ssDNA) and an equal length or slightly shorter linear complementary DNA strand (cDNA) (optionally labelled at both ends with a fluorophore and a matching quencher). These two sequences together create a hybrid circular structure with a ssDNA region and a dsDNA sequence. The skilled person will understand that where the ssDNA region may refer to a ssDNA sequence or ssDNA backbone only. In one embodiment, the hybrid circular structure with a ssDNA region and a dsDNA sequence may comprise a dsDNA sequence joined by a ssDNA backbone (i.e. 0 nt), or a very short ssDNA linker (e.g. 1-7 nt). The skilled person will understand that when a circular DNA molecular construct comprises a ssDNA region being 0 nt in length this refers to a circular dsDNA molecule wherein one of the strands has a free 5′ and free 3′ end; i.e., a circular strand of ssDNA hybridized to complementary strand of ssDNA of equal length—but the ends of that second strand are not joined. The foregoing embodiment may also apply to the circular RNA molecular constructs wherein “DNA” is substituted for “RNA”.

In one embodiment, the Cir-mediator has a total circumference comprising a minimum of 15 nucleotides in length. In another embodiment, the Cir-mediator has a total circumference comprising 30 nucleotides in length or less. In another embodiment, the Cir-mediator has a total circumference comprising, from 16 to 21 nucleotides in length. In another embodiment, the Cir-mediator has a total circumference comprising, from 17 to 20 nucleotides in length. In another embodiment, the Cir-mediator has a total circumference comprising, 15, 16, 17, 18, 19, 20, 21, 22, 23, 24, 25, 26, 27, 28, 29 or 30 nucleotides in length.

In one embodiment, a functional Cir-mediator comprises two parts including a 1-7 nucleotides long ssDNA or ssRNA region, and the remaining part is a either a dsDNA, or ssDNA, dsDNA, dsRNA or DNA/RNA hybrid region, with a complementary sequence to a first gRNA sequence (e.g. including a gRNA which may be utilized in detecting a target nucleic acid sequence with a CRISPR/Cas-based detection system), or a second gRNA of CRISPR/Cas RNPs (e.g. a gRNA different to that employed in detecting a target sequence). In one embodiment the ssDNA or ssRNA region is 1, 2, 3, 4, 5, 6, 7, 8, 9, or 10 nucleotides in length. In one embodiment the ssDNA or ssRNA region is 7 nucleotides or less in length. In one embodiment the ssDNA or ssRNA region is 5 nucleotides or less in length. In one embodiment the ssDNA or ssRNA region is 4 nucleotides or less in length. In one embodiment the ssDNA or ssRNA region is 3 nucleotides or less in length. In one embodiment the ssDNA or ssRNA region is 1 nucleotide in length. In a preferred embodiment, the Cir-mediator comprises a double stranded sequence that is 18 nucleotides in length and a ssDNA or ssRNA sequence that is 2 nucleotides in length. In another embodiment, the Cir-mediator comprises a double stranded sequence that is 18 nucleotides in length and a ssDNA or ssRNA sequence that is 5 nucleotides in length. In one embodiment, the detailed sequence of the Cir-mediator molecular construct preferably comprises a nucleic acid sequence which is not fully (100%) complementary to any genomic sequences existing in Nature. Additionally, the Cir-mediator nucleic acid sequence can also comprise a nucleic acid sequence where at least one of the nucleotides has a modification other than the naturally occurring nucleic acids DNA or RNA, such as having a different sugar backbone. That is, at least one nucleotide, anywhere on the Cir-mediator molecular construct, contains a non-natural sugar (e.g. an XNA). In another embodiment, the Cir-mediator comprises 100% natural or non-modified nucleotides.

As described herein, the inventors have through extensive studies also developed short circular DNA, or hybrid DNA/RNA molecular constructs comprising a short ssDNA sequence that links respective ends of a dsDNA sequence or a short ssRNA sequence that links respective ends of either a dsDNA or ssDNA sequence, wherein the respective ends are labeled such that linearization of the circular DNA, or hybrid DNA/RNA, molecular construct generates a positive detectable signal. In this way, these circular DNA, or hybrid DNA/RNA molecular constructs may be employed as amplifiers (Cir-amplifiers) which not only generate a signal of their own but which also mediate the activation of further Cas RNPs by generating an activating or triggering dsDNA or ssDNA when the ssDNA or ssRNA portion of the circular DNA or RNA molecular construct, respectively, is linearized through the trans-cleavage activity of an already activated CRISPR/Cas RNP (for example: through hybridization of a guide RNA to a target nucleic acid sequence or a synthetic trigger nucleic acid sequence in a sample). In one embodiment, one or more XNAs can replace any of the natural nucleotides of the circular DNA, or hybrid DNA/RNA, molecular constructs. In one embodiment the circular DNA, or hybrid DNA/RNA, molecular construct comprises 100% natural or non-modified nucleotides.

According to one embodiment, the Cir-amplifier is a circular DNA molecular construct comprising both a ssDNA region and a dsDNA sequence. In another embodiment, the Cir-amplifier is circular RNA molecular construct comprising an ssRNA region and a ssDNA sequence (where XNA can replace any of RNA or DNA molecules, single or double strand). In another embodiment, the Cir-amplifier is a circular RNA molecular construct comprising an ssRNA region and a dsDNA sequence (where XNA can replace any of RNA or DNA molecules, single or double strand). In another embodiment, the Cir-amplifier is a circular RNA molecular construct comprising an ssRNA region and a dsRNA sequence. In another embodiment, the Cir-amplifier is a circular RNA molecular construct comprising an ssRNA region and a DNA/RNA hybrid sequence. In one embodiment the circular DNA, or hybrid DNA/RNA, molecular construct comprises 100% natural or non-modified nucleotides.

In one embodiment, the Cir-amplifier of any of the foregoing embodiments has a total circumference comprising a minimum of 15 nucleotides in length. In another embodiment, the Cir-amplifier has a total circumference comprising 30 nucleotides or less in length. In another embodiment, the Cir-amplifier has a total circumference comprising, from 16 to 23 nucleotides in length. In another embodiment, the Cir-amplifier has a total circumference comprising, from 17 to 21 nucleotides in length. In another embodiment, the Cir-amplifier has a total circumference comprising, 15, 16, 17, 18, 19, 20, 21, 22, 23, 24, 25, 26, 27, 28, 29 or 30 nucleotides in length. In one embodiment the ssDNA region is 0 nucleotides in length. In one embodiment the ssDNA or ssRNA region is 1, 2, 3, 4, 5, 6, 7, 8, 9, or 10 nucleotides in length. In one embodiment the ssDNA or ssRNA region is 7 nucleotides or less in length. In one embodiment the ssDNA or ssRNA region is 5 nucleotides or less in length. In one embodiment the ssDNA or ssRNA region is 4 nucleotides or less in length. In one embodiment the ssDNA or ssRNA region is 3 nucleotides or less in length. In one embodiment the ssDNA or ssRNA region is 2 nucleotides or less in length. In one embodiment the ssDNA or ssRNA region is 1 nucleotide in length. In a preferred embodiment, the Cir-amplifier comprises a double stranded sequence that is 18 nucleotides in length and a ssDNA or ssRNA sequence that is 3 nucleotides in length. In another embodiment, the Cir-amplifier comprises a double stranded sequence that is 18 nucleotides in length and a ssDNA or ssRNA sequence that is 5 nucleotides in length. In another embodiment the circular DNA, or hybrid DNA/RNA, molecular construct comprises at least one modified nucleotide. In another embodiment the circular DNA, or hybrid DNA/RNA, molecular construct comprises 100% natural or non-modified nucleotides.

In one embodiment a functional Cir-amplifier comprises two parts including a 2-5 nucleotides long ssDNA or ssRNA region, and the remaining part is a either a dsDNA or ssDNA region, with a complementary sequence to a first gRNA sequence (e.g. including a gRNA which may be utilized in detecting a target nucleic acid sequence with a CRISPR/Cas-based detection system), or a second gRNA of CRISPR/Cas RNPs (e.g. a gRNA different to that employed in detecting a target sequence). In one embodiment, the detailed sequence of the Cir-amplifier molecular construct preferably comprises a nucleic acid sequence which is not fully (100%) complementary to any genomic sequences existing in Nature. In another embodiment, the detailed sequence of the Cir-amplifier molecular construct preferably comprises a nucleic acid sequence which is sufficiently complementary to a gRNA designed to hybridize with a target nucleic acid sequence (e.g. a genomic sequence existing in in nature or a target sequence of interest that has been artificially generated). In one embodiment, the detailed sequence of the Cir-amplifier molecular construct preferably comprises a nucleic acid sequence which is identical to a target sequence to be detected, including naturally occurring (e.g. genomic) sequences. In another embodiment, the Cir-amplifier may comprise a tag and/or detectable moiety (e.g. detectable protein, fluorescent moiety, biotin) which is additional to, or replaces a detectable moiety (e.g. label or fluorophore etc.) or a moiety which blocks, masks, quenches or inhibits the detectable. In another embodiment the tag or detectable moiety is attached via a linking single-stranded nucleic acid structure which can be cleaved by the trans-cleavage activity of an activated Type V or Type VI Cas protein. The skilled person will be able to readily determine the appropriate length and sequence of the linking single-stranded nucleic acid structure. In one embodiment, linking single-stranded nucleic acid structure is a nucleic acid sequence of 1-10 nt in length. In another embodiment the single-stranded nucleic acid structure is 5 nt in length. In another embodiment, the single-stranded nucleic acid structure comprised of identical nucleotides. In one embodiment the tag is biotin linked to the Cir amplifier by an additional sequence of 5 identical nucleotides.

In one embodiment, the Cir-amplifier comprises a fluorophore (e.g. FAM) and a linking single-stranded nucleic acid sequence “tail” comprising biotin on the 3′ end (i.e. “linking” the biotin to the Cir-amplifier). In a preferred embodiment the linking single stranded nucleic acid sequence comprises 5 identical nucleotides. In another preferred embodiment the linking single stranded nucleic acid sequence is ssDNA. In another preferred embodiment, the linking single stranded nucleic acid sequence is CCCCC. In one embodiment the tagged Cir-amplifiers may be employed in a lateral flow assay. In an exemplary arrangements the biotinylated Cir-amplifier is detected by capture by streptavidin immobilised on a “control” line which produces a colour on the control line e.g. due to simultaneous presence of Au NPs in these products, as well as a detection protein conjugate and antibodies thereto (e.g. FAM and anti-FAM antibodies). With further flow of the sample, a secondary antibody on the “test” line captures anti-FAM antibodies on biotin-free products (i.e. in the presence of activated CRISPR/Cas effector protein (e.g. Cas 12a), i.e. when the target is present, the biotinylated single stranded nucleic acid sequence (“tail”) is cleaved and freed and the Cir-amplifier comprising the detection protein conjugate is released to accumulate at the test line of the lateral flow strip for colorimetric signal readout.

Additionally, the Cir-amplifier nucleic acid sequence can also comprise a nucleic acid sequence where at least one of the nucleotides has a modification other than the naturally occurring nucleic acids DNA or RNA, such as having a different sugar backbone. That is, at least one nucleotide, anywhere on the Cir-amplifier molecular construct, contains a non-natural sugar (e.g. an XNA).

Exemplary arrangements of labeled nucleic acid elements that may prevent or mask the generation of a detectable signal will be known to the skilled person and exemplary embodiments are described below, and embodiments of the invention include these or variants thereof. Prior to linearization of a Cir amplifier, or when the Cir amplifier is not in an “active” state, the Cir amplifier can be designed so that the generation or detection of a positive detectable signal is blocked, masked, quenched or inhibited. It will be appreciated that in certain exemplary embodiments, minimal background signal may be generated in the presence of non-linearized Cir amplifiers. The positively detectable signal can be any signal that can be detected using optical, fluorescent, colorimetric, chemiluminescent, electrochemical or other detection methods known in the art. The term “positive detectable signal” is used to distinguish between other detectable signals detectable in the presence of non-linearized Cir amplifiers. For example, in certain embodiments, a first signal (i.e., a negative detectable signal) can be detected when a masking or quenching agent is present, which is then converted to a second signal (e.g., a positive detectable signal) when the target molecule is detected and the masking or quenching agent is removed or translocated distally to the detectable label upon linearization of the Cir amplifier by an activated Cas RNP.

In another embodiment, the Cir-amplifier comprises an electrochemically detectable moiety. In one embodiment the electrochemically detectable moiety is methylene blue.

In another embodiment, the Cir-amplifier may comprise a biotin tag attached via a linking single-stranded nucleic acid structure which can be cleaved by the trans-cleavage activity of an activated Type V or Type VI Cas protein and an electrochemically detectable moiety. In one embodiment the electrochemically detectable moiety is methylene blue.

In certain other exemplary embodiments, the Cir-amplifier may comprise an RNA, a DNA or a modified or RNA or DNA, comprising one or more Xeno Nucleic Acids (XNA) or artificial nucleotides, to which a detectable label is attached and a masking or quenching agent for the detectable label. Examples of such detectable label/masking agent pairs are fluorophores and quenchers of fluorophores. Quenching of a fluorophore can occur due to the formation of a non-fluorescent complex between the fluorophore and another fluorophore or a non-fluorescent molecule. This mechanism is called ground state complex formation, static quenching or contact quenching. Thus, an RNA or DNA oligonucleotide can be designed such that the fluorophore and quencher are sufficiently close for contact quenching to occur. Fluorophores and their associated quenchers are known in the art and can be selected by one of ordinary skill in the art for this purpose. The particular fluorophore/quencher is not critical in the context of the present invention, so long as the fluorophore/quencher pair is selected to ensure masking of the fluorophore. Upon activation of the Cas RNPs disclosed herein, the RNA or DNA or XNA the Cir-amplifier is linearized, thereby severing the proximity between the fluorophore and quencher needed to maintain the contact quenching effect. Thus, detection of a fluorophore can be used to determine the presence of the target molecule in a sample.

In a preferred embodiment the Cir-amplifier comprises a dsDNA sequence labelled with the Fluorophore FAM (e.g. 5′) and a suitable matching quencher BHQ1 (e.g. 3′). In another preferred embodiment, the Cir-amplifier comprises a dsDNA labelled with the Fluorophore Texas Red (e.g. 5′) and a suitable matching quencher BHQ2 (e.g. 3′).

The circular DNA or RNA molecular constructs (Cir mediators or Cir amplifiers) may also be a Xeno nucleic acid (XNA) construct which includes one or more, or consists of Xeno nucleic acids or artificial nucleotides. A Xeno nucleic acid or artificial nucleotide may comprise a non-naturally occurring sugar or nucleobase.

The circular DNA or RNA molecular constructs may be synthesized using methods with which the skilled person will be familiar. In one embodiment the synthesis of the circular DNA or RNA molecular constructs is performed using a DNA ligase. In one embodiment, a linear ssDNA oligo is combined with a ssDNA linker oligo and T4 ligase in an appropriate buffer and a cyclization reaction performed. Unbound linear ssDNA and linker oligos in the product of the cyclization reaction can then be removed with an appropriate exonuclease (e.g. exonuclease III). The circular ssDNA (“Cir-ssDNA”) produced can then be combined with a shorter complementary DNA (cDNA) with PAM sequence, to produce a ssDNA/dsDNA circular DNA molecular construct.

In another embodiment, the circular DNA or RNA molecular constructs may be synthesized using click chemistry. Click chemistry refers to reactions that are high yielding, wide in scope, create only byproducts that can be removed without chromatography, are stereospecific, simple to perform, and can be conducted in easily removable or benign solvents. Several types of reaction have been identified that fulfill these criteria, including conjugate addition, strained ring opening, acylation/sulfonylation, aldehyde capture by α-effect nucleophiles, and copper-mediated azide-alkyne cycloaddition. The inventors have surprisingly found that the binding forces of functional groups used in click chemistry are strong enough to make circular a short and rigid piece of mostly dsDNA. The inventors have further found in developing the Cir-mediators and Cir-amplifiers described herein, that very small circular molecules with a total length of less than 15 nt cannot be formed. In view of this lower limit on the length of Cir-mediators and Cir-amplifiers, on the one hand and an upper limit on the effectiveness of Cir-mediators and Cir-amplifiers to minimally activate Cas nucleases, it is unexpected that there are circular structures that satisfy both these conditions simultaneously—they are suitable to be Cir-mediators and Cir-amplifiers, and that, as presented herein, such short structures can be made by click chemistry.

Accordingly, in one embodiment, the Cir-mediators and Cir-amplifiers described herein are produced using click chemistry. In one embodiment the synthesis of the circular DNA or RNA molecular constructs is performed by immobilizing linear-ssDNA on a solid surface or substrate (e.g. a magnetic bead) subjecting the immobilized ssDNA to a click chemistry reaction. Following removal of excess chemicals from the click chemistry reaction remaining linear ssDNA may then be removed with an appropriate exonuclease (e.g. exonuclease III). The circular ssDNA (“Cir-ssDNA”) produced can then be released from the surface or substrate to which they have been immobilized. The Cir-ssDNA may then be combined with a shorter complementary DNA (cDNA) with PAM sequence, to produce a ssDNA/dsDNA circular DNA molecular construct.

In one aspect, the present invention relates to a method for producing a circular DNA or RNA molecular construct as described herein, comprising: immobilizing a linear-ssDNA on a solid surface or substrate; subjecting the immobilized ssDNA to a click chemistry reaction to form circular ssDNA (Cir-ssDNA); removing excess chemicals from the click chemistry reaction; digesting remaining linear ssDNA with an exonuclease; releasing the circular ssDNA from the surface or substrate to which they have been immobilized. In one embodiment, the method further comprises combining the released Cir-ssDNA with a shorter complementary DNA with a PAM sequence, to produce a ssDNA/dsDNA circular DNA molecular construct. In another embodiment, the ssDNA/dsDNA circular DNA molecular construct, is produced by mixing Cir-ssDNA and a shorter complementary DNA and incubating the mixture at about 95° C. for about 5 min. In one embodiment, the surface or substrate is a magnetic bead. In another embodiment, the surface or substrate streptavidin-modified and the ssDNA biotinylated so as to immobilize the ssDNA. In an embodiment, releasing the circular ssDNA is by heat treatment. In one embodiment, heat treatment comprises subjecting the immobilized ssDNA to heat treatment at about 95° C. for about 30 minutes.

In certain embodiments, the ssDNA/dsDNA Cir-mediators comprises a sequence selected from the “Cir-Medi” and cDNA sequences recited in Table 1 below:

TABLE 1
Exemplary Cir-Mediator sequences

| | | 5′- | 3′- | Internal | SEQ ID |
|---|---|---|---|---|---|
| Example | Sequence | modification | modification | modification | NO: |
| Cir- | T ATG TGC TAT GTC TAAA A | 5′P | | | SEQ ID |
| Medi-1 | | | | | NO: 4 |
| cDNA-1 | TTTA GAC ATA GCA CAT | | | | SEQ ID |
| | | | | | NO: 5 |
| Linker-1 | GCA CAT A T TTTA | | | | SEQ ID |
| | | | | | NO: 6 |
| Cir- | CT ATG TGC TAT GTC TAAA | 5′P | | | SEQ ID |
| Medi-2 | A | | | | NO: 7 |
| cDNA-2 | TTTA GAC ATA GCA CAT A | | | | SEQ ID |
| | | | | | NO: 8 |
| Linker-2 | CA CAT AG T TTTA | | | | SEQ ID |
| | | | | | NO: 9 |
| Cir- | TCT ATG TGC TAT GTC TAAA | 5′P | | | SEQ ID |
| Medi-3 | A | | | | NO: 10 |
| cDNA-3 | TTTA GAC ATA GCA CAT AG | | | | SEQ ID |
| | | | | | NO: 11 |
| Linker-3 | A CAT AGA T TTTA | | | | SEQ ID |
| | | | | | NO: 12 |
| Cir- | TTCTATGTGCTATGTCTAAAA | 5′P | | | SEQ ID |
| Medi-4 | | | | | NO: 13 |
| cDNA-4 | TTTA GAC ATA GCA CAT | | | | SEQ ID |
| | AGA | | | | NO: 14 |
| Linker-4 | CATAGAA T TTTA | | | | SEQ ID |
| | | | | | NO: 15 |
| Cir- | T CAG TCT ATG TGC TAT | 5′P | | | SEQ ID |
| Medi-5 | GTC TAAA A | | | | NO: 16 |
| cDNA-5 | TTTA GAC ATA GCA CAT | | | | SEQ ID |
| | AGA CTG | | | | NO: 17 |
| Linker-5 | AGA CTG A T TTTA G | | | | SEQ ID |
| | | | | | NO: 18 |
| Cir- | T TCT CAG TCT ATG TGC | 5′P | | | SEQ ID |
| Medi-6 | TAT GTC TAAA A | | | | NO: 19 |
| cDNA-6 | TTTA GAC ATA GCA CAT | | | | SEQ ID |
| | AGA CTG AGA | | | | NO: 20 |
| Linker-6 | CTG AGA A T TTTA G | | | | SEQ ID |
| | | | | | NO: 21 |
| Cir- | TATGTGCTATGTCTAAAA | 5′Azide(N3) | 3′CHCH | Int Biotin | SEQ ID |
| Medi-7 | | | | dT | NO: 22 |
| Cir- | CT ATG TGC TAT GTC TAAA | 5′Azide(N3) | 3′CHCH | Int Biotin | SEQ ID |
| Medi-8 | A | | | dT | NO: 23 |
| Cir- | TCT ATG TGC TAT GTC TAAA | 5′Azide(N3) | 3′CHCH | Int Biotin | SEQ ID |
| Medi-9 | A | | | dT | NO: 24 |
| Cir- | TCT CAG TCT ATG TGC | 5′Azide(N3) | 3′CHCH | Int Biotin | SEQ ID |
| Medi-10 | TAT GTC TAA A | | | dT | NO: 25 |
| cDNA- | TTT AGA CAT AGC ACA T | | | | SEQ ID |
| 10 | | | | | NO: 26 |

In certain embodiments, the Cir-amplifiers comprise oligonucleotides, optionally including the same modifications, as those described in Tables S1-S7.

### Palindromic Oligonucleotide Constructs (T-Locker)

As described herein, the inventors have through extensive studies synthesised short single stranded oligonucleotides with a palindromic sequence or quasipalindromic sequence (“palindromic oligo”) which hybridizes via intramolecular binding to form a double stranded structure having a sealed end (e.g. a hairpin structure). In one embodiment the palindromic oligos are an inverted repeats without a centrally spaced nucleotide or nucleotides (i.e. without a “spacer”). In one embodiment the palindromic oligos are inverted repeats including 1-10, preferably 1-3, intervening nucleotides (a “spacer”) or a “quasipalindromic oligo”. As used herein, the term “palindromic oligo” encompasses oligonucleotides having a palindromic sequence and oligonucleotides having a quasipalindromic sequence. The palindromic oligos can form a hairpin secondary structure comprising a double stranded sequence having a sealed end through intramolecular binding of the palindromic sequences. For example, when a spacer is present the 3′ end may be sealed by the 1-10, preferably 1-3, “spacer” nucleotides through intramolecular binding of the palindromic sequences. The inventors have observed that after the 3′ sealed structure is formed, termed the 3′-tail sealed locker for Cas12a activation (T-locker), it was found to prevent the formation of the R-loop structure within the Cas12a RNP, and hence exhibited restricted CRISPR/Cas12a activation. The palindromic oligos may therefore be employed as mediators to control the activation of an additional CRISPR/Cas RNP by generating triggering dsDNA when the 3′ sealed structure is cleaved via the trans-cleavage activity of an already activated CRISPR/Cas RNP (for example: through hybridization of a guide RNA to a target nucleic acid sequence in a sample or a synthetic trigger nucleic acid sequence in a sample). The palindromic oligo constructs may be formed from ssDNA, ssRNA or DNA/RNA hybrids.

In one embodiment, the palindromic oligos may be synthesized as a single stranded molecule comprising the following arrangement: i) a palindromic sequence proximal to each of its terminals, wherein the palindromic sequence optionally includes a PAM sequence (TTTN, TTN, etc.), and optionally ii) a spacer consisting of 1-3 nucleotides disposed between the palindromic sequences, wherein the PAM sequence, when present, is proximal to the terminal ends of the molecule and distal to the spacer, when present. In one embodiment, the palindromic oligonucleotide comprises a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and optionally wherein the first sequence includes a PAM sequence and the second sequence includes the complementary sequence, and wherein the PAM sequence is located towards the 5′ end of the first sequence, and distal to the sealed end, and wherein the first or second sequence is sufficiently identical to a target nucleic acid sequence or a trigger nucleic acid sequence and specifically hybridizes with a guide RNA of a CRISPR/Cas RNP.

In one embodiment, the first sequence and/or second sequence of the palindromic oligo is from 10 to 30 nucleotides in length (i.e. yielding from 10 to 30 bp when secondary structure is formed by intramolecular binding). In another embodiment the first sequence and/or second sequence of the palindromic oligo is from 16 to 21 nucleotides in length. In another embodiment, the first sequence and/or second sequence of the palindromic oligo is from 17 to 20 nucleotides in length. In another embodiment, the first sequence and/or second sequence of the palindromic oligo is 15 nucleotides in length. In another embodiment, the first sequence and/or second sequence of the palindromic oligo is 10, 11, 12, 13, 14, 15, 16, 17, 18, 19, 20, 21, 22, 23, 24, 25, 26, 27, 28, 29 or 30 nucleotides in length.

In one embodiment, the palindromic oligo comprises the following structure (in the 5′-to-3′ direction): a) PAM sequence, b) target sequence, c) 0, or 1-3 spacer nucleotide(s), d) sequence complementary to b), and e) sequence complementary to a). The target sequence may be identical to a target nucleic acid sequence or a trigger sequence to be detected in a sample. In another embodiment, the palindromic oligo further comprises a sequence of nucleotides which precedes a) and/or follows e), optionally wherein the sequence preceding a) and the sequence following e) are complementary.

In one embodiment, the palindromic oligo is represented by the formula:

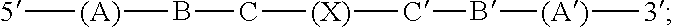

wherein
A is absent or from 1-100 nucleotides in length,
B is a PAM sequence (e.g. 5′-TTTN, 5′-TTN) or absent,
C is a sequence that targets a Cas RNP and is from 8-30 nt in length,
X is either absent or from 1-10, preferably from 1-3, nucleotides in length and not complementary to B, C, B′ or C′,
C′ is at least 75%, at least 80%, at least 85%, at least 90%, at least 95% or 100% complementary to C,
B′ is at least 75%%, at least 80%, at least 85%, at least 90%, at least 95% or 100% complementary to B, and
A′ is absent or from 1-100 nucleotides in length.
   In one embodiment, A′ is fully complementary to A. In one embodiment, A′ is partially complementary to A. In one embodiment A is present, while A′ is absent.

In one embodiment X is present and is 1 nucleotide in length. In another embodiment, X is present and is 2 nucleotides in length. In another embodiment, X is present and is 3 nucleotides in length.

In one embodiment the PAM sequence is selected from the group consisting of TTTA, TTTC, and TTTG.

When the secondary structure of the palindromic oligo is formed, a double stranded PAM will result, Advantageously, once the seal has been cleaved the palindromic oligo remains double stranded sequence which may activate a Cas/RNP enabling effective autocatalysis of Cas/RNPs. Unlike a single stranded trigger which will be effectively trans-cleaved which will, in turn, lead to reduced Cas/RNP activation efficiency, the “unlocked” palindromic oligo remains double stranded and thereby largely unaffected by the trans-cleavage activity of an activated Cas/RNP, leading to effective autocatalysis.

In one embodiment, the target sequence of the palindromic oligo is from 10 to 30 nucleotides in length (i.e. yielding from 10 to 30 bp when secondary structure is formed by intramolecular binding). In another embodiment the target sequence of the palindromic oligo comprises is from 16 to 21 nucleotides in length. In another embodiment, the target sequence of the palindromic oligo is from 17 to 20 nucleotides in length. In another embodiment, the target sequence of the palindromic oligo is 15 nucleotides in length. In another embodiment, the target sequence of the palindromic oligo is 10, 11, 12, 13, 14, 15, 16, 17, 18, 19, 20, 21, 22, 23, 24, 25, 26, 27, 28, 29 or 30 nucleotides in length.

In one embodiment the palindromic oligo comprises 1 nucleotide in the spacer. In one embodiment the palindromic oligo comprises 2 nucleotides in the spacer. In one embodiment the palindromic oligo comprises 3 nucleotides in the spacer.

In one embodiment, the target sequence of the palindromic oligo has a sequence which is complementary to a first gRNA sequence (e.g. including a gRNA which may be utilized in detecting a target nucleic acid sequence in a sample with a CRISPR/Cas-based detection system), or a second gRNA of a second CRISPR/Cas RNP (e.g. a gRNA different to that employed in detecting a target sequence).

In one embodiment, the target sequence of the palindromic oligo preferably comprises a nucleic acid sequence which is not fully (100%) complementary to any genomic sequences existing in Nature. Additionally, the palindromic oligo can also comprise a nucleic acid sequence where at least one of the nucleotides has a modification other than the naturally occurring nucleic acids DNA or RNA, such as having a different sugar backbone. That is, at least one nucleotide, anywhere in oligo, contains a non-natural sugar (e.g. an XNA). In another embodiment, the palindromic oligo comprises 100% natural or non-modified nucleotides.

In one embodiment, the 5′, 3′ and/or any internal nucleotides of the palindromic oligo can be labelled with a chemical group or molecule. In one embodiment, the chemical group or molecule is a tag and/or detectable moiety (e.g. detectable protein, fluorescent moiety, biotin etc.) or a moiety which blocks, masks, quenches or inhibits the detectable moiety. In one embodiment the palindromic oligo comprises a detectable label/masking agent pair. In one embodiment, the detectable label/masking agent pair is a fluorophore and a quencher of the fluorophore. Again, as explained above, fluorophores and their associated quenchers are known in the art and can be selected by one of ordinary skill in the art for this purpose. The particular fluorophore/quencher is not critical in the context of the present invention, so long as the fluorophore/quencher pair is selected to ensure masking of the fluorophore. Upon activation of the Cas RNPs disclosed herein, after the palindromic oligo having a sealed end is “unsealed” via trans-cleavage by an activated Cas RNP, it may then participate in the formation of the R-loop structure within the Cas12a RNP, thereby severing the proximity between the fluorophore and quencher needed to maintain the quenching effect.

In one embodiment the palindromic oligo comprises a DNA sequence labelled with the Fluorophore FAM (e.g. 5′) and a suitable matching quencher BHQ1 disposed downstream (e.g. 3′). In another embodiment, the palindromic oligo comprises a DNA sequence labelled with the Fluorophore Texas Red (e.g. 5′) and a suitable matching quencher BHQ2 disposed downstream (e.g. 3′). In one embodiment, the spacer of a palindromic oligo is labeled (e.g. with a fluorophore or quencher, where the 5′ or 3′ terminal end is labeled with a quencher or fluorophore, respectively).

In another embodiment, the target sequence of the palindromic oligo preferably comprises a nucleic acid sequence which is sufficiently complementary to a gRNA designed to hybridize with a target nucleic acid sequence (e.g. a genomic sequence existing in nature or a target sequence of interest that has been artificially generated). In one embodiment, the target sequence of the palindromic oligo preferably comprises a nucleic acid sequence which is identical to a target sequence to be detected, including naturally occurring (e.g. genomic) sequences.

In one embodiment the palindromic oligo is comprised of DNA. In another embodiment the palindromic oligo is comprised of RNA. In another embodiment the palindromic oligo is a DNA/RNA hybrid. In another embodiment, the palindromic oligo is a Xeno nucleic acid (XNA) construct which includes one or more, or consists of Xeno nucleic acids or artificial nucleotides. A Xeno nucleic acid or artificial nucleotide may comprise a non-naturally occurring sugar or nucleobase.

In one embodiment there is provided a method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a labelled reporter construct, wherein said reporter construct comprises a nucleic acid that is cleavable by the nuclease activity of the activated type V or type VI CRISPR/Cas effector protein; and
(iv) a palindromic oligonucleotide comprising a single stranded sequence of nucleotides comprising a first sequence of nucleotides followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the first guide RNA and also activates the nuclease activity of first type V or type VI CRISPR/Cas effector proteins bound to said first guide RNA following cleavage of the sealed end by the nuclease activity of said first type V or type VI CRISPR/Cas effector protein and;
(b) measuring a detectable signal produced following cleavage of the labelled nucleic acid reporter construct by the first type V or type VI CRISPR/Cas effector protein, thereby detecting the target nucleic acid in the sample.

In another embodiment, there is provided a method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
iii) a second type V or type VI CRISPR/Cas effector protein;
(iv) a second guide RNA, optionally in association with said second type V or type VI CRISPR/Cas effector protein, comprising: a region that binds to said second type V or type VI CRISPR/Cas effector protein, and a guide sequence;
(v) a palindromic oligonucleotide comprising a single stranded sequence of nucleotides comprising a first sequence of nucleotides followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vi) a labelled reporter construct, wherein said reporter construct comprises a nucleic acid that is cleavable by the nuclease activity of the activated type V or type VI CRISPR/Cas effector protein; and
(b) measuring a detectable signal produced following cleavage of the labelled nucleic acid reporter by the first and/or second type V or type VI CRISPR/Cas effector protein, thereby detecting the target in the sample.

In another embodiment, there is provided a method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the first guide RNA and also activates the nuclease activity of said first type V or type VI CRISPR/Cas effector proteins bound to said first guide RNA following cleavage of the sealed end by the nuclease activity of said first type V or type VI CRISPR/Cas effector protein;
(v) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(vi) a target binding construct; and
(b) measuring a detectable signal produced following cleavage of the labelled nucleic acid reporter construct by the first type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

In another embodiment, there is provided a method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence,
(vi) a palindromic oligonucleotide comprising a single stranded sequence of nucleotides comprising a first sequence of nucleotides followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first and second type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

In another embodiment, there is provided a method of enhancing a type V or type VI CRISPR/Cas detection system, which comprises a type V or Type VI CRISPR/Cas effector protein, a first guide RNA, and a labelled nucleic acid reporter, comprising:
adding to a reaction mixture comprising a Type V or Type VI CRISPR/Cas effector protein of the detection system a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with a guide sequence of a guide RNA of said type V or type VI CRISPR/Cas detection system and following cleavage of the sealed end by the nuclease activity of said first type V or type VI CRISPR/Cas effector protein further activates the nuclease activity of said type V or type VI CRISPR/Cas effector proteins bound to said guide RNA; and
measuring a detectable signal produced following linearization of the circular DNA molecular construct, or the circular RNA molecular construct by the type V or type VI CRISPR/Cas effector protein of said detection system.

In another embodiment, there is provided a method of enhancing a type V or type VI CRISPR/Cas detection system, which comprises a first type V or Type VI CRISPR/Cas effector protein, a first guide RNA, and a labelled nucleic acid reporter, comprising:
adding to a reaction mixture comprising at least a first type V or Type VI CRISPR/Cas effector of the system:
(i) a second type V or type VI CRISPR/Cas effector protein;
(ii) a second guide RNA, optionally bound to the second type V or type VI CRISPR/Cas effector protein, said guide RNA comprising: a region that binds to the second type V or type VI CRISPR/Cas effector protein, and a guide sequence,
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with a guide sequence of the second guide RNA and following cleavage of the sealed end by the nuclease activity of said first type V or type VI CRISPR/Cas effector protein further activates the nuclease activity of said second type V or type VI CRISPR/Cas effector proteins bound to said second guide RNA; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein;
and measuring a detectable signal produced following cleavage of the labelled nucleic acid reporter of the detection system, and/or the labelled reporter construct when added, by the first and/or second type V or type VI CRISPR/Cas effector protein.

In one embodiment there is provided a method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a labelled reporter construct, wherein said reporter construct comprises a nucleic acid that is cleavable by the nuclease activity of the activated type V or type VI CRISPR/Cas effector protein; and
(iv) a palindromic oligonucleotide comprising a single stranded sequence of nucleotides comprising a first sequence of nucleotides followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, wherein the palindromic oligo is detectably labelled, and wherein the double stranded structure specifically hybridizes with the first guide RNA and also activates the nuclease activity of first type V or type VI CRISPR/Cas effector proteins bound to said first guide RNA following cleavage of the sealed end by the nuclease activity of said first type V or type VI CRISPR/Cas effector protein, and wherein the palindromic oligo is detectably labelled, and;
(b) measuring a detectable signal produced following cleavage of the palindromic oligo by the first type V or type VI CRISPR/Cas effector protein, thereby detecting the target nucleic acid in the sample.

In another embodiment, there is provided a method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
iii) a second type V or type VI CRISPR/Cas effector protein;
(iv) a second guide RNA, optionally in association with said second type V or type VI CRISPR/Cas effector protein, comprising: a region that binds to said second type V or type VI CRISPR/Cas effector protein, and a guide sequence;
(v) a palindromic oligonucleotide comprising a single stranded sequence of nucleotides comprising a first sequence of nucleotides optionally followed downstream by a spacer consisting of 1-3 nucleotides, followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein, and wherein the palindromic oligo is detectably labelled; and
(b) measuring a detectable signal produced following cleavage of the palindromic oligo by the first and/or second type V or type VI CRISPR/Cas effector protein, thereby detecting the target in the sample.

In another embodiment, there is provided a method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the first guide RNA and also activates the nuclease activity of said first type V or type VI CRISPR/Cas effector proteins bound to said first guide RNA following cleavage of the sealed end by the nuclease activity of said first type V or type VI CRISPR/Cas effector protein, and wherein the palindromic oligo is detectably labelled;
(vii) a target binding construct; and
(b) measuring a detectable signal produced following cleavage of the palindromic oligo by the first type V or type VI CRISPR/Cas effector protein, thereby detecting the target in the sample; wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

In another embodiment, there is provided a method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence,
(vi) a palindromic oligonucleotide comprising a single stranded sequence of nucleotides comprising a first sequence of nucleotides followed downstream by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein, and wherein the palindromic oligo is detectably labelled;
(vii) a target binding construct; and
(b) measuring a detectable signal produced following cleavage of the palindromic oligo by the first and/or second type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

In another embodiment, there is provided a method of enhancing a type V or type VI CRISPR/Cas detection system, which comprises a type V or Type VI CRISPR/Cas effector protein, a first guide RNA, and a labelled nucleic acid reporter, comprising:
adding to a reaction mixture comprising a Type V or Type VI CRISPR/Cas effector protein of the detection system a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with a guide sequence of a guide RNA of said type V or type VI CRISPR/Cas detection system and following cleavage of the sealed end by the nuclease activity of said first type V or type VI CRISPR/Cas effector protein further activates the nuclease activity of said type V or type VI CRISPR/Cas effector proteins bound to said guide RNA, and wherein the palindromic oligo is detectably labelled; and
measuring a detectable signal produced following cleavage of the palindromic oligo and/or trans-cleavage of the labelled nucleic acid reporter of said detection system by the type V or type VI CRISPR/Cas effector protein of said detection system.

In another embodiment, there is provided a method of enhancing a type V or type VI CRISPR/Cas detection system, which comprises a first type V or Type VI CRISPR/Cas effector protein, a first guide RNA, and a labelled nucleic acid reporter, comprising:
adding to a reaction mixture comprising at least a first type V or Type VI CRISPR/Cas effector of the system:
(i) a second type V or type VI CRISPR/Cas effector protein;
(ii) a second guide RNA, optionally bound to the second type V or type VI CRISPR/Cas effector protein, said guide RNA comprising: a region that binds to the second type V or type VI CRISPR/Cas effector protein, and a guide sequence,
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence; and wherein the first sequence includes a PAM sequence and the second sequence includes a sequence complementary to said PAM sequence, and wherein the PAM sequence is distal to the sealed end, and wherein the double stranded structure specifically hybridizes with a guide sequence of the second guide RNA and following cleavage of the sealed end by the nuclease activity of said first type V or type VI CRISPR/Cas effector protein further activates the nuclease activity of said second type V or type VI CRISPR/Cas effector proteins bound to said second guide RNA, and wherein the palindromic oligo is detectably labelled;
and measuring a detectable signal produced following cleavage of the palindromic oligo and/or trans-cleavage of the labelled nucleic acid reporter of the detection system, by the first and/or second type V or type VI CRISPR/Cas effector protein.

In another embodiment of the foregoing methods involving a palindromic oligonucleotide, the palindromic oligonucleotide comprises a spacer consisting of 1-3 nucleotides disposed between said first sequence and said second sequence.

In certain embodiments, the palindromic oligo comprises a sequence selected from the sequences recited in Table 2 below:

TABLE 2
Exemplary T-locker sequences

| | Oligo | | | SEQ |
|---|---|---|---|---|
| Name | type | Sequence 5′-3′* | Modification | ID NO: |
| T-locker | ssDNA | TTTA GAC ATA GCA CAT | 5′-biotin | SEQ ID NO: |
| | | AGA CTG CAG TCT ATG | | 27 |
| | | TGC TAT GTC TAAA | | |
| T-locker+1nt | ssDNA | TTTA GAC ATA GCA CAT | 5′-biotin | SEQ ID NO: |
| | | AGA CTG C CAG TCT ATG | | 28 |
| | | TGC TAT GTC TAAA | | |
| T-locker+2nt | ssDNA | TTTA GAC ATA GCA CAT | 5′-biotin | SEQ ID NO: |
| | | AGA CTG CC CAG TCT | | 29 |
| | | ATG TGC TAT GTC TAAA | | |
| T-locker+3nt | ssDNA | TTTA GAC ATA GCA CAT | 5′-biotin | SEQ ID NO: |
| | | AGA CTG CCC CAG TCT | | 30 |
| | | ATG TGC TAT GTC TAAA | | |
| T-locker-1nt | ssDNA | TTTA GAC ATA GCA CAT | 5′-biotin | SEQ ID NO: |
| | | AGA CT CAG TCT ATG | | 31 |
| | | TGC TAT GTC TAAA | | |
| T-locker-2nt | ssDNA | TTTA GAC ATA GCA CAT | 5′-biotin | SEQ ID NO: |
| | | AGA CT AG TCT ATG TGC | | 32 |
| | | TAT GTC TAAA | | |
| T-locker-3nt | ssDNA | TTTA GAC ATA GCA CAT | 5′-biotin | SEQ ID NO: |
| | | AGA C CG TCT ATG TGC | | 33 |
| | | TAT GTC TAAA | | |
| T-locker | ssDNA | TTTA GAC ATA GCA CAT | 5′-biotin | SEQ ID NO: |
| (−1 bp) + 3 nt | | AGA CG CCC CG TCT ATG | | 34 |
| | | TGC TAT GTC TAAA | | |
| T-locker | ssDNA | TTTA GAC ATA GCA CAT | 5′-biotin | SEQ ID NO: |
| (−3 bp) + 3 nt | | AGC CCC GCT ATG TGC | | 35 |
| | | TAT GTC TAAA | | |
| T-locker-3 nt- | ssDNA | TTTA GAC ATA GCA CAT | 5′-BHQ1; | SEQ ID NO: |
| BHQ1-Int FAM | | AGA C TG TCT ATG TGC | Int 6-FAM- | 36 |
| | | TAT GTC TAAA | dT | |
| T-locker- | ssDNA | TTTAGACATAGCACATAG | 5′Azide(N3); | SEQ ID NO: |
| 3 nt-5′CC | | ACCGTCTATGTGCTATGTC | 3′CHCH | 37 |
| | | TAAA | | |
| T-locker-3 nt- | ssDNA | ACAGTAGCGATCTCATTT | | SEQ ID NO: |
| elong PAM | | AGACATAGCACATAGACT | | 38 |
| | | GTCTATGTGCTATGTCTAA | | |
| | | ATGAGATCGCTACTGT | | |

### Reporter Constructs

As used herein, a “reporter construct” refers to a molecule that can be cleaved or otherwise modified by an activated CRISPR system effector protein described herein and wherein such cleavage/modification of the reporter molecule is detectable. The term “reporter construct” may alternatively also be referred to as a “detector construct”, “probe construct” or “molecular beacon construct”, etc. Depending on the nuclease activity of the CRISPR effector protein, the reporter construct may be an RNA-based construct or a DNA-based construct. The reporter construct may also be a Xeno nucleic acid (XNA) construct which includes one or more, or consists of Xeno nucleic acids or artificial nucleotides. A Xeno nucleic acid or artificial nucleotide may comprise a non-naturally occurring sugar or nucleobase. The nucleic acid-based reporter construct comprises a nucleic acid element that is cleavable by a CRISPR effector protein. Cleavage of the nucleic acid element releases the agent or produces a conformational change of the nucleic acid in the reporter that allows the generation of a detectable signal. Exemplary constructs demonstrating how to use nucleic acid elements to prevent or mask the generation of a detectable signal will be known to the skilled person and exemplary embodiments are described below, and embodiments of the invention include these or variants thereof. Prior to cutting, or when the reporter construct is not in an “active” state, the reporter construct can be designed so that the generation or detection of a positive detectable signal is blocked, masked, quenched or inhibited. It will be appreciated that in certain exemplary embodiments, minimal background signal may be generated in the presence of non-active reporter constructs. The positively detectable signal can be any signal that can be detected using optical, fluorescent, colorimetric, chemiluminescent, electrochemical or other detection methods known in the art. The term “positive detectable signal” is used to distinguish between other detectable signals detectable in the presence of the reporter construct. For example, in certain embodiments, a first signal (i.e., a negative detectable signal) can be detected when a masking or quenching agent is present, which is then converted to a second signal (e.g., a positive detectable signal) when the target molecule is detected and the masking or quenching agent is cleaved or inactivated by the activated CRISPR effector protein.

In certain other exemplary embodiments, the reporter construct may comprise an RNA, a DNA oligonucleotide or a modified or RNA or DNA, comprising one or more Xeno Nucleic Acids (XNA) or artificial nucleotides, to which a detectable label is attached and a masking or quenching agent for the detectable label. Examples of such detectable label/masking agent pairs are fluorophores and quenchers of fluorophores. Quenching of a fluorophore can occur due to the formation of a non-fluorescent complex between the fluorophore and another fluorophore or a non-fluorescent molecule. This mechanism is called ground state complex formation, static quenching or contact quenching. Thus, an RNA or DNA oligonucleotide can be designed such that the fluorophore and quencher are sufficiently close for contact quenching to occur. Fluorophores and their associated quenchers are known in the art and can be selected by one of ordinary skill in the art for this purpose. The particular fluorophore/quencher is not critical in the context of the present invention, so long as the fluorophore/quencher pair is selected to ensure masking of the fluorophore. Upon activation of the effector proteins disclosed herein, the RNA or DNA or XNA oligonucleotides are cleaved, thereby severing the proximity between the fluorophore and quencher needed to maintain the contact quenching effect. Thus, detection of a fluorophore can be used to determine the presence of the target molecule in a sample.

The reporter can be ssDNA, ssRNA or a nucleic acid sequence with at least one non-naturally/modified nucleotide, such as XNA, and have a length >=2 nucleotides in length and any nucleic acid sequences. In a preferred embodiment the reporter construct comprises a sequence selected from the group consisting of TTATT, CCCCCC, CTC TCA TTT TTT TTT TAG AGA G (SEQ ID NO: 39), UUAUU, UUUUU, TTXTT or UUXUU, where X represents an artificial nucleotide may comprise a non-naturally occurring sugar or nucleobase. In another preferred embodiment the foregoing reporter construct is used in combination with Cas12a or Cas13a.

In a preferred embodiment the reporter construct is a ssRNA construct and labelled with the Fluorophore FAM (e.g. 5′) and a suitable matching quencher BHQ1 (e.g. 3′). In another preferred embodiment the report construct has the sequence 5′UUAUU3′. In another preferred embodiment the foregoing ssRNA reporter construct is used in combination with Cas 12a or Cas13a.

In another preferred embodiment, the reporter construct is a ssRNA construct and labelled with the Fluorophore Texas Red (e.g. 5′) and a suitable matching quencher BHQ2 (e.g. 3′). In another preferred embodiment the report construct has the sequence 5′UUAUU3′. In another preferred embodiment the foregoing ssRNA reporter construct is used in combination with Cas 12a or Cas13a.

In a preferred embodiment the reporter construct is a ssDNA construct and labelled with the Fluorophore FAM (e.g. 5′) and a suitable matching quencher BHQ1 (e.g. 3′). In another preferred embodiment the report construct has the sequence 5′TTATT3′. In another preferred embodiment the foregoing ssDNA reporter construct is used in combination with Cas 12a or Cas13a.

In another preferred embodiment, the reporter construct is a ssRNA construct and labelled with the Fluorophore Texas Red (e.g. 5′) and a suitable matching quencher BHQ2 (e.g. 3′). In another preferred embodiment the report construct has the sequence 5′TTATT3′. In another preferred embodiment the foregoing ssDNA reporter construct is used in combination with Cas 12a or Cas13a.

The length of RNA or DNA, or XNA oligonucleotide reporter constructs, based on design, are optimally from 2 to 15 nucleotides in length, however they may be longer. The trans-cleavage activity of activated Cas 12a is random. In contrast, for different Cas13a proteins, the cutting preference is different. For example LwaCas13a has preference for U-U reporter, PsmCas13a has preference for A-A reporter, CcaCas13b has preference for U-A reporter (J. S. Gootenberg et al., Science 10.1126/science.aaq0179 (2018)).

In another embodiment, the reporter construct is a ssDNA or RNA construct labeled with an electrochemically detectable moiety. In a preferred embodiment the electrochemically labelled reporter construct is immobilized on an electrode where it generates the electrochemical signal (due to proximity of the detectable moiety to the electrode), and wherein trans-cleavage activity of activated CRISPR/Cas effector protein liberates the electrochemically detectable moiety leading to a detectable drop in the electrochemical signal.

In another embodiment, the reporter construct may be adapted for endpoint detection via a lateral flow device. The person skilled in the art will appreciate that various arrangements for a lateral flow device may be utilized in connection with the reporter constructs and methods described herein. For example, the reporter construct used in the context of the present invention may comprise a first molecule and a second molecule or entity connected by an RNA linker. The lateral flow strip or device includes a sample area, where the CRISPR/Cas reaction product with cleaved nucleic acid, e.g. labelled reporter, can be added. The lateral flow strip also typically includes a first capture line, typically a horizontal line across the device, although other configurations are possible. The first capture area may be adjacent to the sample loading region and on the same end of the lateral flow device. A first binding agent that specifically binds to a first molecule of the reporter construct is immobilized or otherwise immobilized to the first capture region. The second capture area may be located at an end of the lateral flow substrate opposite the first binding area. The second binding agent is immobilized or otherwise fixed at the second capture area. The second binding agent specifically binds to a second molecule of the reporter construct, or the second binding agent can bind to a detectable ligand. For example, the detectable ligand may be a particle, such as a colloidal particle, that is visually detectable when aggregated. The particles may be modified with an antibody that specifically binds to a second molecule on the reporter construct. If the reporter construct is not cleaved, the detectable ligand will accumulate at the first binding region. If the reporter construct is cleaved, the detectable ligand is released to flow to the second binding region. In such embodiments, the second binding agent is an agent capable of specifically or non-specifically binding a detectable ligand on an antibody on the detectable ligand.

For example, detection may occur via a lateral flow strip based upon degradation of a reporter construct that is labelled on opposing ends with a detection protein and biotin, respectively. The detection protein-biotinylated reporter will attach to gold nanoparticle conjugated mouse antibodies that are specific to the detection protein that are contained within a lateral flow device. If the reporter remains intact, the detection protein-biotin-labelled reporter accumulate at a first line of the strip immobilized by streptavidin (control line). In the presence of activated CRISPR/Cas effector protein (e.g. Cas 12a), i.e. when the target is present, the reporter is cleaved and freed detection protein conjugates are released to accumulate at a second line of the lateral flow strip containing anti-mouse antibody (test line).

In a preferred embodiment, the reporter construct may comprise a Xeno Nucleic Acid (XNA), or consist of XNAs. The inventors have surprisingly discovered that reporter constructs comprising certain XNAs demonstrate compatible and even enhanced performance with a DNA reporter construct. In a preferred embodiment, the XNA included in the reporter construct is selected from deoxyuridine, 2F-RNA reporter, and 5-Aza-2′-deoxycytidine. In a preferred embodiment the report is sequence and structure: TTXTT, where X is the XNA.

In a preferred embodiment the reporter construct is a ssDNA construct and labelled with the Fluorophore FAM (e.g. 5′) and a suitable matching quencher BHQ1 (e.g. 3′). In another preferred embodiment the reporter construct has the sequence 5′TTXTT3′. In a further preferred embodiment X is selected from deoxyuridine, 2F-RNA reporter, and 5-Aza-2′-deoxycytidine. In another preferred embodiment the foregoing ssDNA reporter construct is used in combination with Cas 12a or Cas13a.

In a preferred embodiment the reporter construct is a ssDNA construct and labelled with the Fluorophore Texas Red (e.g. 5′) and a suitable matching quencher BHQ2 (e.g. 3′). In another preferred embodiment the reporter construct has the sequence 5′TTXTT3′. In a further preferred embodiment X is selected from the group consisting of deoxyuridine, 2F-RNA, and 5-Aza-2′-deoxycytidine. In another preferred embodiment the foregoing ssDNA reporter construct is used in combination with Cas 12a or Cas13a.

In another embodiment, the labelled reporter construct comprising a nucleic acid that can be cleaved or otherwise inactivated by an activated CRISPR system effector protein described herein, has an enzyme conjugated to the nucleic acid as the label. In such embodiments the enzyme is compatible with chromogenic, fluorogenic, and chemiluminescent substrates for generation of a detectable signal. In one embodiment, the reporter construct comprises a nucleic acid that can be cleaved or otherwise inactivated by an activated CRISPR system effector protein described herein, conjugated to a Horseradish peroxidase (HRP) or Alkaline Phosphatase (AP) enzyme. In a preferred embodiment reporter construct comprises a nucleic acid that can be cleaved or otherwise inactivated by an activated CRISPR system effector protein described herein, is conjugated to a Horseradish peroxidase (HRP). In another preferred embodiment, the enzyme conjugated nucleic acid reporter construct is also conjugated to a magnetic bead or other particle which facilities removal of uncleaved reporter constructs from a solution or reaction mixture. Such a removal step may be employed when such a reporter construct is employed for the methods described herein. In ai exemplary embodiment a chromogenic, fluorogenic, and chemiluminescent substrate is added to the reaction mixture following a step of removal of magnetic beads and thereby any uncleaved reporter constructs, for the generation of a detectable signal.

In another embodiment, the reporter construct has the following structure: magnetic bead (MB)-nucleic acid-enzyme. In a preferred embodiment the reporter construct has the structure of: MB-ssDNA-HRP. In some embodiments, nucleic acid may comprise a tag and/or fluorescent moiety (e.g. biotin, FAM, etc.). In another embodiment, the conjugation of the enzyme (e.g. HRP of AP) to the nucleic acid may occur through an enzyme labelled antibody which binds to said tag or fluorescent moiety.

### Target Binding Constructs

As used herein the term “target binding construct” refers to a construct comprising a molecule that interacts in a non-covalent fashion to a target. For example, the target binding construct may comprise a polypeptide of a known amino acid sequence capable of binding to a target of interest, usually a protein target, and usually capable of specifically binding. For example, the target binding construct can be selected to contain the amino acid sequence of the binding partner of the target protein of interest. Cell surface receptors and secreted binding proteins (such as growth factors), soluble enzymes, structural proteins (such as collagen and fibronectin), etc., as an exemplary class of target proteins for which the amino acid sequences of binding partners (such as inhibitors) are well known.

In some embodiments, the target binding construct comprises a full length antibody or an antibody fragment containing an antigen binding domain, antigen binding domain fragment or an antigen binding fragment of the antibody (e.g., an antigen binding domain of a single chain) which is capable of binding, especially specific binding, to a target of interest, usually a protein target of interest. In this embodiment the target binding construct contains an antigen binding domain. In such embodiments, the antigen binding domain can be a binding polypeptide such as, but not limited to variable or hypervariable regions of light and/or heavy chains of an antibody (VL, VH), variable fragments (Fv), F(ab′) 2 fragments, Fab fragments, single chain antibodies (scAb), single chain variable regions (scFv), complementarity determining regions (CDR), or other polypeptides known in the art containing an antigen binding domain capable of binding target proteins or epitopes on target proteins. In further embodiments, the target binding construct may be a chimera or hybrid combination containing a first target binding portion that contains an antigen binding domain and a second target binding portion that contains an antigen binding domain such that each antigen binding domain is capable of binding to the same or different target (e.g. bi-specific or multispecific antibody). In some embodiments, the target binding construct is a bispecific antibody or fragment thereof, designed to bind two different antigens. The origin of the antigen binding domain can be a naturally occurring antibody or fragment thereof, a non-naturally occurring antibody or fragment thereof, a synthetic antibody or fragment thereof, a hybrid antibody or fragment thereof, or an engineered antibody or fragment thereof.

Methods for generating an antibody for a given target are well known in the art. The structure of antibodies and fragments thereof, variable regions of heavy and light chains of an antibody (VH and VL), FV, F(ab′) 2, Fab fragments, single chain antibodies (scAb), single chain variable regions (scFv), and complementarity determining regions (CDR) are also well understood. Methods for generating a polypeptide having a desired antigen-binding domain of a target antigen are known in the art. Methods for modifying antibodies to couple additional polypeptides are also well-known in the art.

In certain embodiments, the target binding constructs employed in the methods and kits of the invention are antibodies which specifically bind to a cytokine or small molecule. In other embodiments the target binding constructs are antibodies which specifically bind to other antibodies, such as to antibodies of a different species to that of the antibody (e.g. anti-mouse-IgG, anti-rabbit-IgG etc.). In other embodiments, the target binding constructs may specifically bind to an enzyme or other label, which may themselves be employed on another target binding construct such as a peptide or antibody or a label, tag or other moiety (e.g. anti-HRP, anti-FITC etc.) which may be linked or conjugated to a peptide or antibody. The skilled person will recognize that the target binding constructs, in particular antibodies, which may be employed in the methods and kits of the invention are many and varied.

In certain embodiments the target binding constructs may be tagged or labelled. In one embodiment, the target binding construct is biotinylated. In another embodiment, the target binding construct is conjugated to streptavidin. In one embodiment the target binding construct is linked or conjugated to a type V or type VI CRISPR/Cas effector protein, a trigger nucleic acid sequence, a guide RNA, or a type V or type VI CRISPR/Cas effector protein in combination with the guide RNA, or a type V or type VI CRISPR/Cas effector protein in combination with the guide RNA and the trigger nucleic acid sequence. In one embodiment the target binding construct is linked or conjugated to a trigger nucleic acid sequence as described herein. In another embodiment the target binding construct is linked or conjugated to a guide RNA as described herein. In another embodiment the target binding construct is linked or conjugated to a type V or type VI CRISPR/Cas effector protein as described herein. In another embodiment the conjugation of the type V or type VI CRISPR/Cas effector protein or trigger nucleic acid sequence according to the foregoing embodiments occurs via a streptavidin-biotin interaction.

In a particular embodiment, the target binding construct is attached to solid support or substrate. An immobilized substrate may refer to any material that is suitable for, or may be modified to, the attachment of a polypeptide or polynucleotide. Possible substrates include, but are not limited to, glass and modified functionalized glass, plastic (including acrylics, polystyrene and copolymers of styrene with other materials, polypropylene, polyethylene, polybutylene, polyurethane, Teflon etc.), polysaccharides, nylon or nitrocellulose, ceramics, resins, silica or silica-based materials (including silicon and modified silicon), carbon, metals, inorganic glass, plastics, fiber optic strands, and various other polymers. In some embodiments, the solid support comprises a patterned surface suitable for immobilizing molecules in an ordered pattern. In certain embodiments, a patterned surface refers to an arrangement of distinct regions in or on an exposed layer of a solid support. In some embodiments, the solid support comprises an array of wells (e.g. a microtiter plate) or recesses in the surface. The composition and geometry of the solid support may vary depending on its use. In some embodiments, the solid support is a planar structure, such as a slide, chip, microchip and/or array. Thus, the surface of the substrate may be in the form of a planar layer. In some embodiments, the solid support comprises one or more surfaces of a flow cell. In some embodiments, the solid support or surface thereof is non-planar, such as an inner or outer surface of a tube or container. In some embodiments, the solid support comprises a microsphere or bead. “microsphere,” “bead,” “particle” is intended to mean, in the context of a solid substrate, small discrete particles made from a variety of materials including, but not limited to, plastics, ceramics, glass, and polystyrene. In certain embodiments, the microspheres are magnetic microspheres or beads. Alternatively, or additionally, the beads may be porous. The beads range in size from nanometers (e.g., 100 nm) to millimeters (e.g., 1 mm).

As described herein, the target binding constructs employed in the compositions, methods and kits of the present invention may be conjugated to a type V or type VI CRISPR/Cas effector protein, a trigger nucleic acid sequence, a guide RNA, or the type V or type VI CRISPR/Cas effector protein in combination with the guide RNA optionally in further combination with the trigger nucleic acid. In a preferred embodiment the target binding construct is conjugated to the type V or type VI CRISPR/Cas effector protein in combination with the guide RNA, wherein the type V or type VI CRISPR/Cas effector protein has been combined with or pre-loaded with the guide RNA prior to conjugation to the target binding construct.

As will be appreciated by the skilled person, there are well established methods and commercially available kits enabling the conjugation of wide variety of molecules (e.g. biotin, nucleic acids, enzymes (such as Horse Radish Peroxidase (HRP)), fluorophores, etc.) to target binding constructs (e.g. antibodies) including labels. The use of such methods and materials are applicable for the generation of such conjugates described herein. In a preferred embodiment the conjugated target binding constructs described herein are generated using the methods detailed in the Examples.

### Methods

The Type V and Type VI CRISPR/Cas effector proteins, Guide RNAs, Trigger Nucleic Acid Sequences, Reporter constructs, and Target binding constructs described in embodiments above may be employed in any suitable combination in the methods described and exemplified below. In particular the circular DNA molecular constructs comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence, namely the Cir-mediators and Cir-amplifiers described above may be employed in the methods described below.

In one embodiment, the present invention provides a method for the detection of a target nucleic acid in a sample, the method comprising: (a) contacting the sample with: (i) a first type V or type VI CRISPR/Cas effector protein; (ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein; (iii) a circular DNA molecular construct comprising both a ssDNA region and a dsDNA sequence, or a circular RNA molecular construct comprising both an ssRNA region and either a ssDNA sequence or a dsDNA sequence, or a dsRNA sequence or a DNA/RNA hybrid sequence; (iv) a second type V or type VI CRISPR/Cas effector protein; (v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence of the circular RNA molecular construct, wherein hybridization between the guide sequence of the second guide RNA and the dsDNA, ssDNA, dsRNA or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; (vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; and (b) measuring a detectable signal produced following cleavage of the labelled nucleic acid reporter by the first and/or second type V or type VI CRISPR/Cas effector protein, thereby detecting the target in the sample.

In another embodiment, the present invention provides a method for the detection of a target in a sample, the method comprising: (a) contacting the sample with: (i) a first type V or type VI CRISPR/Cas effector protein; (ii) a trigger nucleic acid sequence; (iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein; (iv) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence; (v) a second type V or type VI CRISPR/Cas effector protein; (vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA sequence of the circular DNA molecular construct or the dsDNA or ssDNA or a dsRNA or DNA/RNA hybrid sequence of the circular RNA molecular construct, wherein hybridization between the guide sequence of the second guide RNA and the dsDNA or ssDNA or dsRNA sequence or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; (vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; (viii) a target binding construct; and (b) measuring a detectable signal produced following cleavage of the labelled nucleic acid reporter by the first and/or second type V or type VI CRISPR/Cas effector protein, thereby detecting the target in the sample; wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.

In one embodiment the first and second type V or type VI CRISPR/Cas effector proteins are the same. In one embodiment, the first and second type V or type VI CRISPR/Cas effector proteins and the first and second gRNAs are the same. In another embodiment, the first and second type V or type VI CRISPR/Cas effector proteins are different.

In one embodiment the first CRISPR/Cas effector protein is type VI and the second CRISPR/Cas effector protein is type V. A CISAL system with such effector proteins is capable of detecting RNA molecules without reverse transcription at high sensitivity.

In one embodiment, contacting the sample with the second effector type V or type VI CRISPR/Cas effector protein and the second guide RNA occurs after the sample has been contacted with the first effector type V or type VI CRISPR/Cas effector protein and the first guide RNA. In one embodiment, contacting the sample with the first effector type V or type VI CRISPR/Cas effector protein and the first guide RNA, and the second effector type V or type VI CRISPR/Cas effector protein and the second guide RNA occurs simultaneously. In one embodiment, contacting the sample with the second effector type V or type VI CRISPR/Cas effector protein and the second guide RNA occurs at least about 1, 2, 3, 4, 5, 6, 7, 8, 9, or 10 minutes after the sample has been contacted with the first effector type V or type VI CRISPR/Cas effector protein and the first guide RNA. In another embodiment, contacting the sample with the second effector type V or type VI CRISPR/Cas effector protein and the second guide RNA occurs within about 15 minutes, 20 minutes, 25 minutes, 30 minutes, 35 minutes, 40 minutes, 45 minutes, 50 minutes, 55 minutes, or 1 hour after the sample has been contacted with the first effector type V or type VI CRISPR/Cas effector protein and the first guide RNA. In another embodiment, the first and/or second guide RNA is bound to the first and/or second type V or type VI CRISPR/Cas effector protein, respectively.

In another embodiment the target binding construct is immobilized on a surface. In another embodiment the first target binding construct is conjugated to the type V or type VI CRISPR/Cas effector protein. In a preferred embodiment the target binding construct is conjugated to the type V or type VI CRISPR/Cas effector protein and the guide RNA, wherein type V or type VI CRISPR/Cas effector protein has been combined with or pre-loaded with the guide RNA prior to conjugation. In a further preferred embodiment said type V or type VI CRISPR/Cas effector protein is Cas12a.

In one embodiment, the present invention provides a method of enhancing a type V or type VI CRISPR/Cas detection system which comprises a type V or Type VI CRISPR/Cas effector protein, comprising: adding to a reaction mixture comprising a type V or Type VI CRISPR/Cas effector protein of the detection system, a circular DNA molecular construct comprising both a ssDNA region and a dsDNA sequence, wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA sequence or dsRNA sequence or DNA/RNA hybrid sequence are detectably labelled; wherein the dsDNA of the circular DNA molecular construct or the dsDNA or ssDNA or dsRNA or DNA/RNA hybrid sequence of the circular RNA molecular construct hybridizes with a guide sequence of a guide RNA of said type V or type VI CRISPR/Cas detection system, and hybridization occurs following linearization of the circular DNA or RNA molecular construct, respectively, by the nuclease activity of the type V or type VI CRISPR/Cas effector protein of the detection system and further activates the nuclease activity of the type V or type VI CRISPR/Cas effector protein; and measuring a detectable signal produced following linearization of the circular DNA molecular construct, or the circular RNA molecular construct by the type V or type VI CRISPR/Cas effector protein of said detection system. In one embodiment, the method further comprises adding to the reaction mixture the same type V or type VI CRISPR/Cas effector protein, and a guide RNA, optionally bound to the added type V or type VI CRISPR/Cas effector protein, said guide RNA being the same as that used in said type V or type VI CRISPR/Cas detection system or comprising: a region that binds to the added type V or type VI CRISPR/Cas effector protein, and a guide sequence that hybridizes with dsDNA of the circular DNA molecular construct or the dsDNA or ssDNA sequence of the circular RNA molecular construct.

In one embodiment, the present invention provides a method of enhancing a type V or type VI CRISPR/Cas detection system which comprises a type V or Type VI CRISPR/Cas effector protein, comprising: adding to a reaction mixture comprising a type V or Type VI CRISPR/Cas effector protein of the detection system: (i) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ss DNA or dsDNA sequence are detectably labelled; (ii) a second type V or type VI CRISPR/Cas effector protein; and (iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the guide RNA and the dsDNA or ssDNA or dsRNA or DNA/RNA hybrid sequence occurs following linearization of the circular DNA or RNA molecular construct, respectively, by the nuclease activity of the type V or type VI CRISPR/Cas effector protein of the detection system and further activates the nuclease activity of the second type V or type VI CRISPR/Cas effector protein or the type V or type VI CRISPR/Cas effector protein of said detection system; and measuring a detectable signal produced following linearization of the circular DNA molecular construct, or the circular RNA molecular construct by the second type V or type VI CRISPR/Cas effector protein or the type V or type VI CRISPR/Cas of said detection system.

In another embodiment, the present invention provides a method of enhancing a type V or type VI CRISPR/Cas detection system comprising adding to a reaction mixture comprising the type V or Type VI CRISPR/Cas effector of the system: (i) a circular DNA molecule comprising a ssDNA sequence and a dsDNA sequence; (ii) a second type V or type VI CRISPR/Cas effector protein; (iii) a guide RNA, optionally bound to the second type V or type VI CRISPR/Cas effector protein, said guide RNA comprising: a region that binds to the type V or type VI CRISPR/Cas effector protein, and a guide sequence that hybridizes with the dsDNA sequence of the circular DNA molecule, wherein hybridization between the guide sequence of the guide RNA and the dsDNA sequence occurs following cleavage of the ssDNA region of the circular DNA molecule by the type V or type VI CRISPR/Cas effector protein of the detection system and further activates the nuclease activity of the second type V or type VI CRISPR/Cas effector protein to said second guide RNA; and (iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein; and measuring a detectable signal produced following cleavage of the labelled nucleic acid reporter by the second type V or type VI CRISPR/Cas effector protein.

According to another embodiment, the present invention provides a method of modifying an immunoassay comprising replacing a labelled target binding construct to be employed for signal generation in said immunoassay with a replacement target binding construct directed to the same target; and a) contacting the sample with a reaction mixture comprising: i) said replacement target binding construct; ii) a first type V or type VI CRISPR/Cas effector protein; (iii) a trigger nucleic acid sequence; (iv) a first guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; v) a circular DNA molecule comprising a ssDNA sequence and a dsDNA sequence; (vi) a second type V or type VI CRISPR/Cas effector protein; (vii) a guide RNA, optionally bound to the second type V or type VI CRISPR/Cas effector protein, said guide RNA comprising: a region that binds to the type V or type VI CRISPR/Cas effector protein, and a guide sequence that hybridizes with the dsDNA sequence of the circular DNA molecule, wherein hybridization between the guide sequence of the guide RNA and the dsDNA sequence occurs following cleavage of the ssDNA region of the circular DNA molecule by the type V or type VI CRISPR/Cas effector protein of the detection system and further activates the nuclease activity of the second type V or type VI CRISPR/Cas effector protein to said second guide RNA; and (viii) a labelled reporter construct, wherein said reporter construct comprises a nucleic acid that does or does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the target; wherein the first type V or type VI CRISPR/Cas effector protein, the trigger nucleic acid sequence, the first guide RNA, or the first type V or type VI CRISPR/Cas effector protein and the first guide RNA, optionally in further combination with the trigger nucleic acid, is conjugated to the replacement binding construct to thereby co-locate the type V or type VI CRISPR/Cas effector protein and the target when present in the sample.

In the method of any of the foregoing embodiments, the circular DNA or RNA molecular construct, may be a Cir amplifier as described herein. For example, in the method of the foregoing methods, the circular DNA or RNA molecular construct may be substituted for a Cir amplifier as described herein. In such instances where the circular DNA or RNA molecular construct is a Cir amplifier as described herein, the use of a separate reporter construct may be optionally omitted. That is, the use of Cir amplifier (which comprises a detectable label) may permit the reaction mixture to exclude a separate labeled reporter construct, since due to the design of the Cir Amplifier, linearization of the Cir amplifier by an activated Cas RNP (i.e. when a target is present) will result in the generation of a detectable signal.

The catalytic efficiency of a Cas RNPs is limited to several turnovers per second. Where cleavage of separate reporters is required for the signal, then this reporter cleavage will compete with the available (finite number) of enzymatic turnovers with the cleavage of molecules required to provide a positive feedback loop. Accordingly, an advantage of the use of Cir-Amplifiers as described herein is that they eliminate such competition. Consequently, signal increase per unit time and per RNP is significantly increased comparative to other embodiments where cleavage of separate and different reporter and “activator” molecules is required.

In one embodiment, a reporter construct may also be used in conjunction with the Cir amplifier. In one embodiment the label of the reporter construct or signal generated from the reporter construct may be the same as that of the Cir amplifier. In another embodiment a reporter construct used in conjunction with a Cir amplifier may have a different label or emit a different signal.

In one embodiment of the aforementioned methods, the steps are conducted at a temperature ranging from 18 to 42 degrees Celsius.

In a preferred embodiment, the steps are conducted at a temperature from 25 to 37 degrees Celsius.

The inventors have determined that addition of a sulfhydryl reductant, and/or a non-ionic surfactant to a reaction mixture comprising the type V or Type VI CRISPR/Cas effector protein can lead to enhancement of trans-cleavage activity of Cas12a and Cas13a. Such enhancement is capable of substantially increasing the detection sensitivity of detection systems utilizing Cas 12a and Cas13a and decrease reaction times. Accordingly, in further embodiments of the methods described above, the method comprises adding a sulfhydryl reductant, and/or a non-ionic surfactant to a reaction mixture comprising the type V or Type VI CRISPR/Cas effector protein.

In one embodiment, the sulfhydryl reductant is selected from Dithiothreitol (DTT), or Tris(2-carboxyethyl) phosphine (TCEP) to and 2-Mercaptoethanol (2-ME). In a preferred embodiment the sulfhydryl reductant is DTT. In a further preferred embodiment DTT is provided at a concentration ranging from 100 μM to 20 mM. In a further preferred embodiment DTT is provided at a concentration of 10 mM for Cas12a, and at 5 mM for Cas13.

The inventors have identified that in particular embodiments, the rate of signal production can be substantially increased through the addition of DTT alone, and further augmentation can occur when the reaction is carried out at about 37° C. Accordingly, in a preferred embodiment of the foregoing methods, DTT is added to the reaction mixture and where reduction of time for the production of a signal is required, the reaction is preferably carried out at about 37° C.

In one embodiment, the non-ionic surfactant is selected from Brij L23 and poly(vinyl alcohol) (PVA). In a preferred embodiment the non-ionic surfactant is PVA. In a further preferred embodiment PVA is 87-90% hydrolyzed, average mol wt 30,000-70,000. In a further preferred embodiment PVA is provided at a concentration ranging from 0.33% to 3.3%. In a further preferred embodiment PVA is provided at a concentration of 1% for Cas12a and 0.05% for Cas13a.

In any of the afore described aspects or embodiments, any of the nucleotides present in the guide RNA sequences, trigger nucleic acid sequences, cir Mediators (i.e. circular DNA molecular constructs or RNA molecular constructs), or the reporter constructs and so on including those employed in any of the methods described herein may be a modified nucleotide having a modification, such as having a different sugar backbone to other than the naturally occurring nucleic acids DNA or RNA. That is any one or more of the nucleotides may be substituted with a XNA.

In another embodiment of any of the methods disclosed herein, the method is performed without a reverse-transcription step or a pre-amplification step.

In another embodiment of any of the methods disclosed herein, the method provides detection of a target nucleic acid with 1 aM sensitivity. In another embodiment, the method provides detection of a target nucleic acid with 1 aM sensitivity within at most about 30 minutes. In another embodiment, the method provides detection of a target nucleic acid with 1 aM sensitivity within at most about 20 minutes. In another embodiment, the method provides detection of a target nucleic acid with 1 aM sensitivity within at most about 15 minutes. In another embodiment, the method provides detection of a target nucleic acid with about 5 aM sensitivity within at most about 10 minutes. In another embodiment, the method provides detection of a target nucleic acid with about 1 pM sensitivity within about 100 seconds.

In another embodiment, of any of the methods disclosed herein, the target nucleic acid is genomic DNA. In another embodiment, of any of the methods disclosed herein, the target nucleic acid is genomic RNA. In another embodiment, the target nucleic acid is a nucleic acid comprising a single nucleotide polymorphism. In the method is capable of detecting a single nucleotide polymorphism at a clinically relevant level.

### EXAMPLES

### Materials and Methods for Examples 1, 2, 3, 4.

T4 ligase (NEB), 10×T4 ligase buffer (NEB), exonuclease III (NEB), LbCas12a (NEB), NEB2.1 buffer (NEB), agarose (ThermoFisher), TBE buffer, SYBR Gold DNA dye (ThermoFisher), 100 bp DNA ladder (ThermoFisher), 10 bp DNA ladder (ThermoFisher), 6×DNA loading dye (ThermoFisher), DTT (ThermoFisher), copper sulfate (CuSO4) (Sigma, 209198), Tris(2-carboxyethyl) phosphine (TCEP) (Sigma, C4706), tris(benzyltriazolylmethyl) amine (TBTA) (ChemSupply, t2993), DNase/RNase free water (ThermoFisher), phosphate buffered saline (PBS) (Sigma, 10 mM, pH=7.4).

All DNA and RNA oligos are synthesized and modified by Sangon Bio-Tech Ltd.

### Synthesis of the Cir-Mediators (the Circular DNA/RNA Molecular Construct) Using DNA Ligase

The synthesis of the Cir-mediator was conducted in the reaction mixture containing 2 μL of the linear ssDNA oligo (100 μM), 4 μL of ssDNA linker oligo (100 μM), 2.5 μL of T4 ligase (NEB), 5 μL of 10×T4 ligase buffer, and 39 μL of DNase/RNase free water. The cyclization reaction was allowed to proceed at 20° C. for 12 h and then 65° C. for 10 min, following by holding at 4° C. For removing unbound linear ssDNA and linker oligos, 1.5 μL of exonuclease III was mixed with 10 μL of the product of the cyclization reaction in 40 μL of 1×NEB2.1 buffer, with incubation at 37° C. for 100 min and then 75° C. for 30 min. The aliquots of the circular ssDNA (Cir-ssDNA) product after degradation were stored at −20° C. for future use.

The Cir-mediator for in vitro DNA detection was immediately prepared before use, by mixing Cir-ssDNA and its shorter complementary DNA (cDNA) with PAM sequence (1 μM) at the volume ratio of 1:1 and incubation at 95° C. for 5 min.

### Synthesis of the Cir-Mediator (the Circular DNA/RNA Molecular Construct) Using Click Chemistry

To synthesize Cir-ssDNA, 40 μL of 0.5% w/v streptavidin-modified magnetic beads (0.74 μm) were first blocked with 1% BSA solution for 1 h to eliminate non-specific binding. After magnetic separation of the blocked beads, 100 μL of 0.5 μM biotinylated linear-ssDNA was incubated with the beads for 1 h following a PBS wash (three times) to remove the residual free linear-ssDNA. Subsequently, 100 μL of the click chemistry reaction solution (1.0 mM CuSO4, 2.0 mM TCEP, 100 μM TBTA, and 100 μL PBS buffer) was added and incubated with the beads for 12 h at room temperature. After synthesis, the magnetic beads were collected and washed with PBS buffer to remove excess chemicals. Subsequently, 100 μL of 1000 units/mL Exonuclease III solution was added and incubated in 37° C. for 30 min to remove all the linear ssDNA. After wash with PBS buffer, the synthesized Cir-ssDNA was released from the streptavidin-modified magnetic beads by heat treatment at 95° C. for 30 min, and the supernatant was collected for further use.

The Cir-mediator for in vitro DNA detection was immediately prepared before use, by mixing Cir-ssDNA with its shorter complementary DNA (cDNA) (1 μM) at the volume ratio of 1:1 with incubation at room temperature for 5 min.

### Verification of the Formation of Cir-Mediator

The formation of Cir-ssDNA, Cir-ssDNA after eliminating free ssDNA/linker oligos and Cir-mediators were verified by using agarose gel electrophoresis. Briefly, 1.5% agarose gel in 1×TBE buffer was premade with SYBR Gold DNA dye (1.5 μL 10,000× into 30 mL agarose gel). 10 μL of Cir-ssDNA aliquoted from each step of synthesis process with 2 μL 6×DNA gel loading dye was loaded into gel for electrophoresis, which was carried out for 40 min at a constant voltage of 100V. 3.5 μL of 10 bp DNA ladder was used for molecular weight reference. Gel images were visualized by using Gel Doc+XR image system (Bio-Rad Laboratories Inc., USA).

### CRISPR/Cas12a Trans-Cleavage Activation by Cir-Mediators

The CRISPR/Cas12a reaction buffer was prepared: 1.5 μL of LbaCas12a endonuclease (10 μM, NEB, M0653T), 7.5 μL of gRNA for Cir-mediator (20 μM), 4.5 μL of labelled ssDNA reporter (Texas red-TTATT-BHQ2, 100 μM), and 27 μL of DTT (1M) were mixed in 2.7 mL of 1×NEB2.1 buffer. The mixture was stored at 4° C. for future use. For each CRISPR/Cas12a activation reaction, 5 μl of Cir-mediator (or other oligos, linear ssDNA, Cir-ssDNA, etc.) was added into 100 μL prepared reaction buffer. The reaction was set at room temperature, and the fluorescence intensity at Ex/Em of 570/615 nm was determined by using a plate reader (iD5 Spectramax, Molecular Devices, USA).

### Evaluating Cas12 Activation Efficiency of Linearized Cir-Mediators

The CRISPR/Cas12a reaction mixture for linearizing Cir-mediators (synthesized by using ligase or click chemistry) was prepared as follows: 1 μL 100 μM (100 pmol) of Cas12a protein was gently mixed with 5 μL 20 μM (100 pmol) of gRNA (complementary for trigger ssDNA) and 72 μL of 2 μM Cir-mediators in 3.6 mL 1×NEB 2.1 buffer. Subsequently, 5 μL of 1 μM target ssDNA was added into 100 μL of the CRISPR/Cas12a reaction mixture for activating trans-cleavage of Cas12a/gRNA-trigger ssDNA and enabling the cleavage of the ssDNA region of Cir-mediators, hence their linearization. The cleavage reaction was incubated at 37° C. for 2 h. Afterwards, 10 μL of the reaction product was added into 100 μL of another CRISPR/Cas12a reaction mixture with gRNA targeting the dsDNA region of the Cir-mediators. A SpectraMax iD5 Spectramax (Molecular Devices) was applied for the detection of fluorescence readout.

### Cir-Mediator-Induced CRISPR/Cas12a Trans-Cleavage Setting Off the Self-Amplification Cascade (Setup 1 Used for Cir-Mediators Created by the Ligase Method)

Preparation of standard CRISPR/Cas12a reaction mixture: 1.5 μL of LbaCas12a endonuclease (10 μM, NEB, M0653T), 7.5 μL of gRNA complementary for the target nucleic acid sequence (20 μM), 4.5 μL of Texas Red reporter (100 μM), and 27 μL of DTT (1M) were mixed in 2.7 mL of 1×NEB2.1 buffer. The mixture was stored at 4° C. for future use.

Preparation of Cir-mediator-enhanced CRISPR/Cas12a reaction mixture: 6 μL of LbaCas12a endonuclease (10 μM, NEB, M0653T), 7.5 μL of gRNA complementary for the target nucleic acid sequence (20 μM), 22.5 μL of gRNA complementary for the Cir-mediator dsDNA sequence (20 μM), 4.5 μL of TR reporter (100 μM), and 12 μL of DTT (1M) were mixed in 2.7 mL of 1×NEB2.1 buffer. The mixture was stored at 4° C. for future use.

ssDNA detection by a standard CRISPR/Cas12a-based system: 10 μL of triggering ssDNA with different concentrations were mixed with 100 μL of the standard CRISPR/Cas12a reaction mixture on the ice. The fluorescence intensity at Ex/Em of 570/615 nm was determined by using a plate reader (iD5 Spectramax, Molecular Devices, USA).

ssDNA detection by Cir-mediator-enhanced CRISPR/Cas12a-based system: 5 μL of triggering ssDNA with different concentrations and 5 μL of Cir-mediator were mixed with 40 L of the Cir-mediator-enhanced CRISPR/Cas12a reaction mixture on ice. The fluorescence intensity measurements were performed by quantitative real-time quantitative reverse transcription polymerase chain reaction (Real-Time qRT-PCR) on a CFX96 Touch Real-Time PCR Detection System (Bio-Rad Laboratories Inc., USA). The mixture was incubated 240 cycles of 37° C. for 30s.

### Cir-Mediator Induced CRISPR/Cas12a Trans-Cleavage Setting Off the Self-Amplification Cascade (Setup 2 Used for Cir-Mediators Synthesized by Click Chemistry)

Preparation of Cir-mediator-enhanced CRISPR/Cas12a reaction mixture: 4 μL 100 μM (100 pmol) of Cas12a protein was gently mixed with 5 μL 20 μM (100 pmol) of gRNA complementary to the target nucleic acid sequence, 15 μL 20 μM (100 pmol) of gRNA complementary to the Cir-mediator dsDNA sequence, and 72 μL of 2 μM synthesized Cir-mediator in 3.6 mL 1×NEB 2.1 buffer. Then, 6 μL of 100 μM (0.6 nmol) of pre-synthesized fluorescent quenched ssDNA reporters (Texas red-TTATT-BHQ2) were added and well mixed. The mixture was stored at 4° C. for future use.

Afterwards, 10 μL of target DNA at different concentrations was added to 100 μL of the Cir-mediator enhanced CRISPR/Cas12a reaction mixture for activating trans-cleavage of Cas12a. A SpectraMax iD5 multi-Mode Microplate Reader (Molecular Devices) was applied for the detection of fluorescence readout.

### Example 1—Formation of the Cir-Mediator Molecular Construct

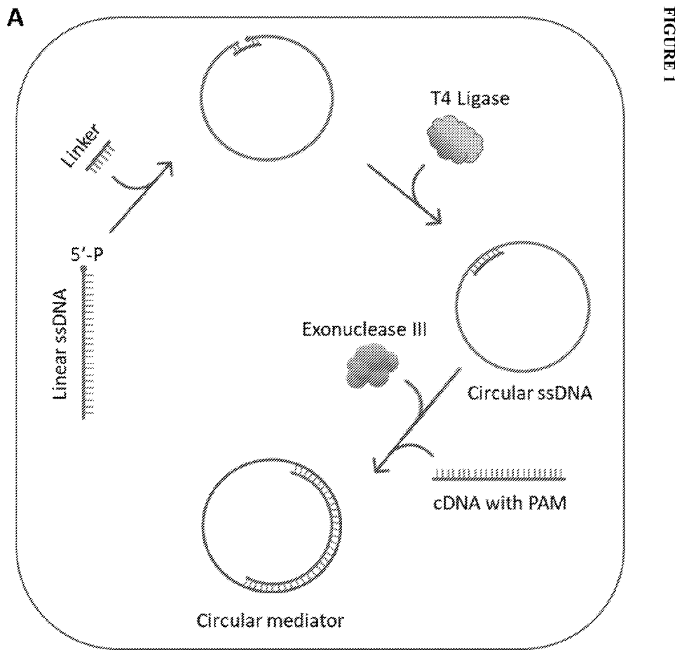

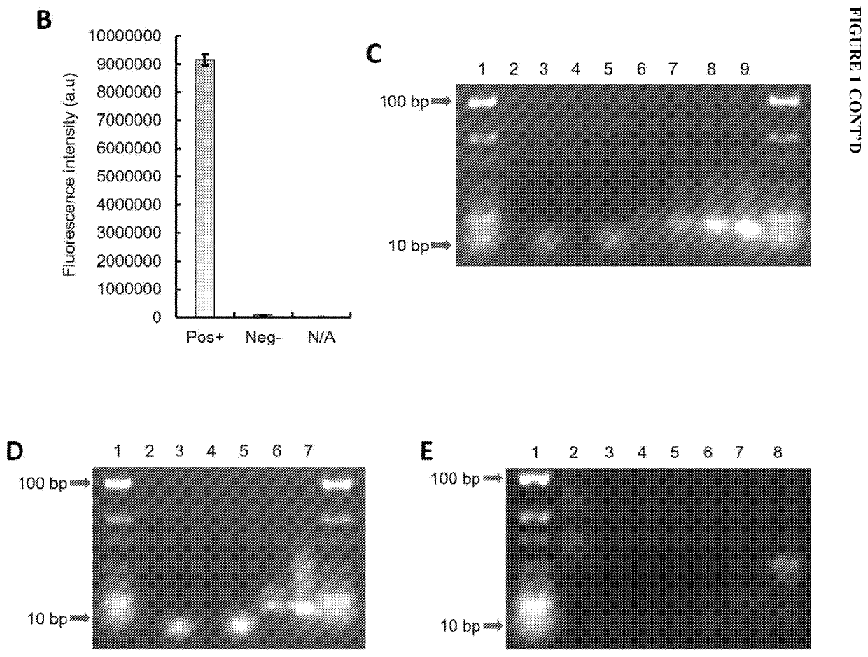

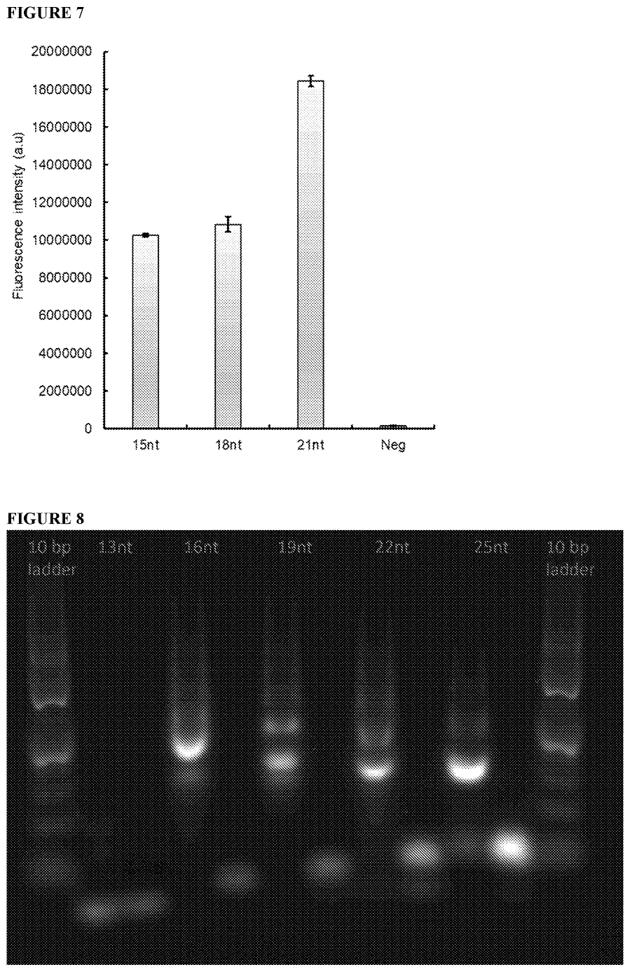

The Cir-mediator is a partially dsDNA-based circular DNA molecular construct, which can be formed through a three steps synthesis protocol (FIG. 1A). Firstly, a 5′-P linear ssDNA oligo was pulled into a circular structure through the help of a short ssDNA linker, which has complementary sequences to both end of the linear ssDNA. Then, the break locus was ligated by T4 DNA ligase to form the basic backbone structure of the Cir-mediator (or the Cir-ssDNA). After the excessive linear ssDNA and linker oligos have been degraded by exonuclease III, a slightly shorter complementary ssDNA oligo (cDNA) was added to form the final Cir-mediator molecular construct. Before the linear ssDNA has been used for Cir-mediator synthesis, its highly effective CRISPR/Cas12a trans-cleavage activation ability has been verified by using Cas12a RNP loaded with guide RNA (gRNA), which has complementary sequence to the designated dsDNA region of the linear ssDNA (FIG. 1B). By increasing the linear ssDNA length, from 15 nt to 21 nt, the activation efficiency has also increased accordingly (FIG. 7). After the Cir-ssDNA has been formed, a delayed movement can be identified on the agarose gel electrophoresis image (FIG. 1C), as the circular ssDNA is characterized by an appreciably slower movement compared to its linear form on 2% agarose gel. By treating the synthesized Cir-ssDNA product with exonuclease III, all linear ssDNA with free 3′ end has been digested, except for the formed circular ssDNA, hence, leading to a clear band on the agarose gel without a smear/tail (FIG. 1D). Finally, by adding the complementary ssDNA oligo, the formed Cir-mediator molecule has been found to show a further delayed movement on the agarose gel due to its increased molecular weight (FIG. 1E).

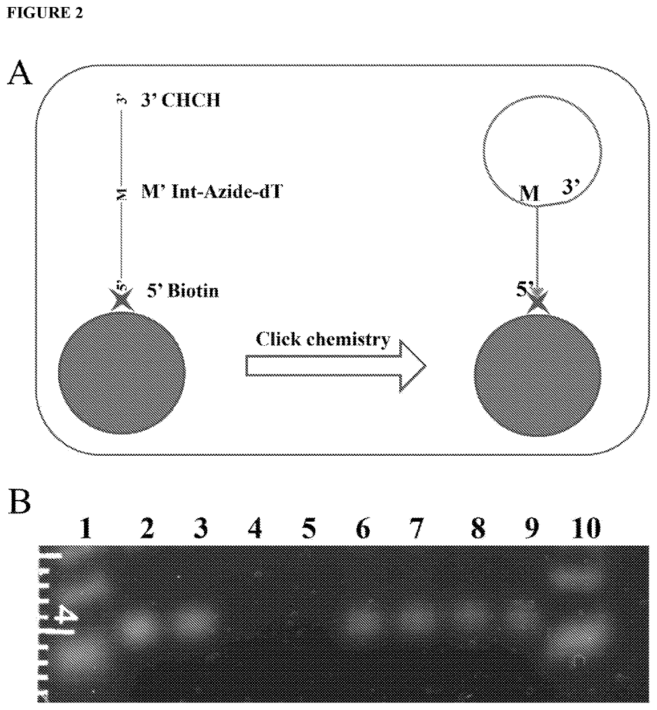

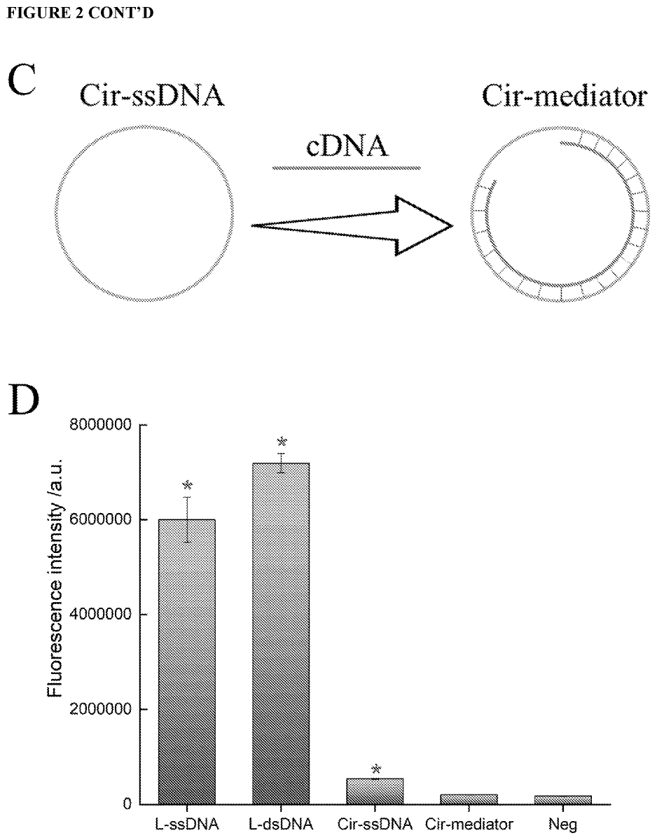

Alternatively, the Cir-mediator molecular construct has been formed by using a click chemistry approach (FIG. 2A). In order to synthesize a single ring Cir-ssDNA, streptavidin-modified magnetic beads were applied to immobilize the biotinylated linear ssDNA (FIG. 2A). After immobilization of ssDNA, the click chemistry approach was applied to form the Cir-ssDNA through the linkage of Azide and alkyne (CHCH) functional groups. In this work, linear ssDNA was applied to synthesize the circular ssDNA which links the Azide group on dT of the middle of the linear nucleic acid sequence to —CHCH on the 3′ end (FIG. 2A). After the Cir-ssDNA is formed, heating to 95° C. was used to release the nucleic acid from the magnetic beads and also used Exonuclease III to cleave the residual linear ssDNA tail and any excessive linear ssDNA in the solution, leaving only the circular ssDNA intact. Then, the thus synthesized Cir-ssDNA was characterized by using the agarose gel electrophoresis assay (FIG. 2B). The bands of linear ssDNA were clearly shown in the gel image (Lanes 2-3). After Exo III treatment, all the bands were found to disappear (Lanes 4-5), indicating that all linear ssDNA has been cleaved by Exonuclease III. In addition, the strips produced by Cir-ssDNA were clearly indicated in the gel image (Lanes 6-7). After Exo III treatment of Cir-ssDNA, the remained bands indicated the successful formation of Cir-ssDNA molecular construct (Lanes 8-9). Finally, the Cir-mediators have been formed by mixing the Cir-ssDNA with its cDNA (FIG. 2C).

In addition, it has been demonstrated that when the linear ssDNA oligo has a total length of more than 15 nucleotides, it can successfully form the circular DNA molecular construct, but when the oligo length is shorter than 16 nt, the circular ssDNA can not be efficiently synthesized (FIG. 7).

### Example 2—A Suitably Designed Cir-Mediator is Unable to Induce Cas12a Enzymatic Trans-Cleavage Activation

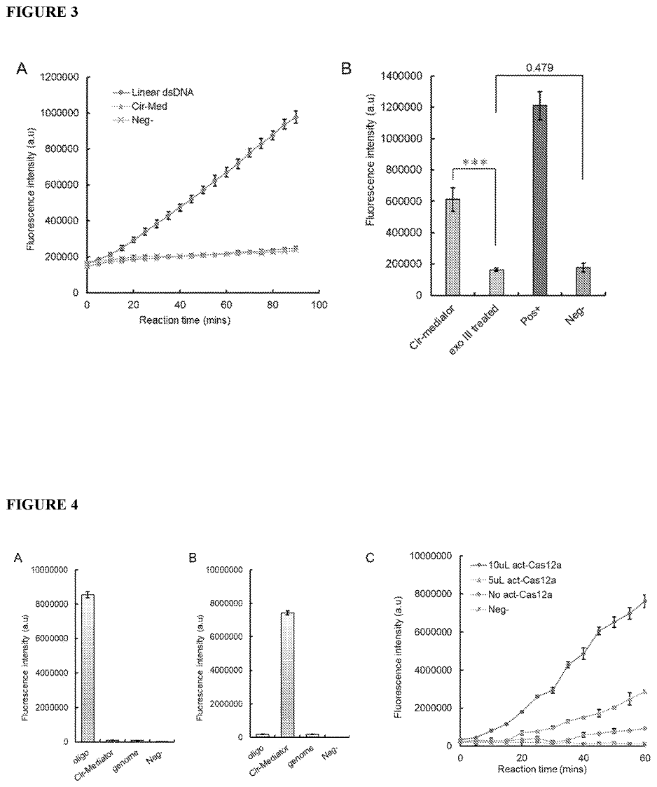

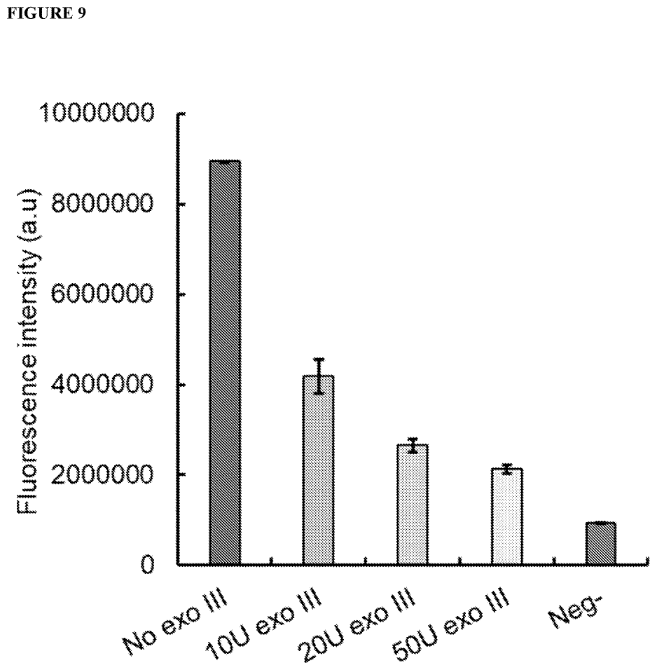

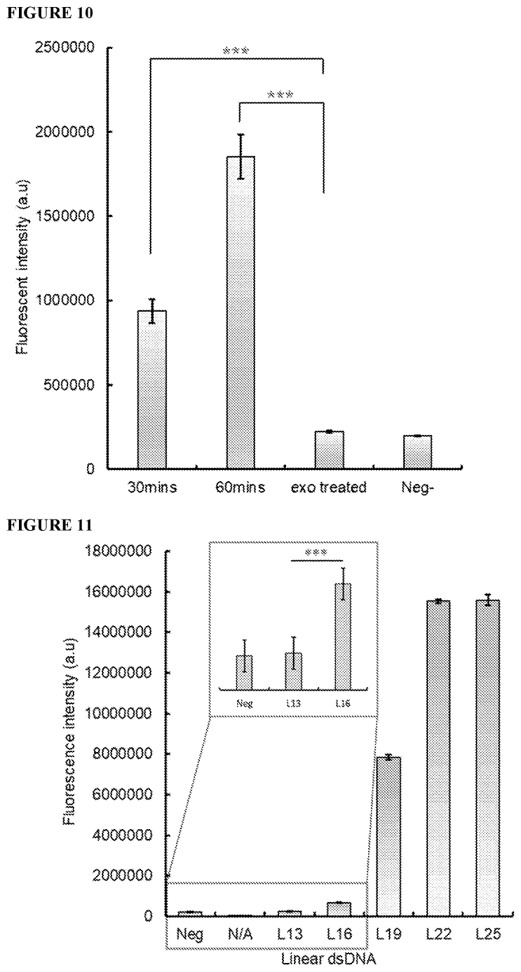

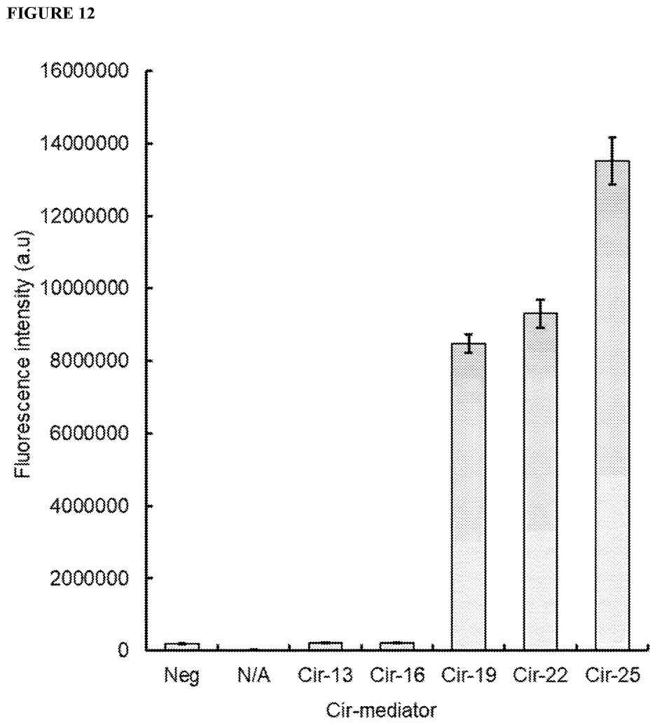

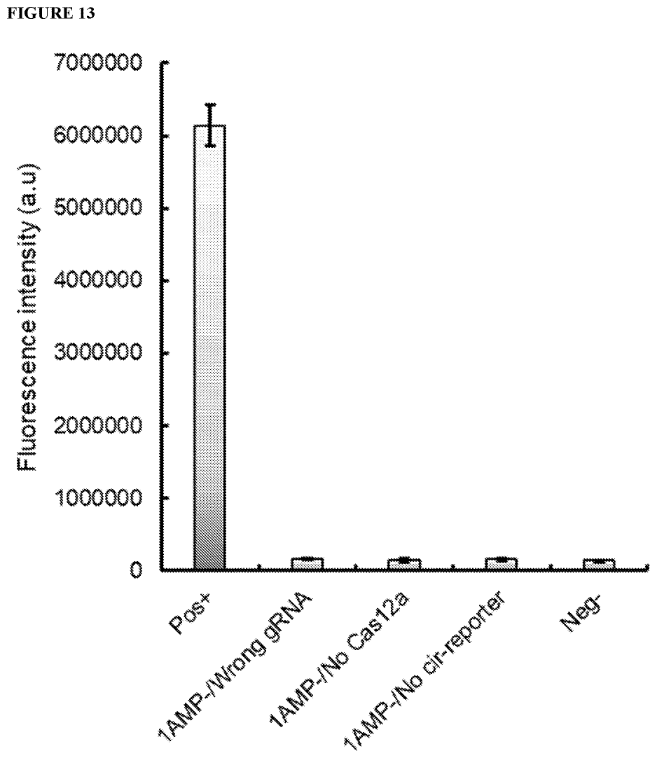

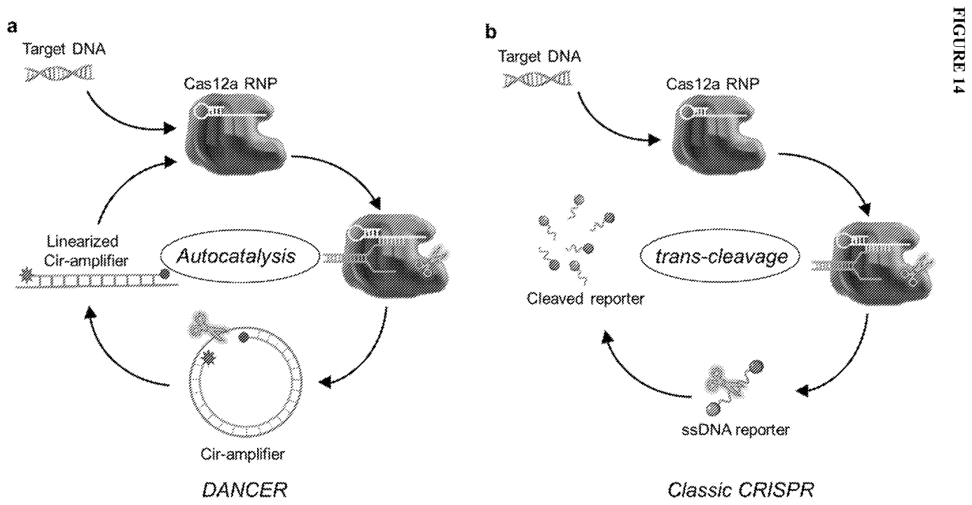

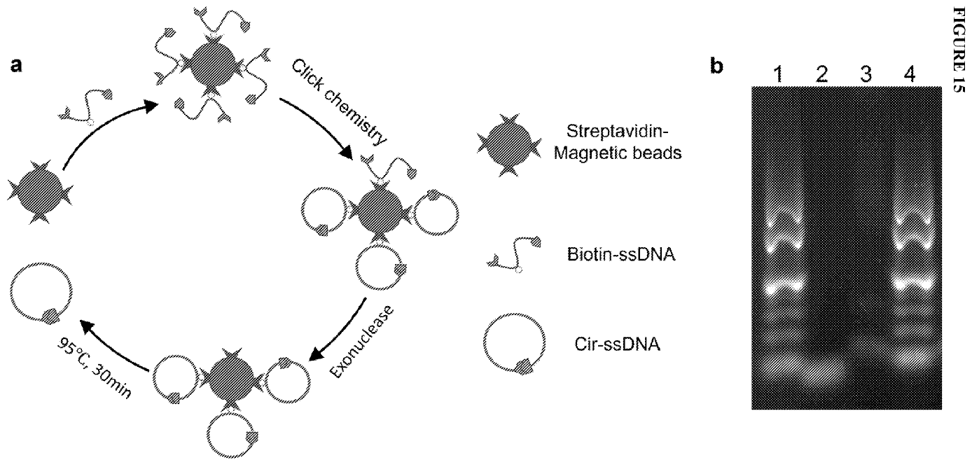

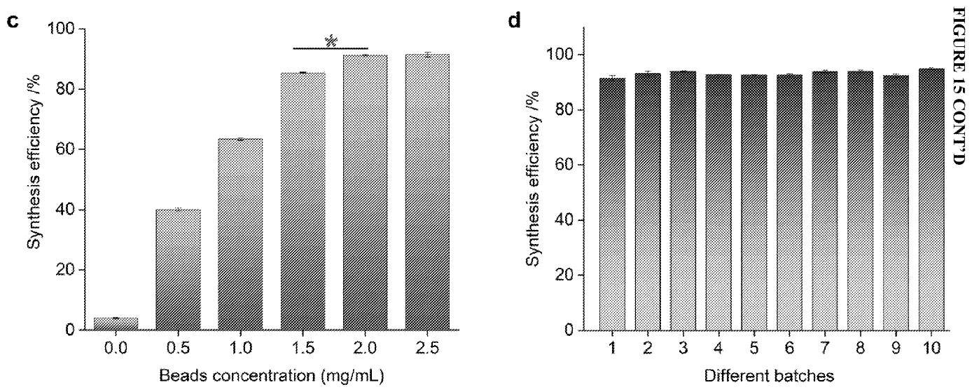

After the Cir-mediator molecular construct has been formed, its capability to trigger trans-cleavage activity in CRISPR/Cas12a RNPs was evaluated. In comparison to its original linear ssDNA form, a properly designed and prepared Cir-mediator molecular construct has been found to be completely inert with respect to Cas12a trans-cleavage activation for at least 1.5 hours, as no significantly increased final fluorescence intensity could be detected (FIG. 3A). A prior exonuclease III treatment of the Cir-mediator solution was necessary for this demonstration, as the elimination of excessive free linear ssDNA prevents the unwanted accidental activation of the Cas12a trans-cleavage (FIGS. 3B, 9 and 10). In addition, the tests of Cas12 activation by using linear dsDNA and Cir-mediators show a positive correlation between activation efficiency and dsDNA length (FIG. 11). In contrast to linear dsDNA, for the corresponding Cir-mediators, the Cir-mediators with total length shorter than 21 nt have been found not to cause significant CRISPR/Cas12a activation of trans-cleavage over certain periods of time (FIG. 12). Hence, the preferable total length for preparing the Cir-mediators is determined to be between 16 to 21 nucleotides.

Similarly, the Cir-mediators prepared by click chemistry approach have also shown the same features as the Cir-mediators produced by the ligase-assisted method. Namely, they did not cause the activation of trans-cleavage of Cas12a, as no significant increase of fluorescent intensity level was detected in a 2-hour reaction (FIG. 2D).

### Example 3—Restoration of Cas12a RNP Activation by Cleaved (Linearized) Cir-Mediators

When a CRISPR/Cas12a RNP has been activated by its designated DNA target, the triggered trans-cleavage is able to cut any surrounding ssDNAs. Therefore, the ssDNA region of the subsequently introduced Cir-mediators can be cut, restoring this circular construct back to its original linear formation. Afterwards, the thus linearized Cir-mediators were used to activate CRISPR/Cas12a RNPs (with gRNA complementary to the dsDNA region of the Cir-mediators) for trans-cleavage activity. To demonstrate this, two ensembles of CRISPR/Cas12a RNPs have been prepared with different gRNA sequences complementary to a target DNA oligo or the dsDNA region of Cir-mediators, respectively. The specificity of these CRISPR/Cas12a RNPs was first verified with respect to non-specific activation and cross-activation between each other (FIG. 4A, 4B, 13). Afterwards, the CRISPR/Cas12a RNP targeting the DNA oligo has firstly been activated separately for 10 mins in the presence of Cir-mediators, and then this mixture was combined with the second CRISPR/Cas12a reaction mixture targeting the dsDNA region of Cir-mediators. The results show that the addition of the pre-activated Cas12a/Cir-mediator reaction product to the unactivated Cas12a reaction mixture which is able to recognize the dsDNA region of the Cir-mediators can lead to the activation of the latter. Furthermore with the increased number of the pre-activated Cas12a RNPs in the first RNP ensemble, the Cas12a activation intensity of the second RNP ensemble has also increased (FIG. 4C).

### Example 4—Cir-Mediator-Induced CRISPR/Cas12a Amplification Cascade for Increased Sensitivity to DNA

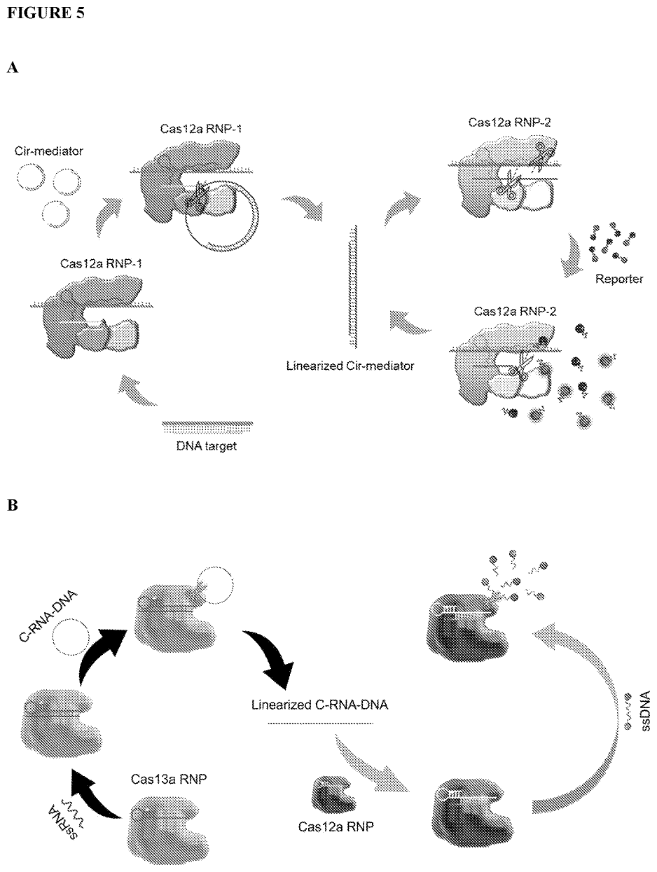

The CRISPR/Cas12a activation ability of Cir-mediators can be restored when they are transformed back to their linear formation through breaking its ssDNA region by pre-activated CRISPR/Cas12a RNPs. The restoration of Cas12a trans-cleavage by cleaved Cir-mediators can be used to establish an amplification cascade for Cas12a RNP activation, thus allowing one target nucleic acid molecule to activate not one but multiple Cas12 RNPs (FIG. 5).

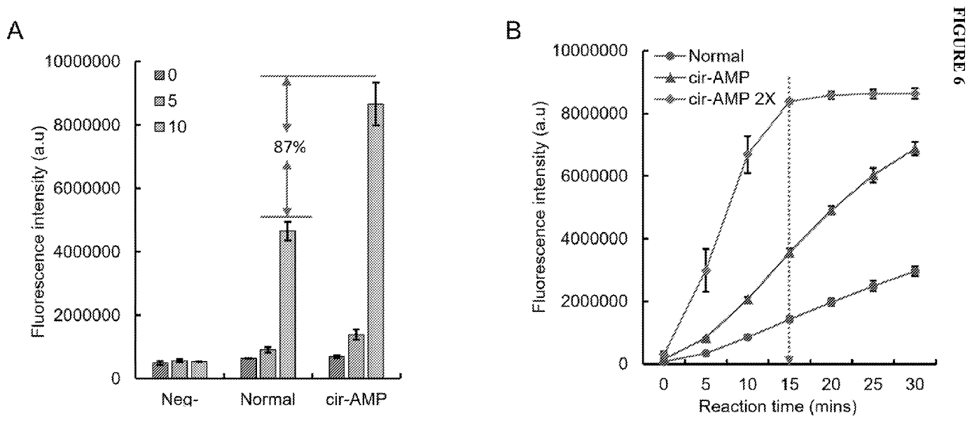

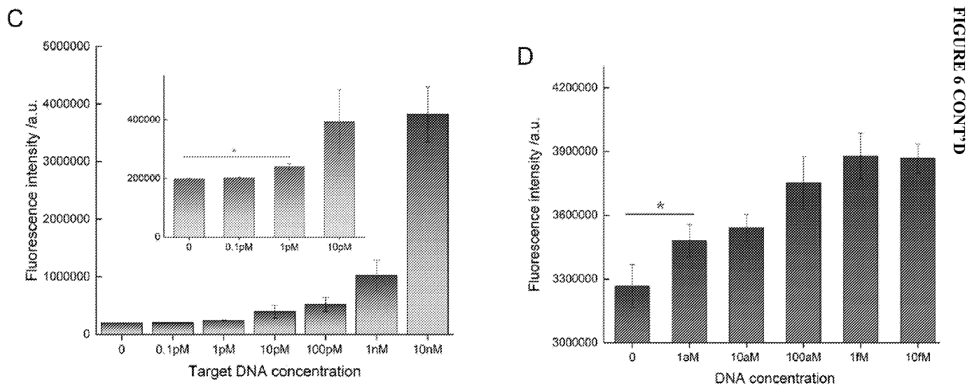

In comparison to CRISPR/Cas12a activation without Cir-mediators, the fluorescence signal in the presence of Cir-mediators significantly increased (FIG. 6A), and this trend is positively correlated with the quantity of Cir-mediators (FIG. 6B). In addition, the presence of Cir-mediators in the CRISPR/Cas system led to much (50%) faster signal saturation (FIG. 6B). When compared with the standard CRISPR/Cas12a-based DNA detection protocol, the Cir-mediator enhanced CRISPR/Cas12a reaction system was more effective to detect low concentration of ssDNA targets, from originally 1 pM target DNA to 1 aM (6 logs) (FIG. 6C-6D). This is equal to the sensitivity of ˜0.6 copy/μL, which is comparable to quantitative PCR.

### Discussion

The programmable nuclease trans-cleavage activity of Cas12 and Cas13 has been utilized here in a novel scheme to detect nucleic acid targets where specific recognition of nucleic acid targets recognition by binding to guide RNA induces a highly efficient signal amplification enabled by trans-cleavage induced by this binding. The nuclease function of Cas12 and Cas13 depends on the binding of gRNA to target DNA with complementary sequence to its spacer region, which leads to the formation of the R-loop structure and opening the cap covering the catalytic domain residue. Therefore, blocking or hindering access of the target DNA sequence to CRISPR/Cas12a gRNA can control the activation of CRISPR/Cas12a RNP. Similar effect is produced by utilizing target sequences which in one (circular) configuration can appear to be shorter than necessary to activate trans-cleavage but are long enough to induce the trans-cleavage in another (linear) configuration. The principle of Cir-mediator-induced CRISPR/Cas amplification cascade is centered around a simple but stable DNA or RNA structure (Cir-mediator) which is not causing CRISPR/Cas activation when it is circular but whose Cas nuclease trans-cleavage activation ability is restored when the Cir-mediator becomes linearized. The Cir-mediators are circular DNA or RNA molecular constructs whose length is close to the minimum length required to form a circular shape and also close to the minimum length required for the activation of trans-cleavage in Cas nuclease. The circular shape of these Cir-mediators leads to a natural stereospecific blockade for access of their dsDNA or ssDNA region to their corresponding gRNA in the Cas RNP which is required for trans-cleavage activation. Our Examples show that the trans-cleavage activity of an initially activated CRISPR/Cas12a RNP, which is triggered by the target nucleic acid allows to break the ssDNA region of the Cir-mediators, hence producing linearized Cir-mediators. The thus released linearized Cir-mediators regain access to their corresponding CRISPR/Cas12a RNPs, then triggering a second round of trans-cleavage activation events. This produces many more activated Cas12a RNPs for cutting the nucleic acid reporters (ssDNA, XNA etc.) which then generate a highly amplified signal. Because any subsequently activated CRISPR/Cas12a RNPs in this cascade can also continue to cut the surrounding Cir-mediators to trigger the secondary amplification circle, a single target nucleic acid molecule is capable of triggering a chain reaction of Cas activation until such time that all provided Cir-mediators are linearized producing a large number of activated RNPs. This cascade enabling a single nucleic acid to activate multiple RNPs which is at the core of the ultra-high sensitivity of this system for the detection of nucleic acid target down to 1 aM (<1 copy/uL).

In comparing to the previously reported blockage DNA structures which rely on the affinity changes between two nucleic acid strands, Cir-mediators requires the breakage of its DNA backbone to recover its activation function, which is more stable under various thermal, chemical and physical factors. More importantly, the signal amplification function of Cir-mediators and its corresponding CRISPR/Cas12a RNP does not depend on additional special reaction environments or specific nucleic acid sequences, hence, the overall system invented here can be directly applied to a majority of existing CRISPR/Cas12a biosensing systems based on trans-cleavage for signal generation in a cost-effective manner. In one aspect, the invention can be regarded as a universal self-amplification strategy for increasing sensitivity of CRISPR sensors without tedious system modification and optimization procedures. Moreover, the triggering principle of Cir-mediator-induced CRISPR/Cas12a amplification cascade reaction also has the potential to be extended towards other types of biosensing systems. In addition, in comparing to the common nucleic acid detection systems depends on the use of polymerase for amplified signal generation, this newly invented system is a fully cleavage-based nucleic acid detection approach which completely eliminates the possibility to generate nucleic acid amplicons, which are the major cause for laboratory and environment nucleic acid contamination.

### Example 5—Bifunctional Circular DNA Autocatalytic Amplifiers

The inventors have designed a bifunctional circular DNA amplifier to report nucleic acid detection events and simultaneously facilitate an autocatalytic reaction with a Cas RNP providing signal amplification without the need for an additional amplification reaction or device. Through the use of the circular DNA amplifiers described herein, a classic CRISPR/Cas assay can be converted into a DNA amplifier-enhanced CRISPR/Cas autocatalytic sensor (DANCER).

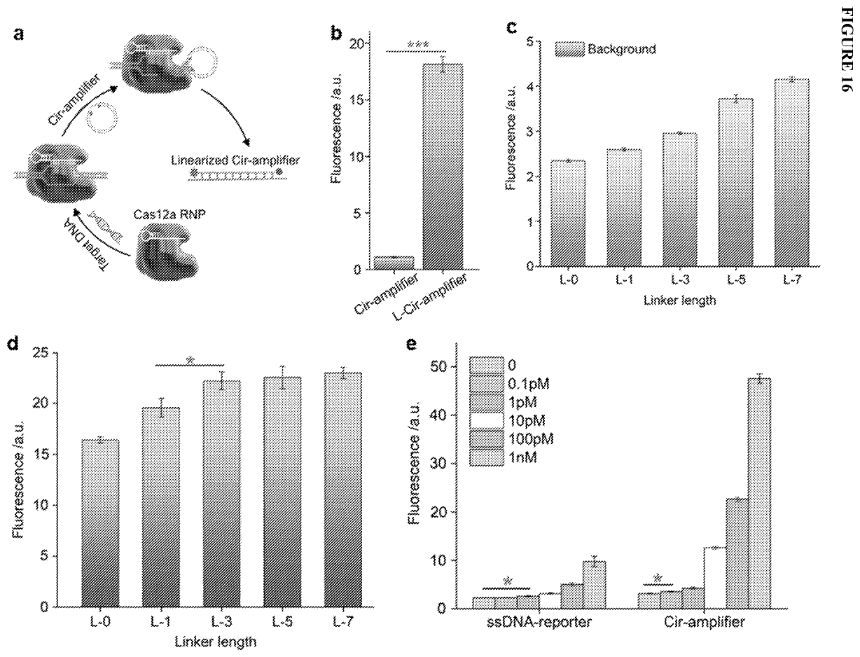

In this example, specially designed circular DNA amplifiers (Cir-amplifiers) were introduced to act both as a highly efficient reporter for signal readout and, simultaneously, to establish an autocatalytic reaction network with Cas12a RNPs (FIG. 14). This DNA Cir-amplifier comprises a circular single strand DNA (ssDNA) and an equal length or slightly shorter linear complementary DNA strand (cDNA) labelled at both ends with a fluorophore and a matching quencher. These two sequences together create a hybrid circular structure with a dsDNA sequence and a ssDNA region, i.e. wherein the dsDNA sequence is joined by a ssDNA backbone (i.e. 0 nt), or a very short ssDNA linker (e.g. 1-5 nt). In the Cir-amplifiers, the dsDNA sequence is the same as the target sequence. When the linker is linearized by the nucleases of an activated Cas RNP, the fluorescence signal restored. Furthermore, upon this cleavage, these now linearized Cir-amplifiers become “fake targets”, due to sequence identity with the real targets. This identity then drives the autocatalysis reaction. In this way, the Cir-amplifier plays a dual role in our system: of a catalytic substrate for trans-cleavage by activated Cas RNP, exactly like the reporter in a classic CRISPR sensor design, and an autocatalytic substrate for the yet-to-be activated Cas RNPs.

### 5.1 Materials and Methods

Synthesis and Characterization of Circular ssDNA

To synthesize Cir-ssDNA, 0.4 mL of 0.5% w/v streptavidin modified magnetic beads (0.74 μm) were first blocked with 1% BSA solution for 1 h to eliminate non-specific binding. Afterwards, 1 mL of 0.5 μM biotinylated linear-ssDNA was incubated with the beads for 1 h following a PBS wash to remove the residual free linear-ssDNA. Subsequently, 1 mL of the click chemistry reaction solution (1.0 mM CuSO4, 2.0 mM TCEP, and 100 μM TBTA) was added and incubated with the beads for 12 h at room temperature. After synthesis, the magnetic beads were collected and washed with PBS buffer to remove excess chemicals. Subsequently, 100 μL of 100 units/mL Exonuclease VII solution was added and incubated at 37° C. for 30 min to remove the linear ssDNA. After washing with PBS buffer, the synthesized Cir-ssDNA was released from the streptavidin-modified magnetic beads by heat treatment at 95° C. for 30 min, and the supernatant was collected for further use. All the Cir-ssDNA used in this research are synthesized based on this approach. The sequence listed in Table S1 is a demonstration example. Nanodrop was utilized to test the concentration of synthesized Cir-ssDNA.

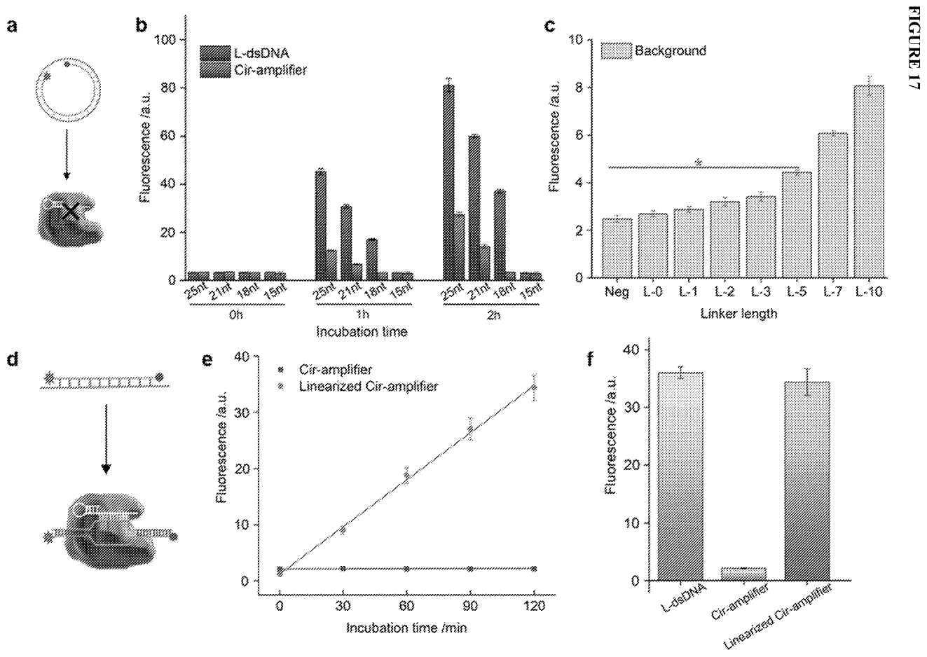

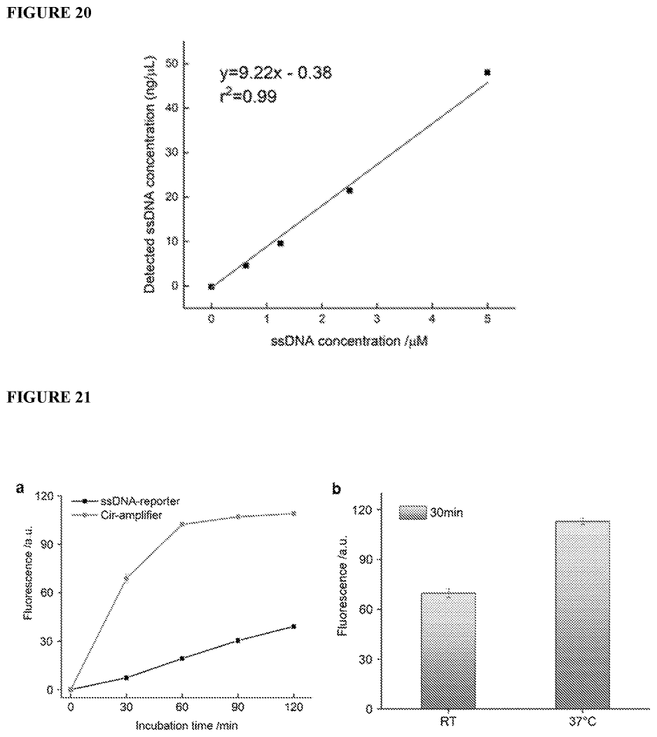

TABLE S1
DNA and RNA oligos used in FIG. 15 & FIG. 20.

| | Oligo | | | |
|---|---|---|---|---|
| Name | type | Sequence 5′-3′ | Modification | SEQ ID NO: |
| Cir-ssDNA- | ssDNA | T CTA/iBio-dT/GT GCT | 5′-Azide (N3); | SEQ ID NO: |
| 19 nt | | ATG TCT AAA | 3′-CHCH | 40 |

The formation of Cir-ssDNA was verified by using denaturing polyacrylamide gel (dPAGE) electrophoresis assay. 10 μL of Cir-ssDNA aliquoted with 2 μL 6×DNA gel loading dye was loaded into the gel for electrophoresis, which was carried out for 40 min at a constant voltage of 100V. 5 μL of 10 bp DNA ladder was used for molecular weight reference. Gel images were visualized by using Gel Doc+XR image system (Bio-Rad Laboratories Inc., USA).

### Investigation of the Reporter Performance of Cir-Amplifiers in a Classic CRISPR/Cas12a Biosensing System

The Cir-amplifier was assembled by mixing Cir-ssDNA with fluorophore labelled cDNA (Texas-cDNA-BHQ2). The Cir-amplifier based CRISPR/Cas12a reaction mixture was prepared as follows: 1 μL 100 μM (100 pmol) of Cas12a protein was gently mixed with 5 μL 20 μM (100 pmol) of gRNA-C in 3.6 mL 1×NEB 2.1 buffer. Then, 120 μL of 5 μM (0.6 nmol) of Cir-amplifier with different linker length (0-7 nt) were added and well mixed to form the standard Cir-amplifier involved reaction mixture. For comparison, linear ssDNA reporter assisted CRISPR/Cas12a reaction mixture was prepared: 1 μL 100 μM (100 pmol) of Cas12a protein was gently mixed with 5 μL 20 μM (100 pmol) of gRNA-C in 3.6 mL 1×NEB 2.1 buffer. Then, 6 μL of 100 μM (0.6 nmol) of pre-synthesized fluorescent quenched ssDNA reporters (Texas red-TTATT-BHQ2) were added and well mixed to form the standard linear ssDNA reaction mixture.

Afterwards, 10 μL of different concentrations (0, 0.1, 1, 10, 100, 1000 μM) of target-C ssDNA were added to 90 μL of the prepared reaction mixture containing either Cir-amplifiers or ssDNA reporters and incubated for 120 min. A SpectraMax iD5 multi-Mode Microplate Reader (Molecular Devices) was used for the detection of fluorescence readout. The Ex/Em of Texas-Cir-amplifier-BHQ2 was 570/615 nm. All the DNA and RNA oligos used in this experiment are listed in Table S2.

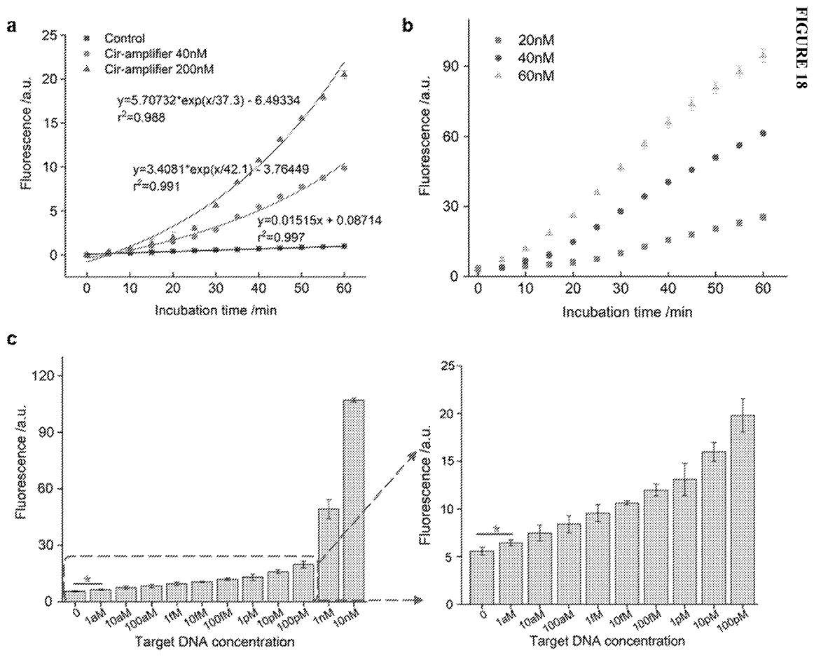

TABLE S2
DNA and RNA oligos used in FIG. 16 & FIG. 21.

| | Oligo | | | |
|---|---|---|---|---|
| Name | type | Sequence 5′-3′ | Modification | SEQ ID NO: |
| 18 nt cDNA | ssDNA | TTT AGA CAT AGC ACA | 5′-Texas Red; | SEQ ID NO: |
| | | TAG | 3′-BHQ2 | 41 |
| Cir-ssDNA- | ssDNA | CTA /iBio-dT/GT GCT ATG | 5′-Azide (N3); | SEQ ID NO: |
| 18 nt (L-0) | | TCT AAA | 3′-CHCH | 42 |
| Cir-ssDNA- | ssDNA | T CTA /iBio-dT/GT GCT ATG | 5′-Azide (N3); | SEQ ID NO: |
| 19 nt (L-1) | | TCT AAA | 3′-CHCH | 40 |
| Cir-ssDNA- | ssDNA | TTT CTA /iBio-dT/GT GCT | 5′-Azide (N3); | SEQ ID NO: |
| 21 nt (L-3) | | ATG TCT AAA | 3′-CHCH | 43 |
| Cir-ssDNA- | ssDNA | TT TTT CTA /iBio-dT/GT | 5′-Azide (N3); | SEQ ID NO: |
| 23 nt (L-5) | | GCT ATG TCT AAA | 3′-CHCH | 44 |
| Cir-ssDNA- | ssDNA | T TTT TTT CTA /iBio-dT/GT | 5′-Azide (N3); | SEQ ID NO: |
| 25 nt (L-7) | | GCT ATG TCT AAA | 3′-CHCH | 45 |
| target-C | ssDNA | GAA GAC ACC CTA CCA | N/A | SEQ ID NO: |
| | | ACC CCC CCC | | 2 |
| gRNA-C | ssRNA | UAA UUU CUA CUA AGU | N/A | SEQ ID NO: |
| | | GUA GAU GGG GGG GGU | | 46 |
| | | UGG UAG GGU GUC | | |
| ssDNA | ssDNA | TTATT | 5′-Texas Red; | N/A |
| reporter | | | 3′-BHQ2 | |

### Investigation of the RNP Activation Ability of Cir-Amplifier in a Classic CRISPR/Cas12a Biosensing System

In this experiment, the CRISPR/Cas12a reaction mixture was prepared as follows: 1 μL 100 μM (100 pmol) of Cas12a protein was gently mixed with 5 μL 20 μM (100 pmol) of gRNA-D in 3.6 mL 1×NEB 2.1 buffer. Then, 6 μL of 100 μM (0.6 nmol) of pre-synthesized fluorescent quenched ssDNA reporters (Texas red-TTATT-BHQ2) were added and well mixed to form the standard reaction mixture.

Afterwards, 10 μL 0.25 μM of a range of Cir-amplifiers with different dsDNA length and different ssDNA linker lengths were added to 90 μL of the prepared reaction mixture and incubated for 120 min. A SpectraMax iD5 multi-Mode Microplate Reader (Molecular Devices) was applied for the detection of fluorescence readout. The Ex/Em of Texas red-TTATT-BHQ2 reporter was 570/615 nm. For comparison, linear dsDNA was also applied to activate the CRISPR/Cas12a reaction mixture under the same conditions. All the DNA and RNA oligos used in this experiment are listed in Table S3 & S4.

TABLE S3
DNA and RNA oligos used in FIG. 17b.

| | Oligo | | | |
|---|---|---|---|---|
| Name | type | Sequence 5′-3′ | Modification | SEQ ID NO: |
| 25 nt cDNA | ssDNA | TTT AGA CAT AGC ACA | N/A | SEQ ID NO: |
| | | TAG ACT GAG A | | 47 |
| Cir-ssDNA- | ssDNA | TTT TCT CAG TCT A/iBio- | 5′-Azide (N3); | SEQ ID NO: |
| 28 nt | | dT/G TGC TAT GTC TAA A | 3′-CHCH | 48 |
| 21 nt cDNA | ssDNA | TTT AGA CAT AGC ACA | N/A | SEQ ID NO: |
| | | TAG ACT | | 49 |
| Cir-ssDNA- | ssDNA | TTT AGT CTA/iBio-dT/GT | 5′-Azide (N3); | SEQ ID NO: |
| 24 nt | | GCT ATG TCT AAA | 3′-CHCH | 50 |
| 18 nt cDNA | ssDNA | TTT AGA CAT AGC ACA | N/A | SEQ ID NO: |
| | | TAG | | 51 |
| Cir-ssDNA- | ssDNA | TTT CTA/iBio-dT/GT GCT | 5′-Azide (N3); | SEQ ID NO: |
| 21 nt | | ATG TCT AAA | 3′-CHCH | 43 |
| 15 nt cDNA | ssDNA | TTT AGA CAT AGC ACA | N/A | SEQ ID NO: |
| | | | | 52 |
| Cir-ssDNA- | ssDNA | TTT/iBio-dT/GT GCT ATG | 5′-Azide (N3); | SEQ ID NO: |
| 18 nt | | TCT AAA | 3′-CHCH | 53 |
| target-D | ssDNA | TCT CAG TCT ATG TGC | N/A | SEQ ID NO: |
| | | TAT GTC | | 54 |
| gRNA-D | ssRNA | UAA UUU CUA CUA AGU | N/A | SEQ ID NO: |
| | | GUA GAU GAC AUA GCA | | 55 |
| | | CAU AGA CUG AGA | | |
| ssDNA | ssDNA | TTATT | 5′-Texas Red; | N/A |
| reporter | | | 3′-BHQ2 | |

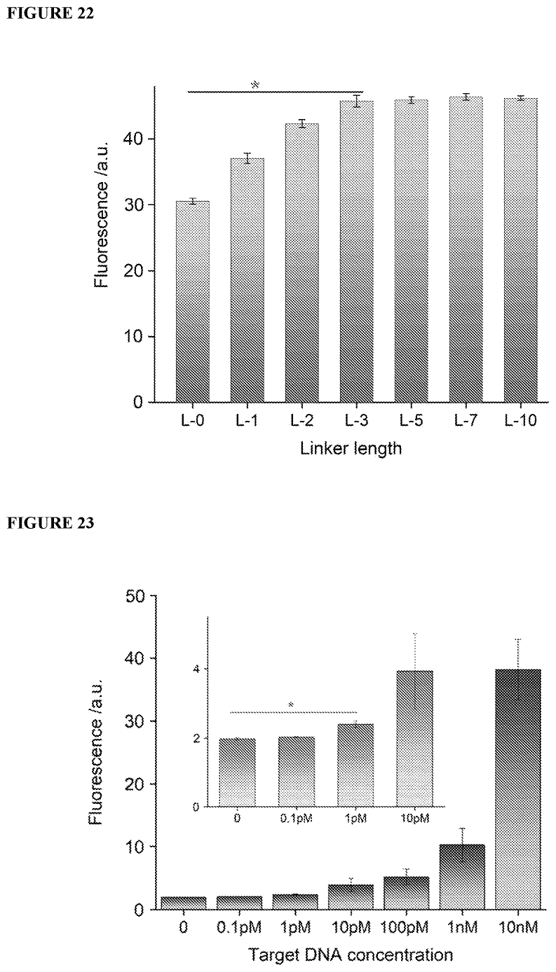

TABLE S4
DNA and RNA oligos used in FIG. 17c & FIG. 22.

| | Oligo | | | |
|---|---|---|---|---|
| Name | type | Sequence 5′-3′ | Modification | SEQ ID NO: |
| 18 nt cDNA | ssDNA | TTT AGA CAT AGC ACA | N/A | SEQ ID NO: |
| | | TAG | | 51 |
| Cir-ssDNA- | ssDNA | CTA/iBio-dT/GT GCT ATG | 5′-Azide (N3); | SEQ ID NO: |
| 18 nt (L-0) | | TCT AAA | 3′-CHCH | 42 |
| Cir-ssDNA- | ssDNA | T CTA/iBio-dT/GT GCT ATG | 5′-Azide (N3); | SEQ ID NO: |
| 19 nt (L-1) | | TCT AAA | 3′-CHCH | 40 |
| Cir-ssDNA- | ssDNA | TT CTA/iBio-dT/GT GCT | 5′-Azide (N3); | SEQ ID NO: |
| 20 nt (L-2) | | ATG TCT AAA | 3′-CHCH | 56 |
| Cir-ssDNA- | ssDNA | TTT CTA/iBio-dT/GT GCT | 5′-Azide (N3); | SEQ ID NO: |
| 21 nt (L-3) | | ATG TCT AAA | 3′-CHCH | 43 |
| Cir-ssDNA- | ssDNA | TT TTT CTA/iBio-dT/GT | 5′-Azide (N3); | SEQ ID NO: |
| 23 nt (L-5) | | GCT ATG TCT AAA | 3′-CHCH | 44 |
| Cir-ssDNA- | ssDNA | T TTT TTT CTA/iBio-dT/GT | 5′-Azide (N3); | SEQ ID NO: |
| 25 nt (L-7) | | GCT ATG TCT AAA | 3′-CHCH | 45 |
| Cir-ssDNA- | ssDNA | TTTT TTT TTT CTA/iBio- | 5′-Azide (N3); | SEQ ID NO: |
| 28 nt (L-10) | | dT/GT GCT ATG TCT AAA | 3′-CHCH | 57 |

### Investigation of the RNP Activation Efficiency of Linearized Cir-Amplifiers in a Classic CRISPR/Cas12a Biosensing System

The RNP activation efficiency of linearized Cir-amplifiers was evaluated using the CRISPR/Cas12a reaction mixture prepared by Method 3. Afterwards, 10 μL 0.25 μM of linearized Cir-amplifiers was added to 90 μL of the prepared reaction mixture and incubated for 120 min. A SpectraMax iD5 multi-Mode Microplate Reader (Molecular Devices) was applied for the detection of fluorescence readout. For comparison, linear dsDNA (18 nt) was also applied to activate the CRISPR/Cas 12a reaction mixture under the same conditions. All the DNA and RNA oligos used in this experiment are listed in Table S4.

### Evaluation and Biosensing Application of DANCER

The DANCER reaction mixture was prepared as follows: 1 μL 100 μM (100 pmol) of Cas12a protein was gently mixed with 5 μL 20 μM (100 pmol) of gRNA-D to form the Cas12a RNP in 5 mL 1×NEB 2.1 buffer. Subsequently, 200 μL of 5 μM (1 nmol) of Cir-amplifier solution was added and well mixed to form the reaction mixture.

Afterwards, 10 μL of target-D ssDNA at different concentrations were added to 90 μL of the prepared reaction mixture for activating trans-cleavage of Cas12a and enabling the CRISPR/Cas autocatalysis biosensing reaction. A SpectraMax iD5 multi-Mode Microplate Reader (Molecular Devices) was applied for the detection of fluorescence readout. The Ex/Em of Texas red-TTATT-BHQ2 reporter was 570/615 nm. All the DNA and RNA oligos used in this experiment are listed in Table S5.

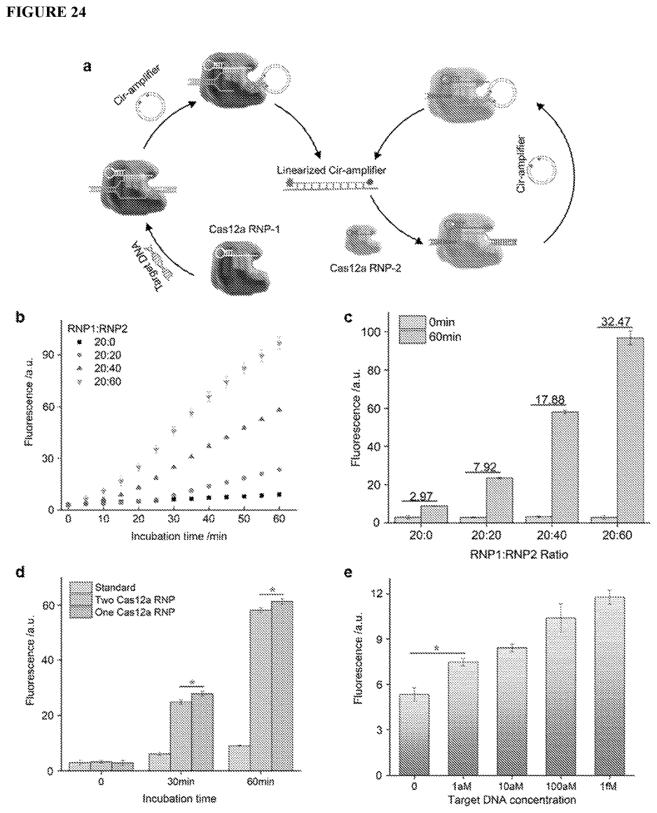

TABLE S5
DNA and RNA oligos used in FIG. 18 & FIG. 24

| | Oligo | | | |
|---|---|---|---|---|
| Name | type | Sequence 5′-3′ | Modification | SEQ ID NO: |
| target-D | ssDNA | TCT CAG TCT ATG TGC | N/A | SEQ ID NO: |
| | | TAT GTC | | 54 |
| gRNA-D | ssRNA | UAA UUU CUA CUA AGU | N/A | SEQ ID NO: |
| | | GUA GAU GAC AUA GCA | | 55 |
| | | CAU AGA CUG AGA | | |
| 18 nt cDNA | ssDNA | TTT AGA CAT AGC ACA | 5′-Texas Red; | SEQ ID NO: |
| | | TAG | 3′-BHQ2 | 41 |
| Cir-ssDNA- | ssDNA | TTT CTA/iBio-dT/GT GCT | 5′-Azide (N3); | SEQ ID NO: |
| 21 nt | | ATG TCT AAA | 3′-CHCH | 43 |
| target-C | ssDNA | GAA GAC ACC CTA CCA | N/A | SEQ ID NO: |
| | | ACC CCC CCC | | 2 |
| gRNA-C | ssRNA | UAA UUU CUA CUA AGU | N/A | SEQ ID NO: |
| | | GUA GAU GGG GGG GGU | | 46 |
| | | UGG UAG GGU GUC | | |
| ssDNA | ssDNA | TTATT | 5′-Texas Red; | N/A |
| reporter | | | 3′-BHQ2 | |

### Establishment of Orthotropic CRC Mouse Model

All animal experiments were approved by the UNSW Animal Care and Ethics Committee (project approval 20/95B, 21/39B, and 21/77B). NOD/SCID (6-8-week-old) mice were provided by Animal Services from the Animal Resources Centre (ARC, Perth, WA). Mice were housed in specific pathogen free conditions at 22° C. with a light/dark cycle of 12 h. Mice were kept in standard ventilated cages and acclimated for one-week following arrival into the UNSW animal facility. Mice were provided food and water ad libitum and their wellbeing was monitored regularly.

An orthotropic CRC mouse model was established by using intra-rectal tumor cell injection method with minor modifications of the previously reported work.31 In brief, 6-8-week-old female NOD/SCID mice were fasted of food for 6 h prior to cancer cell injections, followed by rapid anesthesia induction with 2-4% isoflurane and maintenance at 1-3% with 1 L/min oxygen. Lubricated blunt-tip forceps were used to dilate the anal canal, exposing the distal anal and rectal mucosa. Subsequently, 4×10⁵ HCT-116-Luc2 cells suspended in 10 μL PBS and 10 μL Matrigel were orthotopically inoculated into the distal posterior rectal submucosa, 1-2 mm above the anal canal using a 30-gauge needle (Terumo, Tokyo, Japan). Mice were closely monitored for 1 to 72 h post-injection for early detection of adverse events, with subsequent monitoring occurring at least bi-weekly.

Tumor formation and growth over time were monitored once a week by using the IVIS Spectrum CT imaging system (Perkin Elmer, Waltham, US). Typically, mice were intraperitoneally injected with 150 mg/kg of D-Luciferin. Mice were then anesthetized with isoflurane, with anesthesia maintained throughout imaging using the IVIS spectrum imaging system for bioluminescence detection via Living Image®4.5.2 software. When tumor reached the 100 mm3 volume (equivalent to approximately 4-6×1010 photons/s of bioluminescence signal in this study), one group of mice were treated with X-ray radiation. At 27 days post treatment, the terminal blood collection (500˜750 μL per mouse) was performed by the cardiac puncture technique with 25-gauge needles. K3 EDTA tubes were used for blood samples collection, allowing the isolation of blood plasma through centrifugation (1000×g, 10 min). The isolated mice blood plasma was stored at −80° C. for further use.

Application of DANCER for the Detection of cfDNA in Mouse Plasma

The DANCER reaction mixture for cfDNA detection was prepared as follows: 1 μL 100 μM (100 pmol) of Cas12a protein was gently mixed with 5 μL 20 μM (100 pmol) of gRNA-cf to form the Cas12a RNP in 5 mL 1×NEB 2.1 buffer. Subsequently, 200 μL of 5 μM (1 nmol) of Cir-amplifier solution was added and well mixed to form the reaction mixture.

Afterwards, 10 μL of spiked in sample or collected mouse plasma was added to 90 μL of the prepared reaction mixture for activating trans-cleavage of Cas12a and enabling the CRISPR/Cas biosensing reaction. A SpectraMax iD5 multi-Mode Microplate Reader (Molecular Devices) was applied for the detection of fluorescence readout. The Ex/Em of Tex-Cir-reporter-BHQ2 was 570/615 nm. All the DNA and RNA oligos used in this experiment are listed in Table S6.

Application of Colorimetric Cir-Amplifier Based DANCER for cfDNA Detection in Mouse Plasma Using Lateral Flow Assay

The colorimetric Cir-amplifier based DANCER reaction mixture was prepared as follows: 1 μL 100 μM (100 pmol) of Cas12a protein was gently mixed with 5 μL 20 μM (100 pmol) of gRNA-ct to form the Cas12a RNP in 5 mL 1×NEB 2.1 buffer. Subsequently, 200 μL of 5 μM (1 nmol) of colorimetric Cir-amplifier were added and well mixed to form the reaction mixture.

Afterwards, 10 μL of collected mouse plasma was added to 90 μL of the prepared reaction mixture for activating trans-cleavage of Cas12a and enabling the CRISPR/Cas biosensing reaction. After 15 min reaction, 5 μL of the reaction mixture was added to 95 μL of HybriDetect assay buffer (Milenia) and run on HybriDetect lateral flow strips (Milenia). All the DNA and RNA oligos used in this experiment are listed in Table S6.

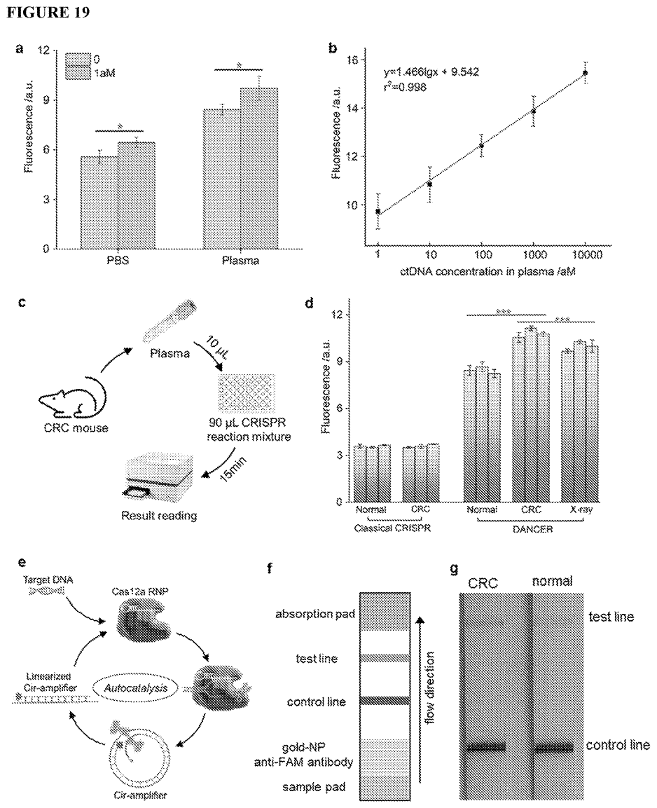

TABLE S6
DNA and RNA oligos used in FIG. 19

| | Oligo | | | |
|---|---|---|---|---|
| Name | type | Sequence 5′-3′ | Modification | SEQ ID NO: |
| cfDNA² | ssDNA | CTC AGT GAT TTT AGA | N/A | SEQ ID NO: |
| | | GAG AGG AT | | 58 |
| gRNA-cf | ssRNA | UAA UUU CUA CUA AGU | N/A | SEQ ID NO: |
| | | GUA GAU CCU CUC UCU | | 59 |
| | | AAA AUC ACU GAG | | |
| Cir-ssDNA | ssDNA | TTT A/iBio-dT/T TTA GAG | 5′-Azide (N3); | SEQ ID NO: |
| | | AGA GGT AAA | 3′-CHCH | 60 |
| cDNA | ssDNA | TTT ACC TCT CTC TAA | 5′-Texas Red; | SEQ ID NO: |
| | | AAT | 3′-BHQ2 | 61 |
| cDNA-extend | ssDNA | TTT ACC TCT CTC TAA | 5′-FAM; 3′- | SEQ ID NO: |
| | | AAT CCC CC | Biotin | 62 |
| ssDNA | ssDNA | TTATT | 5′-Texas Red; | N/A |
| reporter | | | 3′-BHQ2 | |

### Method S1. Materials

Chemicals and biological reagents: EnGen® Lba Cas12a (Cpf1) protein (New England Biolab), 10×NEB 2.1 buffer (New England Biolab), exonuclease VII (New England Biolab), SYBR Gold DNA dye (ThermoFisher), 10 bp DNA ladder (ThermoFisher), 6×DNA loading dye (ThermoFisher), copper sulfate (CuSO₄) (Sigma, 209198), Tris(2-carboxyethyl) phosphine (TCEP) (Sigma, C4706), tris(benzyltriazolylmethyl) amine (TBTA) (ChemSupply, T2993), streptavidin coated magnetic particles (Spherotech, SVM-08-10), DNase/RNase free water (ThermoFisher), phosphate buffered saline (PBS) (Sigma, 10 mM, pH=7.4), and HybriDetect—Universal Lateral Flow Assay Kit (Milenia biotec).

All DNA and RNA oligos are synthesized and modified by Sangon Bio-Tech Ltd.

Method S2. Investigation of the ssDNA Linker Length in Cir-Amplifier.

To investigate the ssDNA linker length in Cir-amplifier, the inventors conducted the following two step experiments. In the first step, the reaction mixture was prepared as follows: 1 μL 100 μM (100 pmol) of Cas12a protein was gently mixed with 5 μL 20 μM (100 pmol) of classic-gRNA in 3.6 mL 1×NEB 2.1 buffer. Then, 180 μL of 5 μM (0.9 nmol) of Cir-amplifier with different linker length were added and well mixed to form the reaction mixture. Afterwards, 10 μL 0.25 μM of the target-C ssDNA was added into 90 μL of the prepared CRISPR/Cas12a reaction mixture for activating trans-cleavage of Cas12a and cleaving Cir-amplifiers to linearized Cir-amplifiers. After one hour incubation at room temperature, the reaction mixture was collected for further use. To eliminate the influence of activated Cas12a RNPs from step 1, the reaction mixture was heated to 65° C. for 10 min to deactivate all the Cas12a RNPs from step 1. This ensures that only linearized Cir-amplifier will be the active trigger for downstream step 2 biosensing system.

In the second step, the reaction mixture was prepared as follows: 1 μL 100 μM (100 pmol) of Cas12a protein was gently mixed with 5 μL 20 μpM (100 pmol) of amplifier-gRNA in 3.6 mL 1×NEB 2.1 buffer. Then, 6 μL of 100 μM (0.6 nmol) of pre-synthesized fluorescent quenched ssDNA reporters (Texas red-TTATT-BHQ2) were added and well mixed to form the reaction mixture. Subsequently, 10 μL of prepared reaction mixture from step 1 (25 nM) was added to 90 μL CRISPR/Cas12a reaction mixture prepared in step 2 and incubated for 120 min. A SpectraMax iD5 multi-Mode Microplate Reader (Molecular Devices) was applied for the detection of fluorescence readout. The Ex/Em of Texas red-TTATT-BHQ2 reporter was 570/615 nm.

### Method S3. The Evaluation and Biosensing Application of Two Cas12a RNP Based Autocatalysis Biosensing System (DANCER-2)

The two Cas12a RNP based autocatalysis reaction mixture was prepared as follows: 1 μL 100 μM (100 pmol) of Cas12a protein was gently mixed with 5 μL 20 μM (100 pmol) of classic-gRNA to form the Cas12a RNP-1. In the meanwhile, 1 μL 100 μM (100 pmol) of Cas12a protein was gently mixed with 5 μL 20 μM (100 pmol) of D-gRNA to form the Cas12a RNP-2. Afterwards, the prepared Cas12a RNP-1 and Cas12a RNP-2 were mixed with 200 μL of 5 μM (1 nmol) of Cir-amplifiers in 5 mL 1×NEB 2.1 buffer to form the standard reaction mixture.

Afterwards, 10 μL of target-C ssDNA at different concentrations were added to 90 μL of the prepared reaction mixture for activating trans-cleavage of Cas12a and enabling the CRISPR/Cas biosensing reaction. A SpectraMax iD5 multi-Mode Microplate Reader (Molecular Devices) was applied for the detection of fluorescence readout.

### 5.2 Results

Synthesis and Characterization of Circular ssDNA.

Cir-amplifiers were synthesized by generating a single ring circular-ssDNA (Cir-ssDNA) using click chemistry as described in the materials and methods section above. Briefly, a single ring circular-ssDNA was synthesized. Streptavidin-modified magnetic beads were utilized to immobilize the biotinylated (linear) ssDNA (FIG. 15a). Subsequently, the click chemistry approach was applied to form the Cir-ssDNA by bonding of azide and alkyne (CHCH) functional groups. The remaining linear ssDNA was degraded by exonuclease, and Cir-ssDNA was released from the streptavidin beads by heating to 95° C. Single ring Cir-ssDNA thus synthesized was characterized by using denaturing polyacrylamide gel (dPAGE) electrophoresis assay (FIG. 15b), where the band of Cir-ssDNA (column 3) was found to move more slowly than the band of linear ssDNA (column 2), confirming the formation of Cir-ssDNA. In addition, only a single band of Cir-ssDNA (column 3) was observed, consistent with the formation of single ring Cir-ssDNA. Synthesis efficiency of Cir-ssDNA was optimized by varying the concentration of magnetic beads, and over 90% synthesis efficiency was achieved (FIG. 15c & FIG. 20). The Cir-ssDNA synthesis method was highly reproducible (FIG. 15d, coefficient of variation of 1.06%).

### Performance of Cir-Amplifiers as Reporters in a Classic CRISPR/Cas12a Biosensing System

The Cir-amplifiers are designed to be a catalytic substrate for activated Cas12a RNPs and produce a fluorescent reporter signal once linearized (FIG. 16a). This requires a significant fluorescence signal difference between Cir-amplifier and linearized Cir-amplifier shown in FIG. 16b. Limited fluorescent background was observed in the case of Cir-amplifiers since the fluorophore-quencher distance (equivalent to the ssDNA linker length) was within the FRET distance of the Texas/BHQ2 pair (5 nt). After cleavage, the fluorescence signal of a linearized Cir-amplifier was restored since the distance of fluorophore and quencher (18 nt, estimated to be 6.12 nm) exceeded the FRET distance of Texas/BHQ2 (over 10 nt). The fluorescence signal of the Cir-amplifier increased over 16.5 times upon linearization, confirming that it can act as a reporter in classical CRISPR/Cas12a biosensing systems.

Optimization of the reporter function in our DNA amplifier was carried out by changing the linker length (FIG. 16c). Higher fluorescent background was observed for longer linker lengths, which was due to increased fluorophore-quencher distance. The inventors then exposed the Cir-amplifiers with different ssDNA linker lengths to activated Cas12a RNPs (FIG. 16d). The fluorescence signal was found to increase for ssDNA linker lengths from 0 to 3, since longer ssDNA linkers could be more easily cleaved by Cas12 RNP. With further increase of ssDNA linker length from 3 nt to 7 nt, the fluorescence signal remained similar. Thus, 3 nt ssDNA linker length was found to be optimal. The optimized Cir-amplifier with 18 nt dsDNA and 3 nt ssDNA linker was also compared with a classic linear ssDNA reporter (TTATT, with identical fluorophore-quencher pairs) in a classic CRISPR/Cas12a biosensing system, and higher fluorescence signal by a factor of 3 was observed for the Cir-amplifier (FIG. 21). Additionally, the limit of detection (LOD) of Cir-amplifier-assisted CRISPR/Cas12a biosensing system (0.1 pM) was found to be 10 times lower than that of linear ssDNA reporter-assisted CRISPR/Cas12a biosensing system (1 pM) (FIG. 16e). Therefore, our Cir-amplifier represents a high-performance reporter for CRISPR/Cas12a biosensors.

### RNP Activation Efficiency of Cir-Amplifier and Linearized Cir-Amplifier in a CRISPR/Cas12a Biosensing System

The inventors assessed the RNP activation efficiency of Cir-amplifiers in a CRISPR/Cas12a biosensing system (FIG. 17a). Since the Cir-amplifier contains two key regions, the dsDNA section and ssDNA linker, the inventors systematically investigated the lengths of each. As shown in FIG. 17b, a certain amount of RNP activation by Cir-amplifiers giving rise to a background signal was observed when the dsDNA length was higher than 18 nt, and RNP activation was significantly reduced when the dsDNA length was lower than 18 nt. Since minimal RNP activation by Cir-amplifiers is desirable, 18 nt dsDNA length was selected for following studies. Afterwards, the inventors investigated the ssDNA linker length in Cir-amplifiers. As shown in FIG. 17c, the RNP activation (background signal) slightly increased from 0-3 nt, and then grew sharply from 5-10 nt, thus the ssDNA linker length below 3 nt was found to be optimal for background control. Additionally, since longer ssDNA linkers are easier to be cleaved than shorter ones (FIG. 22), a 3 nt ssDNA linker was selected for the following work.16

Furthermore, the RNP activation efficiency by linearized Cir-amplifier in a CRISPR/Cas12a biosensing system was investigated (FIG. 17d). A linearized Cir-amplifier with 18 nt dsDNA and 3 nt ssDNA was applied to activate a CRISPR/Cas12a biosensing system. Excellent activation efficiency with over 20 times fluorescence increase was observed (FIG. 17e), which was comparable to that of linear dsDNA (18 nt) (FIG. 17f), indicating that the ability of Cir-amplifier to activate RNPs was fully recovered upon its linearization. This means that a Cir-amplifier with 18 nt dsDNA and 3 nt ssDNA only minimally activates the available Cas12a RNPs but linearized Cir-amplifiers with 18 nt dsDNA and 3 nt ssDNA are able to activate these RNPs just like the original molecular targets.

### Establishment of the DNA Amplifier-Enhanced CRISPR/Cas Autocatalytic Sensor (DANCER)

The CRISPR/Cas autocatalytic sensor (DANCER, also referred to as AutoCAR-2) is established by using two components, Cas12a RNPs and Cir-amplifiers. This seemingly minor modification of replacing linear ssDNA reporters in a classic CRISPR/Cas sensor system with Cir-amplifiers has a profound impact on the reaction network within the sensor. As schematically shown in FIG. 14a, with the introduction of target DNA, the DANCER autocatalysis system is initiated. The target activated RNP linearizes a number of Cir-amplifiers which then continue to generate additional activated Cas12a RNPs, and these create an avalanche of additional linearized Cir-amplifiers—each of which reports the detection. This avalanche continues producing an ever-increasing signal as long as the Cir-amplifier substrate and Cas12a RNPs are not depleted. In comparison with a classical CRISPR/Cas12a sensor (FIG. 14b), whose signal linearly increases with time (FIG. 18a, control), DANCER provides an exponentially increasing signal (FIG. 18a). Additionally, in DANCER for the same amount of Cas12a RNP (20 nM), with increasing supply of Cir-amplifiers, an increasing fluorescence signal was observed (FIG. 18a). Correspondingly, with the same amount of Cir-amplifiers (200 nM), with increasing Cas12a RNP levels, an increasing fluorescence signal was observed as well (FIG. 18b), which is indicative of higher total levels of RNP activation in DANCER compared with a classic CRISPR/Cas sensor. Finally, the biosensing performance of DANCER was investigated (FIG. 18c), and 1 aM of limit of detection (LOD) was achieved, with more than 11 orders of magnitude detection range. This is 6 orders of magnitude higher than the LOD of a classic CRISPR/Cas12a biosensing system (1 pM) (FIG. 23). Therefore, DANCER is an ultrasensitive biosensing system capable of detection of single nucleic acid targets per microliter.

To further expand the biosensing applications of DANCER, the inventors combined a classic CRISPR/Cas12a biosensing system with DANCER to establish a versatile DANCER-2 system (FIG. 24), in which the DANCER system functions as an additional signal amplification loop for a classical CRISPR/Cas12a biosensing system. A comparable performance of DANCER-2 (with LOD of 1 aM) was observed with DANCER system (FIG. 24) confirming its versatility.

The autocatalysis-driven biosensing performance of DANCER was further interpreted by a model system of chemical kinetics rate equations which introduces the autocatalysis loop for Cas12a. It allows to establish that the experimentally observed increase is approximately exponential when the RNPs and Cir-amplifier are sufficiently abundant and do not get noticeably depleted, which has been confirmed by an exponential fit in FIG. 18a.

Application of DANCER for the Point-of-Care Quantification of cfDNA in Plasma of Mice with Human Colorectal Cancer Xenografts

The biosensing performance of DANCER in future clinical settings was evaluated via the detection of cell-free DNA (cfDNA) in mice with orthotopic human colorectal cancer xenografts. Three groups of mice were prepared, including normal mice, mice bearing human colorectal cancer (CRC-mouse), and X-ray treated CRC-mouse. Blood samples were collected from all animal groups for the analyses of cfDNA in blood plasma. Prior to the analysis of cfDNA from animal models, the biosensing performance of DANCER in a synthesized cfDNA spiked plasma sample was first evaluated. As shown in FIG. 19a, 1 aM sensitivity was achieved in a non-diluted plasma sample although higher background signal was observed than in plasma-free controls. A DANCER calibration curve extending over 4 orders of magnitude was obtained by testing of different concentrations of cfDNA spiked into prepared plasma samples (FIG. 19b). The DANCER system was then used for the detection of cfDNA from mouse plasma in a procedure shown in FIG. 19c, in which 10 μL plasma sample was sufficient for a test. The analysis results confirm that the DANCER system was able to distinguish cfDNA from normal and diseased mice (FIG. 19d), while a classic CRISPR biosensing system was not able to realize such detection, confirming superiority of the DANCER system in future clinical settings. In addition, lower cfDNA concentration was observed in X-ray treated CRC-mouse (FIG. 19d), confirming the feasibility to monitor cancer treatment effect by using DANCER. Furthermore, based on the standard calibration curve (FIG. 19b), the cfDNA concentrations in CRC and X-ray treatment groups were estimated to be 74.5 aM and 19.9 aM, respectively, while no cfDNA was found in the normal mouse group.

To further expand the applicability of a fluorescent DANCER system to a point-of-care setting, a new colorimetric Cir-amplifier was established (FIG. 19e). The core structure of colorimetric Cir-amplifier was identical to the fluorescent Cir-amplifier discussed earlier, the only difference was the extension of 18 nt cDNA with 5 nt CCCCC with a biotin on 3′ end. After the DANCER reaction was deemed to be sufficiently executed, a lateral flow assay (LFA) was applied for the colorimetric signal readout of Cir-amplifiers (FIG. 19f). An anti-FAM antibody with gold nanoparticle on the conjugation pad was applied to recognize the fluorophore FAM on the 5′ end of colorimetric Cir-amplifier (FIG. 19e). As the reaction product flowed through the LFA, the antibody & Cir-amplifier complex was captured by the streptavidin on the control line through the binding of biotin on the 3′ end of colorimetric Cir-amplifier (FIG. 19e), and red color appeared on the control line (FIG. 19g). With further flow of the sample, the secondary antibody on the test line captured the anti-FAM antibody for colorimetric signal readout. In terms of the whole DANCER & LFA assay, in the presence of a genuine target, the DANCER system was activated, and colorimetric Cir-amplifiers were cleaved to yield linearized Cir-amplifiers (FIG. 19e), which were further captured by the secondary antibody on the test line for signal readout due to the loss of their 3′-5C biotin tails (positive test). Conversely, without a genuine target, the DANCER system was not activated, and colorimetric Cir-amplifier remained intact (FIG. 19e), and was further captured by the streptavidin on the control line for signal readout (negative test). Finally, the established DANCER & LFA assay was applied to test the plasma cfDNA from normal and CRC-mice (FIG. 19g), and an obvious color intensity difference was observed between the CRC-mouse (74.5 aM of target) and normal mouse (0 aM of target) samples (FIG. 19f), confirming the potential to apply the DANCER system in a point-of-care setting.

### 5.3 Discussion

In this example, the inventors have demonstrated a novel DNA amplifier-enhanced CRISPR/Cas autocatalytic sensor (DANCER) which is capable of ultrasensitive detection of a single copy of nucleic acids without amplification at room temperature (FIG. 18). Additionally, DANCER is able to quantify cfDNA from clinical samples and reveal the presence of abnormal status at a point-of-care setting, demonstrated here in blood plasma of mice with human CRC xenografts using both fluorescent and LFA based colorimetric readout (FIG. 19). DANCER is an elegant autocatalysis system, which only involves two molecular components, Cas RNPs and Cir-amplifiers. The Cir-amplifier as the key component of DANCER system, has the ability of both activating downstream Cas RNPs to drive the autocatalysis reaction system (FIG. 18) and acting as a fluorescent reporter for real-time signal readout (FIG. 16). The formation of Cir-amplifiers is based on intramolecular linkage to form single ring. Herein, the inventors introduced a novel magnetic beads-based click chemistry method for the synthesis of Cir-ssDNA, which shows high efficiency (>90%), excellent reproducibility, and it yields only a single ring circular ssDNA (FIG. 15).

Being a single pot reaction, DANCER is compatible with point-of-care ultrasensitive quantification of nucleic acids. In contrast to conventional nucleic acid amplification technologies, such as PCR, RPA, and LAMP, DANCER provides a comparable sensitivity (1 aM) but it is free from disadvantages existent in currently employed methods such as the requirement for temperature cycling, or the potential for primer polymerization. Additionally, in comparison with other CRISPR biosensing systems which have high sensitivity and specificity, but still require nucleic acid amplification step, DANCER provides a rapid amplification free approach with compatible sensitivity and specificity, but without amplicon contamination. Furthermore, in comparison with other signal amplification technologies assisted CRISPR biosensors, DANCER does not require any sophisticated instrumentation and it can be performed at room temperature with a point-of-care setting. In comparison with other recently established methods, which require more than one hour turnover time due to a complex system design with multiple components, the DANCER system is capable of rapid detection of 1 aM nucleic acids within 15 min (FIG. 18c).

DANCER provides a versatile approach for signal amplification for future biosensing solutions. Through the programmability of Cas nucleases, it is capable of ultrasensitive detection of a broad range of nucleic acid analytes. Additionally, it can be directly integrated into all the type V based CRISPR/Cas biosensing system (based on Cas12 & Cas14) as an additional signal amplifier to enhance their sensitivity, without any additional changes of the original reagents or setup (FIG. 24). In conclusion, DANCER offers a breakthrough approach for rapid, point-of-care, and ultrasensitive quantification of nucleic acids.

### Example 6. Circular-gRNA Mediated CRISPR/Cas12a Autocatalysis Biosensing System

In this example, the inventors established a circular-gRNA (Cir-gRNA) mediated CRISPR/Cas12a autocatalysis biosensing system for the ultrasensitive detection of target nucleic acids at 1 aM sensitivity.

### 6.1 Materials and Methods

### Materials

Chemicals and biological reagents: EnGen® Lba Cas12a (Cpf1) protein (New England Biolab), 10×NEB 2.1 buffer (New England Biolab), copper sulfate (CuSO₄) (Sigma, 209198), Tris(2-carboxyethyl) phosphine (TCEP) (Sigma, C4706), tris(benzyltriazolylmethyl) amine (TBTA) (ChemSupply, t2993), DNase/RNase free water (ThermoFisher), phosphate buffered saline (PBS) (Sigma, 10 mM, pH=7.4).

All DNA and RNA oligos are synthesized and modified by Sangon bio-tech Ltd. For the Texas Red modified oligos, the excitation wavelength was 570 nm and emission wavelength was 615 nm.

TABLE S7
RNA and DNA oligo sequences used in this example.

| | Oligo | | Length | | |
|---|---|---|---|---|---|
| Name | type | Sequence 5′-3′ | (nt/bp) | Modification | SEQ ID NO: |
| Target | ssDNA | GAA GAC ACC CTA | 24 | N/A | SEQ ID NO: |
| DNA | | CCA ACC CCC CCC | | | 2 |
| T-gRNA | ssRNA | UAA UUU CUA | 42 | N/A | SEQ ID NO: |
| | | CUA AGU GUA | | | 46 |
| | | GAU GGG GGG | | | |
| | | GGU UGG UAG | | | |
| | | GGU GUC | | | |
| Circular | ssRNA | UAA UUU CUA | 44 | 5′-Azide; | SEQ ID NO: |
| ssRNA | | CUA AGU GUA | | 3′-CHCH | 63 |
| (Cir- | | GAU GAC AUA | | | |
| gRNA) | | GCA CAU AGA CUG | | | |
| | | AGA/iBiodT//dTdT/ | | | |
| Standard | ssRNA | UAA UUU CUA | 42 | 5′-Azide; | SEQ ID NO: |
| gRNA | | CUA AGU GUA | | 3′-CHCH | 64 |
| | | GAU GAC AUA | | | |
| | | GCA CAU AGA CUG | | | |
| | | AGA | | | |
| Trigger | ssDNA | TCT CAG TCT ATG | 25 | N/A | SEQ ID NO: |
| ssDNA | | TGC TAT GTC TAA | | | 65 |
| | | A | | | |
| cDNA | ssDNA | TTTA GAC ATA | 25 | N/A | SEQ ID NO: |
| | | GCA CAT AGA CTG | | | 20 |
| | | AGA | | | |
| ssDNA | ssDNA | TTATT | 5 | 5′-Texas Red; | N/A |
| reporter | | | | 3′-BHQ2 | |

### Synthesis of Cir-RNA

To synthesize Cir-RNA, 0.4 mL of 0.5% w/v streptavidin modified magnetic beads (0.74 μm) were first blocked with 100 BSA solution for 1 h to eliminate non-specific binding. Afterwards, 1 mL of 0.5 aM biotinylated linear-RNA was incubated with the beads for 1 h following a PBS wash to remove the residual free linear-RNA. Subsequently, 1 mL of the click chemistry reaction solution (1.0 mM CuSO4, 2.0 mM TCEP, and 100 μM TBTA) was added and incubated with the beads for 12 h at room temperature. After synthesis, the magnetic beads were collected and washed with PBS buffer to remove excess chemicals. Afterwards, the synthesized Cir-ssDNA was released from the streptavidin-modified magnetic beads by heat treatment at 95° C. for 30 min, and the supernatant was collected for further use.

### Evaluating the Formation of Cir-gRNA

To evaluate the formation of Cir-gRNA, the synthesized Cir-gRNA was applied to activate the standard CRISPR/Cas12a biosensing system. The standard CRISPR/Cas12a reaction mixture was prepared as follows: 1 μL 100 μM (100 pmol) of Cas12a protein was gently mixed with 6 μL of 100 μM (0.6 nmol) of pre-synthesized fluorescent quenched ssDNA reporters (Texas red-TTATT-BHQ2) in 3.6 mL 1×NEB 2.1 buffer to form the standard reaction mixture.

Afterwards, 5 μL 0.5 μM of Cir-gRNA and 5 μL 0.5 μM of trigger ssDNA were added to 90 μL standard CRISPR/Cas12a reaction mixture and incubated for 120 min. For comparison, standard gRNA was also applied to activate the standard CRISPR/Cas12a biosensing system under the same condition. A SpectraMax i3x multi-Mode Microplate Reader (Molecular Devices) was applied for the detection of fluorescence readout.

### Application of Cir-gRNA for the Development of Cas12a Autocatalysis Biosensing System

The Cir-RNA mediated Cas12a autocatalysis biosensing system was prepared as follows: 1 μL 100 μM (100 pmol) of Cas12a protein was gently mixed with 5 μL 20 μM (100 pmol) of specifically designed T-gRNA to form Cas12a RNP-1 in 5 mL 1×NEB 2.1 buffer. Afterwards, more chemicals were added into the solution, including 1 μL 100 μM (100 pmol) of Cas12a protein, 1 μL 100 μM (100 pmol) trigger ssDNA, 1 μL 100 μM (100 pmol) trigger cDNA, 20 μL 5 μM (100 pmol) synthesized Cir-gRNA, and 6 μL of 100 μM (0.6 nmol) ssDNA reporters (Texas red-TTATT-BHQ2). The Cas12a protein will interact with the linearized Cir-gRNA to form Cas12a RNP-2. To investigate the ratio between RNP-1 and RNP-2, the final concentration of RNP-1 was fixed to be 20 nM, and the final concentration of RNP-2 was changed from 0 to 80 nM.

Afterwards, 10 μL 1 pM of target DNA were added to 90 μL of the Cir-RNA mediated Cas12a autocatalysis reaction mixture for biosensing reaction. A SpectraMax i3x multi-Mode Microplate Reader (Molecular Devices) was applied for the detection of fluorescence readout.

### Evaluating the Biosensing Performance of Cir-gRNA Mediated CRISPR/Cas12a Biosensor

The Cir-RNA mediated Cas12a autocatalysis biosensing system was prepared as follows: 1 μL 100 μM (100 pmol) of Cas12a protein was gently mixed with 5 μL 20 μM (100 pmol) of specifically designed T-gRNA to form Cas12a RNP-1 in 5 mL 1×NEB 2.1 buffer. Afterwards, more chemicals were added into the solution, including 1 μL 100 μM (100 pmol) of Cas12a protein, 1 μL 100 μM (100 pmol) trigger ssDNA, 1 μL 100 μM (100 pmol) trigger cDNA, 20 μL 5 μM (100 pmol) synthesized Cir-gRNA, and 6 μL of 100 μM (0.6 nmol) ssDNA reporters (Texas red-TTATT-BHQ2).

Afterwards, 10 μL of different concentrations of target DNA were added to 90 μL of the Cir-RNA mediated Cas12a autocatalysis reaction mixture for biosensing reaction. A SpectraMax i3x multi-Mode Microplate Reader (Molecular Devices) was applied for the detection of fluorescence readout.

### 6.2 Results

### Design of Cir-RNA Mediated Cas12a Autocatalysis Biosensing System

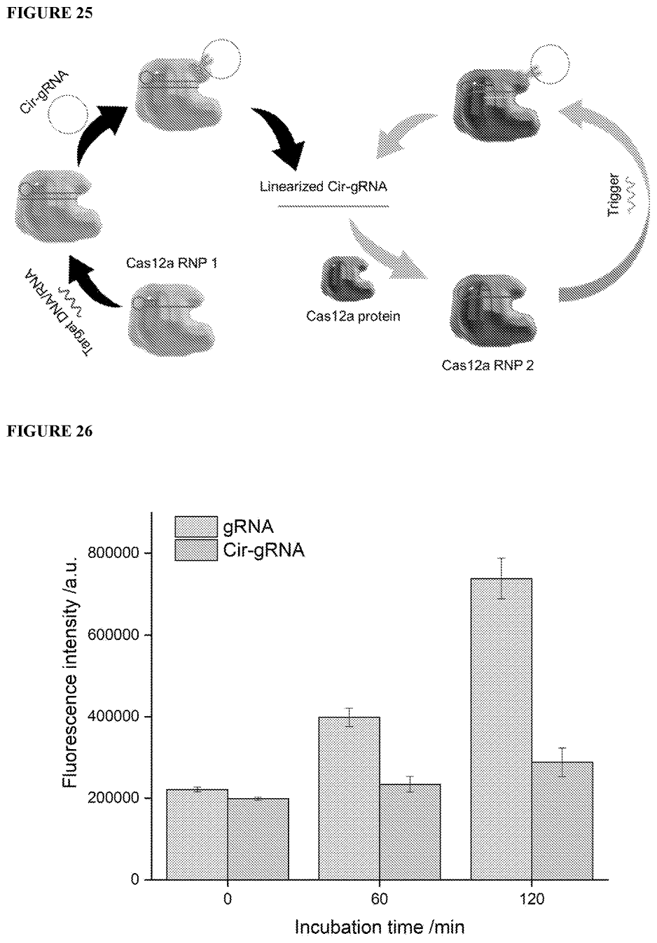

As shown in FIG. 25, two Cas12a RNPs were utilized in this system. The Cas12a RNP-1 (orange) is responsible for the recognition of target DNA, and Cas12a RNP-2 (blue) is responsible for the signal amplification. After introducing the target DNA in the biosensing system, Cas12a RNP1 was activated, followed by the cleavage of Cir-gRNA to form the linearized Cir-gRNA. Afterwards, the linearized Cir-gRNA was loaded into the free Cas12a protein to form a new Cas12a RNP2, which was further be activated by the free trigger DNA to form a new activated Cas12a RNP. Afterwards, the new activated Cas12a RNP will also cleaves the Cir-gRNA to form the autocatalysis reaction system.

### Demonstrating the Formation of Cir-gRNA

To investigate the formation of Cir-gRNA, the synthesized Cir-gRNA was applied to activate the standard CRISPR/Cas12a biosensing system. For comparison, standard gRNA was also applied to activate the standard CRISPR/Cas12a biosensing system under the same condition. As shown in FIG. 26, the fluorescence signal of standard gRNA elevated with the increase of incubation time, however the fluorescence signal of Cir-gRNA kept consistent with the time increase, conforming the formation of Cir-gRNA.

### Establishment of Cir-RNA Mediated Cas12a Autocatalysis Biosensing System

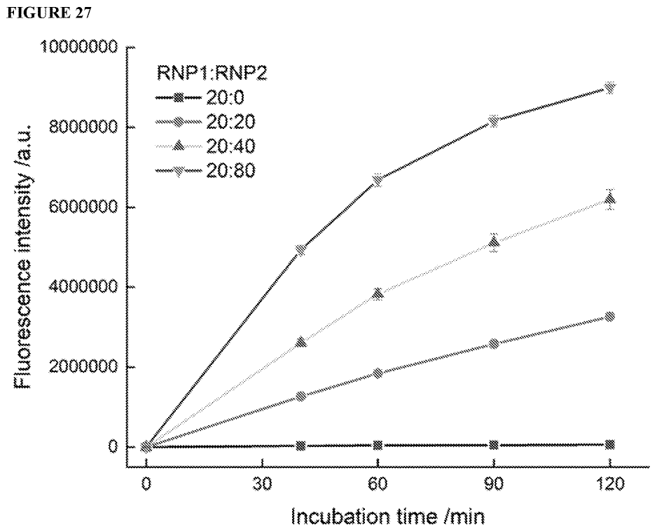

As shown in FIG. 27, Cir-RNA mediated Cas12a autocatalysis system was established. The concentration of RNP1 was fixed to be 20 nM, while the concentration of RNP2 was increased from 0 to 80 nM. When the concentration of RNP2 is 0, the whole biosensing system is the standard Cas12a biosensing system. Comparing the standard Cas12a biosensing system (RNP2=0) with Cas12a autocatalysis system (RNP2>0), higher fluorescence signals were observed in all Cas12a autocatalysis systems, confirming the formation of Cas12a autocatalysis system.

### Performance of Cir-gRNA Mediated CRISPR/Cas12a Autocatalysis Biosensing System

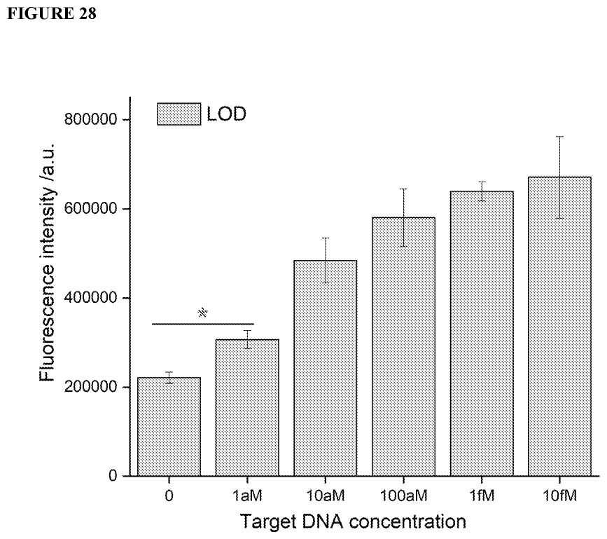

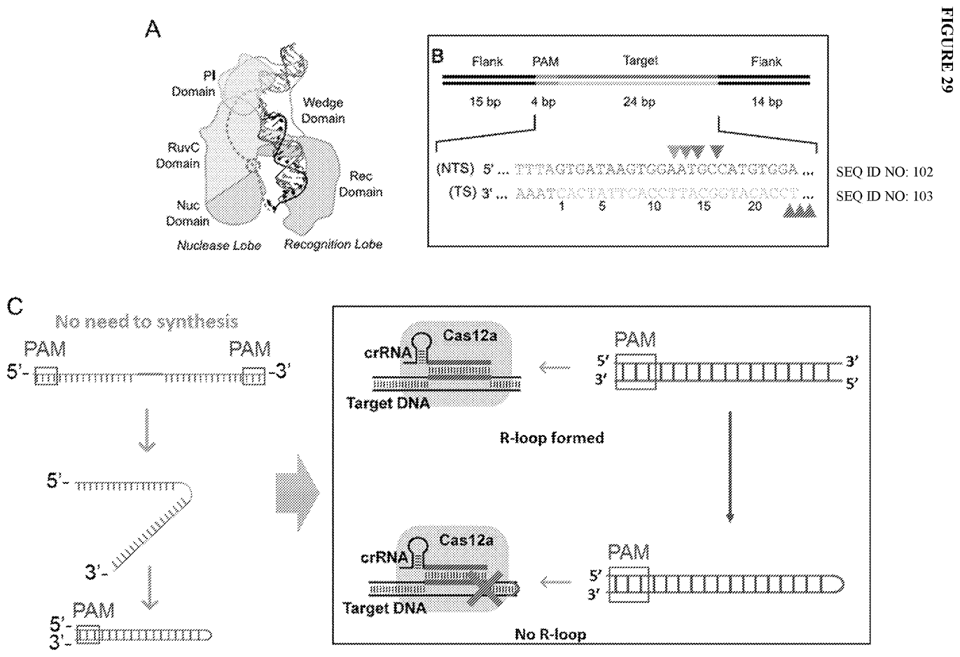

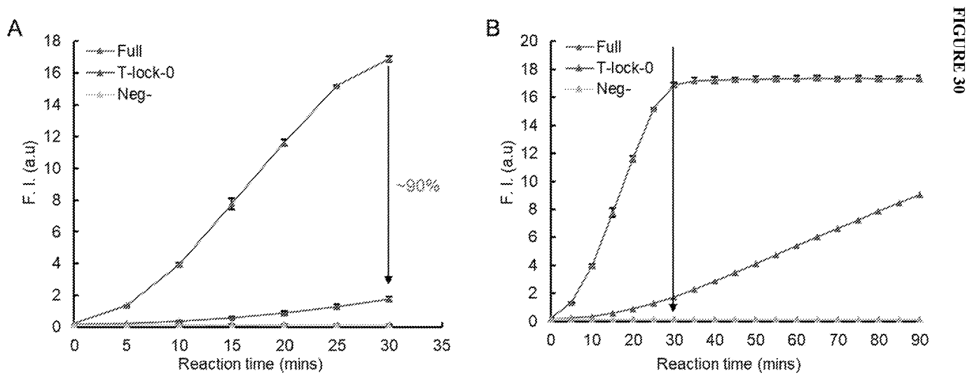

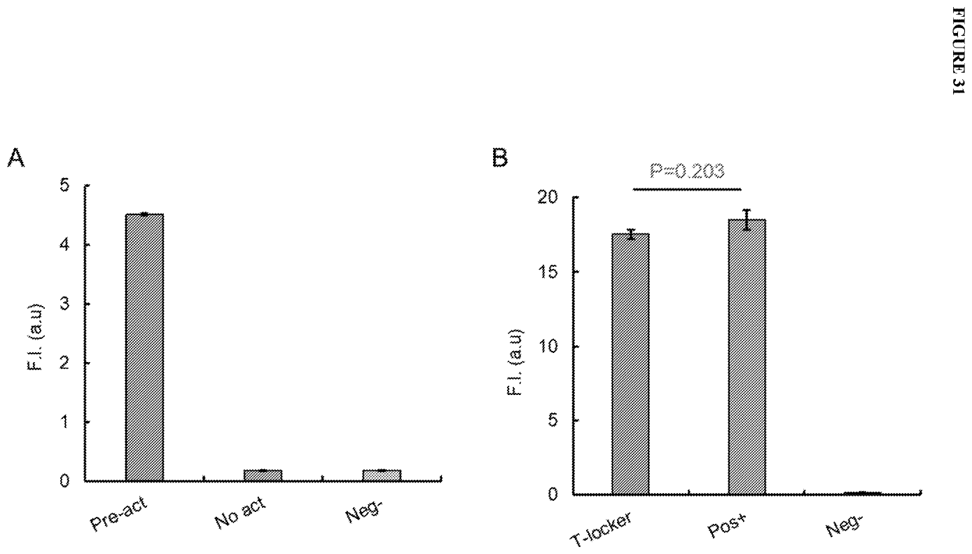

The biosensing performance of Cir-gRNA mediated CRISPR/Cas12a biosensing system was evaluated as shown in FIG. 28. The limit of detection was confirmed to be 1 aM with a detection range from 1 aM to 1 fM.

### 6.3 Conclusion

In this example, a Cir-gRNA mediated CRISPR/Cas12a autocatalysis biosensing system was established. The formation of Cir-gRNA was demonstrated, and the biosensing performance was evaluated with a LOD of 1 aM.

### Example 7—Intramolecularly Bound DNA Autocatalytic Amplifiers (T Lockers)

The inventors have designed a unique DNA molecule which can form a dsDNA structure through intramolecular binding with its 3′ of cis strand linked to 5′ of trans strand.

In this example, the inventors designed a specific nucleic acid nanostructure and discovered a unique interaction pattern between CRISPR/Cas12a RNP and the nanostructure. In the embodiment described in this example the nucleic nanostructure is a dsDNA which has one of its terminals (3′ of the dsDNA cis strand) sealed through intramolecular binding between two complementary palindromic ssDNA sequences present in the nucleic acid. After the 3′ sealed dsDNA structure formed, termed the 3′-tail sealed locker for Cas12a activation (T-locker), it was found to prevent the formation of the R-loop structure within the Cas12a RNP, and hence exhibited restricted CRISPR/Cas12a activation. More importantly, it was observed that the 3′ sealed terminal can be “opened” or “unlocked” by the trans-cleavage activity of a Cas12a that has been activated and thereby become a normal dsDNA target capable of further Cas12a activation. In this example, the T-locker molecule has been successfully used for the development of a novel autocatalysis reaction loop, which can continuously activate multiple Cas12a RNPs through an initial single Cas12a RNP activation by one target nucleic acid molecule. The T-locker induced CRISPR/Cas12a autocatalysis reaction has been used for the detection of DNA, with a sensitivity of 1 aM within a 1 hour reaction at room temperature.

### 7.1 Materials and Methods

### Materials

Exonuclease III (NEB), LbCas12a (NEB), NEB2.1 buffer (NEB), NEB Smart buffer (NEB). All DNA and RNA oligos are synthesized and modified by Sangon Bio-tech Ltd (Table 2A).

TABLE 2A
DNA and RNA oligos used in this Example.

| | Oligo | | | SEQ ID |
|---|---|---|---|---|
| Name | type | Sequence 5′-3′ | Modification | NO: |
| T-locker gRNA | ssRNA | UAA UUU CUA CUA AGU | N/A | SEQ ID |
| | | GUA GAU GAC AUA GCA | | NO: 66 |
| | | CAU AGA CUG AGA CUG | | |
| T-locker | ssDNA | TTTA GAC ATA GCA CAT | 5′-biotin | SEQ ID |
| | | AGA CTG CAG TCT ATG TGC | | NO: 27 |
| | | TAT GTC TAAA | | |
| Trigger ssDNA gRNA | ssRNA | UAA UUU CUA CUA AGU | N/A | SEQ ID |
| | | GUA GAU GGG GGG GGU | | NO: 46 |
| | | UGG UAG GGU GUC | | |
| Trigger ssDNA | ssDNA | GAA GAC ACC CTA CCA ACC | N/A | SEQ ID |
| | | CCC CCC | | NO: 2 |
| Reporter | ssDNA | CCCCCC | 5′-Texas Red; | N/A |
| | | | 3′-BHQ2 | |

### Preparing the T-Locker Molecule Through its Self-Intramolecular Binding

The synthesized ssDNA T-locker oligo was resuspended in MiliQ water to a concentration of 100 μM. After set at room temperature for 20 mins, it was diluted by using MiliQ water to different concentrations between 5 nM to 2 uM. Each of the prepared concentrations were set at room temperature for 5 mins before applying to CRISPR/Cas12a reaction mixture. For long-time storage, the concentration is higher than 2 uM, and stored at −20° C.

### The Activation Efficiency of T-Locker to Cas12a RNP

To prepare the standard CRISPR/Cas12a reaction mixture: 1 μL of 100 μM LbaCas12a (NEB, M0653T) and 5 μL of 20 μM gRNA was mixed at 3.6 mL 1×NEBuffer 2.1 (or 1×NEB Smart buffer), followed by adding of 10 μL of 100 μM Texas Red quenched reporter. The prepared standard CRISPR/Cas12a reaction mixture was stored at 4° C. for future use. For each CRISPR/Cas12a trans-cleavage activation reaction, 10 μL of different concentrations of trigger nucleic acid (trigger ssDNA, T-locker, treated T-locker, etc.) with complementary sequence of gRNA was added into 90 μL prepared reaction buffer. The reaction was carried out at room temperature, and the fluorescence intensity at Ex/Em of 570/615 nm was determined by using a plate reader (iD5 Spectramax, Molecular Devices, USA).

### Trans-Cleavage Pre-Treated T-Locker for Further Cas12a Activation

5 μL of 1 μM trigger ssDNA was added into 90 μL pre-made standard CRISPR/Cas12a reaction mixture targeting the trigger ssDNA molecule. Afterwards, 5 μL of different concentrations of prepared T-locker (2 nM to 2 μM) prepared were mixed. The reaction was set at room temperature for 30 mins. Then, took 10 μL of the reaction product and mixed into 90 μL pre-made standard CRISPR/Cas12a reaction mixture targeting the T-locker molecule. Set the reaction at room temperature, and the fluorescence intensity at Ex/Em of 570/615 nm was determined by using a plate reader (iD5 Spectramax, Molecular Devices, USA).

### Exonuclease III Treated T-Locker for Cas12a Activation

Firstly, 5 μL of 1 μM prepared T-locker was added to 50 μL of 1×NEB 2.1 buffer with 1 μL exonuclease III (100,000 U/mL), or 1 μL PBS as negative control, then reacted at 37° C. for 30 mins. Afterwards, 10 μL of the reaction product was mixed into 90 μL of pre-made standard CRISPR/Cas12a reaction mixture targeting the T-locker molecule. Set the reaction at room temperature, and the fluorescence intensity at Ex/Em of 570/615 nm was determined by using a plate reader (iD5 Spectramax, Molecular Devices, USA).

### T-Locker Induced Cas12a Autocatalysis for Ultra-Sensitive Nucleic Acid Detection

Preparing the T-locker induced Cas12a autocatalysis reaction mixture: 2 μL of 100 μM LbaCas12a (NEB, M0653T) and 5 μL of each 20 μM gRNAs (complementary to trigger ssDNA and T-locker), respectively, was mixed at 3.6 mL 1×NEBuffer 2.1 (or 1×NEB Smart buffer), followed by adding of 10 μL of 100 μM Texas Red quenched reporter and 10 μL of 2 μM prepared T-locker to form the final reaction mixture.

For nucleic acid detection: 10 μL of each concentration of trigger ssDNA (1 aM to 10 pM) were mixed into 90 μL of pre-made T-locker induced Cas12a autocatalysis reaction mixture. Set the reaction at room temperature, and the fluorescence intensity at Ex/Em of 570/615 nm was determined by using a plate reader (iD5 Spectramax, Molecular Devices, USA).

### 7.2 Results

### 7.2.1 Schematics for T-Locker Design

Cas12a RNP has a single nuclease domain, RuvC, which responsible to both its cis-cleavage and trans-cleavage (FIG. 29A). The activation of Cas12a RNP enzymatic activities are highly reliant on the formation of a R-loop structure between the targeted dsDNA strands and the gRNA protospacer region, which has complementary sequence to its target (FIG. 29B). By using a short single strand DNA oligo with palindromic sequences on each of its terminals, the inventors formed an oligo with double stranded DNA oligo with its 3′ end sealed by 1-3 nt ssDNA nucleotides through self-intramolecular binding of complementary sequences (FIG. 29C). When the ssDNA oligo has a suitable length, such as 36 nt to 50 nt, the formed 3′-sealed dsDNA structure could lead to restricted Cas12a RNP activation due to its inner topological barrier to the formation of R-loop within Cas12a RNP (FIG. 29C). This unique dsDNA nanostructure, termed T-locker, can then be applied to establish a controlled Cas12a activation.

7.2.2 Restricted Cas12a Activation Due to 3′-Sealed dsDNA Nanostructure

In compared to a regular dsDNA molecular, the T-locker DNA nanostructure with the same length exhibited significantly reduced fluorescence signal (about 90% decreased after 30 mins reaction at room temperature) that produced by Cas12a RNP trans-cleavage activity (FIG. 30A). As the reaction time increased, the activation of Cas12a through regular dsDNA reached plateau after 30 mins, while the T-locker trigger shown a continuously increased fluorescence signal till 90 mins, which indicated a potential of T-locker structure been reshaped by activated Cas12a for further Cas12a RNP activations (FIG. 30B).

### 7.2.3. Restoration of Cas12a Activation Through Pre-Activated Trans-Cleavage

It is known that Cas12a trans-cleavage can randomly cut surrounding ssDNA molecules even under 2 nucleotides length. To verify if the activated Cas12a RNP trans-cleavage can also cleave the short ssDNA structure on the 3′-sealed T-locker and reopen the T-locker to normal dsDNA molecule formation, the same concentrations of T-locker molecule have been either treated by pre-activated Cas12a RNPs or 1×NEB 2.1 buffer for 30 mins at room temperature. Afterwards, the treated T-lockers were used as target DNA to act Cas12a RNPs with complementary gRNA sequence. The result indicated that the Cas12a trans-cleavage treated T-locker showing its capability of Cas12a RNP activation with significant increased fluorescence signal, while the T-locker without trans-cleavage pre-treatment remained as Cas12a activation restricted (FIG. 31A). When the T-locker was treated with pre-activated trans-cleavage overnight for fully open its 3′ tails, its Cas12a activation efficiency returned to the same level as the same concentration of normal dsDNA, which indicated the fully restored Cas12a activation capacity of T-locker after its 3′-end been fully opened (FIG. 31B).

### 7.2.4. Different T-Locker Status LED to Different Cas12a Interactions

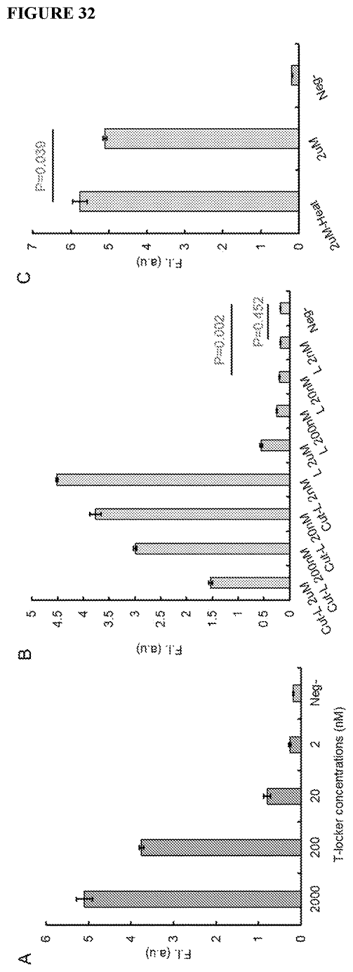

When the T-locker concentrations have been increased from 2 nM to 2000 nM, the corresponded fluorescence signals also increased, which indicated the increased Cas12a RNP activation level (FIG. 32A). However, when comparing to the same concentrations of trans-cleavage pre-treated T-lockers, the overall activation efficiencies are significantly lower, particularly, 2 nM T-locker has no significant activation effects for Cas12a RNPs (FIG. 32B). This indicated that, at proper concentration, T-locker can lead to the Cas12a reaction retained at undetectable stage for a certain period of time, but return to Cas12a activable stage after the 3′-sealed structure been broken by trans-cleavage. Interestingly, the higher concentrations of pre-treated T-locker exhibited lower fluorescence signal, which may due to the additionally consumed Cas12a trans-cleavage power with higher numbers of overall DNA molecule in the reaction mixture (FIG. 32B). In addition, when the T-locker has been pre-heated to 95° C. for 5 mins and cool down to room temperature naturally, the activation efficiency increased significantly when compared to the same concentration of T-locker without heat treatment (FIG. 32C). This may indicate that the way to prepare and form T-locker DNA nanostructure played a critical role in its Cas12a activation restriction function.

### 7.2.5. Characterization of T-Locker to Cas12a Trans-Cleavage

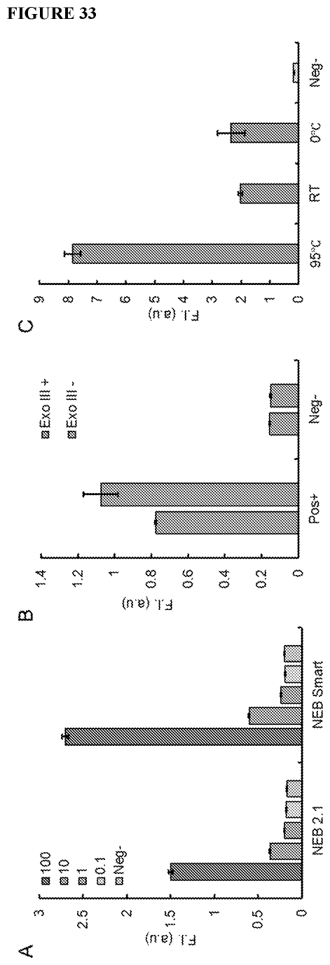

When the T-locker DNA nanostructure been applied to Cas12a activation within different buffers, it shown significant difference in terms of overall trans-cleavage activation efficiency, and T-locker in NEB 2.1 buffer shown a better controlled Cas12a activation in compared to in NEB Smart buffer (FIG. 33A). This indicated that the buffering system can affect the T-locker performances, which could be due to the different structural status of T-locker molecule in different buffering systems. In addition, the stability of T-locker for exonuclease treatment has also been investigated. Compared to T-locker without being treated by exonuclease III, exonuclease III treatment led to significantly decreased Cas12a activation efficiency, which indicated that exonuclease III may cause a considerable level of T-locker degradation (FIG. 33B). However, in comparing to the negative controls, the exonuclease III treated T-locker also exhibited significantly higher fluorescence signal, which indicated its potential capability to resist the exonuclease III for certain period of time (FIG. 33B). Furthermore, the temperature to the T-locker function were further tested at 3 different temperatures, and the results shown no difference for preparing T-locker at room temperature and 0° C., but pre-heated to 95° C. led to significantly increased activation of Cas12a, which may potentially be due to the increased intermolecular binding of the T-locker molecules (FIG. 33C).

### 7.2.6. T-Locker Induced Autocatalysis Reaction for DNA Detection

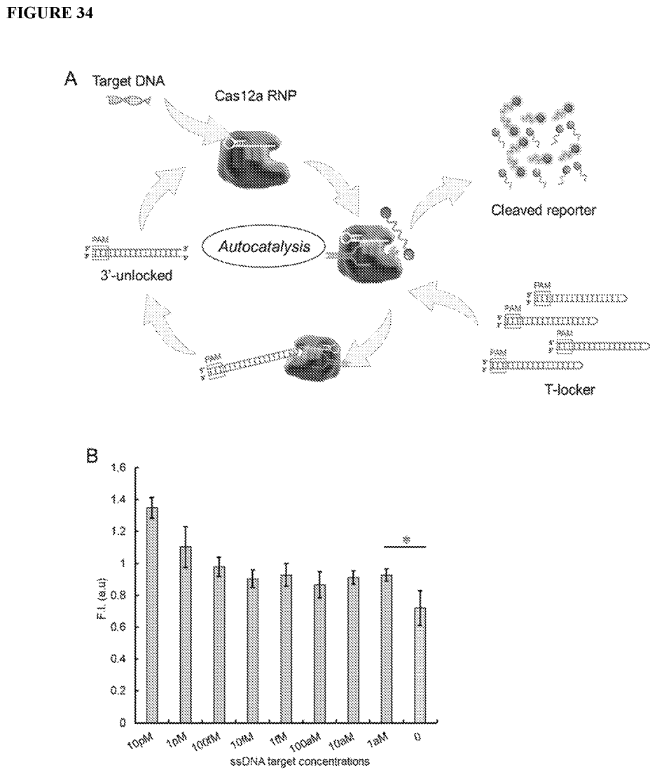

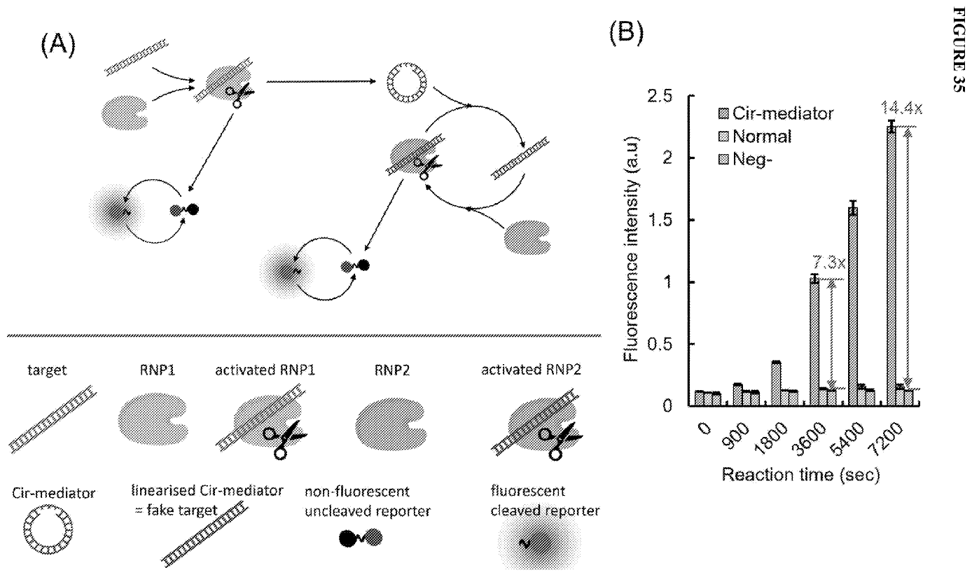

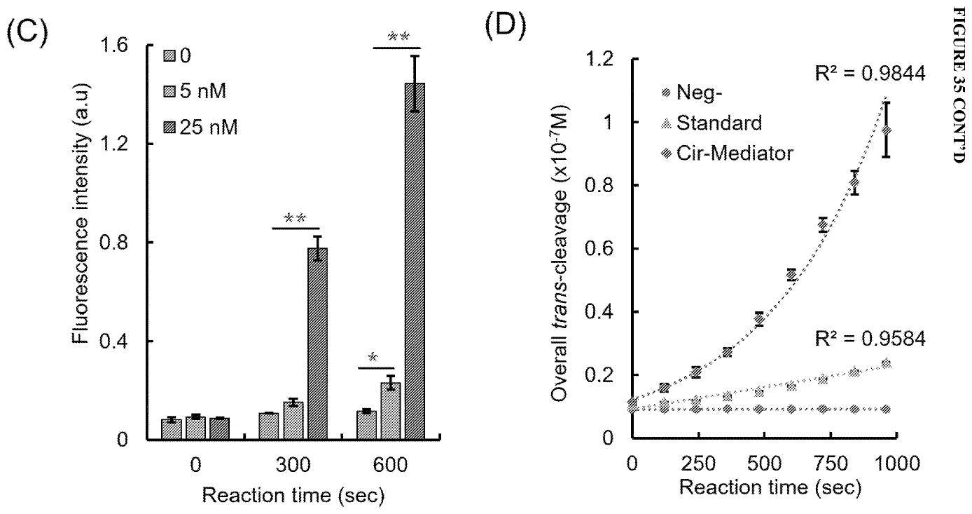

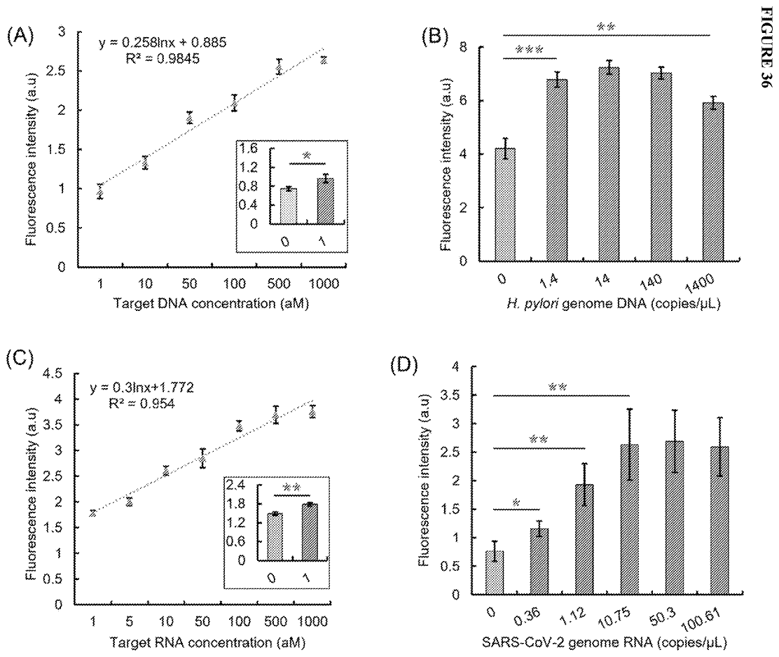

After the T-locker demonstrated its capability to restrict Cas12a RNP activation, and also its ability to regain Cas12a activation function through pre-activated Cas12a RNP trans-cleavage, it is possible to use this T-locker as an intermediator molecule to facilitate a Cas12a self-circularized signal amplification scheme that can response to the presence of extremely low level of target nucleic acid sequence (FIG. 34A). By integrating T-locker into a standard CRISPR/Cas12a reaction system for DNA detection, the results shown its capability to significantly increase the original sensitivity of CRISPR/Cas12a from pM-level to 1 aM (FIG. 34B). This indicated that the introduced T-locker has transferred the standard CRISPR/Cas12a reaction to an autocatalysis reaction.

### 7.3 Discussion

By using a short single strand DNA oligo with palindromic sequences on each of its terminals, a unique double strand DNA nanostructure can be formed through self-intramolecular binding of complementary sequences from each end. Upon the self-intramolecular binding, the original ssDNA oligo became a double strand target DNA molecule (PAM+target sequence) with its one terminal (the 3′ of its cis strand or the non-target strand of Cas12a RNP) sealed by a few ssDNA nucleotides (FIG. 29C). Although the formation of such structure can be affected by various factors, such as temperature, buffer, etc. The successfully formed unique 3′-sealed dsDNA nanostructure, or the T-locker structure, shown an obvious restriction function for Cas12a RNP activation in compared to its normal dsDNA molecule formation where both of its two ends are opened. After verified that such ssDNA sealed terminal of T-locker can be broke by pre-activated Cas12a trans-cleavage, the T-locker returned to its normal dsDNA structure, hence, recovered for further Cas12a RNP activation. By using proper buffering system and at proper concentrations, T-locker can be integrated into a standard CRISPR/Cas12a reaction, and transfer it to an autocatalysis reaction. By using this T-locker integrated CRISPR/Cas12a reaction system, the inventors were able to achieve maximumly 1 aM ssDNA detection within 1 hour at room temperature, without the need for any additional amplification schemes.

Unlike other CRISPR/Cas12a-based ultra-sensitivity nucleic acid detection approaches generally relies on the pre-amplification to boost its sensitivity into aM-level, or with complex nucleic acid 3D structure design and synthesis, this T-locker method provided a simple but effective new way to realize CRISPR/Cas12a-based autocatalysis for detecting the presence of target nucleic acid sequence down to 1 aM concentration. In addition, by simple change its ssDNA oligo design, the T-locker has the potential to be modified to expend its applications in CRISPR/Cas-based biosensor development, including but not limited to: 1) labelling the T-locker molecule with fluorophore and quencher at proper locations, which can fulfill the T-locker with additional function as reporter for CRISPR/Cas reactions, hence, combining the autocatalysis intermediate molecule with signal reporting together to further simplify the overall system; 2) applying molecule labelling and/or modified nucleotides to certain points/regions of the T-locker ssDNA oligo, such as XNA or any other artificially modified nucleotides/molecules, to give additional function for the T-locker, such as nuclease resistant, photosensitive, etc.; 3) redesigning the T-locker sequence by using either DNA or RNA, or partially DNA/RNA, to be able to applied for other type V or VI Cas proteins, such as Cas13, Cas14, etc., for realizing autocatalysis reactions.

### Example 8—Further Characterization of Cir-Mediator Induced Autocatalytic Amplification of Cas12a Trans-Cleavage Activity

In this example the system described in Example 4 (FIG. 5A) also referred to Autocatalytic Cas12a Circular DNA Amplification Reaction (AutoCAR-1) was further characterized (Also depicted in FIG. 35A).

### 8.1 Materials and Methods

### Method 8.1a.

3 μL of 100 μM LbaCas12a endonuclease (NEB, M0653T), 5 μL of 20 μM gRNA1 (for trigger DNA/RNA) and 10 μL of 20 μM gRNA2 (for Cir-mediator) was mixed at 3.6 mL of 1×NEBuffer 2.1 and followed with the adding of 12 μL of 100 μM Texas Red quenched reporter. Afterwards, the prepared Cir-mediator solution was mixed to a final concentration of 50 nM (can be varied between 5-50 nM) before use to form the final AUTOCAR reaction mixture. Then, 10 μL of different concentrations of trigger DNA (ssDNA or dsDNA) with a complementary sequence to gRNA was mixed with 90 μL of the prepared final AUTOCAR reaction mixture to initiate the reaction. The reaction was set at room temperature and the fluorescence intensity at Ex/Em of 570/615 nm was determined by using a plate reader (iD5 Spectramax, Molecular Devices, USA). Alternatively, Cir-mediator can be replaced by 50 nM of the same volume of 1×NEBuffer 2.1 for a comparison with a standard Cas12a reaction as: 1 μL of 100 μM LbaCas12a endonuclease (NEB, M0653T) and 5 μL of 20 μM gRNA was mixed at 3.6 mL 1×NEBuffer 2.1, followed by addition of 6 μL of 100 μM Texas Red quenched reporter. The prepared standard CRISPR/Cas12a reaction mixture was stored at 4° C. for future use.

### Method 8.1b

Exploring the AutoCAR-1 reaction signal intensity with Cir-mediator concentration changes: 3 μL of 100 μM LbaCas12a endonuclease (NEB, M0653T), 5 μL of 20 μM gRNA1 (for trigger DNA/RNA) and 10 μL of 20 μM gRNA2 (for Cir-mediator) was mixed at 3.6 mL 1×NEBuffer 2.1 and followed with the adding of 12 μL of 100 μM Texas Red quenched reporter. Afterwards, the Cir-mediator solution was mixed to a final concentration of 0, 5, 12.5, 25 and 50 nM. Then, 10 μL 10 pM trigger ssDNA was mixed with 90 μL of the prepared final AutoCAR reaction mixture to initiate the reaction. The reaction was set at room temperature and the fluorescence intensity at Ex/Em of 570/615 nm was determined by using a plate reader (iD5 Spectramax, Molecular Devices, USA).

### 8.2 Results

In comparison with a standard Cas12a catalytic system without Cir-mediators, the reporter trans-cleavage increased 7.3 times over 3600 sec (60 mins) and 14.4 times over 7200 sec (120 mins) reactions at room temperature. (FIG. 35 B). The reporter trans-cleavage in AutoCAR-1 increased with increasing Cir-mediator concentration (n=3) (FIG. 35 C). (D) Time dependence of the fluorescence signal for AutoCAR-1 in comparison to a standard CRISPR/Cas12a reaction (n=3). The data show differences in reporter trans-cleavage kinetics in a standard Cas12a catalytic system (linear trend, y=0.014x+9.1129, goodness of fit R²=0.9584) and Cir-mediator-assisted Cas12a autocatalysis reaction (super-linear trend, exponential fit, y=12.102e^{0.0023}x, goodness of fit R²=0.9844). These results indicate that instead of the well-established linear trend, AutoCAR-1 produces a nonlinearly increasing signal. The results provides evidence of rapid assays including an assay producing results in approximately 100 seconds.

### Example 9—Detection of Genomic DNA and Genomic RNA by Using the AutoCAR-1

This Example shows the detection of genomic DNA and genomic RNA by using the AutoCAR-1 scheme from Example 8.

### 9.1 Materials and Methods

### Method 9.1a.

3 μL of 100 μM LbaCas12a endonuclease (NEB, M0653T), 5 μL of 20 μM gRNA1 (for the target DNA sequence) and 10 μL of 20 μM gRNA2 (for the Cir-mediators) was mixed at 3.6 mL 1×NEBuffer 2.1 and followed by adding of 12 μL of 100 μM Texas Red quenched reporter. Afterwards, the prepared Cir-mediator solution was mixed to a final concentration of 50 nM to form the final AutoCAR-1 reaction mixture for DNA detection. Then, 10 μL of different concentrations of target DNA (ssDNA, dsDNA or H. pylori genome DNA) were mixed with 90 μL of the prepared final AutoCAR-1 reaction mixture. The reaction was set at room temperature for 1 hour, and the fluorescence intensity at Ex/Em of 570/615 nm was determined by using a plate reader (iD5 Spectramax, Molecular Devices, USA).

### Method 9.1b

The qPCR reaction for H. pylori glm gene detection: The manufacturer's instruction of QuantiNova SYBR Green PCR kit (QIAGEN, 208052) was used for the qPCR reactions. Briefly, for each 20 μL reaction mixture, 10 μL 2×SYBR master mixture, 2 μL 10 μM Forward-primer and 2 μL 10 μM Reverse-primer were mixed with 4 μL of DNase/RNase free water. Then, 2 μL of sample with difference concentrations of H. pylori genome DNA (0, 1.4, 14, 140, 1400, 14000 copies/μL) was added to form the final 20 μL reaction mixture. Then qPCR reaction was set on a CFX96 Touch Real-Time PCR Detection System (Bio-Rad Laboratories Inc., USA) at the conditions of 95° C. 2 mins, and followed with 35 cycles of amplification at 95° C. 10 sec and 60° C. 20 sec. The default melting curve analysis was also added for validation of the RT-qPCR.

### Method 9.1c

3 μL of 100 μM LbaCas12a endonuclease (NEB, M0653T), 5 μL of 20 μM gRNA1 (for target RNA sequence) and 10 μL of 20 μM gRNA2 (for Cir-mediator) was mixed at 3.6 mL 1×NEBuffer 2.1 and followed by adding of 120 μL of 100 μM Texas Red quenched reporter and 36 μL of 1M DTT. Afterwards, the prepared Cir-mediator solution was mixed to a final concentration of 50 nM before the use to form the final AUTOCAR reaction mixture for RNA. Then, 10 μL different concentration of target RNA (RNA or SARS-CoV-2 genome RNA) was mixed with 90 μL of the prepared final c-Car reaction mixture. The reaction was set at room temperature for 1-1.5 hours, and the fluorescence intensity at Ex/Em of 570/615 nm was determined by using a plate reader (iD5 Spectramax, Molecular Devices, USA).

### 9.2 Results

Although Cas12a RNP is, in principle, able to recognize RNA sequences, direct RNA detection by Cas12a is impractical due to a very low level of RNA-induced trans-cleavage; as a result earlier works relied on the combination of reverse transcription and/or additional amplification strategies. Autocatalysis in our AutoCAR-1 overcomes this limitation, allowing direct RNA detection without reverse transcription or amplification. To demonstrate this, the reporter concentration in AutoCAR-1 was increased and DTT was added as a Cas12a “trans-cleavage enhancer”. In such conditions, AutoCAR-1 detected synthetic RNA, without reverse transcription, at a sensitivity down to 1 aM and with a linear range of 3 orders of magnitude. The inventors also detected genomic RNA extracted from SARS-CoV-2 viral particles. AutoCAR-1 was able to detect the presence of the N-gene of SARS-CoV-2 viral genome RNA at the sensitivity of less than 1 copy/μL (360 copies/mL), which is comparable to the current gold standard RT-qPCR.

### Example 10—Detection of Cancer Mutations in Blood Plasma

This Example shows ctDNA detection from blood plasma using DANCER-2, also referred to herein as AutoCAR-3.

### 10.1 Methods

### Method 10.1a

Patient blood from The Cancer Molecular Screening and Therapeutics (MoST) program were assessed (ACTRN12616000908437). The study was performed in accordance with the Declaration of Helsinki. The program has been approved by the St Vincent's Hospital Sydney Human Research Ethics Committee (reference, HREC/16/SVH/23). All patients provided written informed consent for participation in this study. Eligibility criteria included patients with advanced solid cancers of any histological type, prioritising rare cancers. For each patient enrolled in the MoST program, 10 sections of 4 μm of formalin fixed paraffin embedded (FFPE) tissue underwent DNA extraction. Tissue deparaffination was performed using a deparaffinization solution and DNA extraction using AllPrep DNA/RNA kit (Qiagen, Germantown, MD, USA). Quantification was performed using Qubit double-stranded DNA (dsDNA) high-sensitivity (HS) assay kit (Invitrogen, Waltham, MA, USA).

CGP profiling was undertaken using FFPE archival tumor. Patients were screened at entry into the MoST program. The panel-based assays employed included Illumina TruSight Tumor 170, Illumina TruSight Tumor 500, or the Foundation Medicine (FMI), and conducted using standard protocols. All three panels had PIK3CA H1047R mutation detection capabilities.

Venous blood ˜10 mL was collected by standard phlebotomy techniques into anticoagulant containing tubes. The time from blood collection to processing was 0-5 days (median: 2 days). Blood was kept at room temperature before and during processing. Bloods were centrifuged at 4000 RPM for 10 min, after which plasma was transferred to a new tube and spun for an additional 10 min at 4000 RPM. Plasma was removed leaving behind any residual pellet and aliquots stored at −80° C. until assayed.

Plasma samples of ten patients (n=6 positive and 4 negative for PIK3CA H1047R mutation) were tested by using Method 10.1b below.

### Method 10.1b

The Dancer-2 (AutoCAR-3) reaction mixture for ctDNA (PIK3CA H1047R) detection was prepared as follows: 1 μL 100 μM (100 pmol) of Cas12a protein was gently mixed with 5 μL 20 μM (100 pmol) of gRNA-ct to form the Cas12a RNP-1. Meanwhile, 1 μL 100 μM (100 pmol) of Cas12a protein was gently mixed with 5 μL 20 μM (100 pmol) of gRNA-D to form the Cas12a RNP-2. Afterwards, the prepared Cas12a RNP-1 and Cas12a RNP-2 were mixed with 200 μL of 5 μM (1 nmol) of Cir-reporters in 5 mL 1×NEB 2.1 buffer to form the standard reaction mixture.

Subsequently, 10 μL of spiked in sample or collected mouse plasma, human plasma or human saliva was added to 90 μL of the prepared reaction mixture for activating trans-cleavage of Cas12a and enabling the CRISPR/Cas biosensing reaction. A SpectraMax iD5 multi-Mode Microplate Reader (Molecular Devices) was applied for the detection of fluorescence readout. The Ex/Em of Tex-Cir-reporter-BHQ2 was 570/615 nm. All the DNA and RNA oligos used in this experiment are listed in Table S8.

TABLE S8
DNA and RNA oligos used in Example 10.

| | Oligo | | | |
|---|---|---|---|---|
| Name | type | Sequence 5′-3′ | Modification | SEQ ID NO: |
| H1047R WT | ssDNA | GAA ACA AAT GAA TGA | N/A | SEQ ID NO: |
| Cis | | TGC ACA TCA TGG TGG | | 67 |
| | | CTG GAC AAC AAA AA | | |
| H1047R WT | ssDNA | TT TTT GTT GTC CAG CCA | N/A | SEQ ID NO: |
| trans | | CCA TGA TGT GCA TCA | | 68 |
| | | TTC ATT TGT TTC | | |
| H1047R Mut | ssDNA | GAA ACA AAT GAA TGA | N/A | SEQ ID NO: |
| Cis | | TGC ACG TCA TGG TGG | | 69 |
| | | CTG GAC AAC AAA AA | | |
| H1047R Mut | ssDNA | TT TTT GTT GTC CAG CCA | N/A | SEQ ID NO: |
| trans | | CCA TGA CGT GCA TCA | | 70 |
| | | TTC ATT TGT TTC | | |
| H1047R Mut | ssRNA | UAA UUU CUA CUA AGU | N/A | SEQ ID NO: |
| gRNA | | GUA GAU A UGA UGC ACG | | 71 |
| | | UCA UGG UGG | | |
| H1047R WT | ssRNA | UAA UUU CUA CUA AGU | N/A | SEQ ID NO: |
| gRNA | | GUA GAU A UGA UGC ACA | | 72 |
| | | UCA UGG UGG | | |
| Cir-ssDNA | ssDNA | TTT A/iBio-dT/T TTA GAG | 5′-Azide (N3); | SEQ ID NO: |
| | | AGA GGT AAA | 3′-CHCH | 60 |
| cDNA | ssDNA | TTT ACC TCT CTC TAA | 5′-Texas Red; | SEQ ID NO: |
| | | AAT | 3′-BHQ2 | 61 |
| cDNA-extend | ssDNA | TTT ACC TCT CTC TAA | 5′-FAM; 3′- | SEQ ID NO: |
| | | AAT CCC CC | Biotin | 62 |
| ssDNA | ssDNA | TTATT | 5′-Texas Red; | N/A |
| reporter | | | 3′-BHQ2 | |

All human plasma experiments were approved by the UNSW Ethics Committee (UNSW HC210160), in addition to ACTRN12616000908437. All human saliva experiments were approved by the UNSW Ethics Committee (UNSW HC200568).

### 10.2 Results

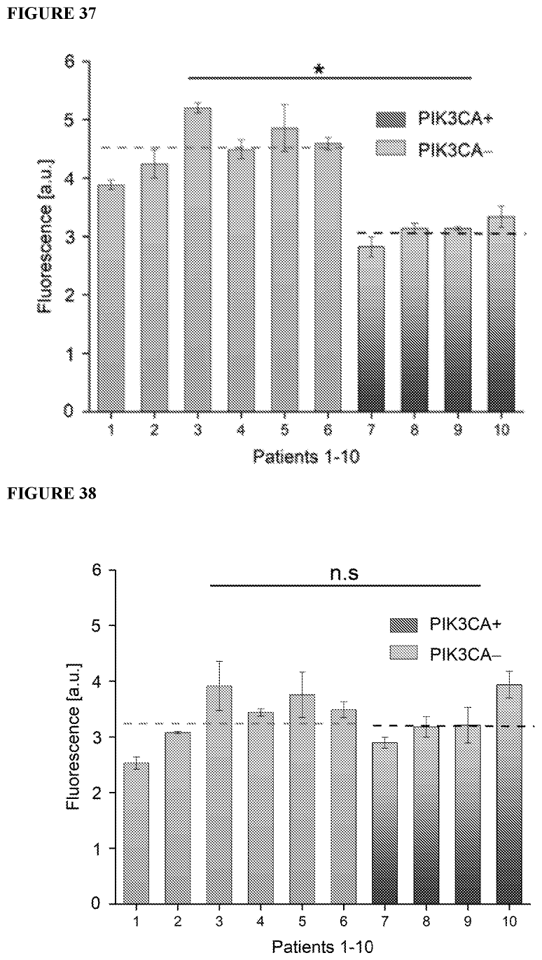

Blood samples were selected from patients with tumors known to harbor PI3KCA mutations, as well as control cancer patients without that mutation, from the Molecular Screening and Therapeutics (MoST) program (ACTRN12616000908437). Plasma samples from patients with advanced cancers harboring the PIK3CA H1047R mutation as determined in tumour biopsies (PIK3CA H1047R+n=6, and PIK3CA H1047R−n=4) (FIG. 37) were subject to detection of circulating PIK3CA mutations in blood plasma using DANCER-2 (AutoCAR-3).

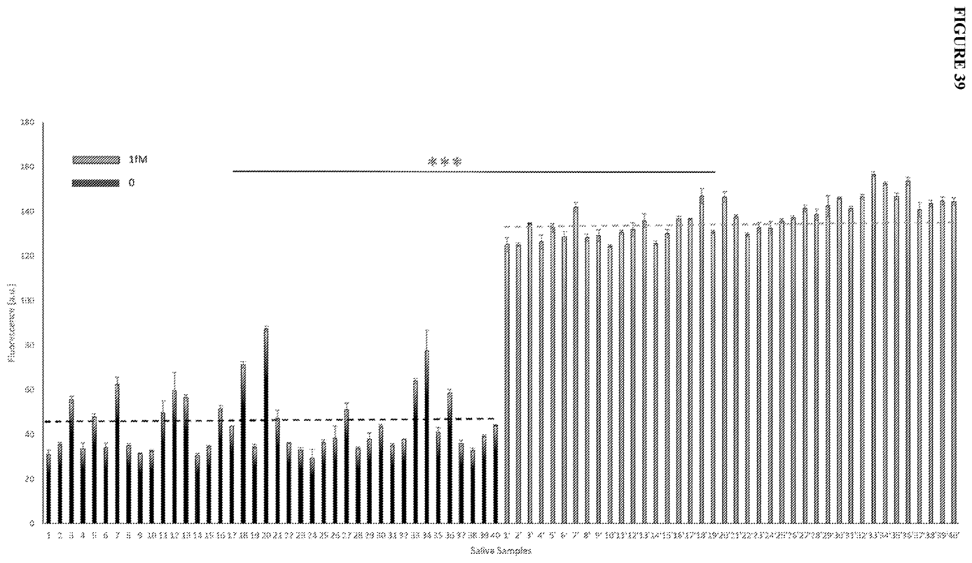

Using Cas12a RNPs targeting the wild type sequence of the PIK3CA gene in the region of the H1047R mutation, the fluorescence signal for the PIK3CA+ and PIK3CA− patient groups (FIG. 38) was indicative of the background signal level due to the wild type PIK3CA gene fragments in the tested plasma samples. The lack of significant difference between two patient groups confirms that our AutoCAR-3 system can specifically detect the PIK3CA H1047R mutation in ctDNA from the patient samples.

The results show statistically significant discrimination between cancer and control samples, which can also serve as proof for the feasibility of DNA single nucleotide polymorphism detection.

### Example 11—Detection in Saliva

This Example demonstrates that detection in saliva is feasible despite the presence of nucleases.

### 11.1 Methods

Method 11.1a—Detection of ctDNA from Saliva Sample

Saliva Ethics: UNSW HC200568. n=40 saliva samples were collected and stored in −20° C. for further processing. The saliva samples were freeze-thawed at room temperature, then centrifuged at 6000 rpm for 20 min. The supernatant was used for detection.

The AutoCAR-3 reaction mixture for ctDNA (PIK3CA H1047R) detection was prepared as follows: 1 μL 100 μM (100 pmol) of Cas12a protein was gently mixed with 5 μL 20 μM (100 pmol) of gRNA-ct to form the Cas12a RNP-1. In the meanwhile, 1 μL 100 μM (100 pmol) of Cas12a protein was gently mixed with 5 μL 20 μM (100 pmol) of gRNA-D to form the Cas12a RNP-2. Afterwards, the prepared Cas12a RNP-1 and Cas12a RNP-2 were mixed with 200 μL of 5 μM (1 nmol) of Cir-reporters in 5 mL 1×NEB 2.1 buffer to form the standard reaction mixture. Subsequently, 200 μL of 5 μM (1 nmol) of Cir-reporter solution was added and well mixed to form the reaction mixture. Afterwards, 10 μL of collected saliva sample was added to 90 μL of the prepared reaction mixture for activating trans-cleavage of Cas12a and enabling the CRISPR/Cas biosensing reaction. A SpectraMax iD5 multi-Mode Microplate Reader (Molecular Devices) was applied for the detection of fluorescence readout. The Ex/Em of Tex-Cir-reporter-BHQ2 was 570/615 nm. The DNA and RNA oligos used in this example are as listed in Table S8 (in Example 10).

### 11.2 Results

The results presented in FIG. 39 show that the presence of targeted PIK3CA H1047R mutation sequence was detected with 100% accuracy in saliva samples spiked with with 1 fM of the PIK3CA H1047R mutation sequence.

### Example 12—Suitability of Different Orthologues of Cas12a

This example investigated the use of another Cas12a orthologue AsCas12a.

### 12.1 Methods

The DANCER (AutoCAR-2) reaction mixture was prepared as follows: 1 μL 100 μM (100 pmol) of Cas12a protein (AsCas12a) was gently mixed with 5 μL 20 μM (100 pmol) of gRNA-D to form the Cas12a RNP in 5 mL 1×NEB 2.1 buffer. Subsequently, 200 μL of 5 μM (1 nmol) of Cir-amplifier solution was added and well mixed to form the reaction mixture. Afterwards, 10 μL of target-D ssDNA at different concentrations were added to 90 μL of the prepared reaction mixture for activating trans-cleavage of Cas12a and enabling the CRISPR/Cas autocatalysis biosensing reaction. A SpectraMax iD5 multi-Mode Microplate Reader (Molecular Devices) was applied for the detection of fluorescence readout. The Ex/Em of Texas red-TTATT-BHQ2 reporter was 570/615 nm. All the DNA and RNA oligos used in this experiment are listed in Table S5.

### 12.2 Results

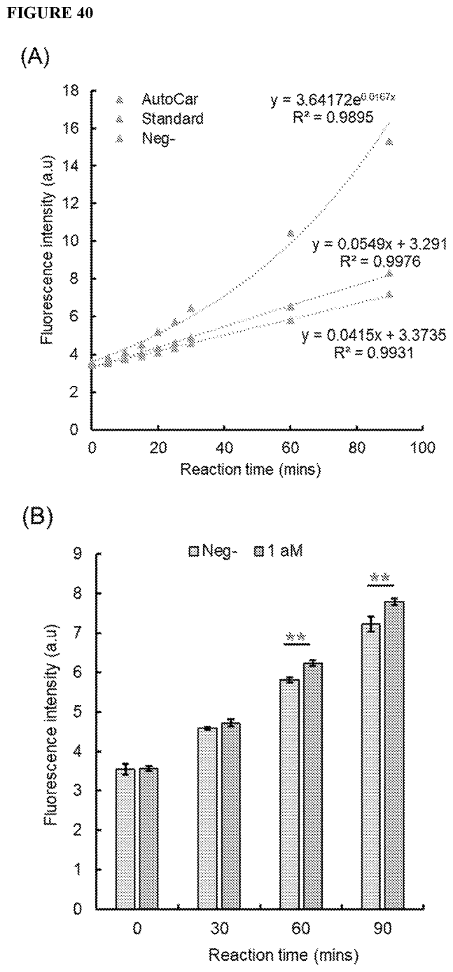

As shown in FIG. 40 This example shows the feasibility of 1 aM detection with cir-mediators and using another Cas12a orthologue AsCas12a.

### Example 13—Attomolar Level Detection Under Various Conditions

This example details further characterization of ultrasensitive (aM level) detection with cir-mediators in an AutoCAR-1 system.

### 13.1 Methods

### Method 13.1a

3 μL of 100 μM LbaCas12a endonuclease (NEB, M0653T), 5 μL of 20 μM gRNA1 (for trigger DNA/RNA) and 10 μL of 20 μM gRNA2 (for Cir-mediator) was mixed at 3.6 mL 1×NEBuffer 2.1 and followed with the adding of 12 μL of 100 μM Texas Red quenched reporter. Afterwards, the prepared Cir-mediator solution was mixed to a final concentration of 50 nM before use to form the final AUTOCAR reaction mixture. Then, 10 μL different concentrations of trigger DNA with a complementary sequence to gRNA was mixed with 90 μL of the prepared final AUTOCAR reaction mixture to initiate the reaction. The reaction was set at room temperature and the fluorescence intensity at Ex/Em of 570/615 nm was determined by using a plate reader (iD5 Spectramax, Molecular Devices, USA).

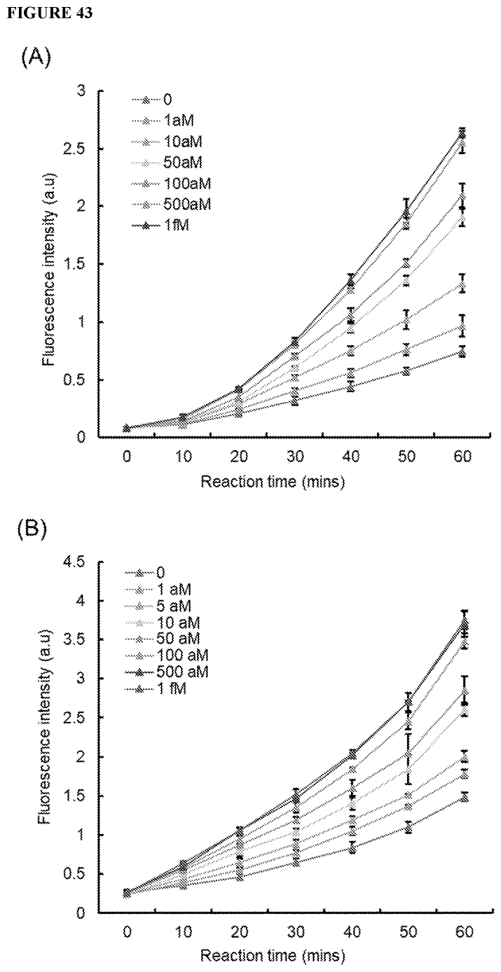

Method 8.1a (DNA) and 9.1c (RNA) were also used (FIG. 43).

### 13.2 Results

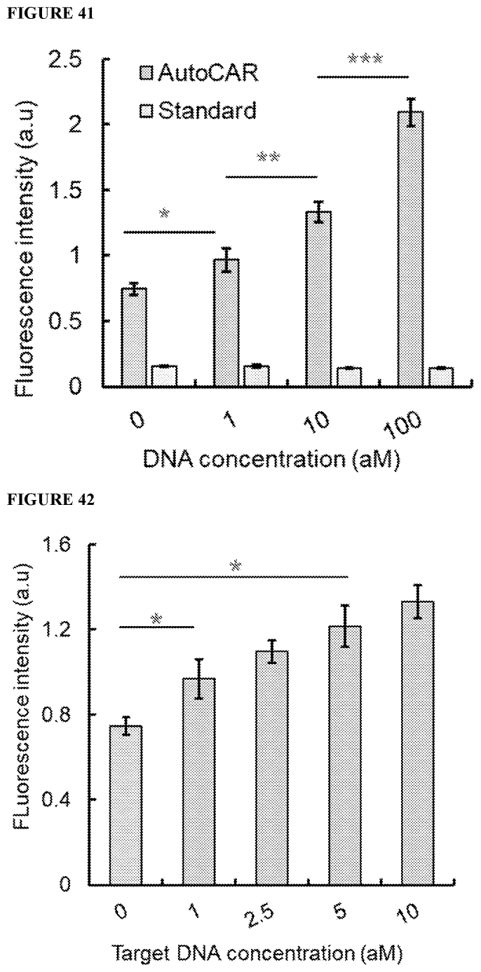

In experimental conditions described AutoCAR-1 specific detection of DNA in the attomolar concentration range, down to 1 aM. While the standard CRISPR/Cas12a system without additional amplification strategy shows no detectable signal differences between the same target concentration ranges (FIG. 40). AutoCar-1 was able to distinguish the changes of target DNA concentrations between 1 to 10 aM (FIG. 41) with detection of both DNA and RNA (FIG. 43).

### Example 14—Rapid Assays Under Various Conditions

This Example assessed the rapid detection of pM and aM levels of DNA and RNA.

### 14.1 Methods

Methods employed were as described in method 8.1a, 9.1a and 9.1c.

### 14. 2 Results

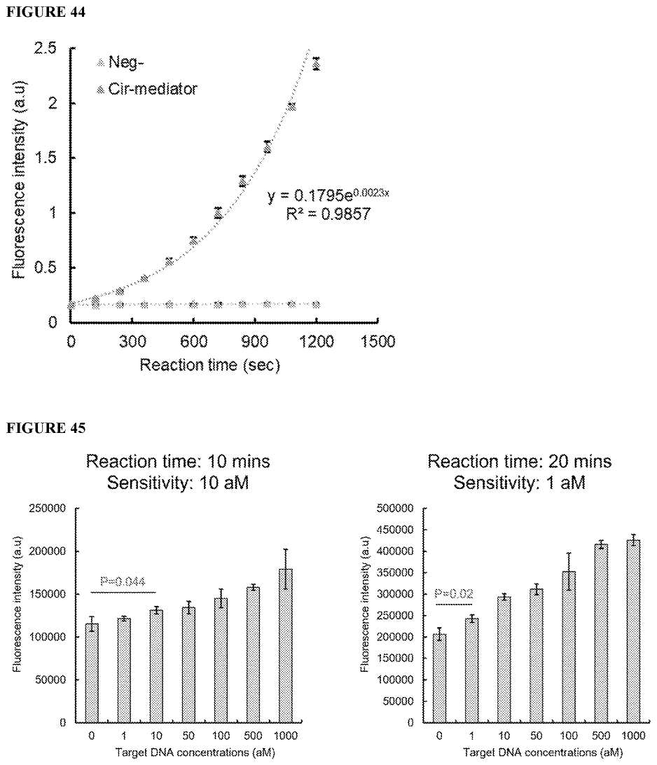

After the autocatalysis loop of AutoCAR-1 has been activated, the fluorescence signal intensity increased strongly with reaction time following a non-linear growth pattern, in response to addition of 1 pM ssDNA (FIG. 44) (Method 8.1a).

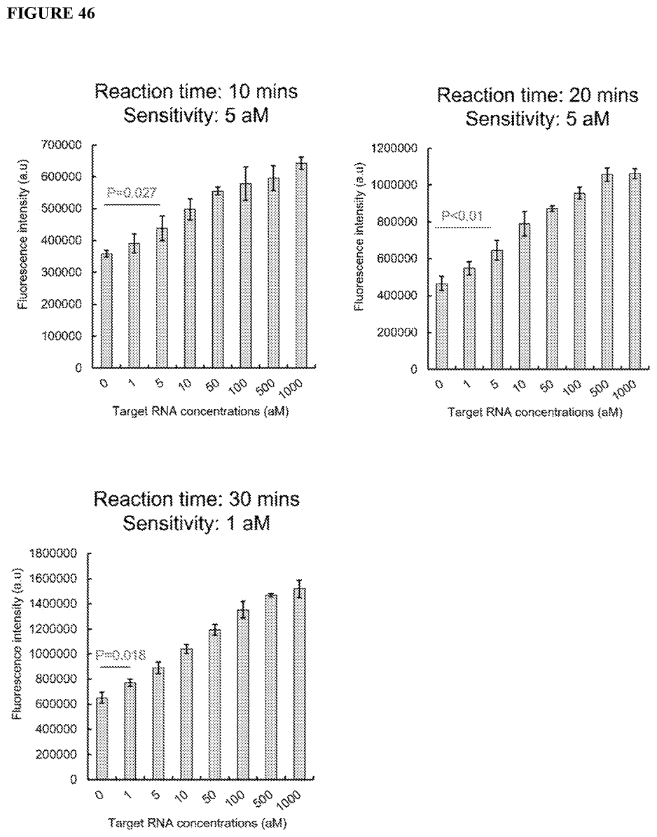

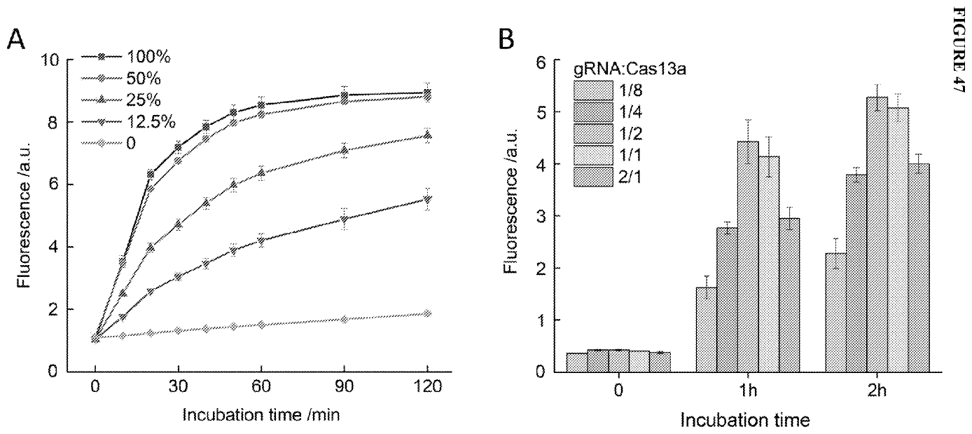

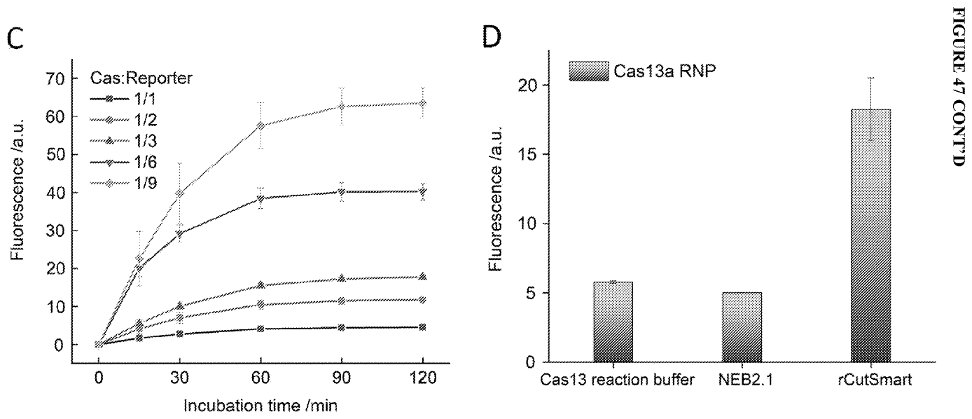

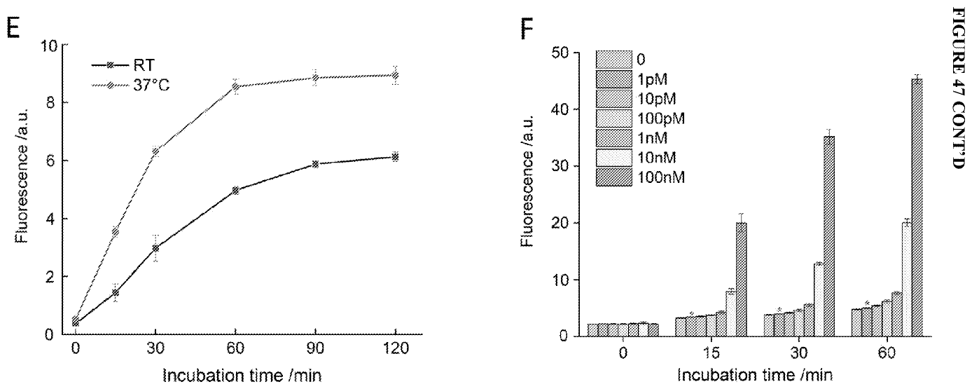

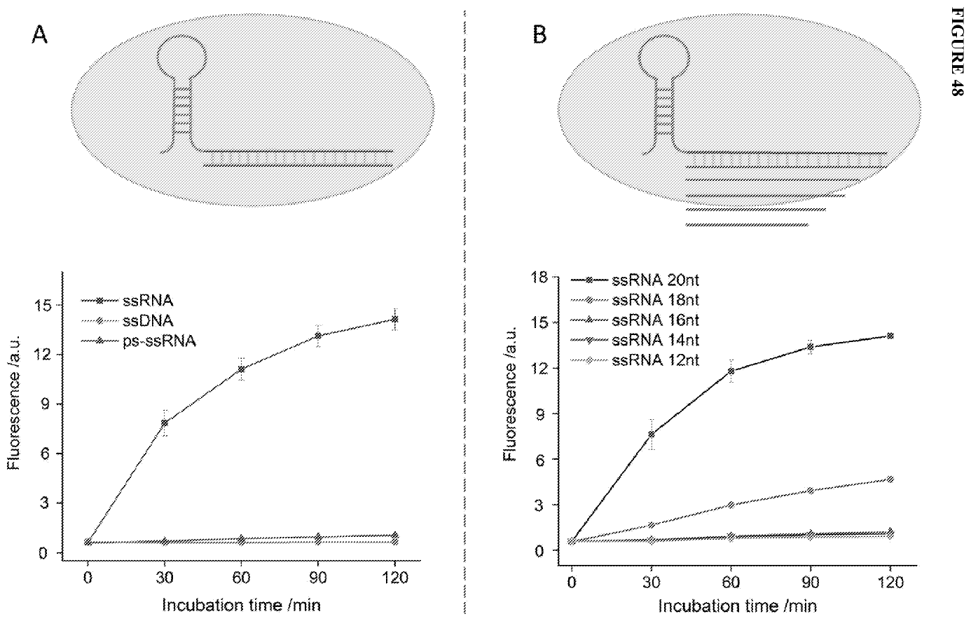

The AutoCAR-1 system is capable of detecting the presence of target DNA at 10 aM sensitivity in a 10 minute reaction, and 1 aM sensitivity in a 20 minute reaction at room temperature (FIG. 45) (Method 9.1a) and detecting the presence of target RNA at 5 aM sensitivity in a 10 minute reaction, and 1 aM sensitivity in a 30 minute reaction at room temperature (FIG. 46) (Method 9.1c).

### Example 15—Modulating Cas13a Trans-Cleavage by Linear-RNA and Circular-RNA: Application to Autocatalytic Sensor Development

In this study, the inventors investigated the trigger ability of ssRNA, dsRNA, and circular RNA and the trans-cleavage ability of Cas13a on ssRNA, dsRNA, and circular RNA.

### 15.1 Materials and Methods

### 15.1.1 Materials

LwCas13a (Magigen), rCutSmart Buffer (New England Biolab), dithiothreitol (Sigma), DNase/RNase free water (ThermoFisher), and phosphate buffered saline (PBS) (Sigma, 10 mM, pH=7.4).

All DNA and RNA oligos are synthesized and modified by Sangon Bio-Tech Ltd.

TABLE S9
DNA and RNA oligos used in Example 15.

| | Oligo | | | SEQ ID |
|---|---|---|---|---|
| Name | type | Sequence 5′-3′ | Modification | NO: |
| gRNA | ssRNA | GGA CCA CCC CAA AAA | N/A | SEQ ID |
| | | CGA AGG GGA CUA AAA | | NO: 73 |
| | | CAC GUU GUU UUG AUC | | |
| | | GCG CCC C | | |
| ssRNA trigger | ssRNA | GGG GCG CGA UCA AAA | N/A | SEQ ID |
| | | CAA CG | | NO: 74 |
| ps-ssRNA | ssRNA | G*G*G* G*C*G* C*G*A* | * | SEQ ID |
| trigger | | U*C*A* A*A*A* C*A*A* | | NO: 75 |
| | | C*G | | |
| ssRNA trigger- | ssRNA | GGG GCG CGA UCA AAA | 5′-FAM | SEQ ID |
| FAM | | CAA CG | | NO: 76 |
| ssRNA trigger- | ssRNA | A AGU GUA GAU GGG GCG | N/A | SEQ ID |
| 40 nt | | CGA UCA AAA CAA CG | | NO: 77 |
| | | GAA CGA AUG U | | |
| ssRNA trigger- | ssRNA | AA UUA GAU GUA AGU | N/A | SEQ ID |
| 60 nt | | GUA GAU GGG GCG CGA | | NO: 78 |
| | | UCA AAA CAA CG GAA | | |
| | | CGA AUG UAC GAA CUG | | |
| | | AG | | |
| ssDNA trigger | ssDNA | GGG GCG CGA TCA AAA | N/A | SEQ ID |
| | | CAA CG | | NO: 79 |
| Trigger cDNA | ssDNA | CG TTG TTT TGA TCG CGC | N/A | SEQ ID |
| | | CCC | | NO: 80 |
| Trigger cDNA- | ssDNA | CG TTG TTT TGA TCG CGC | 5′-FAM; 3′- | SEQ ID |
| FQ | | CCC | BHQ1 | NO: 81 |
| 5U-Reporter | ssRNA | UUUUU | 5′-FAM; 3′- | N/A |
| | | | BHQ1 | |
| 5A-Reporter | ssRNA | AAAAA | 5′-FAM; 3′- | N/A |
| | | | BHQ1 | |
| 5C-Reporter | ssRNA | CCCCC | 5′-FAM; 3′- | N/A |
| | | | BHQ1 | |
| 5G-Reporter | ssRNA | GGGGG | 5′-FAM; 3′- | N/A |
| | | | BHQ1 | |
| RNA reporter | ssRNA | UUAUU | 5′-FAM; 3′- | N/A |
| | | | BHQ1 | |
| DNA reporter | ssDNA | TTATT | 5′-FAM; 3′- | N/A |
| | | | BHQ1 | |
| DNA-RNA | DNA- | TTrATT | 5′-FAM; 3′- | N/A |
| reporter | RNA | | BHQ1 | |
| Trigger cRNA | ssRNA | CG UUG UUU UGA UCG | N/A | SEQ ID |
| | | CGC CCC | | NO: 82 |
| Trigger cRNA- | ssRNA | CG UUG UUU UGA UCG | 3′-BHQ1 | SEQ ID |
| BHQ1 | | CGC CCC | | NO: 83 |
| Trigger cRNA- | ssRNA | CG UUG UUU UGA UCG | 5′-FAM; 3′- | SEQ ID |
| FQ | | CGC CCC | BHQ1 | NO: 84 |
| Trigger cRNA- | ssRNA | UUU UU CG UUG UUU UGA | 5′-Biotin; 3′- | SEQ ID |
| biotin-FAM | | UCG CGC CCC | BHQ1 | NO: 85 |
| Trigger cRNA- | ssRNA | AC AAU CGU UC CG UUG | N/A | SEQ ID |
| 40 nt | | UUU UGA UCG CGC CCC | | NO: 86 |
| | | AUC UAC ACU U | | |
| Trigger cRNA- | ssRNA | CUC AGU UCG UAC AAU | N/A | SEQ ID |
| 60 nt | | CGU UC CG UUG UUU UGA | | NO: 87 |
| | | UCG CGC CCC AUC UAC | | |
| | | ACU UAC AUC UAA UU | | |
| Cir-ssRNA-L3 | ssRNA | GGG GCG CGA UCA AAA | 5′-Azide; 3′- | SEQ ID |
| | | CAA CG/iBio-dT/ UU | CHCH | NO: 88 |
| Cir-ssRNA-L5 | ssRNA | GGG GCG CGA UCA AAA | 5′-Azide; 3′- | SEQ ID |
| | | CAA CG/iBio-dT/ UUU U | CHCH | NO: 89 |
| Cir-ssRNA-L7 | ssRNA | GGG GCG CGA UCA AAA | 5′-Azide; 3′- | SEQ ID |
| | | CAA CG/iBio-dT/UUU | CHCH | NO: 90 |
| | | UUU | | |
| miRNA-21 | ssRNA | UAG CUU AUC AGA CUG | N/A | SEQ ID |
| | | AUG UUG A | | NO: 91 |
| miRNA-21- | ssRNA | GGA CCA CCC CAA AAA | N/A | SEQ ID |
| gRNA | | CGA AGG GGA CUA AAA | | NO: 92 |
| | | CAU CAA CAU CAG UCU | | |
| | | GAU AAG CUA | | |
| T5-L0 | ssRNA | GGG GCG CGA UCA AAA | N/A | SEQ ID |
| | | CAA CGC GUU GUU UUG | | NO: 93 |
| | | AUC GCG CCC C | | |
| T5-L1 | ssRNA | GGG GCG CGA UCA AAA | N/A | SEQ ID |
| | | CAA CGU C GUU GUU UUG | | NO: 94 |
| | | AUC GCG CCC C | | |
| T5-L3 | ssRNA | GGG GCG CGA UCA AAA | N/A | SEQ ID |
| | | CAA CGUUUC GUU GUU | | NO: 95 |
| | | UUG AUC GCG CCC C | | |
| T5-L5 | ssRNA | GGG GCG CGA UCA AAA | N/A | SEQ ID |
| | | CAA CGUUUUUC GUU GUU | | NO: 96 |
| | | UUG AUC GCG CCC C | | |
| T3-L0 | ssRNA | CG UUG UUU UGA UCG | N/A | SEQ ID |
| | | CGC CCCGGG GCG CGA | | NO: 97 |
| | | UCA AAA CAA CG | | |

### 15.1.2 Investigation of the Basic Properties of CRISP/Cas13a Biosensing System

Standard CRISPR/Cas13a reaction mixture: 80 nM Cas13a protein, 40 nM gRNA, 80 nM reporter, and 1× rCutSmart buffer.

Optimization of trigger ssRNA concentration: A variety of trigger ssRNA (0, 5, 10, 20, and 40 nM) was added into 100 μL standard reaction mixture and incubated at room temperature for two hours. The fluorescence signal was tested using ID5 plate reader (Ex 480 nM, and Em 520 nM).

Optimization of gRNA to Cas13a ratio: A variety of gRNA based reaction mixture was prepared (0, 10, 20, 40, 80, 160 nM), 80 nM Cas13a protein, 80 nM reporter, and 1× rCutSmart buffer. Afterwards, 80 nM trigger ssRNA was added into 100 μL standard reaction mixture and incubated at room temperature for two hours. The fluorescence signal was tested using ID5 plate reader (Ex 480 nM, and Em 520 nM).

Optimization of reporter concentration: A variety of reporter was prepared (0, 40, 80, 120, 240, 360 nM), 80 nM Cas13a protein, 40 nM gRNA, and 1× rCutSmart buffer. Afterwards, 80 nM trigger ssRNA was added into 100 μL standard reaction mixture and incubated at room temperature for two hours. The fluorescence signal was tested using ID5 plate reader (Ex 480 nM, and Em 520 nM).

Optimization of buffer: three different buffers were used as the reaction buffer, including Reaction buffer, NEB2.1, and rCutSmart buffer. After preparation of the standard reaction mixture, 80 nM trigger ssRNA was added into 100 μL standard reaction mixture and incubated at room temperature for two hours. The fluorescence signal was tested using ID5 plate reader (Ex 480 nM, and Em 520 nM).

Optimization of temperature: After preparation of the standard reaction mixture, 80 nM trigger ssRNA was added into 100 μL standard reaction mixture and incubated at room temperature or 37° C. for two hours. The fluorescence signal was tested using ID5 plate reader (Ex 480 nM, and Em 520 nM).

Investigation of the limit of detection of standard CRISPR/Cas13a biosensing system: After preparation of the standard reaction mixture, a variety of trigger ssRNA (0, 1 pM, 10 pM, 100 pM, 1 nM, 10 nM, and 100 nM) was added into 100 μL standard reaction mixture and incubated at room temperature for two hours. The fluorescence signal was tested using ID5 plate reader (Ex 480 nM, and Em 520 nM).

### 15.1.3 Investigation of the Basic Properties of Single Strand Trigger for Cas13a RNP.

Standard CRISPR/Cas13a reaction mixture: 40 nM Cas13a protein, 20 nM gRNA, 120 nM reporter, and 1× rCutSmart buffer.

Investigation of different single strand trigger types: 2 μL of 1 μM trigger (ssRNA, ssDNA, and ps-ssRNA) was added into 100 μL standard reaction mixture and incubated at 37° C. for two hours. The fluorescence signal was tested using ID5 plate reader (Ex 480 nM, and Em 520 nM).

Investigation of different trigger length of ssRNA: 2 μL of 1 μM trigger (12 nt, 14 nt, 16 nt, 18 nt, 20 nt, 40 nt, and 60 nt) was added into 100 μL standard reaction mixture and incubated at 37° C. for two hours. The fluorescence signal was tested using ID5 plate reader (Ex 480 nM, and Em 520 nM).

### 15.1.4 Investigation of the Basic Properties of Double Strand Trigger for Cas13a RNP.

Standard CRISPR/Cas13a reaction mixture: 40 nM Cas13a protein, 20 nM gRNA, 120 nM reporter, and 1× rCutSmart buffer.

Investigation of different types of double strand trigger: 2 μL of 1 μM trigger (ssRNA, cRNA, dsRNA, cDNA, ssRNA/cDNA, dsDNA, ps-ssRNA/cDNA) was added into 100 μL standard reaction mixture and incubated at 37° C. for two hours. The fluorescence signal was tested using ID5 plate reader (Ex 480 nM, and Em 520 nM).

Investigation of different length of double strand trigger: 2 μL of 1 μM dsRNA trigger (20 nt, 40 nt, 60 nt, and 80 nt) was added into 100 μL standard reaction mixture and incubated at 37° C. for two hours. The fluorescence signal was tested using ID5 plate reader (Ex 480 nM, and Em 520 nM).

15.1.5 Investigation of Trigger Mechanism of dsRNA Trigger for Cas13a RNP.

To investigate the trigger mechanism, FRET method was first utilized. In brief, a reaction mixture was prepared: 40 nM Cas13a protein, and 20 nM gRNA in 1× rCutSmart buffer. Afterwards, 2 μL of 1 μM fluorescent dsRNA was added into 100 μL prepared reaction mixture. Subsequently, the fluorescence signal was tested using ID5 plate reader (Ex 480 nM, and Em 520 nM). For comparison, 2 μL of 1 μM dsRNA or 2 μL of 1 μM ssRNA-FAM was added into 100 μL 1× rCutSmart buffer.

To further investigate the trigger mechanism, a lock strategy was applied. In brief, a reaction mixture was prepared: 40 nM Cas13a protein, 20 nM gRNA, and 120 nM reporter in 1× rCutSmart buffer. Subsequently, 2 μL of 1 μM trigger (T5-L0, T5-L1, T5-L3, T5-L5, T3-L0) was added into 100 μL standard reaction mixture and incubated at 37° C. for two hours. The fluorescence signal was tested using ID5 plate reader (Ex 480 nM, and Em 520 nM).

### 15.1.6 Investigation of the Basic Trigger Ability of Circular RNA to Cas13a RNP.

The synthesis approach of circular RNA was based on our previous approach (click chemistry). In brief, 0.4 mL of 0.5% w/v streptavidin modified magnetic beads (0.74 μm) were first blocked with 1% BSA solution for 1 h to eliminate non-specific binding. Afterwards, 1 mL of 0.5 μM biotinylated linear-ssRNA was incubated with the beads for 1 h following a PBS wash to remove the residual free linear-ssRNA. Subsequently, 1 mL of the click chemistry reaction solution (1.0 mM CuSO₄, 2.0 mM TCEP, and 100 μM TBTA) was added and incubated with the beads for 12 h at room temperature. After synthesis, the magnetic beads were collected and washed with PBS buffer to remove excess chemicals. Subsequently, 100 μL of 100 units/mL Exonuclease T solution was added and incubated at 37° C. for 30 min to remove the linear ssDNA. After washing with PBS buffer, the synthesized Cir-ssRNA was released from the streptavidin-modified magnetic beads by heat treatment at 95° C. for 30 min, and the supernatant was collected for further use. All the Cir-ssRNA used in this research are synthesized based on this approach. Nanodrop was utilized to test the concentration of synthesized Cir-ssDNA.

The formation of Cir-ssRNA was verified by using agarose gel electrophoresis. In brief, 5% agarose gel in 1×TBE buffer was prepared with SYBR Green DNA dye. 10 μL of Cir-ssRNA was premixed with 2 μL 6×DNA gel loading dye and then loaded into gel for electrophoresis, which was carried out for 40 min at a constant voltage of 100V. 5 μL of 10 bp DNA ladder was used for molecular weight reference. Gel images were visualized by using Gel Doc+XR image system (Bio-Rad Laboratories Inc., USA).

Investigation of trigger ability of Cir-ssRNA or Cir-dsRNA. Standard CRISPR/Cas13a reaction solution was first prepared (40 nM Cas13a protein, 20 nM gRNA, 120 nM reporter, and 1× rCutSmart buffer). Subsequently, 2 μL of 1 μM Cir-ssRNA or Cir-dsRNA trigger was added into 100 μL standard reaction mixture and incubated at 37° C. for two hours. The fluorescence signal was tested using ID5 plate reader (Ex 480 nM, and Em 520 nM).

### 15.1.7 Investigation of the Basic Trans-Cleavage Properties of Cas13a RNP.

To investigate the trans-cleavage properties of Cas13a RNP on single strand nucleic acid, a CRISPR/Cas13a reaction mixture was first prepared: 40 nM Cas13a protein, 20 nM gRNA, and 120 nM reporter (RNA, DNA, RNA-DNA, 5A, 5U, 5C, and 5G) in 1× rCutSmart buffer. Subsequently, 2 μL of 1 μM ssRNA trigger was added into 100 μL standard reaction mixture and incubated at 37° C. for two hours. The fluorescence signal was tested using ID5 plate reader (Ex 480 nM, and Em 520 nM).

To investigate the trans-cleavage properties of Cas13a RNP on double strand nucleic acid, a CRISPR/Cas13a reaction mixture was first prepared: 320 nM Cas13a protein, 160 nM gRNA, and 1 μM dsRNA target in 1× rCutSmart buffer. Subsequently, 16 μL of 1 μM ssRNA trigger was added into 100 μL standard reaction mixture and incubated at 37° C. for two hours. Afterwards, agarose gel electrophoresis was applied to evaluate the conformation of dsRNA. In brief, 5% agarose gel in 1×TBE buffer was prepared with SYBR Green DNA dye. 10 μL of dsRNA was premixed with 2 μL 6×DNA gel loading dye and then loaded into gel for electrophoresis, which was carried out for 40 min at a constant voltage of 100V. 5 μL of 10 bp DNA ladder was used for molecular weight reference. Gel images were visualized by using Gel Doc+XR image system (Bio-Rad Laboratories Inc., USA).

To further investigate the trans-cleavage properties of Cas13a RNP on double strand nucleic acid, a CRISPR/Cas13a reaction mixture was first prepared: 40 nM Cas13a protein, 20 nM gRNA, and 120 nM dsRNA reporters in 1× rCutSmart buffer. Subsequently, 2 μL of 1 μM ssRNA trigger was added into 100 μL standard reaction mixture and incubated at 37° C. for two hours. The fluorescence signal was tested using ID5 plate reader (Ex 480 nM, and Em 520 nM).

### 15.1.8 the Development of RNA Circular-Reporter.

The formation of RNA Cir-reporter was conducted by mixing synthesized Cir-ssRNA with its corresponding fluorescent cRNA at the molar ratio of 1:1. Afterwards, the background of Cir-reporter (120 nM) in rCurSmart buffer was tested using ID5 plate reader (Ex 480 nM, and Em 520 nM).

To investigate the biosensing performance of Cir-reporter assisted CRISPR/Cas13a biosensing system, a CRISPR/Cas13a reaction mixture was first prepared: 40 nM Cas13a protein, 20 nM gRNA, and 120 nM Cir-reporter in 1× rCutSmart buffer. Subsequently, 2 μL of 1 μM ssRNA trigger was added into 100 μL standard reaction mixture and incubated at 37° C. for two hours. The fluorescence signal was tested using ID5 plate reader (Ex 480 nM, and Em 520 nM).

### 15.1.9 the Development of RNA Cir-Amplifier Based Autocatalysis Sensor.

CRISPR/Cas13a reaction mixture was first prepared: 40 nM Cas13a protein, 20 nM gRNA, and 120 nM Cir-reporter in 1× rCutSmart buffer. Subsequently, 2 μL of 1 μM ssRNA trigger was added into 100 μL standard reaction mixture and incubated at 37° C. for two hours. The fluorescence signal was tested using TD5 plate reader (Ex 480 nM, and Em 520 nM).

To investigate the autocatalysis activity, 1 pM ssRNA trigger was added into 100 μL standard reaction mixture and incubated at 37° C. for one hour. The fluorescence signal was tested using ID5 plate reader (Ex 480 nM, and Em 520 nM).

To investigate the sensitivity, different concentrations of ssRNA (0-10 nM) was was added into 100 μL standard reaction mixture and incubated at 37° C. for two hours. The fluorescence signal was tested using ID5 plate reader (Ex 480 nM, and Em 520 nM).

To investigate the specificity, a variety of ssRNA was was added into 100 μL standard reaction mixture and incubated at 37° C. for two hours. The fluorescence signal was tested using ID5 plate reader (Ex 480 nM, and Em 520 nM).

To investigate the stability, 200 nM Cir-reporter (L-5) was incubated rCurSmart buffer, 10% human serum solution, or 10% saliva solution for two hours. The fluorescence signal was tested using ID5 plate reader (Ex 480 nM, and Em 520 nM).

### 15.2 Results

### 15.2.1 Investigation of the Basic Properties of CRISPR/Cas13a Biosensing System

The basic properties of CRISPR/Cas13a biosensing system was investigated (FIG. 47). The fluorescence signal positively increased with the increase of incubation time, and 100% trigger provides the highest fluorescence signal (FIG. 47A). The optimum gRNA to Cas13a ratio was investigated to be 1:2 (FIG. 47B), and higher reporter concentration shows better biosensing performance (FIG. 47C). Afterwards, different reaction buffers were investigated, and rCutSmart shows the optimum performance (FIG. 47D). The reaction temperature was further investigated, and 37° C. shows better performance (FIG. 47E). Finally, the biosensing performance of optimized CRISPR/Cas13a biosensing system was investigated (FIG. 47F), and the limit of detection realized 1 pM in 15 min.

### 15.2.2 Investigation of the Trigger Ability of Single Strand Nucleic Acid

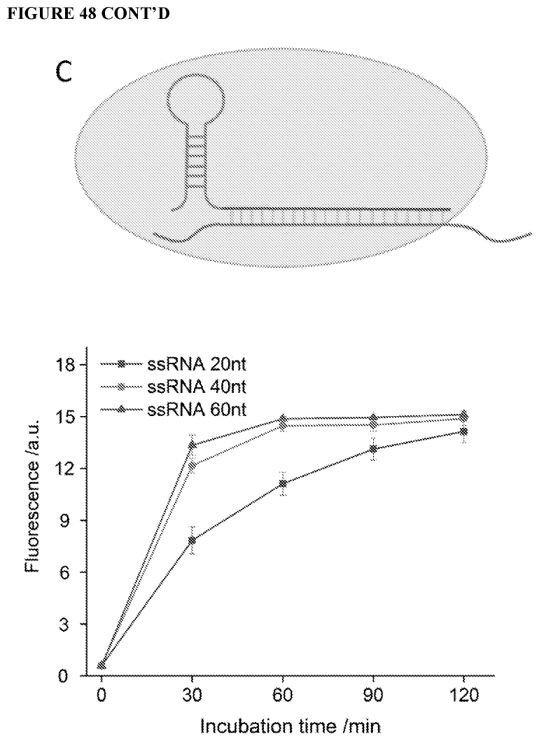

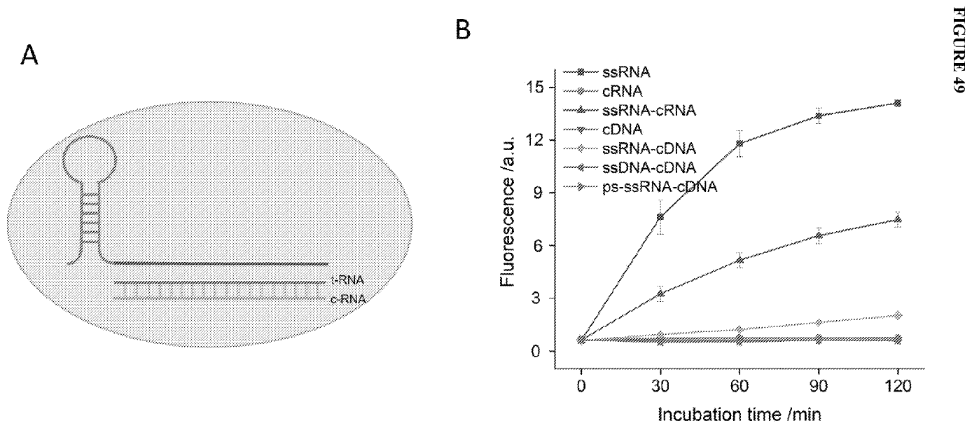

Cas13a RNP has been reported to target single strand RNA. To further investigate the trigger ability of any other type of single strand nucleic acid, single strand DNA and phosphorothioate modified single strand RNA (ps-RNA) were investigated. As shown in FIG. 48A, no trigger ability was observed on ssDNA, and slight signal increase was observed on ps-ssRNA. Therefore, ssRNA was the best trigger for Cas13a RNP. Afterwards, the trigger length of ssRNA was investigated (FIG. 48B), and higher fluorescence signal was observed on longer trigger length, in which 20 nt was the optimum trigger length. With the further increase of trigger length (FIG. 48C), higher fluorescence signal was observed, and 40 nt was the optimum length.

In previous research, 20-bp guide-target RNA duplex is essential for activating the catalytic site of Cas13a within the HEPN domain to cleave target RNA and collateral RNAs. Therefore, in this study, the spacer length of gRNA was 20 nt. As shown in FIG. 48C, the extending of trigger ssRNA results in higher fluorescence signal. This enhancement effect was first observed in Cas13a RNP, although the random extending sequences enhance the trans-cleavage activity of CRISPR/Cas12a has been reported before.

### 15.2.3 Investigation of the Trigger Ability of Double Strand Nucleic Acid

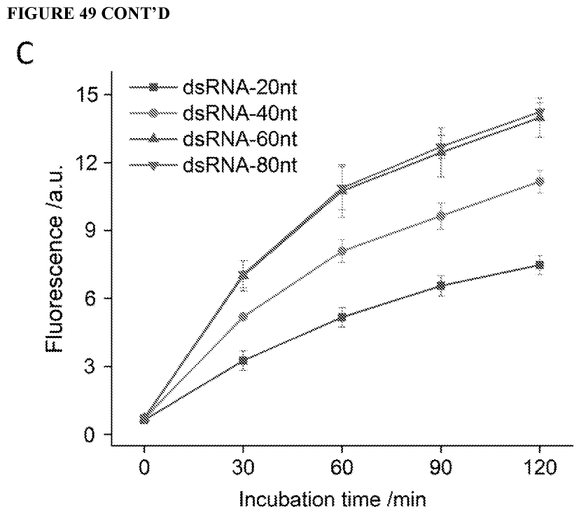

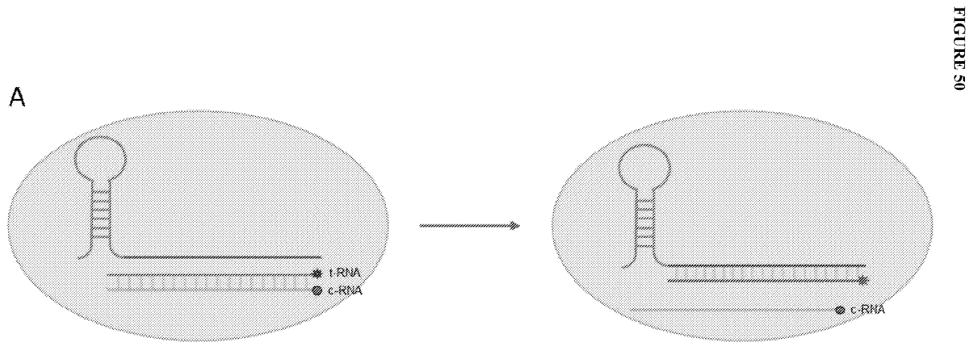

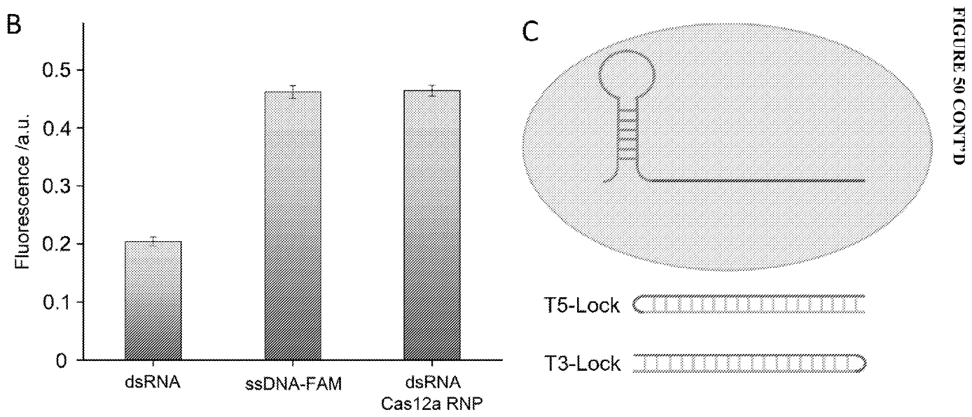

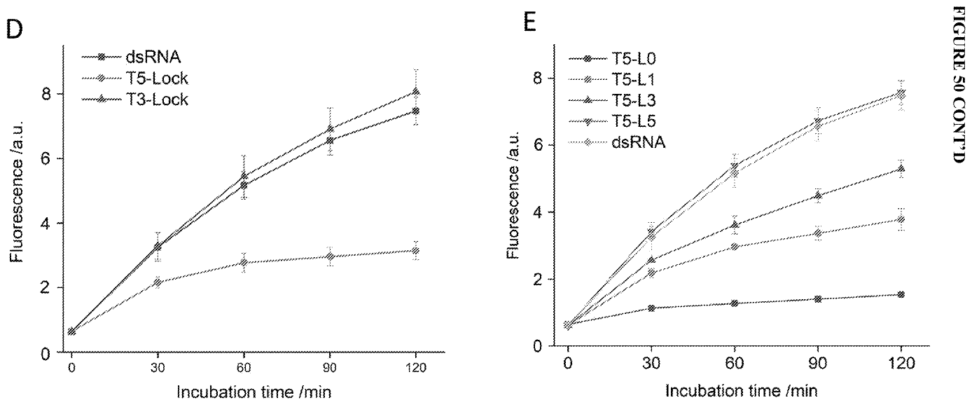

The trigger ability of double strand nucleic acid was investigated (FIG. 49). Double strand RNA (ssRNA/cRNA) shows feasible trigger ability, while RNA/DNA hybrid trigger (ssRNA/cDNA) shows limited trigger ability, and no trigger activity was observed on double strand DNA (ssDNA/cDNA) (FIG. 49B). Since random extending the trigger length could enhance the trans-cleavage activity of Cas13a RNP (FIG. 48C), the inventors further extended the dsRNA trigger length and enhancement effect was observed (FIG. 49C). Therefore, this enhancement effect was effective on both ssRNA trigger and dsRNA trigger.

### 15.2.4 Investigation of the Trigger Mechanism of Double Strand Nucleic Acid

To date, there is no study that reports the trigger ability of dsRNA on Cas13a RNP. To investigate the trigger mechanism of dsRNA for Cas13a RNP, a FRET approach was first applied. As shown in FIG. 50A, the dsRNA was labelled with fluorophore and quencher (t-RNA with FAM, c-RNA with BHQ1). In this condition limited fluorescence signal is expected to be observed. observed. After the dsRNA approaching the Cas13a RNP, the t-RNA will bind to the gRNA of Cas13a RNP, and releasing the cDNA, which leads to the signal increase. This assumption was verified by fluorescent assay (FIG. 50B). Lower fluorescence signal of dsRNA was observed, and higher fluorescence signal of ssRNA-FAM was observed. The fluorescence signal of Cas13a RNP with dsRNA was similar to that of ssRNA-FAM, confirming the binding of t-RNA on the gRNA of Cas13a.

Since the Cas13a RNP is able to separate the t-RNA with c-RNA, the inventors further investigate which sides does it happens (FIG. 50C). The inventors locked the 5′ end of dsRNA (T5-Lock) and 3′ end of dsRNA (T3-Lock) and applied these locked dsRNA to trigger Cas13a RNP. As shown in FIG. 50D, significantly lower signal was observed on T5-Lock, and T3-Lock shows compatible fluorescence signal with dsRNA trigger. Therefore, Cas13a starts to split the dsRNA from 5′ end. To further investigate the mechanism, the inventors increased the locker length from 0 to 5 nt (FIG. 50E), and higher fluorescence was observed with the increase of linker length, confirming the locker effect of 5′-end.

### 15.2.5 Investigation of the Trigger Ability of Circular RNA

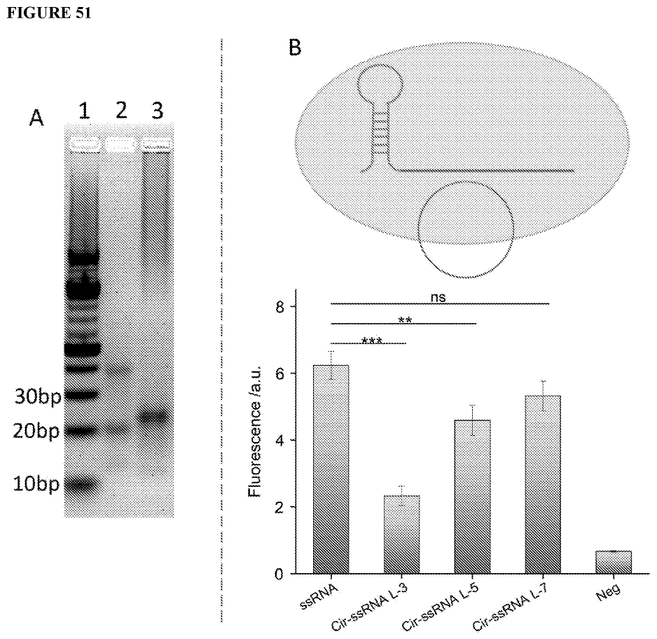

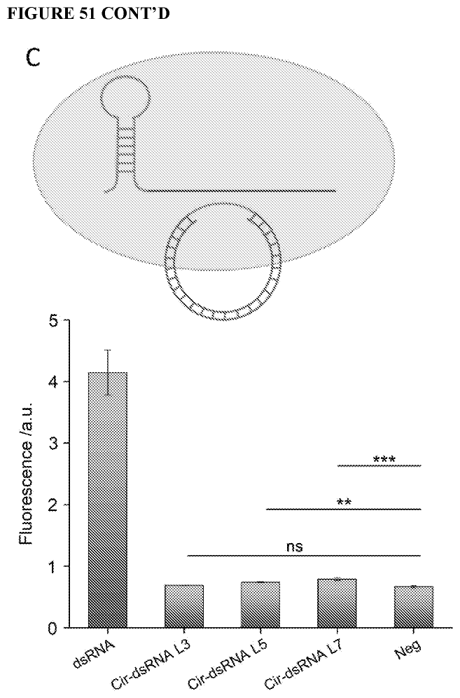

After investigating the trigger ability of single strand nucleic acid and double strand nucleic acid, the inventors further investigated the trigger ability of circular RNA (FIG. 51). The Circular ssRNA was synthesized using click chemistry as described elsewhere herein, and demonstrated using agarose gel electrophoresis (FIG. 51A). Afterwards, the trigger ability of Cir-ssRNA was investigated. As shown in FIG. 51B, lower trigger ability was observed on all Cir-ssRNA, and longer linker length helps the trigger ability recover. The trigger ability of Cir-dsRNA was further investigated, and significant lower fluorescence was observed (FIG. 51C), confirming the lock effect of Circular trigger.

### 15.2.6 Investigation of the Trans-Cleavage Activity of Cas13a

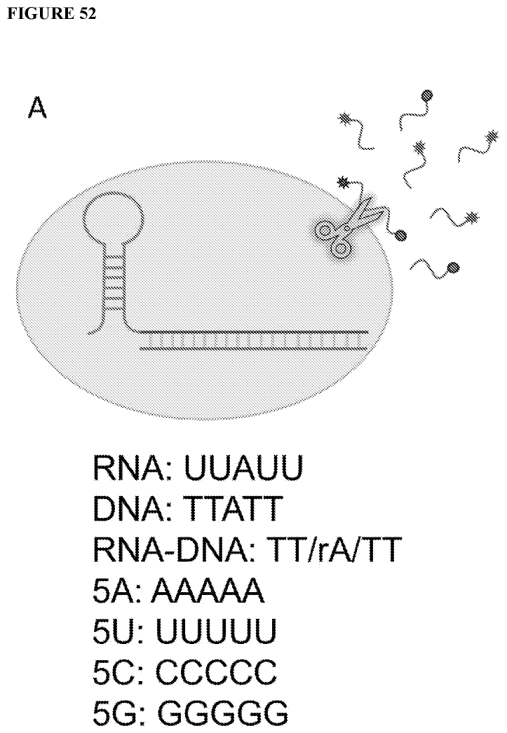

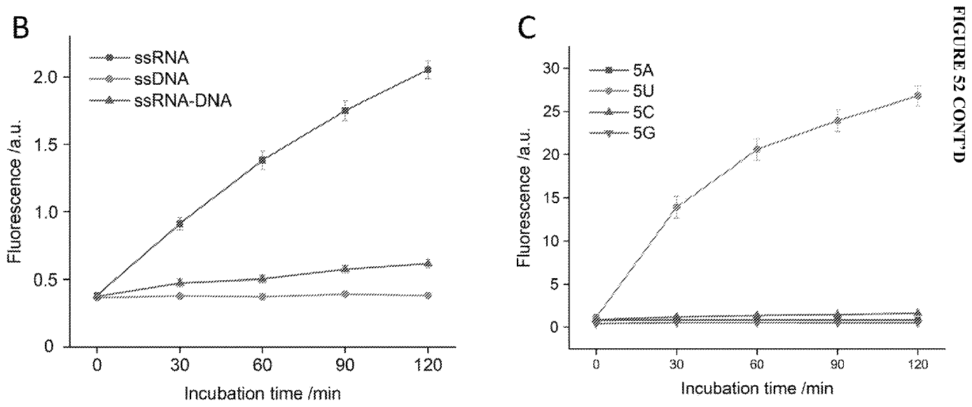

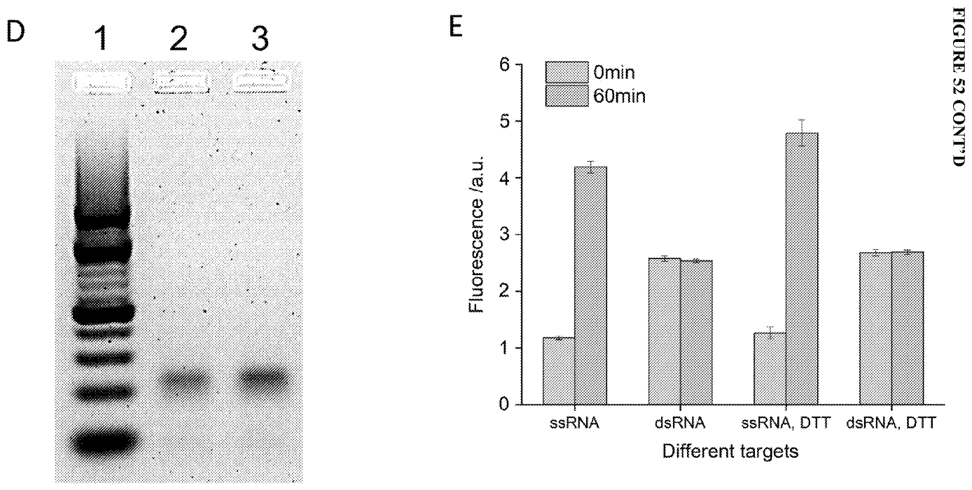

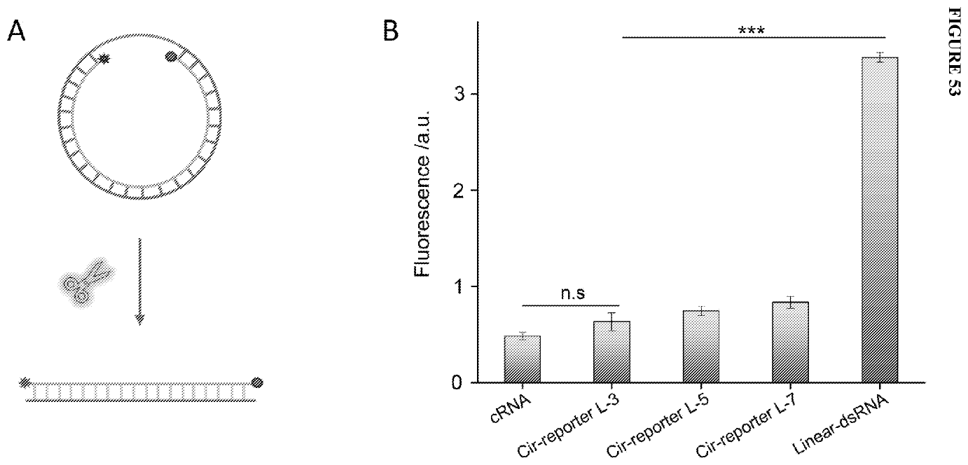

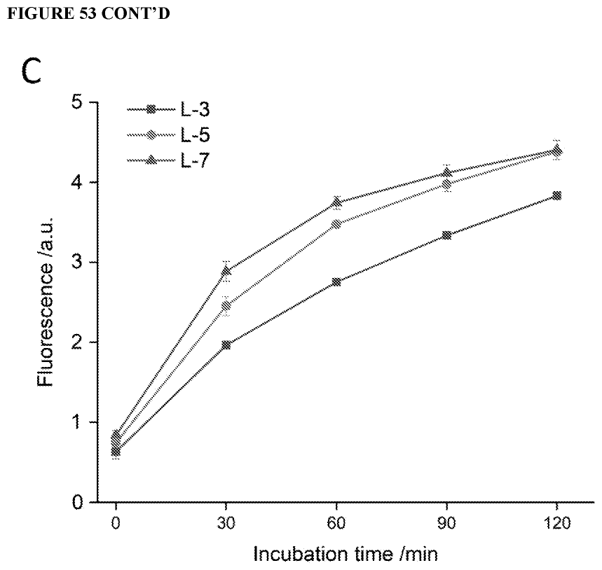

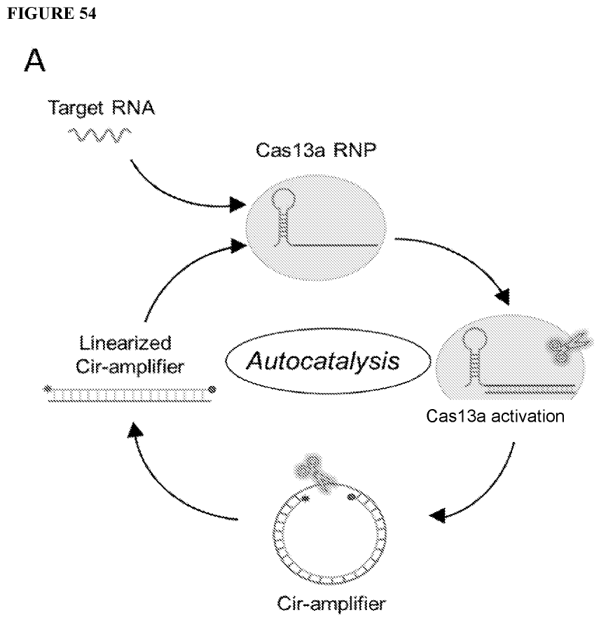

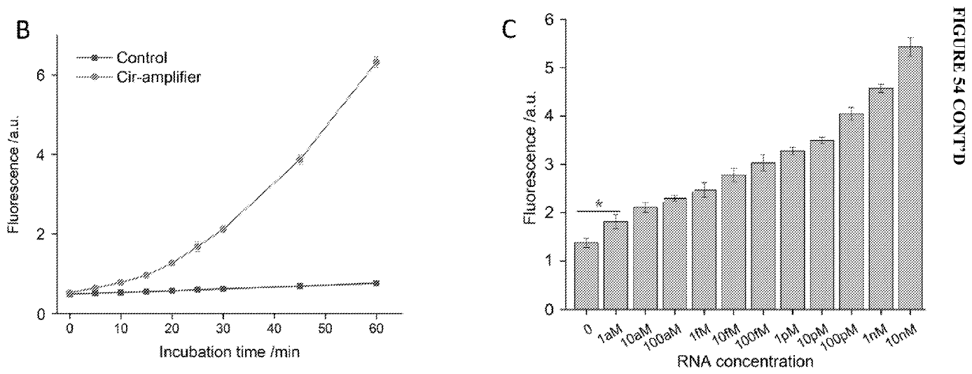

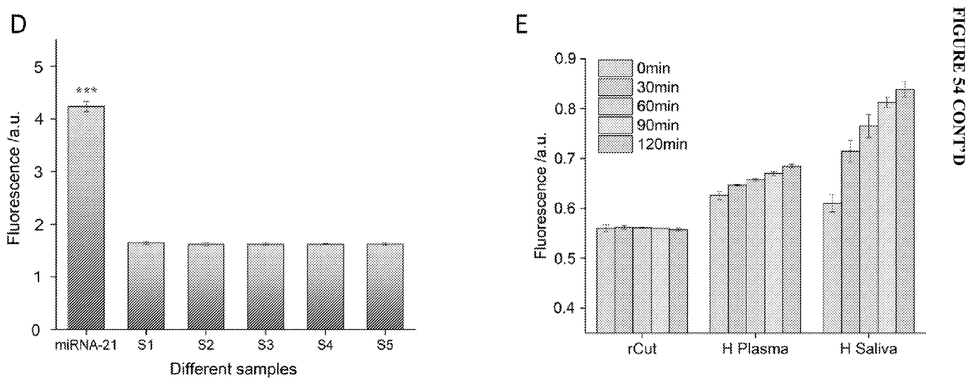

Cas13a has been reported to have exceptional trans-cleavage on single strand RNA. To further investigate the trans-cleavage activity of Cas13a, diverse fluorescence reporters were designed (FIG. 52A). In terms of different types of nucleic acid targets (FIG. 52B), RNA target shows much higher fluorescence increase, while RNA-DNA hybrid target shows slightly fluorescence increase, and no fluorescence increase was observed for DNA, indicating that Cas13a has exceptional trans-cleavage ability on ssRNA, and feasible trans-cleavage ability on RNA-DNA hybrid nucleic acid, and no trans-cleavage ability on DNA. In terms of the ssRNA targets, Cas13a preferably cleaves U over A, C, & G (FIG. 52C).

In addition to the single strand nucleic acid targets, the inventors also investigated the trans-cleavage ability of Cas13a on dsRNA. As shown in FIG. 52D, agarose gel electrophoresis was used to verify to dsRNA (band 2) and Cas13a treated dsRNA (band 3), and no trans-cleavage ability was observed on dsRNA. Furthermore, a double strand fluorescent reporter was applied in CRISPR/Cas13a biosensing system (FIG. 52E), no significant fluorescence increase was observed on dsRNA reporter, demonstrating that Cas13a do not have the trans-cleavage ability on dsRNA.

### 15.2.7 Evaluation of the Biosensing Performance of Cir-Reporter

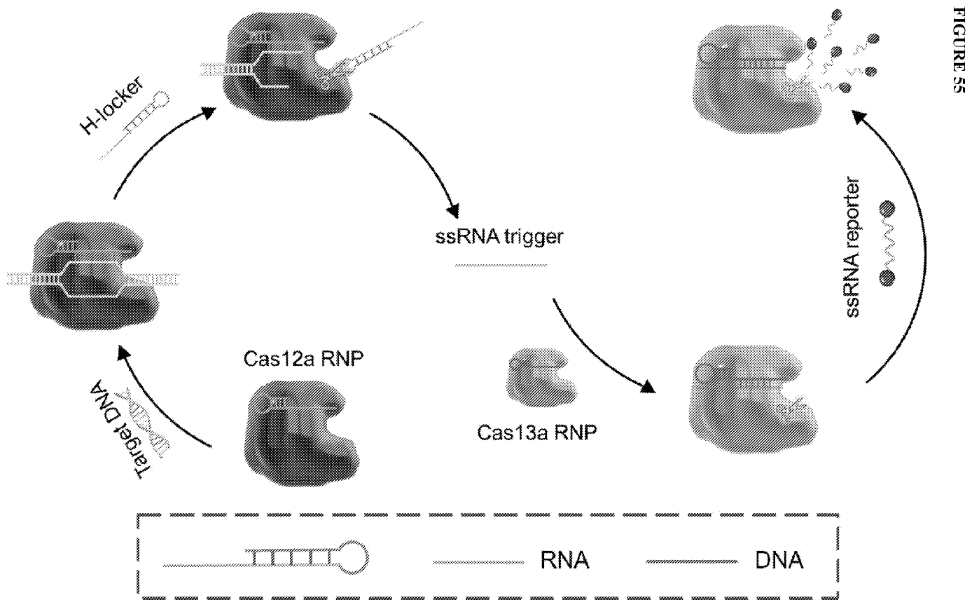

The Cir-reporter was prepared by mixing cir-ssRNA with its corresponding fluorescent cRNA. As shown in FIG. 53A, the Cir-reporter contains a dsRNA region and a ssRNA region, and the fluorescence signal was quenched in Cir-reporter. After the cleavage of the ssRNA region in Cir-reporter, it will become a linearized dsRNA, leading to the release of fluorescence signals. The background of Cir-reporter was tested (FIG. 53B), and longer ssRNA linker length leads to higher background. After cleavage, the fluorescence signal of linearized dsRNA recovered, which is over six times of Cir-reporter. Afterwards, the Cir-reporter was applied as a standard reporter in CRISPR/Cas13a biosensing system (FIG. 53C). After adding trigger ssRNA, the fluorescence signal of Cir-reporter based CRISPR/Cas13a biosensing system continues increased with the increase of incubation time, indicating the feasible performance of Cir-reporter.

### 15.2.8 Establishment of the Cas13a Based Auto-Catalysis Biosensor

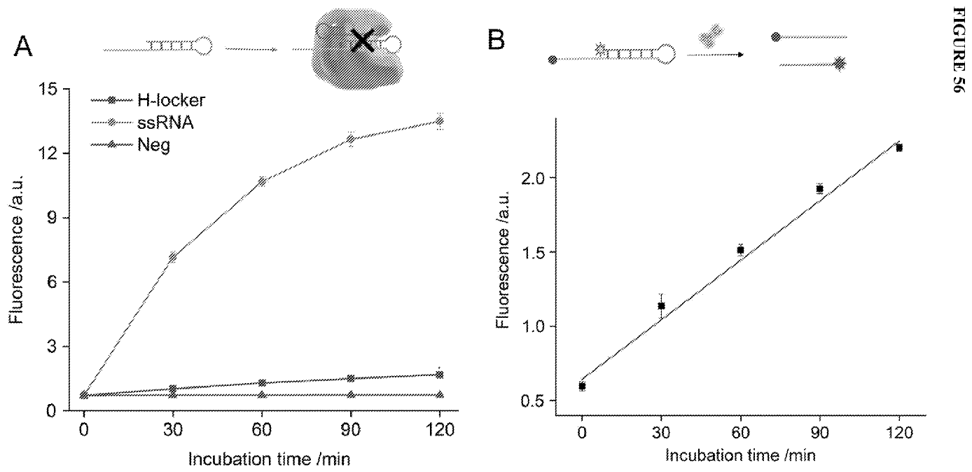

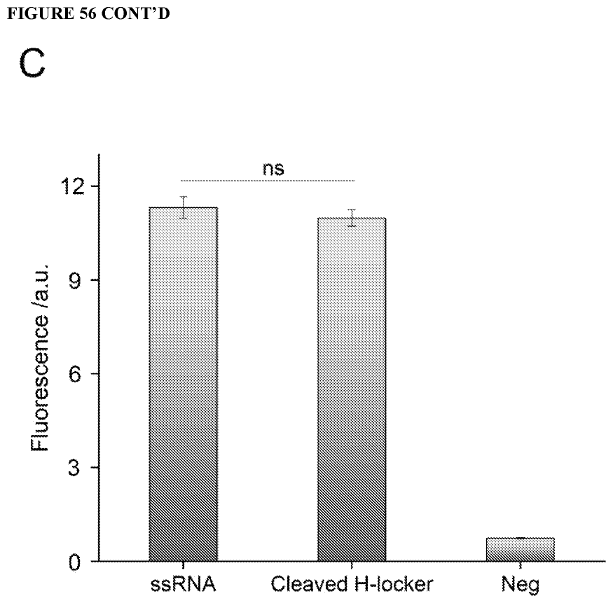

To establish the Cas13a based auto-catalysis biosensor, a Cir-amplifier was established, which contains a dsRNA region and a ssRNA region. The dsRNA region is the fake trigger for Cas13a RNP, and ssRNA is the locker. As shown in FIG. 54A, without genuine target RNA, Cas13a auto-catalysis biosensing system contains two components, Cas13a RNP and Cir-mediator. After introducing genuine target RNA, Cas13a RNP will be activated, then it will trans-cleave the ssRNA region of Cir-amplifier and linearize Cir-amplifier. The Linearized Cir-amplifier will be the fake trigger to activate new Cas13a RNP to form the autocatalysis biosensor. In the meanwhile, the Cir-amplifier could also be a Cir-reporter for signal readout.

The biosensing performance of Cas13a auto-catalysis biosensor was evaluated. In comparison with standard Cas13a biosensor, which shows linear signal increase, Cas13a auto-catalysis biosensor shows an exponential signal increase (FIG. 54B), and the limit of detection of Cas13a auto-catalysis biosensor realized 1 aM (FIG. 54C). In addition, good specificity was demonstrated (FIG. 54D). Furthermore, the stability of Cir-amplifier was investigated (FIG. 54E), and good stability of Cir-amplifier was observed in rCut buffer, while ribonuclease inhibitor is needed for the application of Cir-amplifier in human serum and human saliva samples.

### Example 16—Intramolecularly Bound DNA Autocatalytic Amplifiers (H-Lockers)

In this example, the inventors established a hairpin-locker (H-locker) mediated CRISPR/Cas tandem biosensing system for the ultrasensitive detection of nucleic acids. H-locker was designed to be a linker between two Cas RNPs. After the target DNA/RNA activation, a first Cas RNP will trans-cleave the H-locker to a new trigger for the second Cas RNP. In comparison with standard CRISPR/Cas biosensing system, in which one target will only activate one Cas RNP, H-locker mediated CRISPR/Cas tandem biosensing system enables one target to activate multiple Cas RNPs. The established H-locker mediated CRISPR/Cas tandem biosensing system has the capability of ultrasensitive detection of DNA at 1 aM level.

### 16.1 Methods

### 16.1.1 Materials and Reagents

EnGen® Lba Cas12a (Cpf1) protein (New England Biolab), ENB2.1 buffer (New England Biolab), DNase/RNase free water (ThermoFisher), LwCas13a (Magigen), rCutSmart Buffer (New England Biolab), and phosphate buffered saline (PBS) (Sigma, 10 mM, pH=7.4).

All DNA and RNA oligos are synthesized and modified by Sangon Bio-Tech Ltd.

TABLE S10
DNA and RNA oligos used in this example.

| | Oligo | | | SEQ ID |
|---|---|---|---|---|
| Name | type | Sequence 5′-3′ | Modification | NO: |
| gRNA- | ssRNA | UAA UUU CUA CUA AGU | N/A | SEQ ID |
| Cas12 | | GUA GAU GAC AUA GCA | | NO: 55 |
| | | CAU AGA CUG AGA | | |
| Trigger- | ssDNA | TCT CAG TCT ATG TGC | N/A | SEQ ID |
| Cas12 | | TAT GTC | | NO: 54 |
| gRNA- | ssRNA | GGA CCA CCC CAA AAA | N/A | SEQ ID |
| Cas13 | | CGA AGG GGA CUA AAA | | NO: 73 |
| | | CAC GUU GUU UUG AUC | | |
| | | GCG CCC C | | |
| Trigger- | ssRNA | GGG GCG CGA UCA AAA | N/A | SEQ ID |
| Cas13 | | CAA CG | | NO: 74 |
| Reporter- | ssRNA | UUUUU | 5′-FAM; 3′- | N/A |
| Cas13 | | | BHQ1 | |
| H-locker | DNA/RNA | ATCGC GCCCC CC | 5′-Texas Red; | SEQ ID |
| | | /rGrGrG rGrCrG rCrGrA | 3′-BHQ2 | NO: 98 |
| | | rUrCrA rArArA rCrArA | | |
| | | rCrG/ | | |

### 16.1.2 Investigation of the Trigger Ability of H-Locker.

Standard CRISPR/Cas13a reaction mixture: 40 nM Cas13a protein, 20 nM gRNA-Cas13, 120 nM reporter-Cas13, and 1× rCutSmart buffer.

Afterwards, 2 μL of 1 μM trigger-Cas13 (ssRNA, and H-locker) was added into 100 μL standard reaction mixture and incubated at 37° C. for two hours. The fluorescence signal was tested using ID5 plate reader (Ex 480 nM, and Em 520 nM).

### 16.1.3 Investigation of the Reporter Ability of H-Locker.

Standard CRISPR/Cas12a reaction solution was prepared. In brief, 1 μL of 100 μuM Cas12a protein, 5 μL of 20 μM gRNA-Cas12, and 6 μL of 100 μM H-lock were added into 3.6 mL rCutSmart buffer. The prepared solution was stored in 4° C. before use. Afterwards, 5 μL of 1 μM trigger-Cas12 ssDNA was added to trigger the CRISPR/Cas12a reaction and incubated at room temperature for two hours. The fluorescence signal was tested using SpectraMax iD5 multi-Mode Microplate Reader (λex: 570 nm; λem: 615 nm).

### 16.1.4 Investigation of the Trigger Ability of Cleaved H-Locker.

Standard CRISPR/Cas13a reaction mixture: 40 nM Cas13a protein, 20 nM gRNA-Cas13, 120 nM reporter-Cas13, and 1× rCutSmart buffer.

Afterwards, 2 μL of 1 μM cleaved H-locker was added into 100 μL standard reaction mixture and incubated at 37° C. for two hours. The fluorescence signal was tested using ID5 plate reader (Ex 480 nM, and Em 520 nM).

16.1.5 Investigation of the Biosensing Performance of H-Locker Mediated CRISPR/Cas Tandem biosensing system.

A standard reaction solution was prepared. In brief, 1 μL of 100 μM Cas12a protein, 5 μL of 20 μM gRNA-Cas12, 50 μL of 2 μM Cas13a protein, 5 μL of 20 μM gRNA-Cas13, and 15 μL of 20 μM reporter-Cas13 were added into 3.6 mL rCutSmart buffer. The prepared solution was stored in 4° C. before use. Afterwards, different concentrations of trigger ssDNA were added to trigger the CRISPR/Cas reaction and incubated at 37° C. for two hours. The fluorescence signal was tested using ID5 plate reader (Ex 480 nM, and Em 520 nM).

### 16.2 Results

### 16.2.1 Schematic of H-Locker Mediated CRISPR/Cas Tandem Biosensing System

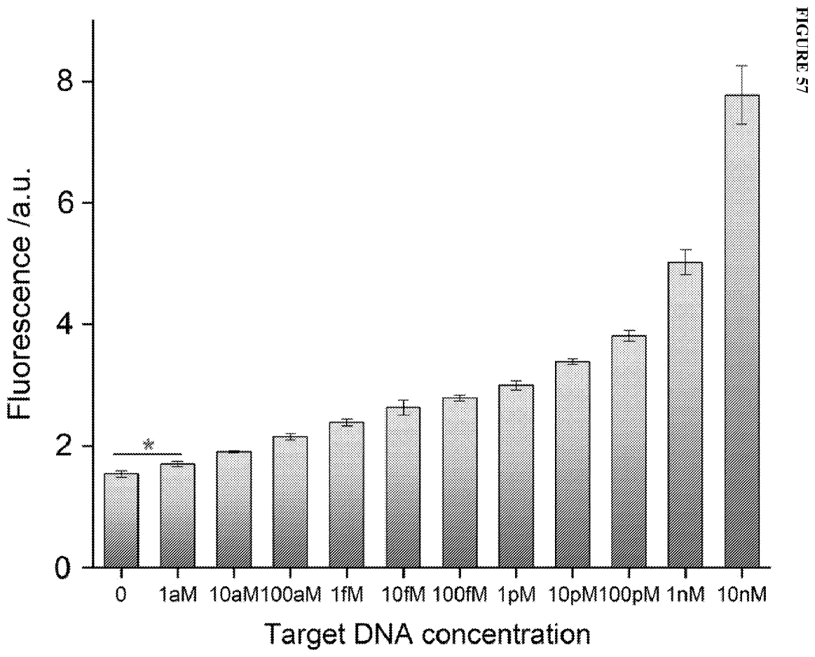

As shown in Figure. 55, H-locker mediated CRISPR/Cas tandem biosensing system was prepared. In this study, the inventors used Cas12-Cas13 as an example. The Cas12a RNP was first activated by target DNA, afterwards it will cleave the loop region of H-locker to release the ssRNA trigger, leading to the activation of Cas13a RNP. In comparison with the standard CRISPR/Cas system, one target will only activate one Cas RNP. In this tandem system, one target will activate one Cas RNP1, which will cleave thousands of H-locker, leading to the activation of thousands of Cas RNP2. In this biosensing system, the H-locker contains two part, a ssRNA part as the trigger for Cas13a, and a ssDNA part as the locker.

### 16.2.2 Establishment of H-Locker Mediated CRISPR/Cas Tandem Biosensing System

The trigger ability of H-locker was first investigated. As shown in FIG. 56A, with the increase of incubation time, the fluorescence signal of H-locker slightly increased. Comparison with the fluorescence of ssRNA, it has been significantly reduced to 7.6% of ssRNA. Therefore, H-lock is an effective locker to block the trigger ability of ssRNA to Cas13a RNP.

Afterwards, the inventors investigated whether the H-locker can be effectively activated by Cas12a RNP. To review the H-locker open process, a fluorophore and a corresponding quencher were immobilized on both ends of H-locker. The fluorescence was quenched in H-locker. After the activation of Cas12a RNP, it will trans-cleave the loop region of H-locker, leading to the recovery of fluorescence signal. As shown in FIG. 56B, with the increase of incubation time, the fluorescence signal continues increasing, conforming the open of H-locker.

The cleaved H-locker was further applied to trigger downstream Cas13a RNP. As shown in FIG. 56C, compatible trigger ability was observed on cleaved H-locker and ssRNA, conforming the recovery of trigger ability.

### 16.2.3 Investigation of the Biosensing Performance of H-Locker Mediated CRISPR/Cas Tandem Biosensing System.

H-locker mediated CRISPR/Cas tandem biosensing system was established (FIG. 57). After one hour incubation, the tandem biosensing system is able to realize 1 aM, confirming the exceptional biosensing performance of tandem system.

### Example 17—Cir-Report-Induced CRISPR/Cas12a Amplification with Electrochemical Detection

In this example utilization of electrochemical detection was established using the Cir-amplifiers (AutoCAR-2).

### 17.1 Methods

17.1a Electrochemical reporters: 1 mm Au electrode was incubation in 5 μM SH-ssDNA-Methylene blue (ssDNA=CCCCC CCCCC; SEQ ID NO: 99) solution overnight. Afterwards, the Au electrode was incubated in 3 mM MCH solution for two hours at room temperature. The prepared electrochemical reporters were stored in 4° C. PBS solution before use.

17.1b Standard CRISPR/Cas12a reaction mixture: 1 μL 100 μM (100 pmol) of Cas12a protein was gently mixed with 5 μL 20 μM (100 pmol) of gRNA-D to form the Cas12a RNP in 5 mL 1×NEB 2.1 buffer.

17.1c AutoCAR-2 reaction mixture: 1 μL 100 μM (100 pmol) of Cas12a protein was gently mixed with 5 μL 20 μM (100 pmol) of gRNA-D to form the Cas12a RNP in 5 mL 1×NEB 2.1 buffer. Subsequently, 200 μL of 5 μM (1 nmol) of Cir-reporter solution was added and well mixed to form the reaction mixture. Afterwards, the prepared electrochemical reporters were incubated in 0.5 mL reaction mixture, followed by adding 1 nM target-D ssDNA to activate the trans-cleavage of Cas12a and enabling the CRISPR/Cas autocatalysis biosensing reaction. The electrochemical signal was recorded using CHI660 Electrochemical workstation (Amp 20 mV, Freq 200 Hz).

### 17.2 Results

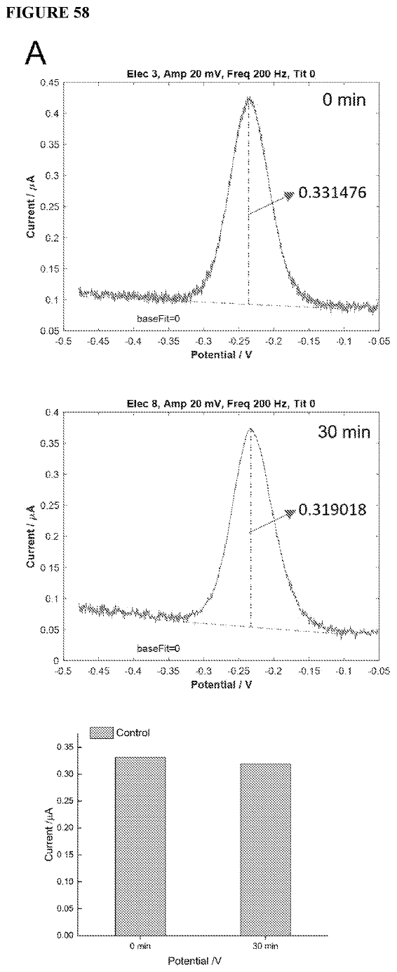

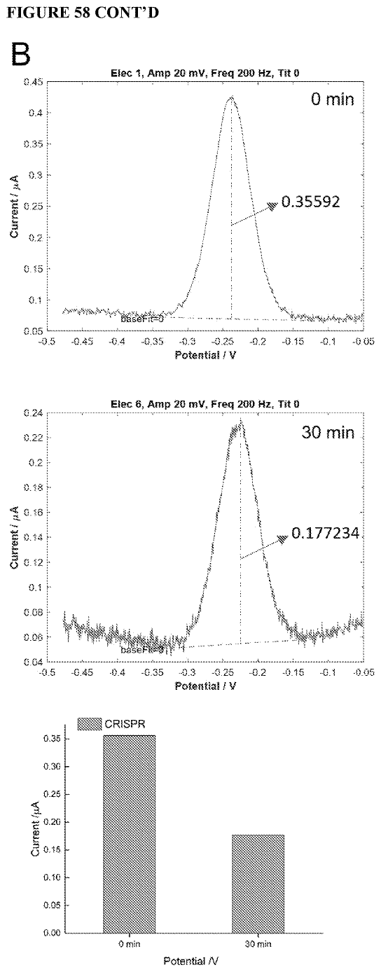

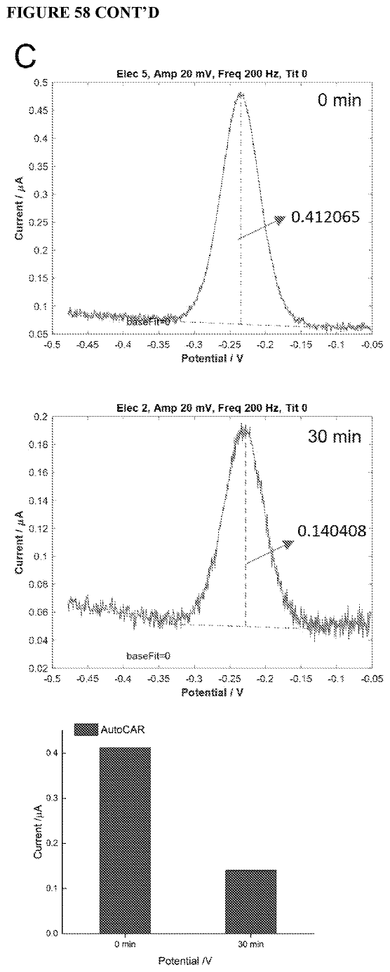

The results demonstrate successful electrochemical signal detection with the AutoCAR system (FIG. 58).

### Example 18: Detection of Target RNA Using Circular RNA-DNA

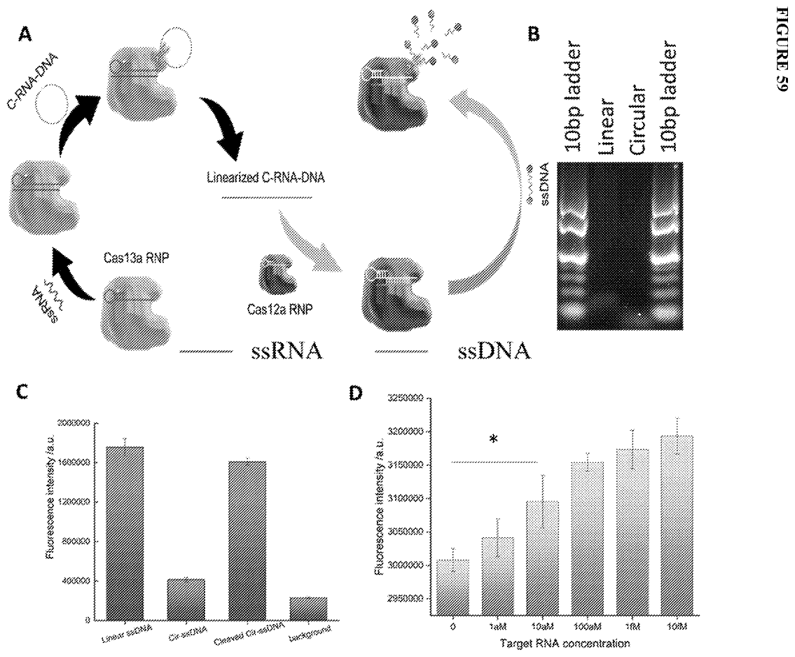

The detection scheme is illustrated in FIG. 59 A.

### 18.1 Methods

Synthesis of Circular ssRNA-DNA

This method was introduced to improve purity of single ring synthesis and to improve product concentration. The use of magnetic beads makes it possible to concentrate Cir-ssRNA-DNA. To synthesize Cir-ssRNA-DNA, 0.4 mL of 0.5% w/v streptavidin modified magnetic beads (0.74 μm) were first blocked with 1% BSA solution for 1 h to eliminate non-specific binding. Afterwards, 1 mL of 0.5 μM biotinylated linear-ssRNA-DNA was incubated with the beads for 1 h following a PBS wash to remove the residual free linear-ssRNA-DNA. Subsequently, 1 mL of the click chemistry reaction solution (1.0 mM CuSO4, 2.0 mM TCEP, and 100 μM TBTA) was added and incubated with the beads for 12 h at room temperature. After synthesis, the magnetic beads were collected and washed with PBS buffer to remove excess chemicals. Subsequently, 100 μL of 100 units/mL Exonuclease VII solution was added and incubated at 37° C. for 30 min to remove the linear-ssRNA-DNA. After washing with PBS buffer, the synthesized Cir-ssRNA-DNA was released from the streptavidin-modified magnetic beads by heat treatment at 95° C. for 30 min, and the supernatant was collected for further use. All the Cir-ssRNA-DNA used in this research are synthesized based on this approach. Nanodrop was utilized to test the concentration of synthesized Cir-ssRNA-DNA.

### Pre-Activation Using Cas13a:

Reaction mixture was prepared to achieve final concentrations of 40 nM Cas13a protein, and 20 nM gRNA in 1× rCutSmart buffer. 90 μL prepared reaction mixture was added to an Eppendorf Afterwards, 10 μL of the trigger RNA at different concentrations was added to the mix. To this 100 μL prepared reaction, 5 μL of 1 μM circular dsRNA-DNA was added. The reaction was incubated at 37° C. for 60 minutes.

### Determination of Target RNA Using Cas12a:

CRISPR/Cas12a reaction mixture was prepared as follows: 1 μL 100 μM (100 pmol) of Cas12a protein was gently mixed with 5 μL 20 μM (100 pmol) of gRNA for Cas12a in 3.6 mL 1×NEB 2.1 buffer with 166 nM of Hairpin reporters. Then, 90 μL of the CRISPR mix was dispensed into 96-well plates. 10 μL of the preactivated Cas13a reaction was added to the mix. The plates were incubated at 37° C. for 60-120 minutes and the fluorescence was measured at 570 nm and 615 nm.

TABLE S11
Sequences used for circular RNA-DNA:

| | Oligo | | | |
|---|---|---|---|---|
| Name | type | Sequence 5′-3′ | Modifications | SEQ ID NO: |
| Cir | ssRNA- | TTT TTT/iAzide-dT/CT ATG | 5′-Biotin; | SEQ ID NO: |
| RNA- | DNA | TGC TAT GTC /rUrUrU/ | 3′-Alkyne | 100 |
| DNA | hybrid | | | |
| cDNA | ssDNA | TTT AGA CAT AGC ACA TAG | | SEQ ID NO: |
| | | | | 51 |
| gRNA | ssRNA | GGA CCA CCC CAA AAA CGA | | SEQ ID NO: |
| for | | AGG GGA CUA AAA CAC GUU | | 73 |
| Cas13a | | G?U UUG AUC GCG CCC C | | |
| gRNA | ssRNA | UAA UUU CUA CUA AGU GUA | | SEQ ID NO: |
| for | | GAU GAC AUA GCA CAU AGA | | 66 |
| Cas12a | | CUG AGA CUG | | |
| Hairpin | ssDNA | CTCTCATTTTTTTTTTAGAGAG | 5′-Texas red; | SEQ ID NO: |
| Reporter | | | 3′-BHQ1 | 101 |
| Target | ssRNA | GGG GCG CGA UCA AAA CAA | | SEQ ID NO: |
| RNA | | CG | | 74 |

### 18.2 Results

Synthesis of Circular ssRNA-DNA

Cir-amplifiers and mediators were successfully synthesized by generating a single ring circular-ssRNA-DNA using click chemistry as described in the materials and methods section below. Circular ssRNA-DNA of different linker lengths was synthesised using click-chemistry (L3 and L5), post the procedure Exonuclease VII was used to degrade the linear ssRNA-DNA in the mix. The difference in the migration pattern between the linear and circular RNA-DNA was evident using gel electrophoresis, confirming the formation of Cir-ssRNA-DNA (shown in FIG. 59 B). The circular ssRNA-DNA synthesised was then combined with complementary DNA of shorter length to form either the amplifier or mediator for the CRISPR Cas13a reaction.

### Performance of RNA Circular Mediators and Amplifiers in CRISPR Cas13a and Cas12 Reaction

The products of preactivated/not activated Cas13a reaction were used for a subsequent Cas12a reaction. A significant difference in the activation of the CRISPR cascade was observed when comparing circular RNA-DNA to linear/trans-cleaved RNA-DNA (referred to as Cir-ssDNA in FIG. 59 C). Within a dual ribonucleoprotein (RNP) system comprising Cas13a and Cas12a, the employment of L3 RNA-DNA circular mediator, preactivated with different RNA concentrations using Cas13a exhibited a limit of detection of 10 attomolar (aM) following the addition of trans-cleaved products in a Cas12a reaction.

### DESCRIPTION OF THE SEQUENCE LISTING SUBMITTED ELECTRONICALLY

The present application contains a sequence listing which has been submitted electronically as an XML document in the ST.26 format and is hereby incorporated by reference in its entirety. Said XML copy, created on 10 Nov. 2023, is named P0048952PCT Seq Listing.xml and is xxx bytes in size.

1. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA sequence or DNA/RNA hybrid sequence first occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA;
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein; and
(b) measuring a detectable signal produced following cleavage of the labelled nucleic acid reporter by the first and second type V or type VI CRISPR/Cas effector protein, thereby detecting the target in the sample.

2. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence
(v) a second type V or type VI CRISPR/Cas effector protein;
(vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA sequence or DNA/RNA hybrid sequence first occurs following cleavage of the ssDNA or ssRNA region of the circular DNA or RNA molecular construct, respectively, by said first type V or type VI CRISPR/Cas effector protein and further activates the trans-cleavage nuclease activity of the second type V or type VI CRISPR/Cas effector protein bound to said second guide RNA;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first and second type V or type VI CRISPR/Cas effector protein, thereby detecting the target in the sample;
wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.

3. The method of claim 2, wherein the trigger nucleic acid sequence is a ssDNA or dsDNA or ssRNA sequence, preferably not having full (100%) complementarity to an existing genomic sequence, more preferably with the length of at least 10 nucleotides.

4. The method of claim 2 or 3, wherein the target binding construct is an antibody or antigen binding fragment thereof.

5. The method of any one of claims 2 to 4, wherein the target binding construct is immobilized on a substrate or conjugated to a magnetic bead or a microparticle or a nanoparticle.

6. The method of claim 5, further comprising the step of performing magnetic separation of the captured target bound to the first target binding construct from the sample when the first target binding construct is conjugated to a magnetic bead.

7. The method of any one of claims 1 to 6, wherein the first and second type V or type VI CRISPR/Cas effector proteins are different.

8. The method according to claim 7, wherein said first type V or type VI CRISPR/Cas effector protein is a Type V CRISPR/Cas effector protein and said second type V or type VI CRISPR/Cas effector protein is a Type VI CRISPR/Cas effector protein.

9. The method of any one of claims 1 to 8, wherein contacting the sample with the second effector type V or type VI CRISPR/Cas effector protein and the second guide RNA occurs after the sample has been contacted with the first effector type V or type VI CRISPR/Cas effector protein and the first guide RNA.

10. The method of claim 9, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.

11. The method of any one of claims 1 to 10, wherein the first and/or second guide RNA is bound to the first and/or second type V or type VI CRISPR/Cas effector protein, respectively.

12. A method of enhancing a type V or type VI CRISPR/Cas detection system comprising
adding to a reaction mixture comprising a first type V or Type VI CRISPR/Cas effector protein, a first guide RNA, and a labelled nucleic acid reporter of the system:
(i) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(ii) a second type V or type VI CRISPR/Cas effector protein;
(iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the guide RNA and the dsDNA or ssDNA or dsRNA or DNA/RNA hybrid sequence first occurs following cleavage of the ssDNA or ssRNA region of the circular DNA or RNA molecular construct, respectively, by the type V or type VI CRISPR/Cas effector protein of the detection system and further activates the nuclease activity of the second type V or type VI CRISPR/Cas effector protein; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein;
and measuring a detectable signal produced following cleavage of the labelled nucleic acid reporter, and/or the labelled reporter construct when added, by the second type V or type VI CRISPR/Cas effector protein.

13. The method of claim 12, wherein the guide RNA is bound to the second type V or type VI CRISPR/Cas effector protein.

14. The method of any one of claims 1 to 13, wherein the circular DNA molecular construct or circular RNA molecular construct has the total length (circumference) from 15 to 30 nucleotides.

15. The method of claim 14 wherein the circular DNA molecular construct or circular RNA molecular construct has a total length of 16, 17, 18, 19, 20, or 21 nucleotides.

16. The method of claim 14 or 15, wherein the circular DNA molecular construct or circular RNA molecular construct comprises two parts including a 2-5 nucleotides ssDNA or ssRNA, respectively, and the remaining part of the molecular construct is dsDNA or ssDNA or dsRNA or DNA/RNA with a complementary sequence to the second guide RNA or the guide RNA which binds to the second type V or type VI CRISPR/Cas effector protein.

17. The method of any one of claims 1 to 16, wherein the sequences of dsDNA or ssDNA are random nucleic acid sequences, preferably not forming complex secondary structures, and preferably not fully (100%) complementary to any naturally existing genomic sequences.

18. The method of any one of claims 1 to 17, wherein the circular DNA molecular construct or circular RNA molecular construct completely or significantly blocks CRISPR/Cas activation for at least 1.5 hours when non-linearized.

19. The method of any one of claims 1 to 18, wherein any one or more of the guide RNA, circular DNA molecular construct or circular RNA molecular construct, the reporter construct, and trigger nucleic acid, when present, comprises at least one nucleotide containing a non-natural modification/substitution.

20. The method of any one of claims 1 to 19, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13

21. The method of any one of claims 1 to 20, wherein the reporter construct is a labelled RNA

22. The method of any one of claims 1 to 20, wherein the reporter construct is a labelled DNA.

23. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
wherein the dsDNA sequence of the circular DNA molecular construct or the dsDNA or ssDNA or dsRNA or DNA/RNA hybrid sequence of the circular RNA molecular construct specifically hybridizes with the first guide RNA following linearization of circular DNA or RNA molecular construct, respectively, by the nuclease activity of said first type V or type VI CRISPR/Cas effector protein and also activates the nuclease activity of first type V or type VI CRISPR/Cas effector proteins bound to said first guide RNA;
(b) measuring a detectable signal produced following linearization of the circular DNA molecular construct, or the circular RNA molecular construct, by the first and/or second type V or type VI CRISPR/Cas effector protein, thereby detecting the target nucleic acid in the sample.

24. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct;
wherein hybridization between the guide sequence of the second guide RNA and the dsDNA or ssDNA sequence first occurs following linearization of circular DNA or RNA molecular construct, respectively, by the nuclease activity of said first type V or type VI CRISPR/Cas effector protein and further activates the nuclease activity of the second type V or type VI CRISPR/Cas effector protein bound to said second guide RNA;
(b) measuring a detectable signal produced following linearization of the circular DNA molecular construct, or the circular RNA molecular construct, by the first and/or second type V or type VI CRISPR/Cas effector protein, thereby detecting the target nucleic in the sample.

25. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
wherein the dsDNA of the circular DNA molecular construct or the dsDNA or ssDNA or dsRNA or DNA/RNA hybrid sequence of the circular RNA molecular construct specifically hybridizes with the first guide RNA following linearization of the circular DNA or RNA molecular construct, respectively, by the nuclease activity of said first type V or type VI CRISPR/Cas effector protein and also activates the nuclease activity of first type V or type VI CRISPR/Cas effector proteins bound to said first guide RNA;
(v) a target binding construct; and
(b) measuring a detectable signal produced following linearization of the circular DNA molecular construct, or the circular RNA molecular construct, by the first type V or type VI CRISPR/Cas effector protein, thereby detecting the target in the sample;
wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.

26. A method for the detection of a target in a sample, the method comprising:
(a) contacting the sample with:
(i) a first type V or type VI CRISPR/Cas effector protein;
(ii) a trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with a trigger nucleic acid sequence, wherein hybridization between the first guide sequence and the trigger nucleic acid sequence activates the nuclease activity of said first type V or type VI CRISPR/Cas effector protein;
(iv) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled;
(v) a second type V or type VI CRISPR/Cas effector protein;
(vi) a second guide RNA, optionally bound to said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA sequence or dsRNA sequence or DNA/RNA hybrid sequence of the circular RNA molecular construct, wherein hybridization between the guide sequence of the second guide RNA and the dsDNA or ssDNA or dsRNA or DNA/RNA hybrid sequence first occurs following linearization of ssDNA or ssRNA sequence of the circular DNA or RNA molecular construct, respectively, by said first type V or type VI CRISPR/Cas effector protein and further activates the nuclease activity of the second type V or type VI CRISPR/Cas effector protein bound to said second guide RNA;
(viii) a target binding construct; and
(b) measuring a detectable signal produced following linearization of the circular DNA molecular construct, or the circular RNA molecular construct, by the first and/or second type V or type VI CRISPR/Cas effector protein, thereby detecting the target in the sample;
wherein the first type V or type VI CRISPR/Cas effector protein, the trigger nucleic acid sequence, the first guide RNA, the first type V or type VI CRISPR/Cas effector protein in combination with the first guide RNA, or the first type V or type VI CRISPR/Cas effector protein in combination with the first guide RNA and the trigger nucleic acid is linked or conjugated to the target binding construct to thereby co-locate the type V or type VI CRISPR/Cas effector protein and the target when present in the sample.

27. The method of claim 25 or 26, wherein the trigger nucleic acid sequence is a ssDNA or dsDNA or ssDNA sequence, preferably not having full (100%) complementarity to an existing genomic sequence, more preferably with the length of at least 10 nucleotides.

28. The method of any one of claims 25-27, wherein the target binding construct is an antibody or antigen binding fragment thereof.

29. The method of any one of claims 25 to 28, wherein the target binding construct is immobilized on a substrate or conjugated to a magnetic bead or a microparticle or a nanoparticle.

30. The method of claim 29, further comprising the step of performing magnetic separation of the captured target bound to the first target binding construct from the sample when the first target binding construct is conjugated to a magnetic bead.

31. The method of any one of claim 24 or 26 to 30, wherein the first and second type V or type VI CRISPR/Cas effector proteins are different.

32. The method according to claim 31, wherein said first type V or type VI CRISPR/Cas effector protein is a Type V CRISPR/Cas effector protein and said second type V or type VI CRISPR/Cas effector protein is a Type VI CRISPR/Cas effector protein.

33. The method of any one of claim 24 or 26 to 32, wherein contacting the sample with the second effector type V or type VI CRISPR/Cas effector protein and the second guide RNA occurs after the sample has been contacted with the first effector type V or type VI CRISPR/Cas effector protein and the first guide RNA.

34. The method of claim 33, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.

35. The method of any one of claims 23 to 34, wherein the first guide RNA is bound to the first type V or type VI CRISPR/Cas effector protein.

36. The method of any one of claim 24 or 26 to 35, wherein the second guide RNA is bound to the second type V or type VI CRISPR/Cas effector protein.

37. A method of enhancing a type V or type VI CRISPR/Cas detection system which comprises a type V or Type VI CRISPR/Cas effector protein, comprising:
adding to a reaction mixture comprising a type V or Type VI CRISPR/Cas effector protein of the detection system: a circular DNA molecular construct comprising both a ssDNA region and a dsDNA sequence, wherein the 5′ end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ssDNA or dsDNA or dsRNA or DNA/RNA hybrid sequence are detectably labelled; wherein the dsDNA of the circular DNA molecular construct or the dsDNA or ssDNA sequence or dsRNA sequence or DNA/RNA hybrid sequence of the circular RNA molecular construct hybridizes with a guide sequence of a guide RNA of said type V or type VI CRISPR/Cas detection system, and hybridization occurs following linearization of the circular DNA or RNA molecular construct, respectively, by the nuclease activity of the type V or type VI CRISPR/Cas effector protein of the detection system and further activates the nuclease activity of the type V or type VI CRISPR/Cas effector protein; and
measuring a detectable signal produced following linearization of the circular DNA molecular construct, or the circular RNA molecular construct by the type V or type VI CRISPR/Cas effector protein of said detection system.

38. A method of enhancing a type V or type VI CRISPR/Cas detection system which comprises a type V or Type VI CRISPR/Cas effector protein, and a first guide RNA, comprising:
adding to a reaction mixture comprising a type V or Type VI CRISPR/Cas effector protein of the detection system:
(i) a circular DNA molecular construct comprising both a ssDNA region and a dsDNA sequence wherein the 5′end and/or the 3′ end of the dsDNA sequence are detectably labelled; or a circular RNA molecular construct comprising both an ssRNA region and either a ssDNA sequence or a dsDNA sequence or a dsRNA sequence or a DNA/RNA hybrid sequence, wherein the 5′end and/or the 3′ end of the ss DNA or dsDNA or dsRNA or DNA/RNA hybrid sequence are detectably labelled;
(ii) a second type V or type VI CRISPR/Cas effector protein; and
(iii) a guide RNA, optionally bound to the second type V or type VI CRISPR/Cas effector protein, said guide RNA comprising: a region that binds to the second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the guide RNA and the dsDNA or ssDNA or dsRNA or DNA/RNA sequence occurs following linearization of the circular DNA or RNA molecular construct, respectively, by the nuclease activity of the type V or type VI CRISPR/Cas effector protein of the detection system and further activates the nuclease activity of the second type V or type VI CRISPR/Cas effector protein or the type V or type VI CRISPR/Cas effector protein of said detection system; and
measuring a detectable signal produced following linearization of the circular DNA molecular construct, or the circular RNA molecular construct by the second type V or type VI CRISPR/Cas effector protein or the type V or type VI CRISPR/Cas of said detection system.

39. The method of any one of claims 23 to 38, wherein the, wherein the circular DNA molecular construct or circular RNA molecular construct has the total length (circumference) from 15 to 30 nucleotides.

40. The method of claim 39 wherein the circular DNA molecular construct or circular RNA molecular construct has a total length of 16, 17, 18, 19, 20, 21, 22, or 23 nucleotides.

41. The method of claim 39 or 40, wherein the circular DNA molecular construct or circular RNA molecular construct comprises two parts including a 2-5 nucleotides ssDNA, or ssRNA, respectively, and the remaining part of the molecular construct is dsDNA or ssDNA or dsRNA or DNA/RNA hybrid with a complementary sequence to either a guide RNA of said type V or type VI CRISPR/Cas detection system or the guide RNA which binds to the second type V or type VI CRISPR/Cas effector protein (when present).

42. The method of any one of claims 39 to 41, wherein any one or more of the guide RNA, circular DNA molecular construct or circular RNA molecular construct, the reporter construct, and trigger nucleic acid, when present, comprises at least one nucleotide containing a non-natural modification/substitution.

43. The method of any one of claims 23 to 42, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.

44. A method for the detection of a target nucleic acid in a sample, the method comprising:
(a) contacting the sample with:
(i) a first type II or type V or type VI CRISPR/Cas effector protein;
(ii) a trigger nucleic acid sequence;
(iii) a first guide RNA, optionally wherein the first guide RNA is in association with said first type II or type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type II or type V or type VI CRISPR/Cas effector protein;
(iv) a second type II or type V or type VI CRISPR/Cas effector protein;
(v) a second guide RNA, wherein the second guide RNA is circular and is susceptible to trans-cleavage nuclease activity of a type II or type V or type VI CRISPR/Cas effector protein, and comprises a region that binds to said second type II or type V or type VI CRISPR/Cas effector protein, and a guide sequence that hybridizes with the trigger nucleic acid sequence, wherein hybridization between the guide sequence and the trigger nucleic acid sequence, first occurs following linearization of the second guide RNA by the nuclease activity of the first type II or type V or type VI CRISPR/Cas effector protein and further activates the trans-cleavage nuclease activity of the second type II or type V or type VI CRISPR/Cas effector protein;
(vi) a labelled reporter construct, wherein said reporter construct comprises a nucleic acid that is cleavable by the nuclease activity of the activated type II or type V or type VI CRISPR/Cas effector protein; and
(b) measuring a detectable signal produced following cleavage of the labelled nucleic acid reporter by the first and/or second type II or type V or type VI CRISPR/Cas effector protein, thereby detecting the target in the sample.

45. A method for the detection of a target in a sample, the method comprising:
(a) contacting the sample with:
(i) a first type II or type V or type VI CRISPR/Cas effector protein;
(ii) a first trigger nucleic acid sequence;
(iii) a first guide RNA, optionally wherein the first guide RNA is bound to said first type II or type V or type VI CRISPR/Cas effector protein, comprising: a region that binds to said first type II or type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type II or type V or type VI CRISPR/Cas effector protein;
(iv) a second type II or type V or type VI CRISPR/Cas effector protein;
(v) a second trigger nucleic acid sequence;
(vi) a second guide RNA, wherein the second guide RNA is circular and is susceptible to trans-cleavage nuclease activity of a type II or type V or type VI CRISPR/Cas effector protein, and comprises a region that binds to said second type II or type V or type VI CRISPR/Cas effector protein, and a guide sequence that hybridizes with the second trigger nucleic acid sequence, wherein hybridization between the guide sequence and the second trigger nucleic acid sequence, first occurs following linearization of the second guide RNA by the nuclease activity of the first type II or type V or type VI CRISPR/Cas effector protein and further activates the trans-cleavage nuclease activity of the second type II or type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type II or type V or type VI CRISPR/Cas effector protein;
(viii) a target binding construct; and
(b) measuring a detectable signal produced following cleavage of the labelled nucleic acid reporter construct by the first and second type II or type V or type VI CRISPR/Cas effector protein, thereby detecting the target in the sample;
wherein the first type II or type V or type VI CRISPR/Cas effector protein, the first trigger nucleic acid sequence, the first guide RNA, the first type TT or type V or type VI CRISPR/Cas effector protein in combination with the first guide RNA, or the first type II or type V or type VI CRISPR/Cas effector protein in combination with the first guide RNA and the first trigger nucleic acid is linked or conjugated to the target binding construct to thereby co-locate the first type II or type V or type VI CRISPR/Cas effector protein and the target when present in the sample.

46. A method of enhancing a type II or type V or type VI CRISPR/Cas detection system, which comprises a first type II or type V or Type VI CRISPR/Cas effector protein, a first guide RNA, and a labelled nucleic acid reporter, comprising:
adding to a reaction mixture comprising at least a first type II or type V or Type VI CRISPR/Cas effector of the system:
(i) a second type II or type V or type VI CRISPR/Cas effector protein;
(ii) a trigger nucleic acid sequence; and
(iii) a circular guide RNA which is susceptible to cis-cleavage and trans-cleavage nuclease activity of a type II or type V or type VI CRISPR/Cas effector protein, wherein the circular guide RNA comprises a region that binds to said second type II or type V or type VI CRISPR/Cas effector protein, and a guide sequence that hybridizes with the trigger nucleic acid sequence, wherein hybridization between the guide sequence and the trigger nucleic acid sequence, first occurs following linearization of the circular guide RNA by the nuclease activity of at least a first type II or type V or Type VI CRISPR/Cas effector of the system and further activates the trans-cleavage nuclease activity of the second type II or type V or type VI CRISPR/Cas effector protein; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type II or type V or type VI CRISPR/Cas effector protein;
and measuring a detectable signal produced following cleavage of the labelled nucleic acid reporter of the detection system, and/or the labelled reporter construct when added, by the second type II or type V or type VI CRISPR/Cas effector protein.

47. The method of claim 45, wherein the target binding construct is an antibody or antigen binding fragment thereof.

48. The method of any one of claim 45 or 47, wherein the target binding construct is immobilized on a substrate or conjugated to a magnetic bead or a microparticle or a nanoparticle.

49. The method of claim 48, further comprising the step of performing magnetic separation of the captured target bound to the first target binding construct from the sample when the first target binding construct is conjugated to a magnetic bead.

50. The method of any one of claims 44 to 49, wherein the trigger nucleic acid sequence is a ssRNA, ssDNA or dsDNA sequence, preferably not having full (100%) complementarity to an existing genomic sequence, more preferably with the length of at least 10 nucleotides.

51. The method of any one of claims 44 to 50, wherein the first and second type II or type V or type VI CRISPR/Cas effector proteins are different.

52. The method according to claim 74, wherein said first type II or type V or type VI CRISPR/Cas effector protein is a Type V CRISPR/Cas effector protein and said second type II or type V or type VI CRISPR/Cas effector protein is a Type VI CRISPR/Cas effector protein.

53. The method of any one of claims 44 to 52, wherein contacting the sample with the second effector type II or type V or type VI CRISPR/Cas effector protein and the second guide RNA occurs after the sample has been contacted with the first effector type II or type V or type VI CRISPR/Cas effector protein and the first guide RNA.

54. The method of claim 53, wherein contacting the sample with the second effector type II or type V or type VI CRISPR/Cas effector protein and the second guide RNA occurs at between 1 minute and 1 hour after the sample has been contacted with the first effector type II or type V or type VI CRISPR/Cas effector protein and the first guide RNA.

55. The method of any one of claims 44 to 54, wherein the circular guide RNA has the total length (circumference) from 40-50 nucleotides.

56. The method of any one of claims 44 to 55, wherein any one or more of the guide RNA, the reporter construct, and trigger nucleic acid, comprises at least one nucleotide containing a non-natural modification/substitution.

57. The method of any one of claims 44 to 56, wherein the type II or type V or type VI CRISPR/Cas effector protein is selected from Cas 9 family: Cas9; Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.

58. The method of any one of claims 44 to 57, wherein the reporter construct is a labelled RNA

59. The method of any one of claims 44 to 57, wherein the reporter construct is a labelled DNA.

60. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a labelled reporter construct, wherein said reporter construct comprises a nucleic acid that is cleavable by the nuclease activity of the activated type V or type VI CRISPR/Cas effector protein; and
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the first guide RNA and also activates the nuclease activity of first type V or type VI CRISPR/Cas effector proteins bound to said first guide RNA following cleavage of the sealed end by the nuclease activity of said first type V or type VI CRISPR/Cas effector protein, and;
(b) measuring a detectable signal produced following cleavage of the labelled nucleic acid reporter construct by the first and second type V or type VI CRISPR/Cas effector protein, thereby detecting the target in the sample.

61. A method for the detection of a target nucleic acid in a sample, the method comprising:
(a) contacting the sample with:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
iii) a second type V or type VI CRISPR/Cas effector protein;
(iv) a second guide RNA, optionally in association with said second type V or type VI CRISPR/Cas effector protein, comprising: a region that binds to said second type V or type VI CRISPR/Cas effector protein, and a guide sequence;
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vi) a labelled reporter construct, wherein said reporter construct comprises a nucleic acid that is cleavable by the nuclease activity of the activated type V or type VI CRISPR/Cas effector protein; and
(b) measuring a detectable signal produced following cleavage of the labelled nucleic acid reporter by the first and/or second type V or type VI CRISPR/Cas effector protein, thereby detecting the target in the sample.

62. A method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the first guide RNA and also activates the nuclease activity of said first type V or type VI CRISPR/Cas effector proteins bound to said first guide RNA following cleavage of the sealed end by the nuclease activity of said first type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

63. A method for the detection of a target in a sample, the method comprising:
(a) contacting the sample with:
(i) a first type V or type VI CRISPR/Cas effector protein;
(ii) a first trigger nucleic acid sequence;
(iii) a first guide RNA, optionally wherein the first guide RNA is bound to said first type V or type VI CRISPR/Cas effector protein, comprising: a region that binds to said first type V or type VI CRISPR/Cas effector protein, and a guide sequence that hybridizes with the trigger nucleic acid sequence, wherein hybridization between the first guide sequence and the trigger nucleic acid sequence activates the nuclease activity of said first type V or type VI CRISPR/Cas effector protein;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence,
(vi) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with the second guide RNA and also activates the nuclease activity of the second type V or type VI CRISPR/Cas effector proteins bound to said second guide RNA following cleavage of the sealed end by the nuclease activity of said first type V or type VI CRISPR/Cas effector protein;
(vii) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein;
(viii) a target binding construct; and
(b) measuring a detectable signal produced following cleavage of the labelled nucleic acid reporter construct by the first and second type V or type VI CRISPR/Cas effector protein, thereby detecting the target in the sample;
wherein the first type V or type VI CRISPR/Cas effector protein, the first trigger nucleic acid sequence, the first guide RNA, the first type V or type VI CRISPR/Cas effector protein in combination with the first guide RNA, or the first type V or type VI CRISPR/Cas effector protein in combination with the first guide RNA and the first trigger nucleic acid is linked or conjugated to the target binding construct to thereby co-locate the first type V or type VI CRISPR/Cas effector protein and the target when present in the sample.

64. The method of claim 61 or 63, wherein the trigger nucleic acid sequence is a ssDNA or dsDNA or ssDNA sequence, preferably not having full (100%) complementarity to an existing genomic sequence, more preferably with the length of at least 10 nucleotides.

65. The method of any one of claims 62-64, wherein the target binding construct is an antibody or antigen binding fragment thereof.

66. The method of any one of claims 61 to 65, wherein the target binding construct is immobilized on a substrate or conjugated to a magnetic bead or a microparticle or a nanoparticle.

67. The method of claim 66, further comprising the step of performing magnetic separation of the captured target bound to the first target binding construct from the sample when the first target binding construct is conjugated to a magnetic bead.

68. The method of any one of claim 61 or 63 to 67, wherein the first and second type V or type VI CRISPR/Cas effector proteins are different.

69. The method according to claim 68, wherein said first type V or type VI CRISPR/Cas effector protein is a Type V CRISPR/Cas effector protein and said second type V or type VI CRISPR/Cas effector protein is a Type VI CRISPR/Cas effector protein.

70. The method of any one of claim 61 or 63 to 69, wherein contacting the sample with the second effector type V or type VI CRISPR/Cas effector protein and the second guide RNA occurs after the sample has been contacted with the first effector type V or type VI CRISPR/Cas effector protein and the first guide RNA.

71. The method of claim 70, wherein contacting the sample with the second effector type V or type VI CRISPR/Cas effector protein and the second guide RNA occurs at between 1 minute and 1 hour after the sample has been contacted with the first effector type V or type VI CRISPR/Cas effector protein and the first guide RNA.

72. The method of any one of claims 60 to 71, wherein the first guide RNA is bound to the first type V or type VI CRISPR/Cas effector protein.

73. The method of any one of claim 61 or 63 to 72, wherein the second guide RNA is bound to the second type V or type VI CRISPR/Cas effector protein.

74. A method of enhancing a type V or type VI CRISPR/Cas detection system, which comprises a type V or Type VI CRISPR/Cas effector protein, a first guide RNA, and a labelled nucleic acid reporter, comprising:
adding to a reaction mixture comprising a Type V or Type VI CRISPR/Cas effector protein of the detection system: a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with a guide sequence of a guide RNA of said type V or type VI CRISPR/Cas detection system and following cleavage of the sealed end by the nuclease activity of said first type V or type VI CRISPR/Cas effector protein further activates the nuclease activity of said type V or type VI CRISPR/Cas effector proteins bound to said guide RNA; and
measuring a detectable signal produced following linearization of the circular DNA molecular construct, or the circular RNA molecular construct by the type V or type VI CRISPR/Cas effector protein of said detection system.

75. A method of enhancing a type V or type VI CRISPR/Cas detection system, which comprises a first type V or Type VI CRISPR/Cas effector protein, a first guide RNA, and a labelled nucleic acid reporter, comprising:
adding to a reaction mixture comprising at least a first type V or Type VI CRISPR/Cas effector of the system:
(i) a second type V or type VI CRISPR/Cas effector protein;
(ii) a second guide RNA, optionally bound to the second type V or type VI CRISPR/Cas effector protein, said guide RNA comprising: a region that binds to the second type V or type VI CRISPR/Cas effector protein, and a guide sequence,
(iii) a palindromic oligonucleotide comprising a single stranded sequence of nucleotides comprising a first sequence of nucleotides, optionally followed downstream by a spacer consisting of 1-3 nucleotides, followed by a second sequence of nucleotides, wherein the first sequence hybridizes with the second sequence to form a double stranded structure having a sealed end, preferably wherein the second sequence is the reverse complement of the first sequence, and optionally wherein the double stranded structure includes a PAM sequence which is distal to the sealed end; and wherein the double stranded structure specifically hybridizes with a guide sequence of the second guide RNA and following cleavage of the sealed end by the nuclease activity of said first type V or type VI CRISPR/Cas effector protein further activates the nuclease activity of said second type V or type VI CRISPR/Cas effector proteins bound to said second guide RNA; and optionally
(iv) a labelled reporter construct, wherein said reporter construct comprises a nucleic acid that may or may not hybridize with the guide sequence of the first guide RNA or the guide sequence of the second guide RNA and is cleavable by the nuclease activity of the activated second type V or type VI CRISPR/Cas effector protein;
and measuring a detectable signal produced following cleavage of the labelled nucleic acid reporter of the detection system, and/or the labelled reporter construct when added, by the first and/or second type V or type VI CRISPR/Cas effector protein.

76. The method of any one of claims 60 to 75, wherein said first sequence of nucleotides and/or said second sequence of nucleotides of the palindromic oligonucleotide is from 10 to 30 nucleotides in length.

77. The method of claim 76, wherein said first sequence of nucleotides and/or said second sequence of nucleotides of the palindromic oligonucleotide is 15 nucleotides in length.

78. The method of any one of claims 60 to 77, wherein said first sequence of nucleotides and said second sequence of nucleotides of the palindromic oligonucleotide are 100% complementary to one another.

79. The method of any one of claims 60 to 78, wherein any one or more of the guide RNA, the palindromic oligonucleotide, and labelled nucleic acid, and the nucleic acid reporter construct and/or trigger nucleic acid, when present, comprises at least one nucleotide containing a non-natural modification/substitution.

80. The method of any one of claims 60 to 79, wherein the type V or type VI CRISPR/Cas effector protein is selected from Cas 12 family: Cas12a, Cas12b, Cas12c; C2c4, C2c8, C2c5, C2c10, and C2c9; CasX (Cas12e), CasY (Cas12d), Cas12g, Cas12j and Cas12k; and Cas13 family including: Cas13a, Cas13b, Cas13c, Cas13d, Cas13X, Cas13Y, and Cas13bt.

81. The method of any one of claims 1 to 80, wherein the steps of the method are conducted at a temperature ranging from 10 to 48 degrees Celsius, preferably, from 25 to 37 degrees Celsius.

82. The method of any one of claims 1 to 81, wherein the sample is also contacted with at least one sulfhydryl reductant.

83. The method of claim 82, wherein the sulfhydryl reductant is selected from the group consisting of Dithiothreitol (DTT), Tris(2-carboxyethyl) phosphine (TCEP) to and 2-Mercaptoethanol (2-ME).

84. The method of claim 83, wherein the sulfhydryl reductant is DTT.

85. The method of claim 82, wherein said contacting occurs at a temperature of 10-48° C.

86. The method of any one of claims 1 to 85, wherein the sample is also contacted with at least one non-ionic surfactant.

87. The method of claim 86, wherein the non-ionic surfactant is selected from the group consisting of Brij L23 and poly(vinyl alcohol) (PVA).

88. The method of claim 87, wherein the non-ionic surfactant is PVA.

89. The method of any one of the preceding claims wherein the labelled nucleic acid reporter, the labelled reporter construct, or labelled circular DNA molecular construct or labelled circular RNA molecular construct comprises a fluorophore and a quencher of the fluorophore.

90. The method of claim 89, wherein the labelled nucleic acid reporter, the labelled reporter construct, or labelled circular DNA molecular construct or labelled circular RNA molecular construct comprises a fluorophore at the 5′ end and a quencher of the fluorophore at the 3′ end.

91. The method of claim 89 or 90, wherein the fluorophore is FAM and the quencher is BHQ1, or wherein the fluorophore is Texas Red and the quencher is BHQ2.

92. The method of any one of claims 1 to 91, wherein said contacting occurs in a reaction mixture comprising a buffer.

93. The method of any one of claims 1 to 92, wherein the target is detected at attomolar sensitivity or lower.

94. The method of any one of claims 1 to 93, wherein the sample is a biological sample or an environmental sample.

95. The method of claim 94, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.

96. The method of claim 95, wherein the sample is blood, plasma, serum or a biopsy obtained from a human patient.

97. The method of claim 94, wherein the sample is a water sample.

98. The method of claim 94, wherein the sample is a crude sample.

99. The method of any of claims 1-97, wherein the sample is a concentrated or purified sample.

100. A circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence, wherein said circular DNA molecular or circular RNA molecular construct comprises a sequence complementary to a guide RNA sequence which binds to a type V or type VI CRISPR/Cas effector protein, and wherein said circular DNA molecular or circular RNA molecular construct only hybridizes with the guide sequence of the guide RNA following linearization by cleavage of the ssDNA region in said circular DNA molecular construct or the ssRNA region in said circular RNA molecular construct.

101. The circular DNA molecular construct or circular RNA molecular construct of claim 104, wherein the 5′end and/or the 3′ end of the dsDNA sequence of the circular DNA molecular construct are detectably labelled; or wherein the 5′end and/or the 3′ end of the ssDNA or dsDNA or dsRNA or DNA/RNA hybrid sequence of the circular RNA molecular construct are detectably labelled.

102. The circular DNA molecular construct or circular RNA molecular construct of claim 100 or 101, wherein the, wherein the construct has the total length (circumference) from 15 to 30 nucleotides.

103. The circular DNA molecular construct or circular RNA molecular construct of 102, wherein the circular DNA molecular construct or circular RNA molecular construct has a total length of 16, 17, 18, 19, 20, 21, 22, or 23 nucleotides.

104. The circular DNA molecular construct or circular RNA molecular construct of any one of claims 100 to 103, wherein the circular DNA molecular construct comprises two parts: i) a region comprising 1-7 nucleotides ssDNA, and ii) the remaining part of the molecular construct is dsDNA with a complementary sequence to a guide RNA sequence which binds to a type V or type VI CRISPR/Cas effector protein; and wherein the circular RNA molecular construct comprises two parts: i) a region comprising 0-7 nucleotides ssRNA, and ii) the remaining part of the molecular construct is ssDNA or dsDNA or dsRNA or DNA/RNA hybrid with a complementary sequence to a guide RNA sequence which binds to a type V or type VI CRISPR/Cas effector protein.

105. The circular DNA molecular construct or circular RNA molecular construct of any one of claims 100 to 104, comprising at least one nucleotide containing a non-natural modification/substitution.

106. The circular DNA molecular construct or circular RNA molecular construct of any one of claims 101 to 105, comprising a fluorophore and a quencher of the fluorophore.

107. The circular DNA molecular construct or circular RNA molecular construct of claim 106, wherein the construct comprises a fluorophore at the 5′ end and a quencher of the fluorophore at the 3′ end.

108. The circular DNA molecular construct or circular RNA molecular construct of claim 106 or 107, wherein the fluorophore is FAM and the quencher is BHQ1, or wherein the fluorophore is Texas Red and the quencher is BHQ2.

109. A circular guide RNA, wherein the circular guide RNA is susceptible to trans-cleavage nuclease activity of a type II or type V or type VI CRISPR/Cas effector protein and comprises a region that binds to a type II or type V or type VI CRISPR/Cas effector protein, and a guide sequence that hybridizes with a target nucleic acid sequence or a trigger nucleic acid sequence, wherein hybridization between the guide sequence and the trigger nucleic acid sequence, only occurs following linearization of the circular guide RNA by the cis-cleavage or trans-cleavage nuclease activity of a type II or type V or type VI CRISPR/Cas effector protein.

110. The circular guide RNA of claim 109, comprising a total length (circumference) from 40-80 nucleotides.

111. The circular guide RNA of claim 109 or 110, wherein the comprising two parts including a 2-5 ssDNA nucleotides or a 14-24 dsDNA nucleotides, and the remaining part of the guide RNA comprises a complementary sequence to a target nucleic acid or trigger nucleic acid sequence a sequence which binds to a type II or type V or type VI CRISPR/Cas effector protein.

112. The circular guide RNA of any one of claims 109 to 111, wherein any one or more of the comprising at least one nucleotide containing a non-natural modification/substitution.

113. A kit for detecting a target in a sample, the kit comprising:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the second guide RNA and the dsDNA or ssDNA or dsRNA or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.

114. A kit for amplifying enhancing a type V or type VI CRISPR/Cas detection system, which comprises a first type V or Type VI CRISPR/Cas effector protein, a guide RNA which binds to the type V or type VI CRISPR/Cas effector protein, and a labelled nucleic acid reporter, the kit comprising: the circular DNA molecular construct or circular RNA molecular construct of any one of claims 100 to 108, or the circular guide RNA of any one of claims 109 to 112, or a palindromic oligonucleotide as described in any one of claims 60 to 79.

115. The kit of claim 114, further comprising one or more of: a type V or Type VI CRISPR/Cas effector protein, a guide RNA, and a labelled nucleic acid reporter.

116. The kit of claim 113 to 115, further comprising one or more of: a target binding construct, a reaction buffer, a washing buffer, and reagents for recovering or releasing immobilized or captured target.

117. The kit of any one of claims 113 to 116, when used for a method for detecting a target in a sample.

118. A reaction mixture comprising:
(i) a first type V or type VI CRISPR/Cas effector protein;
(ii) a first guide RNA, optionally wherein the first guide RNA is in association with said first type V or type VI CRISPR/Cas effector protein, comprising: a region that binds to said first type V or type VI CRISPR/Cas effector protein, and a guide sequence that hybridizes with a target nucleic acid sequence, wherein hybridization between the first guide sequence and the target nucleic acid sequence activates the nuclease activity of said first type V or type VI CRISPR/Cas effector protein;
(iii) a circular DNA molecular construct comprising both a ssDNA region and a dsDNA sequence, or a circular RNA molecular construct comprising both an ssRNA region and either a ssDNA sequence or a dsDNA sequence or a dsRNA sequence or a DNA/RNA hybrid sequence;
(iv) a second type V or type VI CRISPR/Cas effector protein;
(v) a second guide RNA, optionally in association with said second type V or type VI CRISPR/Cas effector protein, comprising: a region that binds to said second type V or type VI CRISPR/Cas effector protein, and a guide sequence that hybridizes with the dsDNA of the circular DNA molecular construct or the dsDNA or ssDNA or dsRNA or DNA/RNA hybrid sequence of the circular RNA molecular construct, wherein hybridization between the guide sequence of the second guide RNA and the dsDNA or ssDNA or dsRNA or DNA/RNA hybrid sequence occurs following cleavage of the ssDNA or ssRNA region of the circular DNA or RNA molecular construct, respectively, by said first type V or type VI CRISPR/Cas effector protein and further activates the nuclease activity of the second type V or type VI CRISPR/Cas effector protein bound to said second guide RNA; and
(vi) a labelled reporter construct, wherein said reporter construct comprises a nucleic acid that may or may not hybridize with the guide sequence of the first guide RNA or the guide sequence of the second guide RNA and is cleavable by the nuclease activity of the activated type V or type VI CRISPR/Cas effector protein.

119. A reaction mixture comprising the circular DNA molecular construct or circular RNA molecular construct of any one of claims 100 to 108, or the circular guide RNA of any one of claims 109 to 112, or the palindromic oligonucleotide as described in any one of claims 60 to 79.

120. The reaction mixture of claim 119, further comprising one or more of: a type V or Type VI CRISPR/Cas effector protein, a guide RNA, and a labelled nucleic acid reporter.

121. The reaction mixture of claim 119 or 120, further comprising a sample.

122. The method according of any one of claims 1 to 99, the kit of any one of claims 113 to 117, or the reaction mixture of any one of claims 118 to 121, wherein the first or second type V or type VI CRISPR/Cas effector protein is immobilized on a substrate or conjugated to a magnetic bead or a microparticle or a nanoparticle.

123. The method of claim 24, wherein the second guide RNA comprises a guide sequence that hybridizes with the dsDNA of the circular DNA molecular construct or the dsDNA or ssDNA sequence of the circular RNA molecular construct, but not the target sequence.

124. The method of any one of claims 1-6, 9-22, 24, 26-30, 33-36, 38-50, 53-59, 61, 63-67, 70-73, 75-99, or 123, wherein the first and second type V or type VI CRISPR/Cas effector proteins are the same.

125. The method of any one of claims 1-8, 11-22, 24, 26-32, 35, 36, 38-52, 55-59, 61, 63-69, 72, 73, 75-99, 123, or 124, wherein contacting the sample with the second effector type V or type VI CRISPR/Cas effector protein and the second guide RNA occurs simultaneously with the sample being contacted with the first effector type V or type VI CRISPR/Cas effector protein and the first guide RNA.


SEQUENCE LISTING (103 sequences)

SEQ ID NO: 1 - DNA, 19 bp, synthetic construct
  source 1..19: mol_type = other DNA
ctatgtgctatgtctaaaa

SEQ ID NO: 2 - DNA, 24 bp, synthetic construct
  source 1..24: mol_type = other DNA
gaagacaccctaccaacccccccc

SEQ ID NO: 3 - DNA, 30 bp, synthetic construct
  source 1..30: mol_type = other DNA
gaagacaccctaccaacccccccctaaacc

SEQ ID NO: 4 - DNA, 18 bp, synthetic construct
  source 1..18: mol_type = other DNA
  misc_feature 1: note = 5'-modification; 5'P
tatgtgctatgtctaaaa

SEQ ID NO: 5 - DNA, 16 bp, synthetic construct
  source 1..16: mol_type = other DNA
tttagacatagcacat

SEQ ID NO: 6 - DNA, 12 bp, synthetic construct
  source 1..12: mol_type = other DNA
gcacatatttta

SEQ ID NO: 7 - DNA, 19 bp, synthetic construct
  source 1..19: mol_type = other DNA
  misc_feature 1: note = 5'-modification; 5'P
ctatgtgctatgtctaaaa

SEQ ID NO: 8 - DNA, 17 bp, synthetic construct
  source 1..17: mol_type = other DNA
tttagacatagcacata

SEQ ID NO: 9 - DNA, 12 bp, synthetic construct
  source 1..12: mol_type = other DNA
  misc_feature 1: note = 5'-modification; 5'P
cacatagtttta

SEQ ID NO: 10 - DNA, 20 bp, synthetic construct
  source 1..20: mol_type = other DNA
tctatgtgctatgtctaaaa

SEQ ID NO: 11 - DNA, 18 bp, synthetic construct
  source 1..18: mol_type = other DNA
tttagacatagcacatag

SEQ ID NO: 12 - DNA, 12 bp, synthetic construct
  source 1..12: mol_type = other DNA
acatagatttta

SEQ ID NO: 13 - DNA, 21 bp, synthetic construct
  source 1..21: mol_type = other DNA
  misc_feature 1: note = 5'-modification; 5'P
ttctatgtgctatgtctaaaa

SEQ ID NO: 14 - DNA, 19 bp, synthetic construct
  source 1..19: mol_type = other DNA
tttagacatagcacataga

SEQ ID NO: 15 - DNA, 12 bp, synthetic construct
  source 1..12: mol_type = other DNA
catagaatttta

SEQ ID NO: 16 - DNA, 24 bp, synthetic construct
  source 1..24: mol_type = other DNA
  misc_feature 1: note = 5'-modification; 5'P
tcagtctatgtgctatgtctaaaa

SEQ ID NO: 17 - DNA, 22 bp, synthetic construct
  source 1..22: mol_type = other DNA
tttagacatagcacatagactg

SEQ ID NO: 18 - DNA, 13 bp, synthetic construct
  source 1..13: mol_type = other DNA
agactgattttag

SEQ ID NO: 19 - DNA, 27 bp, synthetic construct
  source 1..27: mol_type = other DNA
  misc_feature 1: note = 5'-modification; 5'P
ttctcagtctatgtgctatgtctaaaa

SEQ ID NO: 20 - DNA, 25 bp, synthetic construct
  source 1..25: mol_type = other DNA
tttagacatagcacatagactgaga

SEQ ID NO: 21 - DNA, 13 bp, synthetic construct
  source 1..13: mol_type = other DNA
ctgagaattttag

SEQ ID NO: 22 - DNA, 18 bp, synthetic construct
  source 1..18: mol_type = other DNA
  misc_feature 1: note = 5'-modification; 5`Azide(N3)
  misc_feature 18: note = 3'-modification; 3`CHCH
  misc_feature 1..18: note = Internal modification at any position; Int Biotin dT
tatgtgctatgtctaaaa

SEQ ID NO: 23 - DNA, 19 bp, synthetic construct
  source 1..19: mol_type = other DNA
  misc_feature 1: note = 5'-modification; 5`Azide(N3)
  misc_feature 19: note = 3'-modification; 3`CHCH
  misc_feature 1..19: note = Internal modification at any position; Int Biotin dT
ctatgtgctatgtctaaaa

SEQ ID NO: 24 - DNA, 20 bp, synthetic construct
  source 1..20: mol_type = other DNA
  misc_feature 1: note = 5'-modification; 5`Azide(N3)
  misc_feature 20: note = 3'-modification; 3`CHCH
  misc_feature 1..20: note = Internal modification at any position; Int Biotin dT
tctatgtgctatgtctaaaa

SEQ ID NO: 25 - DNA, 25 bp, synthetic construct
  source 1..25: mol_type = other DNA
  misc_feature 1: note = 5'-modification; 5`Azide(N3)
  misc_feature 25: note = 3'-modification; 3`CHCH
  misc_feature 1..25: note = Internal modification at any position; Int Biotin dT
tctcagtctatgtgctatgtctaaa

SEQ ID NO: 26 - DNA, 16 bp, synthetic construct
  source 1..16: mol_type = other DNA
tttagacatagcacat

SEQ ID NO: 27 - DNA, 44 bp, synthetic construct
  source 1..44: mol_type = other DNA
  misc_feature 1: note = 5'-modification; 5'-Biotin
tttagacatagcacatagactgcagtctatgtgctatgtctaaa

SEQ ID NO: 28 - DNA, 45 bp, synthetic construct
  source 1..45: mol_type = other DNA
  misc_feature 1: note = 5'-modification; 5'-Biotin
tttagacatagcacatagactgccagtctatgtgctatgtctaaa

SEQ ID NO: 29 - DNA, 46 bp, synthetic construct
  source 1..46: mol_type = other DNA
  misc_feature 1: note = 5'-modification; 5'-Biotin
tttagacatagcacatagactgcccagtctatgtgctatgtctaaa

SEQ ID NO: 30 - DNA, 47 bp, synthetic construct
  source 1..47: mol_type = other DNA
  misc_feature 1: note = 5'-modification; 5'-Biotin
tttagacatagcacatagactgccccagtctatgtgctatgtctaaa

SEQ ID NO: 31 - DNA, 43 bp, synthetic construct
  source 1..43: mol_type = other DNA
  misc_feature 1: note = 5'-modification; 5'-Biotin
tttagacatagcacatagactcagtctatgtgctatgtctaaa

SEQ ID NO: 32 - DNA, 42 bp, synthetic construct
  source 1..42: mol_type = other DNA
  misc_feature 1: note = 5'-modification; 5'-Biotin
tttagacatagcacatagactagtctatgtgctatgtctaaa

SEQ ID NO: 33 - DNA, 41 bp, synthetic construct
  source 1..41: mol_type = other DNA
  misc_feature 1: note = 5'-modification; 5'-Biotin
tttagacatagcacatagaccgtctatgtgctatgtctaaa

SEQ ID NO: 34 - DNA, 45 bp, synthetic construct
  source 1..45: mol_type = other DNA
  misc_feature 1: note = 5'-modification; 5'-Biotin
tttagacatagcacatagacgccccgtctatgtgctatgtctaaa

SEQ ID NO: 35 - DNA, 41 bp, synthetic construct
  source 1..41: mol_type = other DNA
  misc_feature 1: note = 5'-modification; 5'-Biotin
tttagacatagcacatagccccgctatgtgctatgtctaaa

SEQ ID NO: 36 - DNA, 41 bp, synthetic construct
  source 1..41: mol_type = other DNA
tttagacatagcacatagactgtctatgtgctatgtctaaa

SEQ ID NO: 37 - DNA, 41 bp, synthetic construct; identical to SEQ ID NO: 33
  source 1..41: mol_type = other DNA
  misc_feature 1: note = 5'-modification; 5`Azide(N3)
  misc_feature 41: note = 3'-modification; 3`CHCH

SEQ ID NO: 38 - DNA, 71 bp, synthetic construct
  source 1..71: mol_type = other DNA
acagtagcgatctcatttagacatagcacatagactgtctatgtgctatgtctaaatgagatcgctactgt

SEQ ID NO: 39 - DNA, 22 bp, synthetic construct
  source 1..22: mol_type = other DNA
ctctcattttttttttagagag

SEQ ID NO: 40 - DNA, 19 bp, synthetic construct
  source 1..19: mol_type = other DNA
  misc_feature 5: note = Internal modification; /iBio-dT/
  misc_feature 1: note = 5'-modification; 5`Azide(N3)
  misc_feature 19: note = 3'-modification; 3`CHCH
tctatgtgctatgtctaaa

SEQ ID NO: 41 - DNA, 18 bp, synthetic construct
  source 1..18: mol_type = other DNA
  misc_feature 1: note = 5'-modification; 5'-Texas Red
  misc_feature 18: note = 3'-modification; 3'-BHQ2
tttagacatagcacatag

SEQ ID NO: 42 - DNA, 18 bp, synthetic construct
  source 1..18: mol_type = other DNA
  misc_feature 1: note = 5'-modification; 5`Azide(N3)
  misc_feature 18: note = 3'-modification; 3`CHCH
  misc_feature 4: note = Internal modification; /iBio-dT/
ctatgtgctatgtctaaa

SEQ ID NO: 43 - DNA, 21 bp, synthetic construct
  source 1..21: mol_type = other DNA
  misc_feature 7: note = Internal modification; /iBio-dT/
  misc_feature 1: note = 5'-modification; 5`Azide(N3)
  misc_feature 21: note = 3'-modification; 3`CHCH
tttctatgtgctatgtctaaa

SEQ ID NO: 44 - DNA, 23 bp, synthetic construct
  source 1..23: mol_type = other DNA
  misc_feature 9: note = Internal modification; /iBio-dT/
  misc_feature 1: note = 5'-modification; 5`Azide(N3)
  misc_feature 23: note = 3'-modification; 3`CHCH
tttttctatgtgctatgtctaaa

SEQ ID NO: 45 - DNA, 25 bp, synthetic construct
  source 1..25: mol_type = other DNA
  misc_feature 11: note = Internal modification; /iBio-dT/
  misc_feature 1: note = 5'-modification; 5`Azide(N3)
  misc_feature 25: note = 3'-modification; 3`CHCH
tttttttctatgtgctatgtctaaa

SEQ ID NO: 46 - RNA, 42 nt, synthetic construct
  source 1..42: mol_type = other RNA
taatttctactaagtgtagatggggggggttggtagggtgtc

SEQ ID NO: 47 - DNA, 25 bp, synthetic construct
  source 1..25: mol_type = other DNA
tttagacatagcacatagactgaga

SEQ ID NO: 48 - DNA, 28 bp, synthetic construct
  source 1..28: mol_type = other DNA
  misc_feature 14: note = Internal modification; /iBio-dT/
  misc_feature 1: note = 5'-modification; 5`Azide(N3)
  misc_feature 28: note = 3'-modification; 3`CHCH
ttttctcagtctatgtgctatgtctaaa

SEQ ID NO: 49 - DNA, 21 bp, synthetic construct
  source 1..21: mol_type = other DNA
tttagacatagcacatagact

SEQ ID NO: 50 - DNA, 24 bp, synthetic construct
  source 1..24: mol_type = other DNA
  misc_feature 10: note = Internal modification; /iBio-dT/
  misc_feature 1: note = 5'-modification; 5`Azide(N3)
  misc_feature 24: note = 3'-modification; 3`CHCH
tttagtctatgtgctatgtctaaa

SEQ ID NO: 51 - DNA, 18 bp, synthetic construct
  source 1..18: mol_type = other DNA
tttagacatagcacatag

SEQ ID NO: 52 - DNA, 15 bp, synthetic construct
  source 1..15: mol_type = other DNA
tttagacatagcaca

SEQ ID NO: 53 - DNA, 18 bp, synthetic construct
  source 1..18: mol_type = other DNA
  misc_feature 4: note = Internal modification; /iBio-dT/
  misc_feature 1: note = 5'-modification; 5`Azide(N3)
  misc_feature 18: note = 3'-modification; 3`CHCH
ttttgtgctatgtctaaa

SEQ ID NO: 54 - DNA, 21 bp, synthetic construct
  source 1..21: mol_type = other DNA
tctcagtctatgtgctatgtc

SEQ ID NO: 55 - RNA, 42 nt, synthetic construct
  source 1..42: mol_type = other RNA
taatttctactaagtgtagatgacatagcacatagactgaga

SEQ ID NO: 56 - DNA, 20 bp, synthetic construct
  source 1..20: mol_type = other DNA
  misc_feature 6: note = Internal modification; /iBio-dT/
  misc_feature 1: note = 5'-modification; 5`Azide(N3)
  misc_feature 20: note = 3'-modification; 3`CHCH
ttctatgtgctatgtctaaa

SEQ ID NO: 57 - DNA, 28 bp, synthetic construct
  source 1..28: mol_type = other DNA
  misc_feature 14: note = Internal modification; /iBio-dT/
  misc_feature 1: note = 5'-modification; 5`Azide(N3)
  misc_feature 28: note = 3'-modification; 3`CHCH
ttttttttttctatgtgctatgtctaaa

SEQ ID NO: 58 - DNA, 23 bp, synthetic construct
  source 1..23: mol_type = other DNA
ctcagtgattttagagagaggat

SEQ ID NO: 59 - RNA, 42 nt, synthetic construct
  source 1..42: mol_type = other RNA
taatttctactaagtgtagatcctctctctaaaatcactgag

SEQ ID NO: 60 - DNA, 21 bp, synthetic construct
  source 1..21: mol_type = other DNA
  misc_feature 5: note = Internal modification; /iBio-dT/
  misc_feature 1: note = 5'-modification; 5`Azide(N3)
  misc_feature 21: note = 3'-modification; 3`CHCH
tttattttagagagaggtaaa

SEQ ID NO: 61 - DNA, 18 bp, synthetic construct
  source 1..18: mol_type = other DNA
  misc_feature 1: note = 5'-modification; 5'-Texas Red
  misc_feature 18: note = 3'-modification; 3'-BHQ2
tttacctctctctaaaat

SEQ ID NO: 62 - DNA, 23 bp, synthetic construct
  source 1..23: mol_type = other DNA
  misc_feature 1: note = 5'-modification; 5'-FAM
  misc_feature 23: note = 3'-modification; 3'-Biotin
tttacctctctctaaaatccccc

SEQ ID NO: 63 - DNA, 45 bp, synthetic construct
  misc_feature 44..45: note = DNA Segment
  misc_feature 1..43: note = RNA Segment
  source 1..45: mol_type = other DNA
  misc_feature 43: note = Internal modification; /iBio-dT/
  misc_feature 1: note = 5'-modification; 5'-Azide
  misc_feature 45: note = 3'-modification; 3'-CHCH
taatttctactaagtgtagatgacatagcacatagactgagattt

SEQ ID NO: 64 - RNA, 42 nt, synthetic construct; identical to SEQ ID NO: 55
  source 1..42: mol_type = other RNA
  misc_feature 1: note = 5'-modification; 5`Azide(N3)
  misc_feature 42: note = 3'-modification; 3`CHCH

SEQ ID NO: 65 - DNA, 25 bp, synthetic construct
  source 1..25: mol_type = other DNA
tctcagtctatgtgctatgtctaaa

SEQ ID NO: 66 - RNA, 45 nt, synthetic construct
  source 1..45: mol_type = other RNA
taatttctactaagtgtagatgacatagcacatagactgagactg

SEQ ID NO: 67 - DNA, 44 bp, synthetic construct
  source 1..44: mol_type = other DNA
gaaacaaatgaatgatgcacatcatggtggctggacaacaaaaa

SEQ ID NO: 68 - DNA, 44 bp, synthetic construct
  source 1..44: mol_type = other DNA
tttttgttgtccagccaccatgatgtgcatcattcatttgtttc

SEQ ID NO: 69 - DNA, 44 bp, synthetic construct
  source 1..44: mol_type = other DNA
gaaacaaatgaatgatgcacgtcatggtggctggacaacaaaaa

SEQ ID NO: 70 - DNA, 44 bp, synthetic construct
  source 1..44: mol_type = other DNA
tttttgttgtccagccaccatgacgtgcatcattcatttgtttc

SEQ ID NO: 71 - RNA, 40 nt, synthetic construct
  source 1..40: mol_type = other RNA
taatttctactaagtgtagatatgatgcacgtcatggtgg

SEQ ID NO: 72 - RNA, 40 nt, synthetic construct
  source 1..40: mol_type = other RNA
taatttctactaagtgtagatatgatgcacatcatggtgg

SEQ ID NO: 73 - RNA, 52 nt, synthetic construct
  source 1..52: mol_type = other RNA
ggaccaccccaaaaacgaaggggactaaaacacgttgttttgatcgcgcccc

SEQ ID NO: 74 - RNA, 20 nt, synthetic construct
  source 1..20: mol_type = other RNA
ggggcgcgatcaaaacaacg

SEQ ID NO: 75 - RNA, 20 nt, synthetic construct
  source 1..20: mol_type = other RNA
  misc_feature 1..20: note = Each nucleotide residue is linked by phosphorothioate (PS) bond
ggggcgcgatcaaaacaacg

SEQ ID NO: 76 - RNA, 20 nt, synthetic construct
  source 1..20: mol_type = other RNA
  misc_feature 1: note = 5'-modification; 5'-FAM
ggggcgcgatcaaaacaacg

SEQ ID NO: 77 - RNA, 40 nt, synthetic construct
  source 1..40: mol_type = other RNA
aagtgtagatggggcgcgatcaaaacaacggaacgaatgt

SEQ ID NO: 78 - RNA, 60 nt, synthetic construct
  source 1..60: mol_type = other RNA
aattagatgtaagtgtagatggggcgcgatcaaaacaacggaacgaatgtacgaactgag

SEQ ID NO: 79 - DNA, 20 bp, synthetic construct
  source 1..20: mol_type = other DNA
ggggcgcgatcaaaacaacg

SEQ ID NO: 80 - DNA, 20 bp, synthetic construct
  source 1..20: mol_type = other DNA
cgttgttttgatcgcgcccc

SEQ ID NO: 81 - DNA, 20 bp, synthetic construct
  source 1..20: mol_type = other DNA
  misc_feature 1: note = 5'-modification; 5'-FAM
  misc_feature 20: note = 3'-modification; 3'-BHQ1
cgttgttttgatcgcgcccc

SEQ ID NO: 82 - RNA, 20 nt, synthetic construct
  source 1..20: mol_type = other RNA
cgttgttttgatcgcgcccc

SEQ ID NO: 83 - RNA, 20 nt, synthetic construct
  source 1..20: mol_type = other RNA
  misc_feature 20: note = 3'-modification; 3'-BHQ1
cgttgttttgatcgcgcccc

SEQ ID NO: 84 - RNA, 20 nt, synthetic construct
  source 1..20: mol_type = other RNA
  misc_feature 1: note = 5'-modification; 5'-FAM
  misc_feature 20: note = 3'-modification; 3'-BHQ1
cgttgttttgatcgcgcccc

SEQ ID NO: 85 - RNA, 25 nt, synthetic construct
  source 1..25: mol_type = other RNA
  misc_feature 1: note = 5'-modification; 5'-Biotin
  misc_feature 25: note = 3'-modification; 3'-BHQ1
tttttcgttgttttgatcgcgcccc

SEQ ID NO: 86 - RNA, 40 nt, synthetic construct
  source 1..40: mol_type = other RNA
acaatcgttccgttgttttgatcgcgccccatctacactt

SEQ ID NO: 87 - RNA, 60 nt, synthetic construct
  source 1..60: mol_type = other RNA
ctcagttcgtacaatcgttccgttgttttgatcgcgccccatctacacttacatctaatt

SEQ ID NO: 88 - RNA, 23 nt, synthetic construct
  source 1..23: mol_type = other RNA
  misc_feature 21: note = Internal modification; /iBio-dT/
  misc_feature 1: note = 5'-modification; 5'-Azide
  misc_feature 23: note = 3'-modification; 3'-CHCH
ggggcgcgatcaaaacaacgttt

SEQ ID NO: 89 - RNA, 25 nt, synthetic construct
  source 1..25: mol_type = other RNA
  misc_feature 21: note = Internal modification; /iBio-dT/
  misc_feature 1: note = 5'-modification; 5'-Azide
  misc_feature 25: note = 3'-modification; 3'-CHCH
ggggcgcgatcaaaacaacgttttt

SEQ ID NO: 90 - RNA, 27 nt, synthetic construct
  source 1..27: mol_type = other RNA
  misc_feature 21: note = Internal modification; /iBio-dT/
  misc_feature 1: note = 5'-modification; 5'-Azide
  misc_feature 27: note = 3'-modification; 3'-CHCH
ggggcgcgatcaaaacaacgttttttt

SEQ ID NO: 91 - RNA, 22 nt, synthetic construct
  source 1..22: mol_type = other RNA
tagcttatcagactgatgttga

SEQ ID NO: 92 - RNA, 54 nt, synthetic construct
  source 1..54: mol_type = other RNA
ggaccaccccaaaaacgaaggggactaaaacatcaacatcagtctgataagcta

SEQ ID NO: 93 - RNA, 40 nt, synthetic construct
  source 1..40: mol_type = other RNA
ggggcgcgatcaaaacaacgcgttgttttgatcgcgcccc

SEQ ID NO: 94 - RNA, 41 nt, synthetic construct
  source 1..41: mol_type = other RNA
ggggcgcgatcaaaacaacgtcgttgttttgatcgcgcccc

SEQ ID NO: 95 - RNA, 43 nt, synthetic construct
  source 1..43: mol_type = other RNA
ggggcgcgatcaaaacaacgtttcgttgttttgatcgcgcccc

SEQ ID NO: 96 - RNA, 45 nt, synthetic construct
  source 1..45: mol_type = other RNA
ggggcgcgatcaaaacaacgtttttcgttgttttgatcgcgcccc

SEQ ID NO: 97 - RNA, 40 nt, synthetic construct
  source 1..40: mol_type = other RNA
cgttgttttgatcgcgccccggggcgcgatcaaaacaacg

SEQ ID NO: 98 - DNA, 32 bp, synthetic construct
  source 1..32: mol_type = other DNA
  misc_feature 1..12: note = DNA segment
  misc_feature 13..32: note = RNA Segment
  misc_feature 1: note = 5'-modification; 5'-Texas Red
  misc_feature 32: note = 3'-modification; 3'-BHQ2
atcgcgccccccggggcgcgatcaaaacaacg

SEQ ID NO: 99 - DNA, 10 bp, synthetic construct
  source 1..10: mol_type = other DNA
cccccccccc

SEQ ID NO: 100 - DNA, 24 bp, synthetic construct
  source 1..24: mol_type = other DNA
  misc_feature 7: note = Internal modification; /iAzide-dT/
  misc_feature 1: note = 5'-modification; 5'-Biotin
  misc_feature 24: note = 3'-modification; 3'-Alkyne
  misc_feature 1..21: note = DNA Segment
  misc_feature 22..24: note = RNA Segment
tttttttctatgtgctatgtcttt

SEQ ID NO: 101 - DNA, 22 bp, synthetic construct
  source 1..22: mol_type = other DNA
  misc_feature 1: note = 5'-modification; 5'-Texas Red
  misc_feature 22: note = 3'-modification; 3'-BHQ1
ctctcattttttttttagagag

SEQ ID NO: 102 - DNA, 28 bp, synthetic construct
  source 1..28: mol_type = other DNA
tttagtgataagtggaatgccatgtgga

SEQ ID NO: 103 - DNA, 28 bp, synthetic construct
  source 1..28: mol_type = other DNA
tccacatggcattccacttatcactaaa